(19) United States
(12) Patent Application Publication
(10) Pub. No.: US 2022/0064598 A1
(43) Pub. Date: Mar. 3, 2022

(54) EX VIVO ACTIVATED T-LYMPHOCYTIC COMPOSITIONS AND METHODS OF USING THE SAME

(71) Applicant: CHILDREN'S NATIONAL MEDICAL CENTER, Washington, DC (US)

(72) Inventors: Catherine Mary Bollard, Bethesda, MD (US); Conrad Russell Y. Cruz, Bethesda, MD (US); Patrick Hanley, Washington, DC (US)

(21) Appl. No.: 17/421,337

(22) PCT Filed: Jan. 7, 2020
(86) PCT No.: PCT/US20/12639

§ 371 Date: Jul. 7, 2021

Related U.S. Application Data
(60) Provisional application No. 62/789,489, filed on Jan. 7, 2019.

(51) Int. Cl. C12N 5/0783; A61K 39/00; A61P 35/02

(52) U.S. Cl. CPC C12N 5/0638; C12N 5/0646; A61K 39/001189; A61K 39/001188; A61K 2039/5158; A61K 39/001153; A61K 39/00115; A61P 35/02; A61K 39/001186

(57) ABSTRACT

The disclosure provides T-cell compositions, therapies and processes of manufacture that are tailored to the specific antigenic expression of a subjects' tumor and allowing for changes in expression over time based on either pressure from antineoplastic therapy or natural heterogeneous selection. The disclosure also extends to methods of manufacturing such T-cell compositions and the generation of single antigen T-cell banks from healthy donors to provide an improved personalized T-cell therapy.

### CROSS-REFERENCE TO RELATED APPLICATIONS

This application claims priority to and benefit of U.S. Provisional Application No. 62/789,489, filed Jan. 7, 2019, entitled “Ex Vivo Activated T-Lymphocytic Compositions for Medical Treatment,” the content of which is incorporated herein by reference in its entirety.

### FIELD

The present disclosure provides improved T-cell compositions, therapies, and processes of manufacture that are tailored to the specific antigenic expression of a patient's tumor and allowing for changes in expression over time based on either pressure from antineoplastic therapy or immune editing or immune selection. Certain embodiments include adoptive T-cell compositions and their use and manufacture for the treatment of hematological malignancies or solid tumors. The present disclosure also extends to methods of manufacturing such T-cell compositions and the generation of single antigen T-cell banks from healthy donors to provide an improved personalized T-cell therapy.

### BACKGROUND

Adoptive immunotherapy is an approach used to bolster the ability of the immune system to fight diseases, such as tumor and viral infections. According to this approach, T-cells are collected from a patient or donor, stimulated in the presence of antigen presenting cells bearing tumor or viral-associated antigens, and then expanded ex vivo. These T-cells are given to the patient to help the immune system fight the disease.

Activated T-cell approaches have been reported since the early 2000s. Efforts began with the use of autologous blood that was activated by exposure ex vivo to viral antigens, typically in the context of treatment of patients who had undergone hematopoietic stem cell therapy and needed additional immune capacity, especially to fight viral diseases such as Epstein-Barr virus, cytomegalovirus, adenovirus and herpes simplex virus, as well as respiratory viral infections from RSV (respiratory syncytial virus), parainfluenza, and influenza. The efforts later expanded into allogeneic approaches for stem cell therapy patients followed by various approaches to attempt to use tumor associated antigen activated autologous or allogeneic blood sources. This approach has been shown to have some success clinically in the viral and tumor settings by multiple groups (Hague et al., Blood 110(4):1123-31 (2007), Leen et al., Blood 121(26):5113-23 (2013) and Naik et al, JACI 137(5):1498-1505 (2016). Blood from both naïve and non-naïve donors has been evaluated. A number of groups have also shown clinical success in the viral and tumor setting using a naïve T cell donor source with both single and multiple antigens (Park et al., Blood 108:1770-73 (2006); Hanley et al., Blood 114:1958-67 (2009); Jedema et al., Haematologica 96:1204-12 (2011)).

There are a number of ongoing human clinical trials evaluating a range of T-cell strategies. These include the RESOLVE trial, which is administering allogeneic T-cells to treat leukemia patients; the REST trial, which is evaluating autologous and allogeneic tumor associated antigen lymphocytes for the treatment of solid tumors; the TACTAM trial, which is administering autologous T-cells to treat multiple myeloma patients; the ADSPAM trial, which is administering allogeneic T-cells to treat AML and MDS patients; the MUSTAT trial, which is evaluating autologous and allogeneic T-cells primed with CMV, EBV, and/or adenovirus; the CHAPS trial, which is evaluating allogeneic viral antigen primed T-cells; the NATS trial, which is evaluating a multivalent 6-viral antigen approach for transplant patients; the HXTC and RESIST trials, which is evaluating autologous HIV activated T-cells; the ACTCAT2 trial, which is evaluating cord blood primed with viral antigens; and the CHEERS trial, which is evaluating cord blood activated with multiple viral antigens.

Recent strategies have been developed to generate activated T-cells targeting multiple potential antigens in a single T-cell product. In particular, approaches to generate multi-antigen specific T-cells have focused on priming and activating T-cells with multiple targeted antigen libraries, for example multiple libraries of 15mer peptides overlapping by 11 amino acids spanning the whole amino acid sequence (a “pepmix”) of several target antigens (see for example commercially available pepmix products from JPT Technologies or Miltenyi). The pepmixes include some peptide segments that are immunogenic and others that are not. See generally, US2011/0182870 (Baylor); Hanley et al., Blood, supra; US 2015/0044258 (CellMedica); US 2015/0017723 (Baylor); US2015/0010519 (Baylor); Weber et al., Clin. Cancer Res. 19(18):5079-91 (2013); WO2015/066057 (Baylor); US2015/0359876 (Children's National Medical Center); Ramos et al., J. Immunother. 36(1):66-76 (2013); Ngo et al., J. Immunother. 37(4):193-203 (2014); WO2016/154112 (CNMC); WO2017/075571 (CNMC); and Sung et al., J. Infect. Dis. 212(2):258-63 (2015), each of which is incorporated by reference in its entirety.

In these processes, the individual pepmixes of peptide segments of the selected antigen are generally mixed in equal amounts regardless of the molecular weight of the protein antigen to create the mastermix for T-cell priming, and single batches of T-cells are exposed to the multi-antigen pepmix. While this approach does provide the potential for a “universal” protocol to the generation of multi-TAA-specific T-cells, the mastermix of pepmix peptide segments, however, may not be a good match for the patient's specific tumor expression profile, which decreases the potential efficacy of the therapy. Further, since the peptides have different molecular weights, using the same weight amount of the pepmix for each antigenic protein in the cocktail results in the use of fewer segment duplicates in the pepmixes of the higher molecular weight proteins. Also, it is somewhat random how many active epitopes each protein has so that one pepmix might contain more active epitopes than another pepmix, regardless of molecular weight. Additional causes include the use of nonimmunogenic antigens, which can elicit tolerance or introduce potential avenues for autoimmunity if other unnecessary peptides are cross reactive. Consequently, the approach leads to large variability of primed and activated T-cells to each particular antigen within each generated T-cell product, which may not be reflective of the tumor antigen profile of any particular patient's tumor.

There has been some recognition of this in the art and attempts to compensate. See, e.g., US2015/0017723. Notwithstanding this attempt to compensate for the challenges in using mastermixes of peptide antigens to create a multi-antigen specific T-cell product, issues of lack of optimal targeting and efficacy remain. In fact, in some cases, T-cells primed with a pepmix to an antigen or a mastermix of antigens can be prepared that do not significantly lyse a patient's tumor cells ex vivo, indicating that it would not be effective in vivo (See Weber et al. 2013, supra).

Current non-engineered adoptive cell therapy production methods, e.g., activating T-cells ex vivo in a single batch using multiple tumor associated antigens (TAAs), can result in inconsistent levels of activation to each of the targeted TAAs, as well as a variable product with respect to other lymphocytic cell-populations. Lymphocytic cell compositions lacking a variety of multi-lymphocytic cell subsets, or which rely on an over-representation of certain lymphocytic subsets, are less effective in targeting tumors and patients receiving such compositions are less likely to exhibit epitope spreading.

U.S. Ser. No. 62/660,878 addressed the need to standardize T-cell therapeutic compositions prepared from naïve healthy donor PBMCs, which can naturally have a large range of percentages of lymphocytic cells, which can potentially result in differences in therapeutic potency. U.S. Ser. No. 62/660,878 describes a product that has a fixed ratio of lymphocytic subsets that include CD4⁺ T-cells, CD8⁺ T-cells, CD3⁺/CD56⁺ Natural Killer T-cells (CD3⁺ NKT), and TCR γδ T-cells (γδ T-cells).

U.S. Ser. No. 62/673,745 discloses lymphocytic compositions that include, in the same dosage form, a multiplicity of CD4⁺ and CD8⁺ T-cell subpopulations. Each T-cell subpopulation is specific for a single tumor-associated antigen (TAA). The T-cell subpopulations are chosen specifically based on the TAA expression profile of the patient's tumor, and thus is personalized therapy. These highly standardized T-cell compositions are referred to as “MUltiple Single Tumor ANtiGen” T-cell compositions or “MUSTANG” compositions.

While progress has been made in T-cell therapy, given its importance to tumor therapy, there is a strong need to improve the efficiency and outcomes of the therapy. As one example, there remains a need to improve adoptive immunotherapy for the treatment of disorders, including hematological malignancies and other tumors.

### SUMMARY

Provided herein is a unique approach to manufacture and use an improved composition of ex vivo activated mixed lymphocyte cell products for use in the treatment of cancer. It has been discovered that T-cell therapy to treat human tumors can be significantly improved by administering to a patient in need thereof an effective amount of a T-cell composition that includes in the same dosage form a multiplicity of activated T-cell subpopulations, wherein each activated T-cell subpopulation is specific for a single tumor-associated antigen (TAA), and wherein the T-cell composition is standardized to include a fixed ratio of multiple ex vivo activated lymphocytic T-cell subsets. The present disclosure further allows for, in some embodiments, T-cell subpopulations that comprise the T-cell composition for administration to be chosen specifically based on the TAA expression profile of a patient's tumor. By using separately activated T-cell subpopulations to form the T-cell composition for administration, the T-cell composition as a whole includes individual T-cell subpopulations targeting specific TAAs, resulting in a highly consistent and activated T-cell composition capable of targeting multiple TAAs, and eliminating the potential for administering a T-cell product that does not have activity against a particular targeted TAA. Furthermore, by selecting the T-cell subpopulations based on the patient's TAA expression profile, a highly targeted T-cell composition is administered having increased efficacy, increased level of consistency and characterization, and decreased potential for generating off-target effects from the use of T-cells which target antigens not expressed by the patient's tumor. In addition, by selecting specific fixed ratios of different lymphocytic cell subsets for inclusion in the T-cell composition to be administered, an immune response which is comprehensive and broad in biological and immune effector function is provided, enhancing the ability of the administered cells to mount an effective and robust immune response.

Unlike the random T-cell compositions derived by the use of pooled, multi-TAA pepmixes which result in considerable variability and, in some instances, no activity against one or more TAAs despite their inclusion in the pepmix, the present disclosure avoids the significant variability of these compositions. The T-cell composition, and its use and manufacture, differs from the prior art in that the T-cells are not, as a group, exposed to a mastermix of peptide fragments or pepmixes from multiple TAAs. Instead, T-cell subpopulations are each exposed to single TAA pepmixes or one or more peptides from a single TAA, including and perhaps substantially comprised of selected peptide epitope(s) of the TAA. The therapeutic dosage form of the T-cell composition includes more than one, for example two, three, four, or five T-cell subpopulations, wherein each T-cell subpopulation is specific for a single TAA; that is, the separate T-cell subpopulations that comprise the T-cell composition are each primed to a single tumor antigen, for example each T-cell subpopulation is capable of recognizing one TAA. In some embodiments, the particular T-cell subpopulations that make up the T-cell composition target TAAs that are representative of the TAA expression profile of a patient's tumor. In some embodiments, the percentage of each specific TAA-targeting T-cell subpopulation in the T-cell composition correlates with the tumor-associated antigen expression profile of the tumor in the patient receiving the treatment.

Furthermore, unlike the non-selected, non-fixed ratio of adoptive T-cell compositions, for example as exemplified in FIG. 1B of Weber et al. Generation of tumor antigen-specific T cell lines from pediatric patients with acute lymphoblastic leukemia—implications for immunotherapy, Clin Cancer Res. 2013 Sep 15; 19(18): 5079-5091, the present disclosure avoids the significant lymphocytic cell variability of these compositions allowing for a more efficacious treatment of tumors.

The T-cell subpopulations that comprise the T-cell composition each target a single TAA. The generation of each T-cell subpopulation can be accomplished through the ex vivo priming and activation of the T-cell subpopulation to one or more peptides from a single TAA. If more than one peptide from a single, targeted tumor antigen is used, the peptide segments can be generated by making overlapping peptide fragments of the tumor antigen, as provided for example, in commercially available pepmixes, or can be selected to be limited to, or enriched with, certain antigenic epitopes of the targeted TAA, for example, a single, or multiple specific epitopes of the TAA. In one embodiment, the T-cell subpopulation is primed with a single TAA peptide mix, wherein the peptide mix includes a pepmix that has been further enriched with one or more specific known or identified epitopes expressed by the patient's tumor. In one embodiment, the peptide segments are the same length. In some embodiments, the peptide segments are of varying lengths. In other embodiments, the peptide segments substantially only include known tumor antigenic epitopes. In one embodiment, the T-cell subpopulation is primed and activated with one or more epitopes expressed by the patient's tumor. In one embodiment, the tumor antigen is a neoantigen. In one embodiment, the neoantigen is a mutated form of an endogenous protein derived through a single point mutation, a deletion, an insertion, a frameshift mutation, a fusion, mis-spliced peptide, or intron translation.

Each of the T-cell subpopulations targeting a specific TAA can be combined in a single T-cell composition for administration. In particular, the activated T-cell subpopulations include CD4⁺ T-cells and CD8⁺ T-cells that have been primed and are capable of targeting a specific antigen for tumor killing and/or cross presentation, which can be combined into a single T-cell composition for administration which includes a multiplicity of T-cell subpopulations, with each T-cell subpopulation targeting a specific TAA. The cell composition further comprises activated γδ T-cells and/or activated CD3⁺ NKT cells capable of mediating anti-tumor responses. By providing an optimized, standardized, non-naturally occurring ratio of multiple activated immune effector cells with differing in vivo immune effector and biological functions, long lasting and durable responses to multiple tumor-types are possible, increasing the ability of the administered cell composition to induce tumor specific epitope spreading, and reducing tumor immune surveillance avoidance. The inclusion of activated CD3⁺ NKT-cells and/or γδ T-cells results in the additional release of cytokines that induce bystander T-cell activation and thus recruit other lymphocytes, including CD8+ T-cells, to aid in tumor cytolysis, including in epitope spreading. Furthermore, by producing fixed ratios of activated immune effector cells, consistent and reproducible cell compositions are provided, reducing the variability of administered product received by different patients.

In some aspects of the present disclosure, the T-cell composition provides a fixed ratio as further described herein of a population of different lymphocytic cell subsets comprising one or more subpopulations of CD4⁺ T-cells and CD8⁺ T-cells, and, in addition, CD3⁺ NKT-cells and/or γδ T-cells. Each subpopulation of CD4⁺ T-cells and CD8⁺ T-cells is primed and activated against a single specific target antigen. To the extent more than one CD4+ and CD8+ T-cell subpopulation is present in the T-cell composition, each CD4+/CD8+ subpopulations is primed and activated separately against discrete antigens. The CD3+ NKT-cells and/or γδ T-cells can be activated separately and recombined with the one or more subpopulations of CD4+/CD8+ T-cells to form the T-cell composition. Alternatively, the CD3+ NKT-cells and/or γδ T-cells can be activated in the same cell culture as the CD4+ and CD8+ T-cell subpopulations.

In alternative aspects of the present disclosure, the T-cell composition provides a fixed ratio as further described herein of a population of different lymphocytic cell subsets comprising one or more subpopulations of CD4⁺ T-cells, one or more subpopulations CD8⁺ T-cells, and, in addition, CD3⁺ NKT-cells and/or γδ T-cells. Each subpopulation of CD4⁺ T-cells and CD8⁺ T-cells are primed and activated against a single specific target antigen separately, and, to the extent more than one CD4+ and CD8+ T-cell subpopulation is included in the T-cell composition, no CD4+ T-cell subpopulation is primed and activated to the same antigen as another CD4+ T-cell subpopulation and no CD8+ T-cell subpopulation is primed and activated to the same antigen as another CD8+ T-cell subpopulation. The CD3+ NKT-cells and/or γδ T-cells can be activated separately and recombined with the subpopulations of CD4+ and CD8+ T-cells to form the T-cell composition. Alternatively, the CD3+ NKT-cells and/or γδ T-cells can be activated in the same cell culture as either the CD4+ or CD8+ T-cell subpopulations.

### DETAILED DESCRIPTION

The present disclosure combines the improvement of using a fixed ratio of lymphocytic cells with the MUSTANG approach. The MUSTANG approach is described in, for example, PCT Application Publication Nos. WO 2019/204831 and WO 2019/222760, both are incorpoareted herein by reference. The MUSTANG compositions can be enhanced to have non-naturally occurring advantageous fixed ratios of lymphocytic cell subsets.

By providing an optimized, standardized, non-naturally occurring ratio of multiple activated immune effector cells with differing in vivo immune effector and biological functions, long lasting and durable responses to multiple tumor-types may be possible, increasing the ability of the administered cell composition to induce tumor specific epitope spreading, and reducing tumor immune surveillance avoidance. The inclusion of activated CD3⁺ NKT-cells and/or γδ T-cells results in the additional release of cytokines that induce bystander T-cell activation and thus recruit other lymphocytes, including CD8+ T-cells, to aid in tumor cytolysis, including in epitope spreading.

### Definitions

Unless defined otherwise, all technical and scientific terms used herein have the same meaning as commonly understood by one of ordinary skill in the art to which the present disclosure pertains.

The term “a” and “an” refers to one or to more than one (i.e., to at least one) of the grammatical object of the article. By way of example, “an element” means one element or more than one element.

The term “allogeneic” as used herein refers to medical therapy in which the donor and recipient are different individuals of the same species.

The term “antigen” as used herein refers to molecules, such as polypeptides, peptides, or glyco- or lipo-peptides that are recognized by the immune system, such as by the cellular or humoral arms of the human immune system. The term “antigen” includes antigenic determinants, such as peptides with lengths of 6, 7, 8, 9, 10, 11, 12, 13, 14, 15, 16, 17, 18, 19, 20, 21, 22 or more amino acid residues that bind to MHC molecules, form parts of MHC Class I or II complexes, or that are recognized when complexed with such molecules.

The term “antigen presenting cell” or “APC” as used herein refers to a class of cells capable of presenting one or more antigens in the form of peptide-MHC complex recognizable by specific effector cells of the immune system, and thereby inducing an effective cellular immune response against the antigen or antigens being presented. Examples of professional APCs are dendritic cells and macrophages, though any cell expressing MHC Class I or II molecules can potentially present peptide antigen.

The term “autologous” as used herein refers to medical therapy in which the donor and recipient are the same person.

The term “cord blood” as used herein has its normal meaning in the art and refers to blood that remains in the placenta and umbilical cord after birth and contains hematopoietic stem cells. Cord blood may be fresh, cryopreserved, or obtained from a cord blood bank.

The term “cytokine” as used herein has its normal meaning in the art. Nonlimiting examples of cytokines include IL-2, IL-6, IL-7, IL-12, IL-15, and IL-27.

The term “cytotoxic T-cell” or “cytotoxic T lymphocyte” as used herein is a type of immune cell that bears a CD8⁺ antigen and that can kill certain cells, including foreign cells, tumor cells, and cells infected with a virus. Cytotoxic T-cells can be separated from other blood cells, grown ex vivo, and then given to a patient to kill tumor or viral cells. A cytotoxic T-cell is a type of white blood cell and a type of lymphocyte.

The term “dendritic cell” or “DC” as used herein describes a diverse population of morphologically similar cell types found in a variety of lymphoid and non-lymphoid tissues, see Steinman, Ann. Rev. Immunol. 9:271-296 (1991).

As used herein, “depleting” when referring to one or more particular cell type or cell population, refers to decreasing the number or percentage of the cell type or population, e.g., compared to the total number of cells in or volume of the composition, or relative to other cell types, such as by negative selection based on markers expressed by the population or cell, or by positive selection based on a marker not present on the cell population or cell to be depleted. The term does not require complete removal of the cell, cell type, or population from the composition.

The term “derivative” as used herein, when referring to peptides, means compounds having amino acid structural and functional analogs, for example, peptidomimetics having synthetic or non-natural amino acids (such as a norleucine) or amino acid analogues or non-natural side chains, so long as the derivative shares one or more functions or activities of polypeptides of the disclosure. The term “derivative” therefore include “mimetic” and “peptidomimetic” forms of the polypeptides disclosed herein. As used herein, a “non-natural side chain” is a modified or synthetic chain of atoms joined by covalent bond to the α-carbon atom, β-carbon atom, or γ-carbon atom which does not make up the backbone of the polypeptide chain of amino acids. The peptide analogs may comprise one or a combination of non-natural amino-acids chosen from: norvaline, tert-butylglycine, phenylglycine, He, 7-azatryptophan, 4-fluorophenylalanine, N-methyl-methionine, N-methyl-valine, N-methyl-alanine, sarcosine, N-methyl-tert-butylglycine, N-methyl-leucine, N-methyl-phenylglycine, N-methyl-isoleucine, N-methyl-tryptophan, N-methyl-7-azatryptophan, N-methyl-phenylalanine, N-methyl-4-fluorophenylalanine, N-methyl-threonine, N-methyl-tyrosine, N-methyl-valine, N-methyl-lysine, homocysteine, and Tyr; Xaa2 is absent, or an amino acid selected from the group consisting of Ala, D-Ala, N-methyl-alanine, Glu, N-methyl-glutamate, D-Glu, Gly, sarcosine, norleucine, Lys, D-Lys, Asn, D-Asn, D-Glu, Arg, D-Arg, Phe, D-Phe, N-methyl-phenylalanine, Gin, D-Gln, Asp, D-Asp, Ser, D-Ser, N-methyl-serine, Thr, D-Thr, N-methyl-threonine, D-Pro D-Leu, N-methyl-leucine, D-Ile, N-methyl-isoleucine, D-Val, N-methyl-valine, tert-butylglycine, D-tert-butylglycine, N-methyl-tert-butylglycine, Trp, D-Trp, N-methyl-tryptophan, D-Tyr, N-methyl-tyrosine, 1-aminocyclopropanecarboxylic acid, 1-aminocyclobutanecarboxylic acid, 1-aminocyclopentanecarboxylic acid, 1-aminocyclohexanecarboxylic acid, 4-aminotetrahydro-2H-pyran-4-carboxylic acid, aminoisobutyric acid, (5)-2-amino-3-(1H-tetrazol-5-yl)propanoic acid, Glu, Gly, N-methyl-glutamate, 2-amino pentanoic acid, 2-amino hexanoic acid, 2-amino heptanoic acid, 2-amino octanoic acid, 2-amino nonanoic acid, 2-amino decanoic acid, 2-amino undecanoic acid, 2-amino dodecanoic acid, octylglycine, tranexamic acid, aminovaleric acid, and 2-(2-aminoethoxy)acetic acid. The natural side chain, or R group, of an alanine is a methyl group. In some embodiments, the non-natural side chain of the composition is a methyl group in which one or more of the hydrogen atoms is replaced by a deuterium atom. Non-natural side chains are disclosed in the art in the following publications: WO/2013/172954, WO2013123267, WO/2014/071241, WO/2014/138429, WO/2013/050615, WO/2013/050616, WO/2012/166559, US Application No. 20150094457, Ma, Z., and Hartman, M. C. (2012). In Vitro Selection of Unnatural Cyclic Peptide Libraries via mRNA Display. In J. A. Douthwaite & R. H. Jackson (Eds.), Ribosome Display and Related Technologies: Methods and Protocols (pp. 367-390). Springer New York., all of which are incorporated by reference in their entireties.

The terms “mimetic,” “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics, as further described below. The term “analog” refers to any polypeptide comprising at least one α-amino acid and at least one non-native amino acid residue, wherein the polypeptide is structurally similar to a naturally occurring full-length protein and shares the biochemical or biological activity of the naturally occurring full-length protein upon which the analog is based.

The term “effector cell” as used herein describes a cell that can bind to or otherwise recognize an antigen and mediate an immune response. Tumor, virus, or other antigen-specific T-cells and NKT-cells are examples of effector cells.

The term “endogenous” as used herein refers to any material from or produced inside an organism, cell, tissue or system.

As used herein, “enriching” when referring to one or more particular cell type or cell population, refers to increasing the number or percentage of the cell type or population, e.g., compared to the total number of cells in or volume of the composition, or relative to other cell types, such as by positive selection based on markers expressed by the population or cell, or by negative selection based on a marker not present on the cell population or cell to be depleted. The term does not require complete removal of other cells, cell type, or populations from the composition and does not require that the cells so enriched be present at or even near 100% in the enriched composition.

The term “epitope” or “antigenic determinant” as used herein refers to the part of an antigen that is recognized by the immune system, specifically by antibodies, B-cells, or T-cells.

The term “exogenous” as used herein refers to any material introduced from or produced outside an organism, cell, tissue or system.

As used herein, a statement that a cell or population of cells is or has been “exposed to” a specific antigen means that during ex vivo manufacturing conditions, the specific antigen is included in the culturing conditions.

The terms “functional fragment” means any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain 99%, 98%, 97%, 96%, 95%, 94%, 93%, 92%, 91%, or 90% sequence identity to the wild-type human sequence upon which the sequence is derived. In some embodiments, the functional fragment may retain 85%, 80%, 75%, 70%, 65%, or 60% sequence identity to the wild-type sequence upon which the sequence is derived.

The term “fragment” is meant a portion of a polypeptide or nucleic acid molecule. This portion contains, preferably, at least about 5%, 10%, 20%, 30%, 40%, 50%, 60%, 70%, 80%, or about 90% of the entire length of the reference nucleic acid molecule or polypeptide. A fragment may contain about 5, 10, 20, 30, 40, 50, 60, 70, 80, 90, or 100, 200, 300, 400, 500, 600, 700, 800, 900, 1000 or more nucleotides or amino acids.

The term “HLA” as used herein refers to human leukocyte antigen. There are 7,196 HLA alleles. These are divided into 6 HLA class I and 6 HLA class II alleles for each individual (on two chromosomes). The HLA system or complex is a gene complex encoding the major histocompatibility complex (MHC) proteins in humans. HLAs corresponding to MHC Class I (A, B, or C) present peptides from within the cell and activate CD8⁺ (i.e., cytotoxic) T-cells. HLAs corresponding to MHC Class II (DP, DM, DOA, DOB, DQ and DR) stimulate the multiplication of CD4⁺ T-cells, which stimulate antibody-producing B-cells.

The term “isolated” as used herein means separated from components in which a material is ordinarily associated with, for example, an isolated cord blood mononuclear cell can be separated from red blood cells, plasma, and other components of cord blood.

The term “MUSTANG composition” refers to as a “MUltiple Single Tumor ANtiGen” T-cell composition” composition. The MUSTANG is comprised of two or more T-cell subpopulations, wherein each T-cell subpopulation targets a single tumor-associated antigen. For purposes herein, when referring to combining T-cell subpopulations to comprise the MUSTANG composition, combining is intended to include the situation wherein the T-cells are physically combined into a single dosage form, that is, a single composition. In alternative embodiments, the T-cells subpopulations are kept physically separated but administrated concomitantly and collectively comprise the MUSTANG composition.

A “naïve” T-cell or other immune effector cell as used herein is one that has not been exposed to or primed by an antigen or to an antigen-presenting cell presenting a peptide antigen capable of activating that cell.

As used herein, a statement that a cell or population of cells is “negative” for a particular marker refers to the absence of substantial detectable presence on or in the cell of a particular marker, typically a surface marker, for example a cluster of determination (CD) marker. When referring to a surface marker, the term refers to the absence of surface expression, for example, as detected by flow cytometry, for example, by staining with an antibody that specifically binds to the marker and detecting said antibody, wherein the staining is not detected by flow cytometry at a level substantially above the staining detected carrying out the same procedure with an isotype-matched control or fluorescence minus one (FMO) gating control under otherwise identical conditions, and/or at a level substantially lower than that for cell known to be positive for the marker, and/or at a level substantially similar as compared to that for a cell known to be negative for the marker.

A “peptide library” or “overlapping peptide library” as used herein within the meaning of the application is a complex mixture of peptides which in the aggregate covers the partial or complete sequence of a protein antigen, especially those of opportunistic viruses. Successive peptides within the mixture overlap each other, for example, a peptide library may be constituted of peptides 15 amino acids in length which overlapping adjacent peptides in the library by 11 amino acid residues and which span the entire length of a protein antigen. Peptide libraries are commercially available and may be custom-made for particular antigens. Methods for contacting, pulsing or loading antigen-presenting cells are well known and incorporated by reference to Ngo et al. 2014, supra. Peptide libraries may be obtained from JPT and are incorporated by reference to the web site at https://www pt. com/products/peptrack/peptide-libraries.

A “peripheral blood mononuclear cell” or “PBMC” as used herein is any peripheral blood cell having a round nucleus. These cells consist of lymphocytes (T-cells, B-cells, NK cells) and monocytes. In humans, lymphocytes make up the majority of the PBMC population, followed by monocytes, and only a small percentage of dendritic cells.

As used herein, a statement that a cell or population of cells is “positive” for or “expresses” a particular marker refers to the detectable presence on or in the cell of a particular marker, typically a surface marker, for example a cluster of determination (CD) marker. When referring to a surface marker, the term refers to the presence of surface expression, for example, as detected by flow cytometry, for example, by staining with an antibody that specifically binds to the marker and detecting said antibody, wherein the staining is detectable by flow cytometry at a level substantially above the staining detected carrying out the same procedure with an isotype-matched control or fluorescence minus one (FMO) gating control under otherwise identical conditions and/or at a level substantially similar to that for cell known to be positive for the marker, and/or at a level substantially higher than that for a cell known to be negative for the marker.

The term “precursor cell” as used herein refers to a cell which can differentiate or otherwise be transformed into a particular kind of cell. For example, a “T-cell precursor cell” can differentiate into a T-cell and a “dendritic precursor cell” can differentiate into a dendritic cell.

A “subject” or “host” or “patient” as used herein is a vertebrate, preferably a mammal, more preferably a human. Mammals include, but are not limited to humans, simians, equines, bovines, porcines, canines, felines, murines, other farm animals, sport animals, or pets. Humans include those in need of virus- or other antigen-specific T-cells, such as those with lymphocytopenia, those who have undergone immune system ablation, those undergoing transplantation and/or immunosuppressive regimens, those having naïve or developing immune systems, such as neonates, or those undergoing cord blood or stem cell transplantation. In a typical embodiment, the term “patient” as used herein refers to a human.

A “T-cell population” or “T-cell subpopulation” is intended to include thymocytes, immature T lymphocytes, mature T lymphocytes, resting T lymphocytes and activated T-lymphocytes. The T-cell population or subpopulation can include T-cells, including CD4⁺ T-cells, CD8⁺ T cells, γδ T-cells, Natural Killer T-cells, or any other subset of T-cells.

The terms “treatment” or “treating” as used herein is an approach for obtaining beneficial or desired results including clinical results. For purposes herein, beneficial or desired clinical results include, but are not limited to, one or more of the following: decreasing one or more symptoms resulting from the disease, diminishing the extent of the disease, stabilizing the disease (e.g., preventing or delaying the worsening of the disease), preventing or delaying the spread (e.g., metastasis) of the disease, preventing or delaying the occurrence or recurrence of the disease, delay or slowing the progression of the disease, ameliorating the disease state, providing a remission (whether partial or total) of the disease, decreasing the dose of one or more other medications required to treat the disease, delaying the progression of the disease, increasing the quality of life, and/or prolonging survival.

As used herein, a “therapeutically effective amount” of a compound or composition or combination refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered.

The term “tumor-associated antigen expression profile” or “tumor antigen expression profile” as used herein, refers to a profile of expression levels of tumor-associated antigens within a malignancy or tumor. Tumor-associated antigen expression may be assessed by any suitable method known in the art including, without limitation, quantitative real time polymerase chain reaction (qPCR), cell staining, or other suitable techniques. Non-limiting exemplary methods for determining a tumor-associated antigen expression profile can be found in Ding et al., Cancer Bio. Med. 9:73-76 (2012); Qin etal., Leuk. Res. 33(3):384-90 (2009); Weber etal., Leukemia 23:1634-42 (2009); Liu et al., J. Immunol. 176:3374-82 (2006); Schuster et al., Int. J. Cancer 108:219-27 (2004).

The term “tumor-associated antigen” or “TAA” as used herein is an antigen that is highly correlated with certain tumor cells. They are not usually found, or are found to a lesser extent, on normal cells.

### Cell Populations

The present dislcosure provides isolated lymphocytic cell compositions for the treatment of cancer, including solid tumors and hematological malignancies, comprising a fixed ratio of multiple ex vivo primed and/or activated lymphocytic cell subsets directed to specific tumor associated antigens (TAAs), viral associated tumor antigens (VATA), glycolipids, or a combination thereof, wherein one or more of the primed and/or activated lymphocytic cell subsets comprise a fixed ratio of two or more separately primed and expanded cell subpopulations, each cell subpopulation having (i) specificity for a single tumor associated antigen and (ii) a different single tumor associated antigen specificity from all other cell subpopulations in the composition. The isolated cell compositions provided herein include fixed ratios of different lymphocytic cell subsets, wherein the different lymphocytic cell subsets within the cell composition are selected from a combination of CD4⁺ T-cells, CD8⁺ T-cells, CD3⁺/CD56⁺ Natural Killer T-cells (CD3⁺ NKT), and TCR γδ T-cells.

In some embodiments, the composition of the present disclosure comprises a T-cell population that is generated using a WT1 antigen library comprising a pool of peptides (for example 15 mers) containing amino acid overlap (for example 11 amino acids of overlap) between each sequence formed by scanning the WT1 antigen comprising the amino acid sequence of SEQ ID NO: 1 or functional fragments thereof that comprise at least about 70%, 75%, 80%, 85%, 90%, 95%, 96%, 97%, 98%, or 99% sequence identity to SEQ ID NO: 1. In some embodiments therefore, the T-cells in the composition comprise a TCR that binds specifically to one or a plurality of WT1-specific peptides.

In some embodiments, the composition of the present disclosure comprises a T-cell population that is generated using a PRAME antigen library comprising a pool of peptides (for example 15 mers) containing amino acid overlap (for example 11 amino acids of overlap) between each sequence formed by scanning the PRAMS antigen comprising the amino acid sequence of SEQ ID NO: 2 or functional fragments thereof that comprise at least about 70%, 75%, 80%, 85%, 90%, 95%, 96%, 97%, 98%, or 99% sequence identity to SEQ ID NO: 2. In some embodiments therefore, the T-cells in the composition comprise a TCR that binds specifically to one or a plurality of PRAME-specific peptides.

In some embodiments, the composition of the present disclosure comprises a T-cell population that is generated using a survivin antigen library comprising a pool of peptides (for example 15 mers) containing amino acid overlap (for example 11 amino acids of overlap) between each sequence formed by scanning the survivin antigen comprising the amino acid sequence of SEQ ID NO: 3 or functional fragments thereof that comprise at least about 70%, 75%, 80%, 85%, 90%, 95%, 96%, 97%, 98%, or 99% sequence identity to SEQ ID NO: 3. In some embodiments therefore, the T-cells in the composition comprise a TCR that binds specifically to one or a plurality of survivin-specific peptides.

In some embodiments, the composition of the present disclosure comprises a T-cell population that is generated using a MAGE-A3 antigen library comprising a pool of peptides (for example 15 mers) containing amino acid overlap (for example 11 amino acids of overlap) between each sequence formed by scanning the MAGE-A3 antigen comprising the amino acid sequence of SEQ ID NO: 4 or functional fragments thereof that comprise at least about 70%, 75%, 80%, 85%, 90%, 95%, 96%, 97%, 98%, or 99% sequence identity to SEQ ID NO: 4. In some embodiments therefore, the T-cells in the composition comprise a TCR that binds specifically to one or a plurality of MAGE-A3-specific peptides.

In some embodiments, the composition of the present disclosure comprises a T-cell population that is generated using a NY-ESO-1 antigen library comprising a pool of peptides (for example 15 mers) containing amino acid overlap (for example 11 amino acids of overlap) between each sequence formed by scanning the NY-ESO-1 antigen comprising the amino acid sequence of SEQ ID NO: 5 or functional fragments thereof that comprise at least about 70%, 75%, 80%, 85%, 90%, 95%, 96%, 97%, 98%, or 99% sequence identity to SEQ ID NO: 5. In some embodiments therefore, the T-cells in the composition comprise a TCR that binds specifically to one or a plurality of NY-ESO-1-specific peptides.

As will be understood by one skilled in the art, the TCR comprised in the T-cells of the present disclosure is a disulfide-linked membrane-anchored heterodimeric protein normally consisting of the highly variable alpha (α) and beta (β) chains expressed as part of a complex with the invariant CD3 chain molecules. T-cells expressing this type of receptor are referred to as α:β (or αβ) T-cells, though a minority of T-cells express an alternate receptor, formed by variable gamma (γ) and delta (δ) chains, referred as γδ T-cells. Each chain is composed of two extracellular domains: a variable (V) region and a constant (C) region, both of Immunoglobulin superfamily (IgSF) domain forming antiparallel β-sheets. The constant region is proximal to the cell membrane, followed by a transmembrane region and a short cytoplasmic tail, while the variable region binds to the peptide/MHC complex. The variable domain of both the TCR α-chain and β-chain each have three hypervariable or complementarity determining regions (CDRs). There is also an additional area of hypervariability on the β-chain (HV4) that does not normally contact antigen and, therefore, is not considered a CDR. The constant domain of the TCR consists of short connecting sequences in which a cysteine residue forms disulfide bonds, which form a link between the two chains.

The constant region of the TCR α-chain may comprise the following sequence:

(SEQ ID NO: 6)
IQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDVKLVE
KSFETDTNLNFQNLSVIGFRILLLKVAGFNLLMTLRLWSS.

Thus, in some embodiments, the T-cells of the present disclosure may comprise a constant region in the α-chain comprising at least about 60%, 65%, 70%, 75%, 80%, 85%, 90%, 95%, or 99% sequence identity to SEQ ID NO: 6. In some embodiments, the T-cells of the present disclosure may comprise a constant region in the α-chain comprising the amino acid sequence of SEQ ID NO: 6.

The constant region of the TCR β-chain may comprise the following sequence:

(SEQ ID NO: 7)
DLNKVFPPEVAVFEPSEAEISHTQKATLVCLATGFFPDHVELSWWVNGKE
VHSGVSTDPQPLKEQPALNDSRYCLSSRLRVSATFWQNPRNHFRCQVQFY
GLSENDEWTQDRAKPVTQIVSAEAWGRADCGFTSVSYQQGVLSATILYEI
LLGKATLYAVLVSALVLMAMVKRKDF.

Thus, in some embodiments, the T-cells of the present disclosure may comprise a constant region in the β-chain comprising at least about 60%, 65%, 70%, 75%, 80%, 85%, 90%, 95%, or 99% sequence identity to SEQ ID NO: 7. In some embodiments, the T-cells of the present disclosure may comprise a constant region in the α-chain comprising the amino acid sequence of SEQ ID NO: 7.

In some embodiments, the T-cells of the present disclosure may comprise a constant region in the α-chain comprising at least about 60%, 65%, 70%, 75%, 80%, 85%, 90%, 95%, or 99% sequence identity to SEQ ID NO: 6 and a constant region in the β-chain comprising at least about 60%, 65%, 70%, 75%, 80%, 85%, 90%, 95%, or 99% sequence identity to SEQ ID NO: 7. In some embodiments, the T-cells of the present disclosure may comprise a constant region in the α-chain comprising the amino acid sequence of SEQ ID NO: 6 and a constant region in the β-chain comprising the amino acid sequence of SEQ ID NO: 7.

In some embodiments, the TCR comprised in the T-cells of the present disclosure binds specifically to the antigen used for priming the T-cells with a K_{D} of about 1 μM or less. In some embodiments, the TCR comprised in the T-cells of the present disclosure binds specifically to the antigen used for priming the T-cells with a K_{D} of about ≤1 μM, ≤100 nM, ≤10 nM, ≤1 nM or ≤100 pM. In some embodiments, the TCR comprised in the T-cells of the present disclosure binds specifically to the antigen used for priming the T-cells with a K_{D} of from about 1 nM to about 1 μM. In some embodiments, the TCR comprised in the T-cells of the present disclosure binds specifically to the antigen used for priming the T-cells with a K_{D} of from about 1 nM to about 100 nM, from about 100 nM to about 200 nM, from about 200 nM to about 300 nM, from about 300 nM to about 400 nM, from about 400 nM to about 500 nM, from about 500 nM to about 600 nM, from about 600 nM to about 700 nM, from about 700 nM to about 800 nM, from about 800 nM to about 900 nM, or from about 900 nM to about 1 μM. The K_{D} measurement can be made by any of the known methods. In some embodiments therefore, the T-cells of the present disclosure may be prepared by a method with a step of priming the primary cells for a time period and at a concentration of antigen sufficient to result in any of the aforementioned binding affinities. The resultant T-cells may be further clonally expanded.

CD4⁺ T-Cells

The presently disclosed cell compositions can include CD4⁺ T-cells in ratios described herein. The CD4⁺ T-cells are primed against one or more specific targets, for example one or more TAAs, VATAs, or a combination thereof.

CD4⁺ T-cells are the primary orchestrators of the adaptive immune response, mediating a variety of cellular and humoral responses against pathogens and cancer. Although CD4⁺ T-cells are thought to lack the capacity to directly kill or engulf pathogens, they are powerful activators of effector cells such as macrophages, cytotoxic T cells, and B cells. CD4⁺ T-cells generally do not express or are negative for CD8, CD25, CD44, CD117, CD127, or TCR γ/δ.

CD4⁺ T-cells are crucial in achieving a regulated effective immune response to pathogens and tumors. Naïve CD4⁺ T-cells are activated after interaction with antigen-MHC complex and differentiate into specific subtypes depending mainly on the cytokine milieu of the microenvironment. Besides the classical T-helper 1 (Th1) and T-helper 2 (Th2), other CD4⁺ T-cell subsets have been identified, including T-helper 17 (Th17), regulatory T cell (Treg), follicular helper T-cell (Tfh), and T-helper 9 (Th9), each with a characteristic cytokine profile. For a particular phenotype to be differentiated, a set of cytokine signaling pathways coupled with activation of lineage-specific transcription factors and epigenetic modifications at appropriate genes are required. The effector functions of these cells are mediated by the cytokines secreted by the differentiated cells.

The CD4⁺ T-cells included in the fixed ratios described herein are preferably of the T-helper 1 (Th1)-type. Th1 cells are involved with the elimination of intracellular pathogens and are associated with organ-specific autoimmunity (del Prete, Allergy 47(5):450-55 (1992)). They mainly secrete IFN-γ, lymphotoxin α (Lfα), and IL-2. IFN-γ is essential for the activation of mononuclear phagocytes, including macrophages, microglial cells, thereby resulting in enhanced phagocytic activity (Murray et al., J. Immunol. 134(3)1982-88 (1985)). IFNγ is believed to exert its effect through the activation of IFNy-responsive genes, which account for more than 200 (Boehm et al., Ann. Rev. Immunol. 15:749-95, (1997)). IL-2 promotes proliferation of CD8⁺ T cells with acquisition of cytolytic phenotype (Kim et al., Cytokine Growth Factor Rev. 17(5):349-66 (2006); Gattinoni et al., J. Clin. Invest. 115(6):1616-26 (2005)). Besides its role as T cell growth factor, IL-2 also promotes the development of CD8⁺ memory cells after antigen priming, and thus participating in ensuring a robust secondary immune response (Williams et al., Nature 441(7095):890-93 (2006)). Cell markers typically associated with CD4+ Th1-cells include CD3, CD4, CD119 (IFN-γ Ra), CD183 (CXCR3), CD195 (CCRS), CD218a (IL-18Rα), LTβR, and CD366 (Tim-3). Regulatory T cells (Treg) are a subpopulation of CD4⁺ T-cells that maintain homeostasis and tolerance within the immune system. FOXP3⁺CD25⁺CD4⁺ regulatory T (Treg) cells, which suppress aberrant immune response against self-antigens, also suppress anti-tumor immune responses. Infiltration of a large number of Treg cells into tumor tissues is often associated with poor prognosis. In one embodiment, the CD4⁺ T-cells are depleted of Treg cells. Various cell surface molecules, including chemokine receptors such as CCR4, that are specifically expressed by effector Treg cells can be targeted for the negative selection of Tregs as provided herein. Cell markers typically associated with CD4⁺ Treg-cells include CD3, CD4, CD25 (IL-2Ra), CD39, CD73, CD103, CD152 (CTLA-4), GARP, GITR, and LAP (TGF-β).

CD8⁺ T-Cells

The presently disclosed cell compositions include CD8⁺ T-cells in ratios described herein. The CD8⁺ T-cells are primed against one or more specific targets, for example one or more TAAs, VATAs, or a combination thereof.

CD8⁺ T-cells are a subset of T-cells that express an αβ T-cell receptor (TCR) and are responsible for the direct killing of infected, damaged, and dysfunctional cells, including tumor cells. CD8⁺ T cells, like CD4⁺ Helper T cells, are generated in the thymus. However, rather than the CD4 molecule, cytotoxic T cells express a dimeric co-receptor—CD8—usually composed of one CD8α and one CD8β chain. CD8⁺ T-cells recognize peptides presented by MHC Class I molecules, found on all nucleated cells. The CD8 heterodimer binds to a conserved portion (the α3 region) of MHC Class I during T cell/antigen presenting cell interactions.

CD8⁺ T cells (often called cytotoxic T lymphocytes, or CTLs) are very important for immune defense against intracellular pathogens, including viruses and bacteria, and for tumor surveillance. When a CD8⁺ T cell recognizes its antigen and becomes activated, it has three major mechanisms to kill infected or malignant cells. The first is secretion of cytokines, primarily TNF-α and IFN-γ, which have anti-tumor and anti-viral microbial effects.

The second major function is the production and release of cytotoxic granules. These granules, also found in NK cells, contain two families of proteins—perforin, and granzymes. Perforin forms a pore in the membrane of the target cell, similar to the membrane attack complex of complement. This pore allows the granzymes also contained in the cytotoxic granules to enter the infected or malignant cell. Granzymes are serine proteases which cleave the proteins inside the cell, shutting down the production of viral proteins and ultimately resulting in apoptosis of the target cell.

The cytotoxic granules are released only in the direction of the target cell, aligned along the immune synapse, to avoid non-specific bystander damage to healthy surrounding tissue. CD8⁺ T-cells are able to release their granules, kill an infected cell, then move to a new target and kill again, often referred to as serial killing.

The third major function of CD8⁺ T-cell destruction of infected cells is via Fas/FasL interactions. Activated CD8⁺ T-cells express FasL on the cell surface, which binds to its receptor, Fas, on the surface of the target cell. This binding causes the Fas molecules on the surface of the target cell to trimerize, which pulls together signaling molecules. These signaling molecules result in the activation of the caspase cascade, which also results in apoptosis of the target cell. Because CD8⁺ T-cells can express both molecules, Fas/FasL interactions are a mechanism by which CD8⁺ T-cells can kill each other, called fratricide, to eliminate immune effector cells during the contraction phase at the end of an immune response.

Cell markers typically expressed by CD8⁺ T-cells (or which CD8⁺ T-cells are positive for) include CD3⁺, CD8⁺, and TCR α/β⁺, and which CD8⁺ T-cells are negative for are CD25, CD44, CD117, CD127, and TCR γ/δ.

CD3⁺/CD56⁺ Natural Killer T-Cells (NKT) In certain aspects, the cell compositions described herein include CD3⁺ NKT-cells. The CD3⁺ NKT-cells are activated. In certain embodiments, the CD3⁺ NKT-cells can be primed against one or more specific glycolipid antigens, for example one or more gangliosides. In certain embodiments, the CD3⁺ NKT-cells are exposed to one or more specific antigens. In certain embodiments, the CD3⁺ NKT-cells are exposed to one or more specific antigens and cultured in the same culture as the αβ T-cells, CD4⁺ T-cells, CD8⁺ T-cells, and/or γδ T-cells, or combination thereof, wherein they are activated during culturing. In one embodiment, the CD3⁺ NKT-cells are activated separately from other cells of the composition. In one embodiment, the CD3⁺ NKT-cells are separately activated.

Natural killer T (NKT) cells are a specialized population of T cells that express a semi-invariant T cell receptor (TCR αβ) and surface antigens typically associated with natural killer cells. In humans, the TCRs of NKT cells almost always contain Vα24/Jα18 paired with a TCRβ chain containing Vβ11. The TCR on NKT cells is unique in that it recognizes glycolipid antigens presented by the MHC I-like molecule CD1d. MostNKT cells, known as type INKT cells, express an invariant TCR α-chain and one of a small number of TCR β-chains. The TCRs present on type I NKT cells is capable of recognizing the antigen a-galactosylceramide (a-GalCer). Within this group, distinguishable subpopulations have been identified, including CD4⁺CD8⁻NKT-cells, CD4 CD8⁻ NKT-cells, and CD4⁻CD8⁺ T-cells.

NKT-cells also include a smaller population of NKT cells, known as type II NKT-cells (or noninvariant NKT-cells), which express a wider range of TCR α-chains, but do not recognize the α-GalCer antigen.

NKT-cells contribute to antibacterial and antiviral immune responses and promote tumor-related immunosurveillance or immunosuppression. Like natural killer cells, NKT-cells can also induce perforin-, Fas-, and TNF-related cytotoxicity. Activated NKT-cells are capable of producing IFN-γ and IL-4.

Cell markers typically expressed by NKT-cells (or which NKT-cells are positive for) include CD16, CD94, NKG2D, CD3, and CD56. NKT-cells generally do not express or are negative for CD14 and CD33.

αβ T-cells

The presently disclosed cell compositions can include αβ T-cells in ratios described herein. The αβ T-cells, which include CD4⁺ and CD8⁺ T-cells, are primed against one or more specific targets, for example one or more TAAs, VATAs, or a combination thereof.

There are two types of T-cell receptor αβ and γδ. The dominant type is αβ which is associated with the two main T-cell populations: CD4⁺ helper T cells and CD8⁺ cytotoxic T cells. The αβ TCR can only recognize short linear peptides in association with molecules from the major histocompatability complex (MHC). Cells with the αβ TCR generally express CD4 or CD8 subset markers and mostly fall into helper or cytotoxic/effector subsets. Cell markers typically associated with αβ T-cells or which αβ T-cells are positive for include TCR αβ, CD2, CD3, CD7, CD16, CXCR4, NKG2D, and are TCR αβ-.

γδ T-Cells

In certain aspects, the cell compositions described herein include γδ T-cells. The γδ T-cells are activated. In certain embodiments, the γδ T-cells are exposed to one or more specific antigens.

In certain embodiments, the γδ T-cells are exposed to one or more specific antigens and cultured in the same culture as the CD3⁺ NKT-cells, CD4⁺ T-cells, and/or CD8⁺ T-cells, or combination thereof, wherein they are activated during culturing. In one embodiment, the γδ T-cells are activated separately from other cells of the composition. In one embodiment, the γδ T-cells cells are separately activated.

γδ T-cells are a subset of T-cells defined by the genetic composition of their T Cell Receptor (TCR). γδ T-cells account for up to 10% of circulating lymphocytes and operate at the interface between innate and adaptive immunity. γδ T-cells recognize genomic, metabolic, and signaling perturbations associated with the transformed state. γδ T-cells release perforin and granzymes, express both FAS and TRAIL, engage in Fc receptor-dependent effector functions and produce a range of immunomodulatory cytokines, including tumor necrosis factor (TNF) and interferon (IFN)-γ. γδ T-cells act as efficient antigen-presenting cells, enabling the perpetuation of immune attack through adaptive mechanisms. Finally, since these cells are not HLA-restricted, they do not elicit graft versus host disease. Vγ9Vδ2 cells have endogenous cytotoxicity against various tumors; following activation, they can acquire phenotypic characteristics of professional antigen-presenting cells (γδ-APCs), including capacity for cross presentation of tumor-associated antigens. γδ T-cells of the Vδ1 subtype have acnaturally more naïve memory (Tnaïve) phenotype, a reduced susceptibility to activation-induced cell death, and their natural residency in tissues.

Unlike αβ T-cells, most γδ T cells lack CD4 and CD8 and share a number of markers associated with natural killer cells or antigen-presenting cells such as Fc gamma RIII/CD16 and Toll-like receptors. Cell markers typically associated with γδ T-cells or which γδ T-cells are positive for include TCR γδ, CD2, CD3, CD7, CD16, CXCR4, and NKG2D. γδ T-cells do not express or are negative for TCR α/β.

### Fixed Ratios of Different Lymphocytic Cell Subsets

The isolated cell compositions provided herein include fixed ratios of different lymphocytic cell subsets, wherein the different lymphocytic cell subsets within the cell composition are selected from a combination of CD4⁺ T-cells, CD8⁺ T-cells, CD3⁺/CD56⁺ Natural Killer T-cells (CD3⁺ NKT), and TCR γδ T-cells (each a “T-cell component”). By providing a balanced ratio of multiple primed and/or activated immune effector cells with differing biological functions, long lasting and durable responses to multiple tumor-types are possible, increasing the ability of the administered cell composition to induce tumor specific epitope spreading, and reducing tumor immune surveillance avoidance. Furthermore, by producing fixed ratios of primed and/or activated immune effector cells, consistent and reproducible homogeneous compositions are provided, reducing the variability of administered product received by different patients.

The ratios and percentages of cells as described herein are with reference to cell numbers. For example, a ratio of about 1:1:1 (+/−5%) provides for about an equal number of cells (+/−5%) from each identified cell subset contained in the cell composition.

CD4⁺ T-Cell, CD8⁺ T-Cell, and CD3⁺ NKT-Cell Composition

In one aspect, the composition provides a fixed ratio of a population of different lymphocytic cell subsets comprising CD4⁺ T-cells, CD8⁺ T-cells, and CD3⁺ NKT-cells exposed ex vivo to one or more specific target antigens. In one embodiment, the CD4⁺ T-cells and CD8⁺ T-cells of the cell composition are primed against the one or more specific target antigens, while the CD3⁺ NKT-cells are activated. In certain embodiments, the cells have been further exposed to one or more glycolipids, for example one or more gangliosides. In one embodiment, the CD3⁺ NKT-cells are primed against against one or more glycolipids, for example, a ganglioside. In one embodiment, a-GalCer and other “galactosylsphingamide a-GalCer analaogues” can be used to stimulate NKT.

In one embodiment, the composition comprises about a 1:1:1 ratio (+/−5%) of CD4⁺ T-cells:CD8⁺ T-cells:CD3⁺ NKT-cells. In one embodiment, the composition comprises between about 15% and about 25% CD4⁺ T-cells, between about 45% and about 55% CD8⁺ T-cells, and between about 25% and about 35% CD3⁺ NKT-cells. For example, in one embodiment, the composition comprises about 15%, 16%, 17%, 18%, 19%, 20%, 21%, 22%, 23%, 24%, or 25% CD4⁺ T-cells; about 25%, 26%, 27%, 28%, 29%, 30%, 31%, 32%, 33%, 34%, or 35% CD8⁺ T-cells; and about 45%, 46%, 47%, 48%, 49%, 50%, 51%, 52%, 53%, 54%, or 55% CD3⁺ NKT-cells.

In one embodiment, the composition comprises about 20% CD4⁺ T-cells, about 50% CD8⁺ T-cells, and about 30% CD3⁺ NKT-cells, resulting in a cell composition comprising about a 0.2:0.5:0.3 ratio of CD4⁺ T-cells:CD8⁺ T-cells:CD3⁺ NKT-cells.

In an alternative embodiment, the cell composition comprises at least about 30% CD8⁺ T-cells, at least about 15% CD4⁺ T-cells, and at least about 10% CD3⁺ NKT-cells. In one embodiment, the cell composition comprises between about 30% and about 40% CD8⁺ T-cells, about 15% to about 25% CD4⁺ T-cells, and between about 10% and about 20% CD3⁺ NKT-cells. In one embodiment, the cell composition comprises between about 35% CD8⁺ T-cells, about 20% CD4⁺ T-cells, and 15% CD3⁺ NKT-cells.

In one embodiment, the CD4⁺ T-cells of the composition are primarily CD4⁺ Th1-cells. For example, the CD4⁺ Th1-cells of the composition make up about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 84%, 85%, 86%, 87%, 88%, 89%, 90% or more of the total of CD4⁺ T-cells in the composition.

In one embodiment, the composition is comprised of little or minimal CD4⁺ Treg-cells. For example, CD4⁺ Treg-cells make up less than about 5%, 4%, 3%, 2%, or 1% of the population of CD4⁺ T-cells.

The CD3⁺ NKT-cells of the composition can be CD8⁺, CD4⁺, or CD8⁻/CD4⁻, or a mixture thereof. In one embodiment, the CD3⁺ NKT-cells are primarily type I NKT-cells. For example, in one embodiment, type I NKT-cells comprise about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 84%, 85%, 86%, 87%, 88%, 89%, 90% or more of the total CD3⁺ NKT-cells in the composition.

In one embodiment, the cell composition consists of only CD4⁺ T-cells, CD8⁺ T-cells, and CD3⁺ NKT-cells.

In one embodiment, the cell composition comprises primarily CD4⁺ T-cells, CD8⁺ T-cells, and CD3⁺ NKT-cells.

In one embodiment, the cells have been exposed to and/or primed against one or more targeted antigens selected from a TAA, a VATA, glycolipid, or a combination thereof. In one embodiment, the CD8⁺ and CD4⁺ T-cells can be primed to one or more specific antigens, for example one or more TAAs, and the CD3⁺ NKT-cells are exposed to the same antigens. In one embodiment, the CD8⁺ and CD4⁺ T-cells can be primed to one or more specific antigens, for example one or more TAAs, and the CD3⁺ NKT-cells are exposed to the same antigens, while all of the cells are further exposed to one or more glycolipids. In an alternative embodiment, the CD8⁺ and CD4⁺ T-cells can be primed to one or more specific antigens, for example one or more TAAs, and the CD3⁺ NKT-cells are exposed to the same antigens, and the CD3⁺ NKT-cells are further exposed and/or primed to one or more glycolipids.

In one embodiment, the lymphocytic cell subsets are naïve to one or more of the targeted antigens to which they are exposed. In one embodiment, the lymphocytic cell subsets are naïve to all of the targeted antigens to which they are exposed.

TCR αβT-Cell and TCR γδ T-Cell Composition

In an alternative aspect, the composition provides a fixed ratio of a population of different lymphocytic cell subsets comprising TCR αβ T-cells and TCR γδ T-cells. In one embodiment, the cells have been exposed ex vivo against one or more specific target antigens. In one embodiment, only the αβ T-cells are exposed to the one or more specific target antigens. The αβ T-cells of the cell composition are primed against the one or more specific target antigens, while the γδ T-cells are activated.

In one embodiment, the composition comprises about a 1:1 ratio (+/−5%) of αβ T-cells:γδ T-cells.

In one embodiment, the composition comprises between about 55% and 65% αβ T-cells and between about 35% and 45% γδ T-cells. For example, in one embodiment the composition comprises about 55%, 56%, 57%, 58%, 59%, 60%, 61%, 62%, 63%, 65%, or 65% αβ T-cells and about 35%, 36%, 37%, 38%, 39%, 40%, 41%, 42%, 43%, 44%, or 45% γδ T-cells.

In one embodiment, the composition comprises about 60% αβ T-cells and about 40% γδ T-cells.

In an alternative embodiment, the cell composition comprises at least about 40% αβ T-cells, and at least about 35% γδ T-cells. In one embodiment, the composition comprises between about 35% and about 45% αβ T-cells, and between about 30% and about 40% γδ T-cells. In one embodiment, the composition comprises about 40% αβ T-cells and about 35% γδ T-cells.

The αβ T-cells of the composition may comprise varying ratios of CD8⁺ and CD4⁺ T-cells. For example, the αβ T-cells of the composition may comprise fixed ratios of CD8⁺ and CD4⁺ T-cells for example about a 1:1 ratio (+/−5%) of CD8⁺ T-cells:CD4⁺ T-cells; about 1.5:1 ratio (+/5%) of CD8⁺ T-cells:CD4⁺ T-cells; about a 2:1 ratio (+/−5%) of CD8⁺ T-cells:CD4⁺ T-cells; about 2.5:1 ratio (+/−5%) of CD8⁺ T-cells:CD4⁺ T-cells; about 3:1 ratio (+/−5%) of CD8⁺ T-cells:CD4⁺ T-cells; about 3.5:1 ratio (+/−5%) of CD8⁺ T-cells:CD4 T-cells; about 4:1 ratio (+/−5%) of CD8⁺ T-cells:CD4⁺ T-cells.

In one embodiment, the cell composition comprising αβ T-cells and γδ T-cells includes αβ T-cells that are between about 55% to about 65% of CD8⁺ T-cells and between about 35% to about 45% of CD4⁺ T-cells. For example, in one embodiment the composition comprises about 55%, 56%, 57%, 58%, 59%, 60%, 61%, 62%, 63%, 65%, or 65% CD8⁺ T-cells and about 35%, 36%, 37%, 38%, 39%, 40%, 41%, 42%, 43%, 44%, or 45% CD4⁺ T-cells.

In one embodiment, the cell composition comprising αβ T-cells and γδ T-cells includes αβ T-cells that are between about 60% CD8⁺ T-cells and about 40% of CD4⁺ T-cells.

In one embodiment, the CD4⁺ T-cells of the composition are primarily CD4⁺ Th1-cells. For example, the CD4⁺ Th1-cells of the composition make up about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 84%, 85%, 86%, 87%, 88%, 89%, 90% or more of the total CD4 T-cells in the composition.

In one embodiment, the composition is comprised of little or minimal CD4⁺ Treg-cells. For example, CD4⁺ Treg-cells make up less than about 5%, 4%, 3%, 2%, or 1% of the population of CD4⁺ T-cells.

In one embodiment, the γδ T-cells are predominately Vγ9Vδ2 T-cells, for example, at least about 70%, 71%, 72%, 73%, 74%, 75%, 76%, 77%, 78%, 79%, 80%, 81%, 82%, 83%, 84%, 84%, 85%, 86%, 87%, 88%, 89%, 90% or more of the γδ T-cells are Vγ9Vδ2T-cells.

In one embodiment, the cell composition consists of only αβ T-cells and γδ T-cells.

In one embodiment, the cell composition comprises primarily αβ T-cells and γδ T-cells.

In one embodiment, the cells are exposed to one or more targeted antigens selected from a TAA, a VATA, or a combination thereof, and the αβ T-cells are primed against the same target antigens. In one embodiment, the lymphocytic cell subsets are naïve to one or more of the targeted antigens to which they are exposed. In one embodiment, the lymphocytic cell subsets are naïve to all of the targeted antigens to which they are exposed.

αβT-Cell, γδ T-Cell, and CD3⁺ NKT-Cell

In still another alternative aspect, the composition provides a fixed ratio of a population of different lymphocytic cell subsets comprising αβ T-cells, γδ T-cells, and CD3⁺ NKT-cells. In one embodiment, all of the cells are exposed to one or more specific target antigens. In one embodiment, only the αβ T-cells are exposed to one or more specific target antigens. The αβ T-cells of the cell composition are primed against the one or more specific target antigens, while the CD3⁺ NKT-cells and γδ T-cells are activated.

In one embodiment, the composition comprises about a 1:1:1 ratio (+/−5%) of αβ T-cells:γδ T-cells:CD3⁺ NKT-cells.

In one embodiment, the composition comprises between about 25% and about 35% αβ T-cells, between about 25% and about 35% γδ T-cells, and between about 35% and about 45% CD3⁺ NKT-cells. For example, in one embodiment the composition comprises about 25%, 26%, 27%, 28%, 29%, 30%, 31%, 32%, 33%, 34%, or 35% αβ T-cells; about 25%, 26%, 27%, 28%, 29%, 30%, 31%, 32%, 33%, 34%, or 35% γδ T-cells; and about 35%, 36%, 37%, 38%, 39%, 40%, 41%, 42%, 43%, 44%, or 45% of CD3⁺ NKT-cells.

In one embodiment, the composition comprises about 30% αβ T-cells, about 30% γδ T-cells, and about 40% CD3⁺ NKT-cells, resulting in a cell composition comprising about a 0.3:0.3:0.4 ratio of αβ T-cells:γδ T-cells:CD3⁺ NKT-cells. In one embodiment, the αβ T-cells are comprised of a 1:1 ratio (+/−5%) of CD4⁺ T-cells:CD8⁺ T-cells, resulting in a cell composition comprising about a 0.15:0.15:0.3:0.4 ratio of CD8⁺ T-cells:CD4⁺ T-cells:γδ T-cells:CD3⁺ NKT-cells.

In one embodiment, the αβ T-cells are comprised of between about 55% to about 65% of CD8⁺ T-cells and between about 35% to about 45% of CD4⁺ T-cells. For example, the composition is comprised of about 55%, 56%, 57%, 58%, 59%, 60%, 61%, 62%, 63%, 64%, or 65% CD8⁺ T-cells, and about 35%, 365, 37%, 38%, 39%, 40%, 41%, 42%, 43%, 44%, or 45% CD4⁺ T-cells.

In one embodiment, the αβ T-cells are comprised of about 60% CD8⁺ T-cells and about 40% of CD4⁺ T-cells, resulting in a cell composition comprising about a 0.18:0.12:0.3:0.4 ratio of CD8⁺ T-cells:CD4⁺ T-cells:γδ T-cells:CD3⁺ NKT-cells.

The αβ T-cells of the composition may comprise varying ratios of CD8⁺ and CD4⁺ T-cells. For example, the αβ T-cells of the composition may comprise fixed ratios of CD8⁺ and CD4⁺ T-cells for example about a 1:1 ratio (+/−5%) of CD8⁺ T-cells:CD4⁺ T-cells; about 1.5:1 ratio (+/5%) of CD8⁺ T-cells:CD4⁺ T-cells; about a 2:1 ratio (+/−5%) of CD8⁺ T-cells:CD4⁺ T-cells; about 2.5:1 ratio (+/−5%) of CD8⁺ T-cells:CD4⁺ T-cells; about 3:1 ratio (+/−5%) of CD8⁺ T-cells:CD4⁺ T-cells; about 3.5:1 ratio (+/−5%) of CD8⁺ T-cells:CD4⁺ T-cells; about 4:1 ratio (+/−5%) of CD8⁺ T-cells:CD4⁺ T-cells.

In one embodiment, the cell composition comprising αβ T-cells and γδ T-cells includes αβ T-cells that are between about 55% to about 65% of CD8⁺ T-cells and between about 35% to about 45% of CD4⁺ T-cells. For example, in one embodiment the composition comprises about 55%, 56%, 57%, 58%, 59%, 60%, 61%, 62%, 63%, 65%, or 65% CD8⁺ T-cells and about 35%, 36%, 37%, 38%, 39%, 40%, 41%, 42%, 43%, 44%, or 45% CD4⁺ T-cells.

In one embodiment, the cell composition comprising αβ T-cells and γδ T-cells includes αβ T-cells that are between about 60% CD8⁺ T-cells and about 40% of CD4⁺ T-cells, resulting in a cell composition comprising about a 0.6:0.4:1 ratio of CD8⁺ -cells:CD4⁺ T-cells:γδ T-cells.

In an alternative embodiment, the cell composition comprises at least about 35% αβ T-cells, at least about 30% γδ T-cells, and at least about 10% CD3⁺ NKT-cells. In one embodiment, the composition comprises between about 35% and 45% αβ T-cells, between about 30% and 40% γδ T-cells, and between about 10% and 20% CD3⁺ NKT-cells. In one embodiment, the composition comprises about 40% αβ T-cells, about 35% γδ T-cells, and about 15% CD3⁺ NKT-cells. In one embodiment, the αβ T-cells are comprised of a 1:1 ratio (+/−5%) of CD8⁺ T-cells:CD4⁺ T-cells. In one embodiment, the αβ T-cells are comprised of between about 55% to about 65% of CD8⁺ T-cells and between about 35% to about 45% of CD4⁺ T-cells. In one embodiment, the αβ T-cells are comprised of about 60% CD8⁺ T-cells and about 40% of CD4⁺ T-cells.

In one embodiment, the CD4⁺ T-cells of the composition are primarily CD4⁺ Th1-cells. For example, the CD4 Th1-cells of the composition make up about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 84%, 85%, 86%, 87%, 88%, 89%, 90% or more of the total CD4⁺ T-cells in the composition.

In one embodiment, the composition is comprised of little or minimal CD4⁺ Treg-cells. For example, CD4⁺ Treg-cells make up less than about 5%, 4%, 3%, 2%, or 1% of the population of CD4⁺ T-cells.

In one embodiment, the γδ T-cells are predominately Vγ9Vδ2 T-cells, for example, at least about 70%, 71%, 72%, 73%, 74%, 75%, 76%, 77%, 78%, 79%, 80%, 81%, 82%, 83%, 84%, 84%, 85%, 86%, 87%, 88%, 89%, 90% or more of the γδ T-cells are Vγ9Vδ2T-cells.

The CD3⁺ NKT-cells of the composition can be CD8⁺ NKT-cells, CD4⁺ NKT-cells, or CD8⁻/CD4⁻NKT-cells, or a mixture thereof. In one embodiment, the CDK3⁺ NKT-cells are primarily type I NKT-cells. For example, in one embodiment, type I NKT-cells comprise about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 84%, 85%, 86%, 87%, 88%, 89%, 90% or more of the CD3⁺ NKT-cells of the composition.

In one embodiment, the cell composition consists of only αβ T-cells, γδ T-cells, and CD3⁺ NKT-cells.

In one embodiment, the cell composition primarily αβ T-cells, γδ T-cells, and CD3⁺ NKT-cells.

In one embodiment, the αβ T-cells can be primed to one or more specific antigens, for example one or more TAAs, and the CD3⁺ NKT-cells and γδ T-cells are exposed to the same antigens. In one embodiment, the αβ T-cells can be primed to one or more specific antigens, for example one or more TAAs, and the CD3⁺ NKT-cells and γδ T-cells are exposed to the same antigens, while all of the cells are further exposed to one or more glycolipids. In an alternative embodiment, the αβ T-cells can be primed to one or more specific antigens, for example one or more TAAs, the CD3⁺ NKT-cells and γδ T-cells are exposed to the same antigens, and the CD3⁺ NKT-cells are further exposed and/or primed to one or more glycolipids.

In one embodiment, the lymphocytic cell subsets are naïve to one or more of the targeted antigens to which they are exposed. In one embodiment, the lymphocytic cell subsets are naïve to all of the targeted antigens to which they are exposed.

Exhaustion Markers

In one aspect, the cell compositions may be further selected (or conditioned) for the presence or lack of one or more markers associated with, for example, maturation or exhaustion.

T-cell exhaustion (Tex) is a state of dysfunction that results from persistent antigen and inflammation, both of which commonly occur in cancer tissue. The reversal or prevention of exhaustion is a major area of research for cancer immunotherapy. Tex cell populations can be analyzed using multiple phenotypic parameters, either alone or in combination.

In one aspect, the cell composition in the fixed ratios described herein has less than about 15% of cells expressing a marker associated with Tex. In one embodiment, the cell compositions have less than about 10% of cells expressing a marker associated with Tex. In one embodiment, the cell composition has less than about 5% of cells expressing a marker associated with Tex. In one embodiment, the cell composition has less than about 5%, 4%, 3%, 2%, 1% or less of cells expressing a marker associated with Tex.

Hallmarks commonly used to monitor T-cell exhaustion are known in the art and include, but are not limited to, programmed cell death-1 (PD-1), CTLA-4/CD152 (Cytotoxic T-Lymphocyte Antigen 4), LAG-3 (Lymphocyte activation gene-3; CD223), TIM-3 (T cell immunoglobulin and mucin domain-3), 2B4/CD244/SLAMF4, CD160, and TIGIT (T cell Immunoreceptor with Ig and ITIM domains).

PD-1 (Programmed Death-1 receptor) is a key regulator of the threshold of immune response and peripheral immune tolerance. It is expressed on activated T cells, B cells, monocytes, and dendritic cells and binds to PD-L1 or PD-L2. PD-1 ligation induces co-inhibitory signals in T cells promoting their apoptosis, anergy, and functional exhaustion.

In one aspect, provided herein is a cell composition in the fixed ratios described herein, wherein the population has less than about 15% of cells expressing PD-1. In one embodiment, the composition has less than about 10% of cells expressing PD-1. In one embodiment, the composition of has less than about 5% of cells expressing PD-1. In one embodiment, the composition has less than about 5%, 4%, 3%, 2%, 1% or less of cells expressing PD-1.

CTLA-4/CD152 (Cytotoxic T-Lymphocyte Antigen 4) is a transmembrane T cell inhibitory molecule that is expressed as a covalent homodimer. CTLA-4 is recruited from intracellular vesicles to the immunological synapse beginning 1-2 days after T cell activation. It forms a linear lattice with B7-1 on APC, inducing negative regulatory signals and ending CD28-dependent T cell activation. Mice deleted for CTLA-4 develop lethal autoimmune reactions due to continued T cell activation and poor control by regulatory T cells which constitutively express CTLA-4.

In one aspect, provided herein is a cell composition in the fixed ratios described herein wherein the population has less than about 15% of cells expressing CTLA-4. In one embodiment, the composition has less than about 10% of cells expressing CTLA-4. In one embodiment, the composition has less than about 5% of cells expressing CTLA-4. In one embodiment, the composition has less than about 5%, 4%, 3%, 2%, 1% or less of cells expressing CTLA-4.

LAG-3 (Lymphocyte activation gene-3; CD223) is a transmembrane protein that binds to MHC class II molecules and negatively regulates T cell receptor signaling. It is expressed on activated T cells, NK cells, and plasmacytoid dendritic cells (pDC). LAG-3 limits the expansion of activated T cells and pDC in response to select stimuli. Proteolytic shedding of LAG-3 enables normal T cell activation by removing the negative regulation. Binding of a homodimerized soluble LAG-3/Ig fusion protein to MHC class II molecules induces maturation of immature DC as well as secretion of pro-inflammatory cytokines by cytotoxic CD8⁺ T cells and NK cells.

In one aspect, provided herein is a cell composition in the fixed ratios described herein wherein the population of cells has less than about 15% of cells expressing LAG-3. In one embodiment, the composition has less than about 10% of cells expressing LAG-3. In one embodiment, the composition has less than about 5% of cells expressing LAG-3. In one embodiment, the composition has less than about 5%, 4%, 3%, 2%, 1% or less of cells expressing LAG-3.

TIM-3 (T-cell immunoglobulin and mucin domain-3), also known as HAVCR2 is an immunosuppressive protein that enhances tolerance and inhibits anti-tumor immunity. It is upregulated on several populations of activated myeloid cells (macrophage, monocyte, dendritic cell, microglia, mast cell) and T-cells (Th1, CD8⁺, NK, Treg). TIM-3 ligation by Galectin-9 attenuates CD8⁺ and Th1 cell responses and promotes the activity of Treg and myeloid derived suppressor cells. Dendritic cell-expressed TIM-3 dampens inflammation by enabling the phagocytosis of apoptotic cells and the cross-presentation of apoptotic cell antigens. TIM-3 also binds the alarmin HMGBL thereby preventing the activation of TLRs in response to released tumor cell DNA.

In one aspect, provided herein is a cell composition in the fixed ratios described herein wherein the population s has less than about 15% of cells expressing TIM-3. In one embodiment, the composition has less than about 10% of cells expressing TIM-3. In one embodiment, the composition has less than about 5% of cells expressing TIM-3. In one embodiment, the composition has less than about 5%, 4%, 3%, 2%, 1% or less of cells expressing TIM-3.

2B4, also known as CD244, is a cell surface glycoprotein belonging to the CD2 subgroup of the immunoglobulin superfamily. It acts as a high-affinity receptor for CD48. It is expressed by natural killer (NK) cells and CD8⁺ T cell subsets. It can regulate killing by CD8⁺ T cells and

NK cells, and IFN-gamma secretion by NK cells. It may also regulate NK cell and T cell proliferation.

In one aspect, provided herein is a cell composition in the fixed ratios described herein, wherein the population has less than about 15% of cells expressing 2B4. In one embodiment, the composition has less than about 10% of cells expressing 2B4. In one embodiment, the composition has less than about 5% of cells expressing 2B4. In one embodiment, the composition has less than about 5%, 4%, 3%, 2%, 1% or less of cells expressing 2B4.

CD160 is a GPI-anchored glycoprotein with one Ig-like V-type domain. On a subpopulation of cytolytic T cells and NK cells, CD160 functions as a broad specificity receptor for MEW class I and related molecules. When expressed on vascular endothelial cells, CD160 propagates anti-angiogenic signals and promotes apoptosis.

In one aspect, provided herein is a cell composition in the fixed ratios described herein, wherein the cell population has less than about 15% of cells expressing CD160. In one embodiment, the composition has less than about 10% of cells expressing CD160. In one embodiment, the composition has less than about 5% of cells expressing CD160. In one embodiment, the composition has less than about 5%, 4%, 3%, 2%, 1% or less of cells expressing CD160.

TIGIT (T-cell Immunoreceptor with Ig and ITIM domains), also called Vstm3, Vsig9, and WUCAM, is a transmembrane protein in the CD28 family of the Ig superfamily proteins. TIGIT is expressed on NK cells and subsets of activated, memory and regulatory T cells, and particularly on follicular helper T cells within secondary lymphoid organs. It binds to CD155/PVR/Nec1-5 and Nectin-2/CD112/PVRL2 on dendritic cells (DC) and endothelium. Binding of TIGIT by DC induces IL-10 release and inhibits IL-12 production. Ligation of TIGIT on T cells downregulates TCR-mediated activation and subsequent proliferation, while NK cell TIGIT ligation blocks NK cell cytotoxicity. CD155 and Nectin-2 also interact with DNAM-1/CD226 and CD96/Tactile, and TIGIT binding to CD155 can antagonize the effects of DNAM-1. Soluble TIGIT is able to compete with DNAM-1 for CD155 binding and attenuates T cell responses, while mice lacking TIGIT show increased T cell responses and susceptibility to autoimmune challenges.

In one aspect, provided herein is a cell composition in the fixed ratios described herein, wherein the population has less than about 15% of cells expressing TIGIT. In one embodiment, the composition has less than about 10% of cells expressing TIGIT. In one embodiment, the composition has less than about 5% of cells expressing TIGIT. In one embodiment, the composition has less than about 5%, 4%, 3%, 2%, 1% or less of cells expressing TIGIT.

In one aspect, provided herein is a cell composition in a fixed ratio as described herein, wherein the cell population has less than about 15% of cells expressing a marker associated with Tex. In one embodiment, the composition has less than about 10% of cells expressing a marker associated with Tex. In one embodiment, the composition has less than about 5% of cells expressing a marker associated with Tex. In one embodiment, the composition has less than about 5%, 4%, 3%, 2%, 1% or less of cells expressing a marker associated with Tex. In one embodiment, the Tex marker is PD-1. In one embodiment, the Tex marking is CTLA-4. In one embodiment, the Tex marker is TIM3. In one embodiment, the Tex is Lag3. In one embodiment, the Tex is 2B4. In one embodiment, the Tex is CD160. In one embodiment, the Tex is TIGIT. In one embodiment, the composition comprises less than about 10% of TAA-Ls expressing one of PD-1, CTLA-4, TIM3, LAG3, 2B4, CD160, TIGIT, or a combination thereof. In one embodiment, the composition comprises less than about 5% of TAA-Ls expressing one of PD-1, CTLA-4, TIM3, LAG3, 2B4, CD160, TIGIT, or a combination thereof. In one embodiment, the composition comprises less than about 5%, 4%, 3%, 2%, 1% or less of the cell population expressing one of PD-1, CTLA-4, TIM3, LAG3, 2B4, CD160, TIGIT, or a combination thereof.

Methods for identifying cells having these particular markers are well known in the art.

Tumor-Associated Antigens

Antigens used herein for immunotherapy should be intentionally selected based on either uniqueness to tumor cells, greater expression in tumor cells as compared to normal cells, or ability of normal cells with antigen expression to be adversely affected without significant compromise to normal cells or tissue. As a non-limiting example, Wilms tumor gene (WT1) is found in post-natal kidney, pancreas, fat, gonads and hematopoietic stem cells. In healthy hematopoietic stem cells WT1 encodes a transcription factor, which regulates cell proliferation, cell death and differentiation. WT1 is overexpressed in Wilms tumor, soft tissue sarcomas, rhabdomyosarcoma, ovarian, and prostate cancers. The WT1 gene was initially identified as a tumor suppressor gene due to its inactivation in Wilms' tumor (nephroblastoma), the most common pediatric kidney tumor. However, recent findings have shown that WT1 acts as an oncogene in ovarian and other tumors. In addition, several studies have reported that high expression of WT1 correlates with the aggressiveness of cancers and a poor outcome in leukemia, breast cancer, germ-cell tumor, prostate cancer, soft tissue sarcomas, rhabdomyosarcoma and head and neck squamous cell carcinoma. There are several studies describing WT1 expression in ovarian cancers. A positive expression has been primarily observed in serous adenocarcinoma, and WT1 is more frequently expressed in high-grade serous carcinoma, which stands-out from other sub-types due to its aggressive nature and because it harbors unique genetic alterations. Patients with WT1-positive tumors tend to have a higher grade and stage of tumor.

Preferentially expressed antigen of melanoma (PRAME), initially identified in melanoma, has been associated with other tumors including neuroblastoma, osteosarcoma, soft tissue sarcomas, head and neck, lung and renal cancer including Wilms tumor. In neuroblastoma and osteosarcoma, PRAME expression was associated with advanced disease and a poor prognosis. PRAME is also highly expressed in leukemic cells and its expression levels are correlated with relapse and remission. The function in healthy tissue is not well understood, although studies suggest PRAME is involved in proliferation and survival in leukemia cells.

Survivin is highly expressed during normal fetal development but is absent in most mature tissues. It is thought to regulate apoptosis and proliferation of hematopoietic stem cells. Overexpression of survivin has been reported in almost all human malignancies including bladder cancer, lung cancer, breast cancer, stomach, esophagus, liver, ovarian cancers and hematological cancers. Survivin has been associated with chemotherapy resistance, increased tumor recurrence and decreased survival.

Tumor-associated antigens (TAA) can be loosely categorized as oncofetal (typically only expressed in fetal tissues and in cancerous somatic cells), oncoviral (encoded by tumorigenic transforming viruses), overexpressed/accumulated (expressed by both normal and neoplastic tissue, with the level of expression highly elevated in neoplasia), cancer-testis (expressed only by cancer cells and adult reproductive tissues such as testis and placenta), lineage-restricted (expressed largely by a single cancer histotype), mutated (only expressed by cancer as a result of genetic mutation or alteration in transcription), post-translationally altered (tumor-associated alterations in glycosylation, etc.), or idiotypic (highly polymorphic genes where a tumor cell expresses a specific “clonotype”, i.e., as in B cell, T-cell lymphoma/leukemia resulting from clonal aberrancies). Although they are preferentially expressed by tumor cells, TAAs are oftentimes found in normal tissues. However, their expression differs from that of normal tissues by their degree of expression in the tumor, alterations in their protein structure in comparison with their normal counterparts or by their aberrant subcellular localization within malignant or tumor cells.

Examples of oncofetal tumor associated antigens include Carcinoembryonic antigen (CEA), immature laminin receptor, and tumor-associated glycoprotein (TAG) 72. Examples of overexpressed/accumulated include BING-4, calcium-activated chloride channel (CLCA) 2, Cyclin B 1, 9D7, epithelial cell adhesion molecule (Ep-Cam), EphA3, Her2/neu, telomerase, mesothelin, orphan tyrosine kinase receptor (ROR1), stomach cancer-associated protein tyrosine phosphatase 1 (SAP-1), and survivin.

Examples of cancer-testis antigens include the b melanoma antigen (BAGE) family, cancer-associated gene (CAGE) family, G antigen (GAGE) family, melanoma antigen (MAGE) family, sarcoma antigen (SAGE) family and X antigen (XAGE) family, CT9, CT10, NY-ESO-1, L antigen (LAGE) 1, Melanoma antigen preferentially expressed in tumors (PRAME), and synovial sarcoma X (SSX) 2.

Examples of lineage restricted tumor antigens include melanoma antigen recognized by T-cells-1/2 (Melan-A/MART-1/2), Gp100/pme117, tyrosine-related protein (TRP) 1 and 2, P. polypeptide, melanocortin 1 receptor (MC1R), and prostate-specific antigen. Examples of mutated tumor antigens include β-catenin, breast cancer antigen (BRCA) 1/2, cyclin-dependent kinase (CDK) 4, chronic myelogenous leukemia antigen (CML) 66, fibronectin, p53, Ras, and TGF-βRII. An example of a post-translationally altered tumor antigen is mucin (MUC) 1. Examples of idiotypic tumor antigens include immunoglobulin (Ig) and T cell receptor (TCR).

In some embodiments, the antigen associated with the disease or disorder is selected from the group consisting of BCMA, CD19, CD20, CD22, hepatitis B surface antigen, anti-folate receptor, CD23, CD24, CD30, CD33, CD38, CD44, CD138, CS1, EGFR, EGP-2, EGP-4, 0EPHa2, ErbB2, 3, or 4, FBP, fetal acetylcholine receptor, HMW-MAA, IL-22R-alpha, IL-13R-alpha, kdr, kappa light chain, Lewis Y, L1-cell adhesion molecule, MAGE-A1, MUC1, MUC16 (CA-125), PSCA, NKG2D Ligands, oncofetal antigen, VEGF-R2, PSMA, XBP-1, estrogen receptor, progesterone receptor, ephrinB2, CD123, CS-1, c-Met and/or biotinylated molecules, and/or molecules expressed by HIV, HCV, HBV or other pathogens.

Exemplary tumor antigens include at least the following: carcinoembryonic antigen (CEA) for bowel cancers; CA-125 for ovarian cancer; MUC1 or epithelial tumor antigen (ETA) or CA15-3 for breast cancer; tyrosinase or melanoma-associated antigen (MAGE) for malignant melanoma; and abnormal products of ras, p53 for a variety of types of tumors; alphafetoprotein for hepatoma, ovarian, or testicular cancer; beta subunit of hCG for men with testicular cancer; prostate specific antigen for prostate cancer; beta 2 microglobulin for multiple myeloma and in some lymphomas; CA19-9 for colorectal, bile duct, and pancreatic cancer; chromogranin A for lung and prostate cancer; TA90 for melanoma, soft tissue sarcomas, and breast, colon, and lung cancer. Examples of TAAs are known in the art, for example in Vigneron, “Human Tumor Antigens and Cancer Immunotherapy,” Biomed Res. Int., vol. 2015, Article ID 948501, 17 pages, 2015. doi:10.1155/2015/948501; Ilyas et al., J. Immunol. 195(11): 117-22 (2015); Coulie et al., Nat. Rev. Cancer 14:135-46 (2014); Cheever et al., Clin. Cancer Res. 15(17):5323-37 (2009), which are incorporated by reference herein in its entirety.

Examples of oncoviral TAAs include human papilloma virus (HPV) L1, E6 and E7, Epstein-Barr Virus (EBV) Epstein-Barr nuclear antigen (EBNA), EBV viral capsid antigen (VCA) Igm or IgG, EBV early antigen (EA), latent membrane protein (LMP) 1 and 2, hepatitis B surface antigen (HBsAg), hepatitis B e antigen (HBeAg), hepatitis B core antigen (HBcAg), hepatitis B x antigen (HBxAg), hepatitis C core antigen (HCV core Ag), Human T-Lymphotropic Virus Type 1 core antigen (HTLV-1 core antigen), HTLV-1 Tax antigen, HTLV-1 Group specific (Gag) antigens, HTLV-1 envelope (Env), HTLV-1 protease antigens (Pro), HTLV-1 Tof, HTLV-1 Rof, HTLV-1 polymerase (Pro) antigen, Human T-Lymphotropic Virus Type 2 core antigen (HTLV-2 core antigen), HTLV-2 Tax antigen, HTLV-2 Group specific (Gag) antigens, HTLV-2 envelope (Env), HTLV-2 protease antigens (Pro), HTLV-2 Tof, HTLV-2 Rof, HTLV-2 polymerase (Pro) antigen, latency-associated nuclear antigen (LANA), human herpesvirus-8 (HHV-8) K8.1, Merkel cell polyomavirus large T antigen (LTAg), and Merkel cell polyomavirus small T antigen (sTAg).

Elevated expression of certain types of glycolipids, for example gangliosides, is associated with the promotion of tumor survival in certain types of cancers. Examples of gangliosides include, for example, GM1b, GD1c, GM3, GM2, GM1a, GD1a, GT1a, GD3, GD2, GD1b, GT1b, GQ1b, GT3, GT2, GT1c, GQ1c, and GP1c. Examples of ganglioside derivatives include, for example, 9-O-Ac-GD3, 9-O-Ac-GD2, 5-N-de-GM3, N-glycolyl GM3, NeuGcGM3, and fucosyl-GM1. Exemplary gangliosides that are often present in higher levels in tumors, for example melanoma, small-cell lung cancer, sarcoma, and neuroblastoma, include GD3, GM2, and GD2.

In addition to the TAAs described above, another class of TAAs is tumor-specific neoantigens, which arise via mutations that alter amino acid coding sequences (non-synonymous somatic mutations). Some of these mutated peptides can be expressed, processed and presented on the cell surface, and subsequently recognized by T cells. Because normal tissues do not possess these somatic mutations, neoantigen-specific T cells are not subject to central and peripheral tolerance, and also lack the ability to induce normal tissue destruction. See, e.g., Lu & Robbins, Semin. Immunol. 28(1):22-27 (2016), incorporated herein by reference.

In one embodiment, one or more T-cell subpopulation of one or more T-cell components comprising the MUSTANG composition is specific to an oncofetal TAA selected from a group consisting of Carcinoembryonic antigen (CEA), immature laminin receptor, orphan tyrosine kinase receptor (ROR1), and tumor-associated glycoprotein (TAG) 72. In one embodiment, at least one T-cell subpopulation is specific to CEA. In one embodiment, one or more T-cell subpopulation of one or more T-Cell components is specific to immature laminin receptor. In one embodiment, one or more T-cell subpopulation of one or more T-cell components is specific to ROR1. In one embodiment, at least one T-cell subpopulation is specific is specific to TAG72.

In one embodiment, one or more T-cell subpopulation of one or more T-cell components comprising the MUSTANG composition is specific to an oncoviral TAA selected from a group consisting of human papilloma virus (HPV) E6 and E7, Epstein-Barr Virus (EBV) Epstein-Barr nuclear antigen (EBNA), latent membrane protein (LMP) 1, and LMP2. In one embodiment, one or more T-cell subpopulation of one or more T-cell components is specific to HPV E6. In one embodiment, at least one T-cell subpopulation is specific to HPV E7. In one embodiment, one or more T-cell subpopulation of one or more T-cell components is specific to EBV. In one embodiment, at least one T-cell subpopulation is specific to EBNA. In one embodiment, one or more T-cell subpopulation of one or more T-cell components is specific to LMP1. In one embodiment, one or more T-cell subpopulation of one or more T-cell components is specific to LMP2.

In one embodiment, one or more T-cell subpopulation of one or more T-cell components comprising the MUSTANG composition is specific to an overexpressed/accumulated TAA selected from a group consisting of BCMA, BING-4, calcium-activated chloride channel (CLCA) 2, CD138, Cyclin Bi, CS1, 9D7, epithelial cell adhesion molecule (Ep-Cam), EphA3, Her2/neu, L1 cell adhesion molecule (L1-Cam), telomerase, mesothelin, stomach cancer-associated protein tyrosine phosphatase 1 (SAP-1), survivin, and XBP-1. In one embodiment, one or more T-cell subpopulation of one or more T-cell components is specific to BCMA. In one embodiment, one or more T-cell subpopulation of one or more T-cell components is specific to BING-4. In one embodiment, at least one T-cell subpopulation is specific to CLCA2. In one embodiment, one or more T-cell subpopulation of one or more T-cell components is specific to CD138. In one embodiment, one or more T-cell subpopulation of one or more T-cell components is specific to Cyclin Bi. In one embodiment, one or more T-cell subpopulation of one or more T-cell components is specific to CS1. In one embodiment, one or more T-cell subpopulation of one or more T-cell components is specific to 9D7. In one embodiment, one or more T-cell subpopulation of one or more T-cell components is specific Ep-Cam. In one embodiment, one or more T-cell subpopulation of one or more T-cell components is specific to EphA3. In one embodiment, at least one T-cell subpopulation is specific to Her2/neu. In one embodiment, one or more T-cell subpopulation of one or more T-cell components is specific to L1-Cam. In one embodiment, one or more T-cell subpopulation of one or more T-cell components is specific to telomerase. In one embodiment, at least one T-cell subpopulation is specific to mesothelin. In one embodiment, one or more T-cell subpopulation of one or more T-cell components is specific to SAP-1. In one embodiment, one or more T-cell subpopulation of one or more T-cell components is specific to survivin. In one embodiment, one or more T-cell subpopulation of one or more T-cell components is specific to XBP-1.

In one embodiment, one or more T-cell subpopulation of one or more T-cell components comprising the MUSTANG composition is specific to a cancer-testis antigen selected from the group consisting of the b melanoma antigen (BAGE) family, cancer-associated gene (CAGE) family, G antigen (GAGE) family, melanoma antigen (MAGE) family, sarcoma antigen (SAGE) family and X antigen (XAGE) family, cutaneous T cell lymphoma associated antigen family (cTAGE), Interleukin-13 receptor subunit alpha-1 (IL13RA), CT9, Putative tumor antigen NA88-A, leucine zipper protein 4 (LUZP4), NY-ESO-1, L antigen (LAGE) 1, helicase antigen (HAGE), lipase I (LIPI), Melanoma antigen preferentially expressed in tumors (PRAME), synovial sarcoma X (SSX) family, sperm protein associated with the nucleus on the chromosome X (SPANX) family, cancer/testis antigen 2 (CTAG2), calcium-binding tyrosine phosphorylation-regulated fibrous sheath protein (CABYR), acrosin binding protein (ACRBP), centrosomal protein 55 (CEP55) and Synaptonemal Complex Protein 1 (SYCP1). In one embodiment, one or more T-cell subpopulation of one or more T-cell components is specific to the BAGE family. In one embodiment, one or more T-cell subpopulation of one or more T-cell components is specific to the CAGE family. In one embodiment, one or more T-cell subpopulation of one or more T-cell components is specific to the GAGE family. In one embodiment, one or more T-cell subpopulation of one or more T-cell components is specific to the MAGE family. In one embodiment, one or more T-cell subpopulation of one or more T-cell components is specific to the SAGE family. In one embodiment, one or more T-cell subpopulation of one or more T-cell components is specific to the XAGE family. In one embodiment, one or more T-cell subpopulation of one or more T-cell components is specific to the cTAGE family. In one embodiment, one or more T-cell subpopulation of one or more T-cell components is specific to IL13RA. In one embodiment, one or more T-cell subpopulation of one or more T-cell components is specific to CT9. In one embodiment, one or more T-cell subpopulation of one or more T-cell components is specific to NA88-A. In one embodiment, one or more T-cell subpopulation of one or more T-cell components is specific to LUZP4. In one embodiment, one or more T-cell subpopulation of one or more T-cell components is specific to NY-ESO-1. In one embodiment, one or more T-cell subpopulation of one or more T-cell components is specific to LAGE-1. In one embodiment, one or more T-cell subpopulation of one or more T-cell components is specific to HAGE. In one embodiment, one or more T-cell subpopulation of one or more T-cell components is specific to LIPI. In one embodiment, one or more T-cell subpopulation of one or more T-cell components is specific to PRAME. In one embodiment, one or more T-cell subpopulation of one or more T-cell components is specific to the SSX family. In one embodiment, one or more T-cell subpopulation of one or more T-cell components is specific to the SPANX family. In one embodiment, one or more T-cell subpopulation of one or more T-cell components is specific to CTAG2. In one embodiment, one or more T-cell subpopulation of one or more T-cell components is specific to CABYR. In one embodiment, one or more T-cell subpopulation of one or more T-cell components is specific to ACRBP. In one embodiment, one or more T-cell subpopulation of one or more T-cell components is specific to CEP55. In one embodiment, one or more T-cell subpopulation of one or more T-cell components is specific to SYCP1.

In one embodiment, one or more T-cell subpopulation of one or more T-cell components comprising the MUSTANG composition is specific to a lineage restricted tumor antigen selected from the group consisting of melanoma antigen recognized by T-cells-1/2 (Melan-A/MART-1/2), Gp100/pme117, tyrosinase, tyrosine-related protein (TRP) 1 and 2, P. polypeptide, melanocortin 1 receptor (MC1R), and prostate-specific antigen. In one embodiment, one or more T-cell subpopulation of one or more T-cell components is specific to Melan-A/MART-1/2. In one embodiment, one or more T-cell subpopulation of one or more T-cell components is specific to Gp100/pme117. In one embodiment, one or more T-cell subpopulation of one or more T-cell components is specific to tyrosinase. In one embodiment, one or more T-cell subpopulation of one or more T-cell components is specific to TRP1. In one embodiment, one or more T-cell subpopulation of one or more T-cell components is specific to TRP2. In one embodiment, one or more T-cell subpopulation of one or more T-cell components is specific to P. polypeptide. In one embodiment, one or more T-cell subpopulation of one or more T-cell components is specific to MC1R. In one embodiment, one or more T-cell subpopulation of one or more T-cell components is specific to prostate-specific antigen.

In one embodiment, one or more T-cell subpopulation of one or more T-cell components comprising the MUSTANG composition is specific to a mutated TAA selected from a group consisting of β-catenin, breast cancer antigen (BRCA) 1/2, cyclin-dependent kinase (CDK) 4, chronic myelogenous leukemia antigen (CML) 66, fibronectin, MART-2, p53, Ras, TGF-βRII, and truncated epithelial growth factor (tEGFR). In one embodiment, one or more T-cell subpopulation of one or more T-cell components is specific to β-catenin. In one embodiment, one or more T-cell subpopulation of one or more T-cell components is specific to BRCA1. In one embodiment, one or more T-cell subpopulation of one or more T-cell components is specific to BRCA2. In one embodiment, one or more T-cell subpopulation of one or more T-cell components is specific to CDK4. In one embodiment, one or more T-cell subpopulation of one or more T-cell components is specific to CML66. In one embodiment, one or more T-cell subpopulation of one or more T-cell components is specific to fibronectin. In one embodiment, one or more T-cell subpopulation of one or more T-cell components is specific to MART-2. In one embodiment, one or more T-cell subpopulation of one or more T-cell components is specific to p53. In one embodiment, one or more T-cell subpopulation of one or more T-cell components is specific to Ras. In one embodiment, one or more T-cell subpopulation of one or more T-cell components is specific to TGF-βRII. In one embodiment, one or more T-cell subpopulation of one or more T-cell components is specific to tEGFR.

In one embodiment, one or more T-cell subpopulation of one or more T-cell components comprising the MUSTANG composition is specific to the post-translationally altered TAA mucin (MUC) 1. In one embodiment, one or more T-cell subpopulation of one or more T-cell components is specific to MUC1.

In one embodiment, single antigen T-cell subpopulations are specific to an idiotypic TAA selected from a group consisting of immunoglobulin (Ig) and T cell receptor (TCR). In one embodiment, one or more T-cell subpopulation of one or more T-cell components is specific to Ig.

In one embodiment, one or more T-cell subpopulation of one or more T-cell components is specific to TCR.

### Generation of Targeted Tumor-Associated Antigen Peptides for Use in Activating T-Cell Subpopulations

T-cell subpopulations targeting a single TAA can be prepared by pulsing antigen presenting cells with a single peptide or epitope, several peptides or epitopes, or with peptide libraries of the selected antigen, that for example, include peptides that are about 7, 8, 9, 10, 11, 12, 13, 14, 15, 16 or more amino acids long and overlapping one another by about 5, 6, 7, 8, or 9 amino acids, in certain aspects. GMP-quality pepmixes directed to a number of tumor-associated antigens are commercially available, for example, through JPT Technologies and/or Miltenyi Biotec. In particular embodiments, the peptides are 7, 8, 9, 10, 11, 12, 13, 14, 15, 16, 17, 18, 19, 20, 21, 22, 23, 24, 25, 26, 27, 28, 29, 30, 31, 32, 33, 34, or 35 or more amino acids in length, for example, and there is overlap of 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acids in length.

In one embodiment, the T-cell subpopulation is specific to one or more known epitopes of the targeted single TAA. Much work has been done to determine specific epitopes of TAAs and the HLA alleles they are associated with. Non-limiting examples of specific epitopes of TAAs and the alleles they are associated with can be found in Kessler et al., J. Exp. Med. 193(1):73-88 (2001); Oka et al., Immunogenetics 51(2):99-107 (2000); Ohminami et al., Blood 95(1):286-93 (2000); Schmitz et al., Cancer Res. 60(17):4845-59 (2000); and Bachinsky et al., Cancer Immunol. Res. 5:6 (2005), which are each incorporated herein by reference.

In some embodiments, the TAA peptides used to prime and expand a T-cell subpopulation includes specifically selected HLA-restricted peptides generated by determining the HLA profile of the donor source, and including peptides derived from the targeted TAA that best match the donor's HLA type. By including specifically selected donor HLA-restricted peptides in the peptide mix for priming and expanding T-cell subpopulations, a T-cell subpopulation can be generated that provides greater TAA targeted activity through more than one donor HLA, improving potential efficacy of the T-cell subpopulation. In addition, by generating a T-cell subpopulation with TAA targeted activity through more than one donor HLA allele, a single donor

T-cell subpopulation may be included in a MUSTANG composition for multiple recipients with different HLA profiles by matching one or more donor HLAs showing TAA-activity. In some embodiments, the TAA peptides used to prime and expand a T-cell subpopulation are derived from HLA-restricted peptides selected from at least one or more of an HLA-A restricted peptide, HLA-B restricted peptide, or HLA-DR restricted peptide. In some embodiments, the HLA-restricted epitopes are specific to at least one or more of a cell donor's HLA-A alleles, HLA-B alleles, or HLA-DR alleles. In some embodiments, the HLA-A alleles are selected from a group comprising HLA-A*01, HLA-A*02:01, HLA-A*03, HLA-A*11:01, HLA-A*24:02, HLA-A*26, or HLA-A*68:01. In some embodiments, the HLA-B alleles are selected from a group comprising HLA-B*07:02, HLA-B*08, HLA-B*15:01 (B62), HLA-B*18, HLA-B*27:05, HLA-B*35:01, orHLA-B*58:02. In some embodiments, the HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b). Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595. In some embodiments, the mastermix of peptides includes both an overlapping peptide library and specifically selected HLA-restricted peptides generated by determining the HLA profile of the donor source.

This focused approach to activation can increase the effectiveness of the activated T-cell subpopulation, and ultimately, the lymphocytic cell composition. While the skilled artisan can enrich a peptide mixture with an epitope in a multi-tumor-associated antigen approach, this disclosure provides a new platform for optimizing therapy by targeted activation of T-cell subpopulations with peptides that are most likely to cause activation, and can each be tested for confirmation, prior to being combined in the lymphocytic cell composition.

WT-1 Antigenic Peptides

In some embodiments, the composition of the present disclosure includes WT-1 specific T-cells. WT1 specific T-cells can be generated as described below using one or more antigenic peptides to WT1. In some embodiments, the WT1 specific T-cells are generated using one or more antigenic peptides to WT1, or a modified or heteroclitic peptide derived from a WT1 peptide. In some embodiments, WT1 specific T-cells are generated using a WT1 antigen library comprising a pool of peptides (for example 15 mers) containing amino acid overlap (for example 11 amino acids of overlap) between each sequence formed by scanning the protein amino acid sequence SEQ ID NO: 1 (UniProtKB-P19544 (WT1_HUMAN)):

MGSDVRDLNALLPAVPSLGGGGGCALPVSGAAQWAPVLDFAPPGASAYGS
LGGPAPPPAPPPPPPPPPHSFIKQEPSWGGAEPHEEQCLSAFTVHFSGQF
TGTAGACRYGPFGPPPPSQASSGQARMFPNAPYLPSCLESQPAIRNQGYS
TVTEDGTPSYGHTPSHHAAQFPNHSFKHEDPMGQQGSLGEQQYSVPPPVY
GCHTPTDSCTGSQALLLRTPYSSDNLYQMTSQLECMTWNQMNLGATLKGV
AAGSSSSVKWTEGQSNHSTGYESDNHTTPILCGAQYRIHTHGVERGIQDV
RRVPGVAPTLVRSASETSEKRPFMCAYPGCNKRYFKLSHLQMHSRKHTGE
KPYQCDFKDCERRFSRSDQLKRHQRRHTGVKPFQCKTCQRKFSRSDHLKT
HTRTHTGKTSEKPFSCRWPSCQKKFARSDELVRHHNMHQRNMTKLQLAL

In some embodiments, the WT1 specific T-cells are generated using one or more antigenic peptides to WT1, or a modified or heteroclitic peptide derived from a WT1 peptide,

In some embodiments, the WT1 specific T-cells are generated using one or more antigenic peptides to WT1, or a modified or heteroclitic peptide derived from a WT1 peptide. In some embodiments, the WT1 specific T-cells are generated with peptides that recognize class I MHC molecules. In some embodiments, the WT1 specific T-cells are generated with peptides that recognize class II MHC molecules. In some embodiments, the WT1 specific T-cells are generated with peptides that recognize both class I and class II MHC molecules.

In some embodiments, the WT1 specific T-cells are generated with peptides that recognize class I MHC molecules. In some embodiments, the WT1 specific T-cells are generated with peptides that recognize class II MHC molecules. In some embodiments, the WT1 specific T-cells are generated with peptides that recognize both class I and class II MHC molecules.

In some embodiments, the WT1 peptides used to prime and expand a T-cell subpopulation includes specifically selected HLA-restricted peptides generated by determining the HLA profile of the donor source, and including peptides derived from WT1 that best match the donor's HLA. In some embodiments, the WT1 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting WT1 derived, wherein the T-cell subpopulation is primed and expanded using a group of peptides that are HLA-restricted to the donor's HLA profile. In certain embodiments, the T-cell subpopulation is exposed to a peptide mix that includes one ore more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 1-7 , the HLA-B peptides are selected from the peptides of Tables 8-14, and the HLA-DR peptides are selected from the peptides of Tables 15-20. For example, if the donor cell source has an HLA profile that is HLA-A*01/*02:01; HLA-B*15:01/*18; and HLA-DRB1*0101/*0301, then the WT1 peptides used to prime and expand the WT1 specific T-cell subpopulation are restricted to the specific HLA profile, and may include the peptides identified in Table 1 (SEQ ID NO: 2-11) for HLA-A*01; Table 2 (SEQ ID NO: 12-21) for HLA-A*02:01; Table 10 (SEQ ID NO: 92-101) for HLA-B*15:01; Table 11 (SEQ ID NO: 102-111) for HLA-B*18; Table 15 (SEQ ID NO: 142-151) for HLA-DRB1*0101; and Table 16 (SEQ ID NO: 152-159) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the WT1 targeted T-cell subpopulation is primed and expanded with one or more WT1-derived peptides selected from Table 1 (SEQ ID NO: 2-11). In some embodiments, the donor cell source is HLA-A*01, and the WT1 targeted T-cell subpopulation is primed and expanded with WT1-derived peptides selected from Table 1 (SEQ ID NO: 2-11). In some embodiments, the donor cell source is HLA-A*01, and the WT1 targeted T-cell subpopulation is primed and expanded with WT1-derived peptides comprising the peptides of Table 1 (SEQ ID NO: 2-11). In some embodiments, the donor cell source is HLA-A*01, and the WT1 targeted T-cell subpopulation is primed and expanded with WT1-derived peptides comprising the peptides of Table 1 (SEQ ID NO: 2-11) and at least one additional set of peptides based on the donor cell source HLA-A profile, wherein the at least one additional set of peptides are selected from the peptides of Tables 2-7. In some embodiments, the WT1-derived peptides also include one or more sets of HLA-B and HLA-DR restricted peptides selected from Tables 8-20 (SEQ ID NO: 72-198).

TABLE 1
WT1 HLA-A*01 Epitope Peptides

| SEQ ID NO: | Sequence |
|---|---|
| 2 | TSEKRPFMCAY |
| 3 | STVTFDGTPSY |
| 4 | HTTPILCGAQY |
| 5 | ESQPAIRNQGY |
| 6 | GSQALLLRTPY |
| 7 | HSRKHTGEKPY |
| 8 | FTGTAGACRY |
| 9 | RTPYSSDNLY |
| 10 | TTPILCGAQY |
| 11 | VTFDGTPSY |

In some embodiments, the donor cell source is HLA-A*02:01, and the WT1 targeted T-cell subpopulation is primed and expanded with one or more WT1-derived peptides selected from Table 2 (SEQ ID NO: 12-21). In some embodiments, the donor cell source is HLA-A*02:01, and the WT1 targeted T-cell subpopulation is primed and expanded with WT1-derived peptides selected from Table 2 (SEQ ID NO: 12-21). In some embodiments, the donor cell source is HLA-A*02:01, and the WT1 targeted T-cell subpopulation is primed and expanded with WT1-derived peptides comprising the peptides of Table 2 (SEQ ID NO: 12-21). In some embodiments, the donor cell source is HLA-A*02:01, and the WT1 targeted T-cell subpopulation is primed and expanded with WT1-derived peptides comprising the peptides of Table 2 (SEQ ID NO: 12-21) and at least one additional set of peptides based on the donor cell source HLA-A profile, wherein the at least one additional set of peptides are selected from the peptides of Tables 1, and 3-7. In some embodiments, the WT1-derived peptides also include one or more sets of HLA-B and HLA-DR restricted peptides selected from Tables 8-20 (SEQ ID NO: 72-198).

TABLE 2
WT1 HLA-A*02:01 Epitope Peptides

| SEQ ID NO: | Sequence |
|---|---|
| 12 | SLGGGGGCAL |
| 13 | NALLPAVPSL |
| 14 | AIRNQGYSTV |
| 15 | NMHQRNMTKL |
| 16 | ALLPAVPSL |
| 17 | DLNALLPAV |
| 18 | SLGEQQYSV |
| 19 | NLGATLKGV |
| 20 | NLYQMTSQL |
| 21 | ILCGAQYRI |

In some embodiments, the donor cell source is HLA-A*03, and the WT1 targeted T-cell subpopulation is primed and expanded with one or more WT1-derived peptides selected from Table 3 (SEQ ID NO: 22-31). In some embodiments, the donor cell source is HLA-A*03, and the WT1 targeted T-cell subpopulation is primed and expanded with WT1-derived peptides selected from Table 3 (SEQ ID NO: 22-31). In some embodiments, the donor cell source is HLA-A*03, and the WT1 targeted T-cell subpopulation is primed and expanded with WT1-derived peptides comprising the peptides of Table 3 (SEQ ID NO: 22-31). In some embodiments, the donor cell source is HLA-A*03, and the WT1 targeted T-cell subpopulation is primed and expanded with WT1-derived peptides comprising the peptides of Table 3 (SEQ ID NO: 22-31) and at least one additional set of peptides based on the donor cell source HLA-A profile, wherein the at least one additional set of peptides are selected from the peptides of Tables 1-2 and 4-7. In some embodiments, the WT1-derived peptides also include one or more sets of HLA-B and HLA-DR restricted peptides selected from Tables 8-20 (SEQ ID NO: 72-198) .

TABLE 3
WT1 HLA-A*03 Epitope Peptides

| SEQ ID NO: | Sequence |
|---|---|
| 22 | DVRRVPGVAP |
| 23 | ALLPAVPSLG |
| 24 | ALPVSGAAQW |
| 25 | AIRNQGYSTV |
| 26 | RHQRRHTGVK |
| 27 | GVFRGIQDVR |
| 28 | RVPGVAPTL |
| 29 | RIHTHGVFR |
| 30 | DVRRVPGVA |
| 31 | HQRRHTGVK |

In some embodiments, the donor cell source is HLA-A*11:01, and the WT1 targeted T-cell subpopulation is primed and expanded with one or more WT1-derived peptides selected from Table 4 (SEQ ID NO: 32-41). In some embodiments, the donor cell source is HLA-A*11:01, and the WT1 targeted T-cell subpopulation is primed and expanded with WT1-derived peptides selected from Table 4 (SEQ ID NO: 32-41). In some embodiments, the donor cell source is HLA-A*11:01, and the WT1 targeted T-cell subpopulation is primed and expanded with WT1-derived peptides comprising the peptides of Table 4 (SEQ ID NO: 32-41). In some embodiments, the donor cell source is HLA-A*11:01, and the WT1 targeted T-cell subpopulation is primed and expanded with WT1-derived peptides comprising the peptides of Table 4 (SEQ ID NO: 32-41) and at least one additional set of peptides based on the donor cell source HLA-A profile, wherein the at least one additional set of peptides are selected from the peptides of Tables 1-3 and 5-7. In some embodiments, the WT1-derived peptides also include one or more sets of HLA-B and HLA-DR restricted peptides selected from Tables 8-20 (SEQ ID NO: 72-198).

TABLE 4
WT1 HLA-A*11:01 Epitope Peptides

| SEQ ID NO: | Sequence |
|---|---|
| 32 | CTGSQALLLR |
| 33 | GVFRGIQDVR |
| 34 | HTGVKPFQCK |
| 35 | RTHTGKTSEK |
| 36 | KTHTRTHTGK |
| 37 | RSASETSEKR |
| 38 | LSHLQMHSRK |
| 39 | FSCRWPSCQK |
| 40 | RSASETSEK |
| 41 | FSRSDQLKR |

In some embodiments, the donor cell source is HLA-A*24:02, and the WT1 targeted T-cell subpopulation is primed and expanded with one or more WT1-derived peptides selected from Table 5 (SEQ ID NO: 42-51). In some embodiments, the donor cell source is HLA-A*24:02, and the WT1 targeted T-cell subpopulation is primed and expanded with WT1-derived peptides selected from Table 5 (SEQ ID NO: 42-51). In some embodiments, the donor cell source is HLA-A*24:02, and the WT1 targeted T-cell subpopulation is primed and expanded with WT1-derived peptides comprising the peptides of Table 5 (SEQ ID NO: 42-51). In some embodiments, the donor cell source is HLA-A*24:02, and the WT1 targeted T-cell subpopulation is primed and expanded with WT1-derived peptides comprising the peptides of Table 5 (SEQ ID NO: 42-51) and at least one additional set of peptides based on the donor cell source HLA-A profile, wherein the at least one additional set of peptides are selected from the peptides of Tables 1-4 and 6-7. In some embodiments, the WT1-derived peptides also include one or more sets of HLA-B and HLA-DR restricted peptides selected from Tables 8-20 (SEQ ID NO: 72-198).

TABLE 5
WT1 HLA-A*24:02 Epitope Peptides

| SEQ ID NO: | Sequence |
|---|---|
| 42 | AYPGCNKRYF |
| 43 | QYRIHTHGVF |
| 44 | AFTVHFSGQF |
| 45 | PPPPPPPHSF |
| 46 | PPPPPPHSFI |
| 47 | PYLPSCLESQ |
| 48 | DFKDCERRF |
| 49 | GCNKRYFKL |
| 50 | ALLPAVPSL |
| 51 | PPPPPPHSF |

In some embodiments, the donor cell source is HLA-A*26, and the WT1 targeted T-cell subpopulation is primed and expanded with one or more WT1-derived peptides selected from Table 6 (SEQ ID NO: 52-61). In some embodiments, the donor cell source is HLA-A*26, and the WT1 targeted T-cell subpopulation is primed and expanded with WT1-derived peptides selected from Table 6 (SEQ ID NO: 52-61). In some embodiments, the donor cell source is HLA-A*26, and the WT1 targeted T-cell subpopulation is primed and expanded with WT1-derived peptides comprising the peptides of Table 6 (SEQ ID NO: 52-61). In some embodiments, the donor cell source is HLA-A*26, and the WT1 targeted T-cell subpopulation is primed and expanded with WT1-derived peptides comprising the peptides of Table 6 (SEQ ID NO: 52-61) and at least one additional set of peptides based on the donor cell source HLA-A profile, wherein the at least one additional set of peptides are selected from the peptides of Tables 1-5 and 7. In some embodiments, the WT1-derived peptides also include one or more sets of HLA-B and HLA-DR restricted peptides selected from Tables 8-20 (SEQ ID NO: 72-198).

TABLE 6
WT1 HLA-A*26 Epitopes Peptides

| SEQ ID NO: | Sequence |
|---|---|
| 52 | TVTFDGTPSY |
| 53 | DFAPPGASAY |
| 54 | EGQSNHSTGY |
| 55 | TTPILCGAQY |
| 56 | ETSEKRPFMC |
| 57 | DVRDLNALL |
| 58 | VTFDGTPSY |
| 59 | FTVHFSGQF |
| 60 | EKRPFMCAY |
| 61 | ETSEKRPFM |

In some embodiments, the donor cell source is HLA-A*68:01, and the WT1 targeted T-cell subpopulation is primed and expanded with one or more WT1-derived peptides selected from Table 7 (SEQ ID NO: 62-71). In some embodiments, the donor cell source is HLA-A*68:01, and the WT1 targeted T-cell subpopulation is primed and expanded with WT1-derived peptides selected from Table 7 (SEQ ID NO: 62-71). In some embodiments, the donor cell source is HLA-A*68:01, and the WT1 targeted T-cell subpopulation is primed and expanded with WT1-derived peptides comprising the peptides of Table 7 (SEQ ID NO: 62-71). In some embodiments, the donor cell source is HLA-A*68:01, and the WT1 targeted T-cell subpopulation is primed and expanded with WT1-derived peptides comprising the peptides of Table 7 (SEQ ID NO: 62-71) and at least one additional set of peptides based on the donor cell source HLA-A profile, wherein the at least one additional set of peptides are selected from the peptides of Tables 1-6. In some embodiments, the WT1-derived peptides also include one or more sets of HLA-B and HLA-DR restricted peptides selected from Tables 8-20 (SEQ ID NO: 72-198).

TABLE 7
WT1 HLA-A*68:01 Epitope Peptides

| SEQ ID NO: | Sequence |
|---|---|
| 62 | GVFRGIQDVRR |
| 63 | TTPILCGAQYR |
| 64 | ELVRHHNMHQR |
| 65 | PSCLESQPAIR |
| 66 | CTGSQALLLR |
| 67 | GVFRGIQDVR |
| 68 | KTHTRTHTGK |
| 69 | LVRHHNMHQR |
| 70 | FTGTAGACR |
| 71 | RIHTHGVFR |

In some embodiments, the donor cell source is HLA-B*07:02, and the WT1 targeted T-cell subpopulation is primed and expanded with one or more WT1-derived peptides selected from Table 8 (SEQ ID NO: 72-81). In some embodiments, the donor cell source is HLA-B*07:02, and the WT1 targeted T-cell subpopulation is primed and expanded with WT1-derived peptides selected from Table 8 (SEQ ID NO: 72-81). In some embodiments, the donor cell source is HLA-B*07:02, and the WT1 targeted T-cell subpopulation is primed and expanded with WT1-derived peptides comprising the peptides of Table 8 (SEQ ID NO: 72-81). In some embodiments, the donor cell source is HLA-B*07:02, and the WT1 targeted T-cell subpopulation is primed and expanded with WT1-derived peptides comprising the peptides of Table 8 (SEQ ID NO: 72-81) and at least one additional set of peptides based on the donor cell source HLA-B profile, wherein the at least one additional set of peptides are selected from the peptides of Tables 9-14. In some embodiments, the WT1-derived peptides also include one or more sets of HLA-A and HLA-DR restricted peptides selected from Tables 1-7 and 15-20 (SEQ ID NO: 1-71 and 142-198).

TABLE 8
WT1 HLA-B*07:02 Epitope Peptides

| SEQ ID NO: | Sequence |
|---|---|
| 72 | PPGASAYGSL |
| 73 | EPHEEQCLSA |
| 74 | LPSCLESQPA |
| 75 | PPPPPPHSFI |
| 76 | PPSQASSGQA |
| 77 | DPMGQQGSL |
| 78 | PPPPPHSFI |
| 79 | PPPPPPHSF |
| 80 | TPSHHAAQF |
| 81 | WPSCQKKFA |

In some embodiments, the donor cell source is HLA-B*08, and the WT1 targeted T-cell subpopulation is primed and expanded with one or more WT1-derived peptides selected from Table 9 (SEQ ID NO: 82-91). In some embodiments, the donor cell source is HLA-B*08, and the WT1 targeted T-cell subpopulation is primed and expanded with WT1-derived peptides selected from Table 9 (SEQ ID NO: 82-91). In some embodiments, the donor cell source is HLA-B*08, and the WT1 targeted T-cell subpopulation is primed and expanded with WT1-derived peptides comprising the peptides of Table 9 (SEQ ID NO: 82-91). In some embodiments, the donor cell source is HLA-B*08, and the WT1 targeted T-cell subpopulation is primed and expanded with WT1-derived peptides comprising the peptides of Table 9 (SEQ ID NO: 82-91) and at least one additional set of peptides based on the donor cell source HLA-B profile, wherein the at least one additional set of peptides are selected from the peptides of Tables 8 and 10-14. In some embodiments, the WT1-derived peptides also include one or more sets of HLA-A and HLA-DR restricted peptides selected from Tables 1-7 and 15-20 (SEQ ID NO: 1-71 and 142-198).

TABLE 9
WT1 HLA-B*08 Epitope Peptides

| SEQ ID NO: | Sequence |
|---|---|
| 82 | KRYFKLSHL |
| 83 | GCNKRYFKL |
| 84 | KKFARSDEL |
| 85 | GATLKGVAA |
| 86 | RRFSRSDQL |
| 87 | MTKLQLAL |
| 88 | EPHEEQCL |
| 89 | ETSEKRPF |
| 90 | CNKRYFKL |
| 91 | RNMTKLQL |

In some embodiments, the donor cell source is HLA-B*15:01, and the WT1 targeted T-cell subpopulation is primed and expanded with one or more WT1-derived peptides selected from Table 10 (SEQ ID NO: 92-101). In some embodiments, the donor cell source is HLA-B*15:01, and the WT1 targeted T-cell subpopulation is primed and expanded with WT1-derived peptides selected from Table 10 (SEQ ID NO: 92-101). In some embodiments, the donor cell source is HLA-B*15:01, and the WT1 targeted T-cell subpopulation is primed and expanded with WT1-derived peptides comprising the peptides of Table 10 (SEQ ID NO: 92-101). In some embodiments, the donor cell source is HLA-B*15:01, and the WT1 targeted T-cell subpopulation is primed and expanded with WT1-derived peptides comprising the peptides of Table 10 (SEQ ID NO: 92-101) and at least one additional set of peptides based on the donor cell source HLA-B profile, wherein the at least one additional set of peptides are selected from the peptides of Tables 8-9 and 11-14. In some embodiments, the WT1-derived peptides also include one or more sets of HLA-A and HLA-DR restricted peptides selected from Tables 1-7 and 15-20 (SEQ ID NO: 1-71 and 142-198).

TABLE 10
WT1 HLA-B*15:01 (B62) Epitope Peptides

| SEQ ID NO: | Sequence |
|---|---|
| 92 | QQYSVPPPVY |
| 93 | TVTFDGTPSY |
| 94 | QQGSLGEQQY |
| 95 | SQALLLRTPY |
| 96 | SQPAIRNQGY |
| 97 | FQCKTCQRKF |
| 98 | AQWAPVLDF |
| 99 | GQSNHSTGY |
| 100 | NQGYSTVTF |
| 101 | CLSAFTVHF |

In some embodiments, the donor cell source is HLA-B*18, and the WT1 targeted T-cell subpopulation is primed and expanded with one or more WT1-derived peptides selected from Table 11 (SEQ ID NO: 102-111). In some embodiments, the donor cell source is HLA-B*18, and the WT1 targeted T-cell subpopulation is primed and expanded with WT1-derived peptides selected from Table 11 (SEQ ID NO: 102-111). In some embodiments, the donor cell source is HLA-B*18, and the WT1 targeted T-cell subpopulation is primed and expanded with WT1-derived peptides comprising the peptides of Table 11 (SEQ ID NO: 102-111). In some embodiments, the donor cell source is HLA-B*18, and the WT1 targeted T-cell subpopulation is primed and expanded with WT1-derived peptides comprising the peptides of Table 11 (SEQ ID NO: 102-111) and at least one additional set of peptides based on the donor cell source HLA-B profile, wherein the at least one additional set of peptides are selected from the peptides of Tables 8-10 and 12-14. In some embodiments, the WT1-derived peptides also include one or more sets of HLA-A and HLA-DR restricted peptides selected from Tables 1-7 and 15-20 (SEQ ID NO: 1-71 and 142-198).

TABLE 11
WT1 HLA-B*18 Epitope Peptides

| SEQ ID NO: | Sequence |
|---|---|
| 102 | HEEQCLSAF |
| 103 | SETSEKRPF |
| 104 | GEKPYQCDF |
| 105 | SEKPFSCRW |
| 106 | AEPHEEQCL |
| 107 | DVRDLNALL |
| 108 | QALLLRTPY |
| 109 | EEQCLSAF |
| 110 | ETSEKRPF |
| 111 | DELVRHHN |

In some embodiments, the donor cell source is HLA-B*27:05, and the WT1 targeted T-cell subpopulation is primed and expanded with one or more WT1-derived peptides selected from Table 12 (SEQ ID NO: 112-121). In some embodiments, the donor cell source is HLA-B*27:05, and the WT1 targeted T-cell subpopulation is primed and expanded with WT1-derived peptides selected from Table 12 (SEQ ID NO: 112-121). In some embodiments, the donor cell source is HLA-B*27:05, and the WT1 targeted T-cell subpopulation is primed and expanded with WT1-derived peptides comprising the peptides of Table 12 (SEQ ID NO: 112-121). In some embodiments, the donor cell source is HLA-B*27:05, and the WT1 targeted T-cell subpopulation is primed and expanded with WT1-derived peptides comprising the peptides of Table 12 (SEQ ID NO: 112-121) and at least one additional set of peptides based on the donor cell source HLA-B profile, wherein the at least one additional set of peptides are selected from the peptides of Tables 8-11 and 13-14. In some embodiments, the WT1-derived peptides also include one or more sets of HLA-A and HLA-DR restricted peptides selected from Tables 1-7 and 15-20 (SEQ ID NO: 1-71 and 142-198).

TABLE 12
WT1 HLA-B*27:05 Epitope Peptides

| SEQ ID NO: | Sequence |
|---|---|
| 112 | RRVPGVAPTL |
| 113 | RRFSRSDQLK |
| 114 | CRWPSCQKKF |
| 115 | LRTPYSSDNL |
| 116 | RRFSRSDQL |
| 117 | KRYFKLSHL |
| 118 | RRHTGVKPF |
| 119 | FRGIQDVRR |
| 120 | CRWPSCQKK |
| 121 | ARSDELVRH |

In some embodiments, the donor cell source is HLA-B*35:01, and the WT1 targeted T-cell subpopulation is primed and expanded with one or more WT1-derived peptides selected from Table 13 (SEQ ID NO: 122-131). In some embodiments, the donor cell source is HLA-B*35:01, and the WT1 targeted T-cell subpopulation is primed and expanded with WT1-derived peptides selected from Table 13 (SEQ ID NO: 122-131). In some embodiments, the donor cell source is HLA-B*35:01, and the WT1 targeted T-cell subpopulation is primed and expanded with WT1-derived peptides comprising the peptides of Table 13 (SEQ ID NO: 122-131). In some embodiments, the donor cell source is HLA-B*35:01, and the WT1 targeted T-cell subpopulation is primed and expanded with WT1-derived peptides comprising the peptides of Table 13 (SEQ ID NO: 122-131) and at least one additional set of peptides based on the donor cell source HLA-B profile, wherein the at least one additional set of peptides are selected from the peptides of Tables 8-12 and 14. In some embodiments, the WT1-derived peptides also include one or more sets of HLA-A and HLA-DR restricted peptides selected from Tables 1-7 and 15-20 (SEQ ID NO: 1-71 and 142-198).

TABLE 13
WT1 HLA-B*35:01 Epitope Peptides

| SEQ ID NO: | Sequence |
|---|---|
| 122 | PPGASAYGSL |
| 123 | PPPPPPPHSF |
| 124 | PPPPPPHSFI |
| 125 | TPYSSDNLY |
| 126 | QPAIRNQGY |
| 127 | DPMGQQGSL |
| 128 | TPILCGAQY |
| 129 | TPSHHAAQF |
| 130 | PPPPPPHSF |
| 131 | YPGCNKRYF |

In some embodiments, the donor cell source is HLA-B*58:02, and the WT1 targeted T-cell subpopulation is primed and expanded with one or more WT1-derived peptides selected from Table 14 (SEQ ID NO: 132-141). In some embodiments, the donor cell source is HLA-B*58:02, and the WT1 targeted T-cell subpopulation is primed and expanded with WT1-derived peptides selected from Table 14 (SEQ ID NO: 132-141). In some embodiments, the donor cell source is HLA-B*58:02, and the WT1 targeted T-cell subpopulation is primed and expanded with WT1-derived peptides comprising the peptides of Table 14 (SEQ ID NO: 132-141). In some embodiments, the donor cell source is HLA-B*58:02, and the WT1 targeted T-cell subpopulation is primed and expanded with WT1-derived peptides comprising the peptides of Table 14 (SEQ ID NO: 132-141) and at least one additional set of peptides based on the donor cell source HLA-B profile, wherein the at least one additional set of peptides are selected from the peptides of Tables 8-13. In some embodiments, the WT1-derived peptides also include one or more sets of HLA-A and HLA-DR restricted peptides selected from Tables 1-7 and 15-20 (SEQ ID NO: 1-71 and 142-198).

TABLE 14
WT1 HLA-B*58:02 Epitope Peptides

| SEQ ID NO: | Sequence |
|---|---|
| 132 | ASETSEKRPF |
| 133 | QASSGQARMF |
| 134 | RTPYSSDNLY |
| 135 | DSCTGSQALL |
| 136 | ASSGQARMF |
| 137 | RVPGVAPTL |
| 138 | TSQLECMTW |
| 139 | HTHGVFRGI |
| 140 | RTPYSSDNL |
| 141 | RSDELVRHH |

In some embodiments, the donor cell source is HLA-DRB1*0101, and the WT1 targeted T-cell subpopulation is primed and expanded with one or more WT1-derived peptides selected from Table 15 (SEQ ID NO: 142-151). In some embodiments, the donor cell source is HLA-DRB1*0101, and the WT1 targeted T-cell subpopulation is primed and expanded with WT1-derived peptides selected from Table 15(SEQ ID NO: 142-151). In some embodiments, the donor cell source is HLA-DRB1*0101, and the WT1 targeted T-cell subpopulation is primed and expanded with WT1-derived peptides comprising the peptides of Table 15 (SEQ ID NO: 142-151). In some embodiments, the donor cell source is HLA-DRB1*0101, and the WT1 targeted T-cell subpopulation is primed and expanded with WT1-derived peptides comprising the peptides of Table 15 (SEQ ID NO: 142-151) and at least one additional set of peptides based on the donor cell source HLA-DR profile, wherein the at least one additional set of peptides are selected from the peptides of Tables 16-20. In some embodiments, the WT1-derived peptides also include one or more sets of HLA-A and HLA-B restricted peptides selected from Tables 1-14 (SEQ ID NO: 1-141).

TABLE 15
WT1 HLA-DRB1*0101 Epitope Peptides

| SEQ ID NO: | Sequence |
|---|---|
| 142 | ASAYGSLGGPAPPPA |
| 143 | GSDVRDLNALLPAVP |
| 144 | IQDVRRVPGVAPTLV |
| 145 | VRDLNALLPAVPSLG |
| 146 | GATLKGVAAGSSSSV |
| 147 | TVHFSGQFTGTAGAC |
| 148 | VRRVPGVAPTLVRSA |
| 149 | NKRYFKLSHLQMHSR |
| 150 | LPAVPSLGGGGGCAL |
| 151 | RDLNALLPAVPSLGG |

In some embodiments, the donor cell source is HLA-DRB1*0301, and the WT1 targeted T-cell subpopulation is primed and expanded with one or more WT1-derived peptides selected from Table 16 (SEQ ID NO: 152-159). In some embodiments, the donor cell source is HLA-DRB1*0301, and the WT1 targeted T-cell subpopulation is primed and expanded with WT1-derived peptides selected from Table 16 (SEQ ID NO: 152-159). In some embodiments, the donor cell source is HLA-DRB1*0301, and the WT1 targeted T-cell subpopulation is primed and expanded with WT1-derived peptides comprising the peptides of Table 16 (SEQ ID NO: 152-159). In some embodiments, the donor cell source is HLA-DRB1*0301, and the WT1 targeted T-cell subpopulation is primed and expanded with WT1-derived peptides comprising the peptides of Table 16 (SEQ ID NO: 152-159) and at least one additional set of peptides based on the donor cell source HLA-DR profile, wherein the at least one additional set of peptides are selected from the peptides of Tables 15 and 17-20. In some embodiments, the WT1-derived peptides also include one or more sets of HLA-A and HLA-B restricted peptides selected from Tables 1-14 (SEQ ID NO: 1-141).

TABLE 16
WT1 HLA-DRB1*0301 Epitope Peptides

| SEQ ID NO: | Sequence |
|---|---|
| 152 | YSTVTFDGTPSYGHT |
| 153 | MGSDVRDLNALLPAV |
| 154 | YQCDFKDCERRFSRS |
| 155 | VPSLGGGGGCALPVS |
| 156 | VLDFAPPGASAYGSL |
| 157 | LYQMTSQLECMTWNQ |
| 158 | PTLVRSASETSEKRP |
| 159 | HHNMHQRNMTKLQLA |

In some embodiments, the donor cell source is HLA-DRB1*0401, and the WT1 targeted T-cell subpopulation is primed and expanded with one or more WT1-derived peptides selected from Table 17 (SEQ ID NO: 160-169). In some embodiments, the donor cell source is HLA-DRB1*0401, and the WT1 targeted T-cell subpopulation is primed and expanded with WT1-derived peptides selected from Table 17 (SEQ ID NO: 160-169). In some embodiments, the donor cell source is HLA-DRB1*0401, and the WT1 targeted T-cell subpopulation is primed and expanded with WT1-derived peptides comprising the peptides of Table 17 (SEQ ID NO: 160-169). In some embodiments, the donor cell source is HLA-DRB1*0401, and the WT1 targeted T-cell subpopulation is primed and expanded with WT1-derived peptides comprising the peptides of Table 17 (SEQ ID NO: 160-169) and at least one additional set of peptides based on the donor cell source HLA-DR profile, wherein the at least one additional set of peptides are selected from the peptides of Tables 15-16 and 18-20. In some embodiments, the WT1-derived peptides also include one or more sets of HLA-A and HLA-B restricted peptides selected from Tables 1-14 (SEQ ID NO: 1-141).

TABLE 17
WT1 HLA-DRB1*0401 (DR4Dw4) Epitope Peptides

| SEQ ID NO: | Sequence |
|---|---|
| 160 | NKRYFKLSHLQMHSR |
| 161 | TVHFSGQFTGTAGAC |
| 162 | ARMFPNAPYLPSCLE |
| 163 | NQGYSTVTFDGTPSY |
| 164 | TPSYGHTPSHHAAQF |
| 165 | NHSFKHEDPMGQQGS |
| 166 | RTPYSSDNLYQMTSQ |
| 167 | SVKWTEGQSNHSTGY |
| 168 | STGYESDNHTTPILC |
| 169 | KRPFMCAYPGCNKRY |

In some embodiments, the donor cell source is HLA-DRB1*0701, and the WT1 targeted T-cell subpopulation is primed and expanded with one or more WT1-derived peptides selected from Table 18 (SEQ ID NO: 170-179). In some embodiments, the donor cell source is HLA-DRB1*0701, and the WT1 targeted T-cell subpopulation is primed and expanded with WT1-derived peptides selected from Table 18 (SEQ ID NO: 170-179). In some embodiments, the donor cell source is HLA-DRB1*0701, and the WT1 targeted T-cell subpopulation is primed and expanded with WT1-derived peptides comprising the peptides of Table 18 (SEQ ID NO: 170-179). In some embodiments, the donor cell source is HLA-DRB1*0701, and the WT1 targeted T-cell subpopulation is primed and expanded with WT1-derived peptides comprising the peptides of Table 18 (SEQ ID NO: 170-179) and at least one additional set of peptides based on the donor cell source HLA-DR profile, wherein the at least one additional set of peptides are selected from the peptides of Tables 15-17 and 19-20. In some embodiments, the WT1-derived peptides also include one or more sets of HLA-A and HLA-B restricted peptides selected from Tables 1-14 (SEQ ID NO: 1-141).

TABLE 18
WT1 HLA-DRB1*0701 Epitope Peptides

| SEQ ID NO: | Sequence |
|---|---|
| 170 | TPSYGHTPSHHAAQF |
| 171 | TVTFDGTPSYGHTPS |
| 172 | LSAFTVHFSGQFTGT |
| 173 | TPTDSCTGSQALLLR |
| 174 | LKGVAAGSSSSVKWT |
| 175 | TVHFSGQFTGTAGAC |
| 176 | YSTVTFDGTPSYGHT |
| 177 | CGAQYRIHTHGVFRG |
| 178 | HGVFRGIQDVRRVPG |
| 179 | APTLVRSASETSEKR |

In some embodiments, the donor cell source is HLA-DRB1*1101, and the WT1 targeted T-cell subpopulation is primed and expanded with one or more WT1-derived peptides selected from Table 19 (SEQ ID NO: 180-188). In some embodiments, the donor cell source is HLA-DRB1*1101, and the WT1 targeted T-cell subpopulation is primed and expanded with WT1-derived peptides selected from Table 19 (SEQ ID NO: 180-188). In some embodiments, the donor cell source is HLA-DRB1*1101, and the WT1 targeted T-cell subpopulation is primed and expanded with WT1-derived peptides comprising the peptides of Table 19 (SEQ ID NO: 180-188). In some embodiments, the donor cell source is HLA-DRB1*1101, and the WT1 targeted T-cell subpopulation is primed and expanded with WT1-derived peptides comprising the peptides of Table 19 (SEQ ID NO: 180-188) and at least one additional set of peptides based on the donor cell source HLA-DR profile, wherein the at least one additional set of peptides are selected from the peptides of Tables 15-18 and 20. In some embodiments, the WT1-derived peptides also include one or more sets of HLA-A and HLA-B restricted peptides selected from Tables 1-14 (SEQ ID NO: 1-141).

TABLE 19
WT1 HLA-DRB1*1101 Epitope Peptides

| SEQ ID NO: | Sequence |
|---|---|
| 180 | FRGIQDVRRVPGVAP |
| 181 | NKRYFKLSHLQMHSR |
| 182 | QCDFKDCERRFSRSD |
| 183 | STGYESDNHTTPILC |
| 184 | SCRWPSCQKKFARSD |
| 185 | AAQWAPVLDFAPPGA |
| 186 | ASAYGSLGGPAPPPA |
| 187 | PGVAPTLVRSASETS |
| 188 | QMNLGATLKGVAAGS |

In some embodiments, the donor cell source is HLA-DRB1*1501, and the WT1 targeted T-cell subpopulation is primed and expanded with one or more WT1-derived peptides selected from Table 20 (SEQ ID NO: 189-198). In some embodiments, the donor cell source is HLA-DRB1*1501, and the WT1 targeted T-cell subpopulation is primed and expanded with WT1-derived peptides selected from Table 20 (SEQ ID NO: 189-198). In some embodiments, the donor cell source is HLA-DRB1*1501, and the WT1 targeted T-cell subpopulation is primed and expanded with WT1-derived peptides comprising the peptides of Table 20 (SEQ ID NO: 189-198). In some embodiments, the donor cell source is HLA-DRB1*1501, and the WT1 targeted T-cell subpopulation is primed and expanded with WT1-derived peptides comprising the peptides of Table 20 (SEQ ID NO: 189-198) and at least one additional set of peptides based on the donor cell source HLA-DR profile, wherein the at least one additional set of peptides are selected from the peptides of Tables 15-19. In some embodiments, the WT1-derived peptides also include one or more sets of HLA-A and HLA-B restricted peptides selected from Tables 1-14 (SEQ ID NO: 1-141).

TABLE 20
WT1 HLA-DRB1*1501 (DR2b) Epitope Peptides

| SEQ ID NO: | Sequence |
|---|---|
| 189 | WAPVLDFAPPGASAY |
| 190 | RPFMCAYPGCNKRYF |
| 191 | GSDVRDLNALLPAVP |
| 192 | NALLPAVPSLGGGGG |
| 193 | PPGASAYGSLGGPAP |
| 194 | EQCLSAFTVHFSGQF |
| 195 | TAGACRYGPFGPPPP |
| 196 | PSCLESQPAIRNQGY |
| 197 | WNQMNLGATLKGVAA |
| 198 | IQDVRRVPGVAPTLV |

PRAME Antigenic Peptides

In some embodiments, the MUSTANG composition includes PRAME specific T-cells. PRAME specific T-cells can be generated as described below using one or more antigenic peptides to PRAME. In some embodiments, the PRAME specific T-cells are generated using one or more antigenic peptides to PRAME, or a modified or heteroclitic peptide derived from a PRAME peptide. In some embodiments, PRAME specific T-cells are generated using a PRAME antigen library comprising a pool of peptides (for example 15 mers) containing amino acid overlap (for example 11 amino acids of overlap) between each sequence formed by scanning the protein amino acid sequence SEQ ID NO: 199 (UniProt KB-P78395) for human melanoma antigen preferentially expressed in tumors (PRAME):

MERRRLWGSIQSRYISMSVWTSPRRLVELAGQSLLKDEALAIAALELLPR
ELFPPLFMAAFDGRHSQTLKAMVQAWPFTCLPLGVLMKGQHLHLETFKAV
LDGLDVLLAQEVRPRRWKLQVLDLRKNSHQDFWTVWSGNRASLYSFPEPE
AAQPMTKKRKVDGLSTEAEQPFIPVEVLVDLFLKEGACDELFSYLIEKVK
RKKNVLRLCCKKLKIFAMPMQDIKMILKMVQLDSIEDLEVTCTWKLPTLA
KFSPYLGQMINLRRLLLSHIHASSYISPEKEEQYIAQFTSQFLSLQCLQA
LYVDSLFFLRGRLDQLLRHVMNPLETLSITNCRLSEGDVMHLSQSPSVSQ
LSVLSLSGVMLTDVSPEPLQALLERASATLQDLVFDECGITDDQLLALLP
SLSHCSQLTTLSFYGNSISISALQSLLQHLIGLSNLTHVLYPVPLESYED
IHGTLHLERLAYLHARLRELLCELGRPSMVWLSANPCPHCGDRTFYDPEP
ILCPCFMPN

Overlapping antigenic libraries are commercially available, for example, from JPT (Product code: PM-OIP4 Pep Mix™ Human (Prame/OIP4)). In some embodiments, the PRAME specific T-cells are generated using a commercially available overlapping antigenic library made up of PRAME peptides.

In some embodiments, the PRAME specific T-cells are generated using one or more antigenic peptides to PRAME, or a modified or heteroclitic peptide derived from a PRAME peptide. In some embodiments, the PRAME specific T-cells are generated with peptides that recognize class I MHC molecules. In some embodiments, the PRAME specific T-cells are generated with peptides that recognize class II MEW molecules. In some embodiments, the PRAME specific T-cells are generated with peptides that recognize both class I and class II MEW molecules.

In some embodiments, the PRAME peptides used to prime and expand a T-cell subpopulation includes specifically selected HLA-restricted peptides generated by determining the HLA profile of the donor source, and including peptides derived from PRAME that best match the donor's HLA. In some embodiments, the PRAME peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting PRAME derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 21-27 , the HLA-B peptides are selected from the peptides of Tables 28-34, and the HLA-DR peptides are selected from the peptides of Tables 35-40. For example, if the donor cell source has an HLA profile that is HLA-A*01/*02:01; HLA-B*15:01/*18; and HLA-DRB1*0101/*0301, then the PRAME peptides used to prime and expand the PRAME specific T-cell subpopulation are restricted to the specific HLA profile, and may include the peptides identified in Table 21 (SEQ ID NO: 200-209) for HLA-A*01; Table 22 (SEQ ID NO: 210-219) for HLA-A*02:01; Table 30 (SEQ ID NO: 289-298) for HLA-B*15:01; Table 31 (SEQ ID NO: 299-308) for HLA-B*18; Table 35 (SEQ ID NO: 339-348) for HLA-DRB1*0101; and Table 36 (SEQ ID NO: 349-358) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the PRAME targeted T-cell subpopulation is primed and expanded with one or more PRAME-derived peptides selected from Table 21 (SEQ ID NO: 200-209). In some embodiments, the donor cell source is HLA-A*01, and the PRAME targeted T-cell subpopulation is primed and expanded with PRAME-derived peptides selected from Table 21 (SEQ ID NO: 200-209). In some embodiments, the donor cell source is HLA-A*01, and the PRAME targeted T-cell subpopulation is primed and expanded with PRAME-derived peptides comprising the peptides of Table 21 (SEQ ID NO: 200-209). In some embodiments, the donor cell source is HLA-A*01, and the PRAME targeted T-cell subpopulation is primed and expanded with PRAME-derived peptides comprising the peptides of Table 21 (SEQ ID NO: 200-209) and at least one additional set of peptides based on the donor cell source HLA-A profile, wherein the at least one additional set of peptides are selected from the peptides of Tables 22-27. In some embodiments, the PRAME-derived peptides also include one or more sets of HLA-B and HLA-DR restricted peptides selected from Tables 28-40 (SEQ ID NO: 269-398).

TABLE 21
PRAME HLA-A*01 Epitope Peptides

| SEQ ID NO: | Sequence |
|---|---|
| 200 | LTDVSPEPLQA |
| 201 | ITDDQLLALLP |
| 202 | HGTLHLERLAY |
| 203 | GTLHLERLAY |
| 204 | CSQLTTLSFY |
| 205 | LSLQCLQALY |
| 206 | PTLAKFSPY |
| 207 | LSNLTHVLY |
| 208 | WSGNRASLY |
| 209 | LSHIHASSY |

In some embodiments, the donor cell source is HLA-A*02:01, and the PRAME targeted T-cell subpopulation is primed and expanded with one or more PRAME-derived peptides selected from Table 22 (SEQ ID NO: 210-219). In some embodiments, the donor cell source is HLA-A*02:01, and the PRAME targeted T-cell subpopulation is primed and expanded with PRAME-derived peptides selected from Table 22 (SEQ ID NO: 210-219). In some embodiments, the donor cell source is HLA-A*02:01, and the PRAME targeted T-cell subpopulation is primed and expanded with PRAME-derived peptides comprising the peptides of Table 22 (SEQ ID NO: 210-219). In some embodiments, the donor cell source is HLA-A*02:01, and the PRAME targeted T-cell subpopulation is primed and expanded with PRAME-derived peptides comprising the peptides of Table 22 (SEQ ID NO: 210-219) and at least one additional set of peptides based on the donor cell source HLA-A profile, wherein the at least one additional set of peptides are selected from the peptides of Tables 21, and 23-27. In some embodiments, the PRAME-derived peptides also include one or more sets of HLA-B and HLA-DR restricted peptides selected from Tables 28-40 (SEQ ID NO: 269-398).

TABLE 22
PRAME HLA-A*02:01 Epitope Peptides

| SEQ ID NO: | Sequence |
|---|---|
| 210 | ALLERASATL |
| 211 | ALAIAALELL |
| 212 | SLSGVMLTDV |
| 213 | ALYVDSLFFL |
| 214 | QLLALLPSL |
| 215 | SLLQHLIGL |
| 216 | RLRELLCEL |
| 217 | YLHARLREL |
| 218 | ALAIAALEL |
| 219 | FLRGRLDQL |

In some embodiments, the donor cell source is HLA-A*03, and the PRAME targeted T-cell subpopulation is primed and expanded with one or more PRAME-derived peptides selected from Table 23 (SEQ ID NO: 220-229). In some embodiments, the donor cell source is HLA-A*03, and the PRAME targeted T-cell subpopulation is primed and expanded with PRAME-derived peptides selected from Table 23 (SEQ ID NO: 220-229). In some embodiments, the donor cell source is HLA-A*03, and the PRAME targeted T-cell subpopulation is primed and expanded with PRAME-derived peptides comprising the peptides of Table 23 (SEQ ID NO: 220-229). In some embodiments, the donor cell source is HLA-A*03, and the PRAME targeted T-cell subpopulation is primed and expanded with PRAME-derived peptides comprising the peptides of Table 23 (SEQ ID NO: 220-229) and at least one additional set of peptides based on the donor cell source HLA-A profile, wherein the at least one additional set of peptides are selected from the peptides of Tables 21-22 and 24-27. In some embodiments, the PRAME-derived peptides also include one or more sets of HLA-B and HLA-DR restricted peptides selected from Tables 28-40 (SEQ ID NO: 269-398).

TABLE 23
PRAME HLA-A*03 Epitope Peptides

| SEQ ID NO: | Sequence |
|---|---|
| 220 | HLIGLSNLTH |
| 221 | RLWGSIQSRY |
| 222 | KVKRKKNVLR |
| 223 | VLYPVPLESY |
| 224 | CLPLGVLMK |
| 225 | ELAGQSLLK |
| 226 | KLQVLDLRK |
| 227 | RLSEGDVMH |
| 228 | YLIEKVKRK |
| 229 | NVLRLCCKK |

In some embodiments, the donor cell source is HLA-A*11:01, and the PRAME targeted T-cell subpopulation is primed and expanded with one or more PRAME-derived peptides selected from Table 24 (SEQ ID NO: 230-239). In some embodiments, the donor cell source is HLA-A*11:01, and the PRAME targeted T-cell subpopulation is primed and expanded with PRAME-derived peptides selected from Table 24 (SEQ ID NO: 230-239). In some embodiments, the donor cell source is HLA-A*11:01, and the PRAME targeted T-cell subpopulation is primed and expanded with PRAME-derived peptides comprising the peptides of Table 24 (SEQ ID NO: 230-239). In some embodiments, the donor cell source is HLA-A*11:01, and the PRAME targeted T-cell subpopulation is primed and expanded with PRAME-derived peptides comprising the peptides of Table 24 (SEQ ID NO: 230-239), and at least one additional set of peptides based on the donor cell source HLA-A profile, wherein the at least one additional set of peptides are selected from the peptides of Tables 21-23 and 25-27. In some embodiments, the PRAME-derived peptides also include one or more sets of HLA-B and HLA-DR restricted peptides selected from Tables 28-40 (SEQ ID NO: 269-398).

TABLE 24
PRAME HLA-A*11:01 Epitope Peptides

| SEQ ID NO: | Sequence |
|---|---|
| 230 | KVKRKKNVLR |
| 231 | PMQDIKMILK |
| 232 | CTWKLPTLAK |
| 233 | AIAALELLPR |
| 234 | AVLDGLDVLL |
| 235 | FSYLIEKVKR |
| 236 | ELAGQSLLK |
| 237 | EVLVDLFLK |
| 238 | ASSYISPEK |
| 239 | ELFSYLIEK |

In some embodiments, the donor cell source is HLA-A*24:02, and the PRAME targeted T-cell subpopulation is primed and expanded with one or more PRAME-derived peptides selected from Table 25 (SEQ ID NO: 240-249). In some embodiments, the donor cell source is HLA-A*24:02, and the PRAME targeted T-cell subpopulation is primed and expanded with PRAME-derived peptides selected from Table 25 (SEQ ID NO: 240-249). In some embodiments, the donor cell source is HLA-A*24:02, and the PRAME targeted T-cell subpopulation is primed and expanded with PRAME-derived peptides comprising the peptides of Table 25 (SEQ ID NO: 240-249). In some embodiments, the donor cell source is HLA-A*24:02, and the PRAME targeted T-cell subpopulation is primed and expanded with PRAME-derived peptides comprising the peptides of Table 25 (SEQ ID NO: 240-249), and at least one additional set of peptides based on the donor cell source HLA-A profile, wherein the at least one additional set of peptides are selected from the peptides of Tables 21-24 and 26-27. In some embodiments, the PRAME-derived peptides also include one or more sets of HLA-B and HLA-DR restricted peptides selected from Tables 28-40 (SEQ ID NO: 269-398).

TABLE 25
PRAME HLA-A*24:02 Epitope Peptides

| SEQ ID NO: | Sequence |
|---|---|
| 240 | QYIAQFTSQF |
| 241 | AYLHARLREL |
| 242 | LFPPLFMAAF |
| 243 | KFSPYLGQMI |
| 244 | FFLRGRLDQL |
| 245 | VSPEPLQALL |
| 246 | SYEDIHGTL |
| 247 | PYLGQMINL |
| 248 | LYVDSLFFL |
| 249 | TFYDPEPIL |

In some embodiments, the donor cell source is HLA-A*26, and the PRAME targeted T-cell subpopulation is primed and expanded with one or more PRAME-derived peptides selected from Table 26 (SEQ ID NO: 250-258). In some embodiments, the donor cell source is HLA-A*26, and the PRAME targeted T-cell subpopulation is primed and expanded with PRAME-derived peptides selected from Table 26 (SEQ ID NO: 250-258). In some embodiments, the donor cell source is HLA-A*26, and the PRAME targeted T-cell subpopulation is primed and expanded with PRAME-derived peptides comprising the peptides of Table 26 (SEQ ID NO: 250-258). In some embodiments, the donor cell source is HLA-A*26, and the PRAME targeted T-cell subpopulation is primed and expanded with PRAME-derived peptides comprising the peptides of Table 26 (SEQ ID NO: 250-258) and at least one additional set of peptides based on the donor cell source HLA-A profile, wherein the at least one additional set of peptides are selected from the peptides of Tables 21-25 and 27. In some embodiments, the PRAME-derived peptides also include one or more sets of HLA-B and HLA-DR restricted peptides selected from Tables 28-40 (SEQ ID NO: 269-398).

TABLE 26
PRAME HLA-A*26 Epitope Peptides

| SEQ ID NO: | Sequence |
|---|---|
| 250 | ETFKAVLDGL |
| 251 | DVSPEPLQAL |
| 252 | ETLSITNCRL |
| 253 | EGACDELFSY |
| 254 | EKEEQYIAQF |
| 255 | SVSQLSVLSL |
| 256 | EVRPRRWKL |
| 257 | ETFKAVLDG |
| 258 | EVLVDLFLK |

In some embodiments, the donor cell source is HLA-A*68:01, and the PRAME targeted T-cell subpopulation is primed and expanded with one or more PRAME-derived peptides selected from Table 27 (SEQ ID NO: 259-268). In some embodiments, the donor cell source is HLA-A*68:01, and the PRAME targeted T-cell subpopulation is primed and expanded with PRAME-derived peptides selected from Table 27 (SEQ ID NO: 259-268). In some embodiments, the donor cell source is HLA-A*68:01, and the PRAME targeted T-cell subpopulation is primed and expanded with PRAME-derived peptides comprising the peptides of Table 27 (SEQ ID NO: 259-268). In some embodiments, the donor cell source is HLA-A*68:01, and the PRAME targeted T-cell subpopulation is primed and expanded with PRAME-derived peptides comprising the peptides of Table 27 (SEQ ID NO: 259-268), and at least one additional set of peptides based on the donor cell source HLA-A profile, wherein the at least one additional set of peptides are selected from the peptides of Tables 21-26. In some embodiments, the PRAME-derived peptides also include one or more sets of HLA-B and HLA-DR restricted peptides selected from Tables 28-40 (SEQ ID NO: 269-398).

TABLE 27
PRAME HLA-A*68:01 Epitope Peptides

| SEQ ID NO: | Sequence |
|---|---|
| 259 | DVLLAQEVRPR |
| 260 | EAAQPMTKKR |
| 261 | KVKRKKNVLR |
| 262 | EAAQPMTKK |
| 263 | EVLVDLFLK |
| 264 | ELFSYLIEK |
| 265 | ETLSITNCR |
| 266 | DVLLAQEVR |
| 267 | DSLFFLRGR |
| 268 | IAALELLPR |

In some embodiments, the donor cell source is HLA-B*07:02, and the PRAME targeted T-cell subpopulation is primed and expanded with one or more PRAME-derived peptides selected from Table 28 (SEQ ID NO: 269-278). In some embodiments, the donor cell source is HLA-B*07:02, and the PRAME targeted T-cell subpopulation is primed and expanded with PRAME-derived peptides selected from Table 28 (SEQ ID NO: 269-278). In some embodiments, the donor cell source is HLA-B*07:02, and the PRAME targeted T-cell subpopulation is primed and expanded with PRAME-derived peptides comprising the peptides of Table 28 (SEQ ID NO: 269-278). In some embodiments, the donor cell source is HLA-B*07:02, and the PRAME targeted T-cell subpopulation is primed and expanded with PRAME-derived peptides comprising the peptides of Table 28 (SEQ ID NO: 269-278), and at least one additional set of peptides based on the donor cell source HLA-B profile, wherein the at least one additional set of peptides are selected from the peptides of Tables 29-34. In some embodiments, the PRAME-derived peptides also include one or more sets of HLA-A and HLA-DR restricted peptides selected from Tables 21-27 and 35-40 (SEQ ID NO: 200-268 and 339-398).

TABLE 28
PRAME HLA-B*07:02 Epitope Peptides

| SEQ ID NO: | Sequence |
|---|---|
| 269 | RPRRWKLQVL |
| 270 | SPSVSQLSVL |
| 271 | LPSLSHCSQL |
| 272 | MPMQDIKMIL |
| 273 | LPRELFPPL |
| 274 | QPFIPVEVL |
| 275 | IPVEVLVDL |
| 276 | SPEPLQALL |
| 277 | RPRRWKLQV |
| 278 | RPSMVWLSA |

In some embodiments, the donor cell source is HLA-B*08, and the PRAME targeted T-cell subpopulation is primed and expanded with one or more PRAME-derived peptides selected from Table 29 (SEQ ID NO: 279-288). In some embodiments, the donor cell source is HLA-B*08, and the PRAME targeted T-cell subpopulation is primed and expanded with PRAME-derived peptides selected from Table 29 (SEQ ID NO: 279-288). In some embodiments, the donor cell source is HLA-B*08, and the PRAME targeted T-cell subpopulation is primed and expanded with PRAME-derived peptides comprising the peptides of Table 29 (SEQ ID NO: 279-288). In some embodiments, the donor cell source is HLA-B*08, and the PRAME targeted T-cell subpopulation is primed and expanded with PRAME-derived peptides comprising the peptides of Table 29 (SEQ ID NO: 279-288) and at least one additional set of peptides based on the donor cell source HLA-B profile, wherein the at least one additional set of peptides are selected from the peptides of Tables 28 and 30-34. In some embodiments, the PRAME-derived peptides also include one or more sets of HLA-A and HLA-DR restricted peptides selected from Tables 21-27 and 35-40 (SEQ ID NO: 200-268 and 339-398).

TABLE 29
PRAME HLA-B*08 Epitope Peptides

| SEQ ID NO: | Sequence |
|---|---|
| 279 | TKKRKVDGL |
| 280 | FLRGRLDQL |
| 281 | KVKRKKNVL |
| 282 | EVRPRRWKL |
| 283 | PRRWKLQVL |
| 284 | VLRLCCKKL |
| 285 | YLHARLREL |
| 286 | RLRELLCEL |
| 287 | HARLRELL |
| 288 | VKRKKNVL |

In some embodiments, the donor cell source is HLA-B*15:01, and the PRAME targeted T-cell subpopulation is primed and expanded with one or more PRAME-derived peptides selected from Table 30 (SEQ ID NO: 289-298). In some embodiments, the donor cell source is HLA-B*15:01, and the PRAME targeted T-cell subpopulation is primed and expanded with PRAME-derived peptides selected from Table 30 (SEQ ID NO: 289-298). In some embodiments, the donor cell source is HLA-B*15:01, and the PRAME targeted T-cell subpopulation is primed and expanded with PRAME-derived peptides comprising the peptides of Table 30 (SEQ ID NO: 289-298). In some embodiments, the donor cell source is HLA-B*15:01, and the PRAME targeted T-cell subpopulation is primed and expanded with PRAME-derived peptides comprising the peptides of Table 30 (SEQ ID NO: 289-298) and at least one additional set of peptides based on the donor cell source HLA-B profile, wherein the at least one additional set of peptides are selected from the peptides of Tables 28-29 and 31-34. In some embodiments, the PRAME-derived peptides also include one or more sets of HLA-A and HLA-DR restricted peptides selected from Tables 21-27 and 35-40 (SEQ ID NO: 200-268 and 339-398).

TABLE 30
PRAME HLA-B*15:01 (B62) Epitope Peptides

| SEQ ID NO: | Sequence |
|---|---|
| 289 | VLYPVPLESY |
| 290 | RLWGSIQSRY |
| 291 | GLSNLTHVLY |
| 292 | RLCCKKLKIF |
| 293 | LLSHIHASSY |
| 294 | TLHLERLAY |
| 295 | GQHLHLETF |
| 296 | SLQCLQALY |
| 297 | ALYVDSLFF |
| 298 | SQLTTLSFY |

In some embodiments, the donor cell source is HLA-B*18, and the PRAME targeted T-cell subpopulation is primed and expanded with one or more PRAME-derived peptides selected from Table 31 (SEQ ID NO: 299-308). In some embodiments, the donor cell source is HLA-B*18, and the PRAME targeted T-cell subpopulation is primed and expanded with PRAME-derived peptides selected from Table 31 (SEQ ID NO: 299-308). In some embodiments, the donor cell source is HLA-B*18, and the PRAME targeted T-cell subpopulation is primed and expanded with PRAME-derived peptides comprising the peptides of Table 31 (SEQ ID NO: 299-308). In some embodiments, the donor cell source is HLA-B*18, and the PRAME targeted T-cell subpopulation is primed and expanded with PRAME-derived peptides comprising the peptides of Table 31 (SEQ ID NO: 299-308) and at least one additional set of peptides based on the donor cell source HLA-B profile, wherein the at least one additional set of peptides are selected from the peptides of Tables 28-30 and 32-34. In some embodiments, the PRAME-derived peptides also include one or more sets of HLA-A and HLA-DR restricted peptides selected from Tables 21-27 and 35-40 (SEQ ID NO: 200-268 and 339-398).

TABLE 31
PRAME HLA-B*18 Epitope Peptides

| SEQ ID NO: | Sequence |
|---|---|
| 299 | DEALAIAAL |
| 300 | LELLPRELF |
| 301 | KEGACDELF |
| 302 | PEPILCPCF |
| 303 | VEVLVDLF |
| 304 | EEQYIAQF |
| 305 | LELLPREL |
| 306 | RELFPPLF |
| 307 | SEGDVMHL |
| 308 | LERASATL |

In some embodiments, the donor cell source is HLA-B*27:05, and the PRAME targeted T-cell subpopulation is primed and expanded with one or more PRAME-derived peptides selected from Table 32 (SEQ ID NO: 309-318). In some embodiments, the donor cell source is HLA-B*27:05, and the PRAME targeted T-cell subpopulation is primed and expanded with PRAME-derived peptides selected from Table 32 (SEQ ID NO: 309-318). In some embodiments, the donor cell source is HLA-B*27:05, and the PRAME targeted T-cell subpopulation is primed and expanded with PRAME-derived peptides comprising the peptides of Table 32 (SEQ ID NO: 309-318). In some embodiments, the donor cell source is HLA-B*27:05, and the PRAME targeted T-cell subpopulation is primed and expanded with PRAME-derived peptides comprising the peptides of Table 32 (SEQ ID NO: 309-318) and at least one additional set of peptides based on the donor cell source HLA-B profile, wherein the at least one additional set of peptides are selected from the peptides of Tables 28-31 and 33-34. In some embodiments, the PRAME-derived peptides also include one or more sets of HLA-A and HLA-DR restricted peptides selected from Tables 21-27 and 35-40 (SEQ ID NO: 200-268 and 339-398).

TABLE 32
PRAME HLA-B*27:05 Epitope Peptides

| SEQ ID NO: | Sequence |
|---|---|
| 309 | RRLWGSIQSR |
| 310 | RRWKLQVLDL |
| 311 | ERLAYLHARL |
| 312 | ARLRELLCEL |
| 313 | KRKKNVLRL |
| 314 | RRLLLSHIH |
| 315 | GRLDQLLRH |
| 316 | PRRWKLQVL |
| 317 | LRLCCKKLK |
| 318 | ERLAYLHAR |

In some embodiments, the donor cell source is HLA-B*35:01, and the PRAME targeted T-cell subpopulation is primed and expanded with one or more PRAME-derived peptides selected from Table 33 (SEQ ID NO: 319-328). In some embodiments, the donor cell source is HLA-B*35:01, and the PRAME targeted T-cell subpopulation is primed and expanded with PRAME-derived peptides selected from Table 33 (SEQ ID NO: 319-328). In some embodiments, the donor cell source is HLA-B*35:01, and the PRAME targeted T-cell subpopulation is primed and expanded with PRAME-derived peptides comprising the peptides of Table 33 (SEQ ID NO: 319-328). In some embodiments, the donor cell source is HLA-B*35:01, and the PRAME targeted T-cell subpopulation is primed and expanded with PRAME-derived peptides comprising the peptides of Table 33 (SEQ ID NO: 319-328) and at least one additional set of peptides based on the donor cell source HLA-B profile, wherein the at least one additional set of peptides are selected from the peptides of Tables 28-32 and 34. In some embodiments, the PRAME-derived peptides also include one or more sets of HLA-A and HLA-DR restricted peptides selected from Tables 21-27 and 35-40 (SEQ ID NO: 200-268 and 339-398).

TABLE 33
PRAME HLA-B*35:01 Epitope Peptides

| SEQ ID NO: | Sequence |
|---|---|
| 319 | RPRRWKLQVL |
| 320 | SPSVSQLSVL |
| 321 | LPRELFPPLF |
| 322 | IPVEVLVDLF |
| 323 | MPMQDIKMIL |
| 324 | LPTLAKFSPY |
| 325 | IPVEVLVDL |
| 326 | LPRELFPPL |
| 327 | SPEPLQALL |
| 328 | QPFIPVEVL |

In some embodiments, the donor cell source is HLA-B*58:02, and the PRAME targeted T-cell subpopulation is primed and expanded with one or more PRAME-derived peptides selected from Table 34 (SEQ ID NO: 329-338). In some embodiments, the donor cell source is HLA-B*58:02, and the PRAME targeted T-cell subpopulation is primed and expanded with PRAME-derived peptides selected from Table 34 (SEQ ID NO: 329-338). In some embodiments, the donor cell source is HLA-B*58:02, and the PRAME targeted T-cell subpopulation is primed and expanded with PRAME-derived peptides comprising the peptides of Table 34 (SEQ ID NO: 329-338). In some embodiments, the donor cell source is HLA-B*58:02, and the PRAME targeted T-cell subpopulation is primed and expanded with PRAME-derived peptides comprising the peptides of Table 34 (SEQ ID NO: 329-338) and at least one additional set of peptides based on the donor cell source HLA-B profile, wherein the at least one additional set of peptides are selected from the peptides of Tables 28-33. In some embodiments, the PRAME-derived peptides also include one or more sets of HLA-A and HLA-DR restricted peptides selected from Tables 21-27 and 35-40 (SEQ ID NO: 200-268 and 339-398).

TABLE 34
PRAME HLA-B*58:02 Epitope Peptides

| SEQ ID NO: | Sequence |
|---|---|
| 329 | MSVWTSPRRL |
| 330 | AALELLPREL |
| 331 | KAVLDGLDVL |
| 332 | LAQEVRPRRW |
| 333 | ESYEDIHGTL |
| 334 | LSLQCLQALY |
| 335 | VSPEPLQALL |
| 336 | LSHCSQLTTL |
| 337 | KAMVQAWPF |
| 338 | KVKRKKNVL |

In some embodiments, the donor cell source is HLA-DRB1*0101, and the PRAME targeted T-cell subpopulation is primed and expanded with one or more PRAME-derived peptides selected from Table 35 (SEQ ID NO: 339-348). In some embodiments, the donor cell source is HLA-DRB1*0101, and the PRAME targeted T-cell subpopulation is primed and expanded with PRAME-derived peptides selected from Table 35 (SEQ ID NO: 339-348). In some embodiments, the donor cell source is HLA-DRB1*0101, and the PRAME targeted T-cell subpopulation is primed and expanded with PRAME-derived peptides comprising the peptides of Table 35 (SEQ ID NO: 339-348). In some embodiments, the donor cell source is HLA-DRB1*0101, and the PRAME targeted T-cell subpopulation is primed and expanded with PRAME-derived peptides comprising the peptides of Table 35 (SEQ ID NO: 339-348) and at least one additional set of peptides based on the donor cell source HLA-DR profile, wherein the at least one additional set of peptides are selected from the peptides of Tables 36-40. In some embodiments, the PRAME-derived peptides also include one or more sets of HLA-A and HLA-B restricted peptides selected from Tables 21-34 (SEQ ID NO: 200-338).

TABLE 35
PRAME HLA-DRB1*0101 Epitope Peptides

| SEQ ID NO: | Sequence |
|---|---|
| 339 | PRRLVELAGQSLLKD |
| 340 | LDGLDVLLAQEVRPR |
| 341 | FLSLQCLQALYVDSL |
| 342 | RHVMNPLETLSITNC |
| 343 | QLSVLSLSGVMLTDV |
| 344 | RRLWGSIQSRYISMS |
| 345 | EEQYIAQFTSQFLSL |
| 346 | DDQLLALLPSLSHCS |
| 347 | GVMLTDVSPEPLQAL |
| 348 | GQSLLKDEALAIAAL |

In some embodiments, the donor cell source is HLA-DRB1*0301, and the PRAME targeted T-cell subpopulation is primed and expanded with one or more PRAME-derived peptides selected from Table 36 (SEQ ID NO: 349-358). In some embodiments, the donor cell source is HLA-DRB1*0301, and the PRAME targeted T-cell subpopulation is primed and expanded with PRAME-derived peptides selected from Table 36 (SEQ ID NO: 349-358). In some embodiments, the donor cell source is HLA-DRB1*0301, and the PRAME targeted T-cell subpopulation is primed and expanded with PRAME-derived peptides comprising the peptides of Table 36 (SEQ ID NO: 349-358). In some embodiments, the donor cell source is HLA-DRB1*0301, and the PRAME targeted T-cell subpopulation is primed and expanded with PRAME-derived peptides comprising the peptides of Table 36 (SEQ ID NO: 349-358) and at least one additional set of peptides based on the donor cell source HLA-DR profile, wherein the at least one additional set of peptides are selected from the peptides of Tables 35 and 37-40. In some embodiments, the PRAME-derived peptides also include one or more sets of HLA-A and HLA-B restricted peptides selected from Tables 21-34 (SEQ ID NO: 200-338).

TABLE 36
PRAME HLA-DRB1*0301 (DR17) Epitope Peptides

| SEQ ID NO: | Sequence |
|---|---|
| 349 | ECGITDDQLLALLPS |
| 350 | LKMVQLDSIEDLEVT |
| 351 | LQALYVDSLFFLRGR |
| 352 | RRLVELAGQSLLKDE |
| 353 | IAALELLPRELFPPL |
| 354 | LGQMINLRRLLLSHI |
| 355 | FWTVWSGNRASLYSF |
| 356 | SSYISPEKEEQYIAQ |
| 357 | LAYLHARLRELLCEL |
| 358 | GQSLLKDEALAIAAL |

In some embodiments, the donor cell source is HLA-DRB1*0401, and the PRAME targeted T-cell subpopulation is primed and expanded with one or more PRAME-derived peptides selected from Table 37 (SEQ ID NO: 359-368). In some embodiments, the donor cell source is HLA-DRB1*0401, and the PRAME targeted T-cell subpopulation is primed and expanded with PRAME-derived peptides selected from Table 37 (SEQ ID NO: 359-368). In some embodiments, the donor cell source is HLA-DRB1*0401, and the PRAME targeted T-cell subpopulation is primed and expanded with PRAME-derived peptides comprising the peptides of Table 37 (SEQ ID NO: 359-368). In some embodiments, the donor cell source is HLA-DRB1*0401, and the PRAME targeted T-cell subpopulation is primed and expanded with PRAME-derived peptides comprising the peptides of Table 37 (SEQ ID NO: 359-368) and at least one additional set of peptides based on the donor cell source HLA-DR profile, wherein the at least one additional set of peptides are selected from the peptides of Tables 35-36 and 38-40. In some embodiments, the PRAME-derived peptides also include one or more sets of HLA-A and HLA-B restricted peptides selected from Tables 21-34 (SEQ ID NO: 200-338).

TABLE 37
PRAME HLA-DRB1*0401 (DR4Dw4) Epitope Peptides

| SEQ ID NO: | Sequence |
|---|---|
| 359 | RRLWGSIQSRYISMS |
| 360 | RRLVELAGQSLLKDE |
| 361 | SYLIEKVKRKKNVLR |
| 362 | LGQMINLRRLLLSHI |
| 363 | EQYIAQFTSQFLSLQ |
| 364 | RGRLDQLLRHVMNPL |
| 365 | RHVMNPLETLSITNC |
| 366 | EGDVMHLSQSPSVSQ |
| 367 | LALLPSLSHCSQLTT |
| 368 | SISISALQSLLQHLI |

In some embodiments, the donor cell source is HLA-DRB1*0701, and the PRAME targeted T-cell subpopulation is primed and expanded with one or more PRAME-derived peptides selected from Table 38 (SEQ ID NO: 369-378). In some embodiments, the donor cell source is HLA-DRB1*0701, and the PRAME targeted T-cell subpopulation is primed and expanded with PRAME-derived peptides selected from Table 38 (SEQ ID NO: 369-378). In some embodiments, the donor cell source is HLA-DRB1*0701, and the PRAME targeted T-cell subpopulation is primed and expanded with PRAME-derived peptides comprising the peptides of Table 38 (SEQ ID NO: 369-378). In some embodiments, the donor cell source is HLA-DRB1*0701, and the PRAME targeted T-cell subpopulation is primed and expanded with PRAME-derived peptides comprising the peptides of Table 38 (SEQ ID NO: 369-378) and at least one additional set of peptides based on the donor cell source HLA-DR profile, wherein the at least one additional set of peptides are selected from the peptides of Tables 35-37 and 39-40. In some embodiments, the PRAME-derived peptides also include one or more sets of HLA-A and HLA-B restricted peptides selected from Tables 21-34 (SEQ ID NO: 200-338).

TABLE 38
PRAME HLA-DRB1*0701 Epitope Peptides

| SEQ ID NO: | Sequence |
|---|---|
| 369 | RRLWGSIQSRYISMS |
| 370 | IEDLEVTCTWKLPTL |
| 371 | GDVMHLSQSPSVSQL |
| 372 | MVQLDSIEDLEVTCT |
| 373 | LSFYGNSISISALQS |
| 374 | MAAFDGRHSQTLKAM |
| 375 | EEQYIAQFTSQFLSL |
| 376 | EQYIAQFTSQFLSLQ |
| 377 | RHVMNPLETLSITNC |
| 378 | LQALLERASATLQDL |

In some embodiments, the donor cell source is HLA-DRB1*1101, and the PRAME targeted T-cell subpopulation is primed and expanded with one or more PRAME-derived peptides selected from Table 39 (SEQ ID NO: 379-388). In some embodiments, the donor cell source is HLA-DRB1*1101, and the PRAME targeted T-cell subpopulation is primed and expanded with PRAME-derived peptides selected from Table 39 (SEQ ID NO: 379-388). In some embodiments, the donor cell source is HLA-DRB1*1101, and the PRAME targeted T-cell subpopulation is primed and expanded with PRAME-derived peptides comprising the peptides of Table 39 (SEQ ID NO: 379-388). In some embodiments, the donor cell source is HLA-DRB1*1101, and the PRAME targeted T-cell subpopulation is primed and expanded with PRAME-derived peptides comprising the peptides of Table 39 (SEQ ID NO: 379-388) and at least one additional set of peptides based on the donor cell source HLA-DR profile, wherein the at least one additional set of peptides are selected from the peptides of Tables 35-38 and 40. In some embodiments, the PRAME-derived peptides also include one or more sets of HLA-A and HLA-B restricted peptides selected from Tables 21-34 (SEQ ID NO: 200-338).

TABLE 39
PRAME HLA-DRB1*1101 Epitope Peptides

| SEQ ID NO: | Sequence |
|---|---|
| 379 | TWKLPTLAKFSPYLG |
| 380 | QSRYISMSVWTSPRR |
| 381 | AQPMTKKRKVDGLST |
| 382 | TSQFLSLQCLQALYV |
| 383 | MSVWTSPRRLVELAG |
| 384 | IAALELLPRELFPPL |
| 385 | CLPLGVLMKGQHLHL |
| 386 | QDFWTVWSGNRASLY |
| 387 | SYLIEKVKRKKNVLR |
| 388 | MQDIKMILKMVQLDS |

In some embodiments, the donor cell source is HLA-DRB1*1501, and the PRAME targeted T-cell subpopulation is primed and expanded with one or more PRAME-derived peptides selected from Table 40 (SEQ ID NO: 389-398). In some embodiments, the donor cell source is HLA-DRB1*1501, and the PRAME targeted T-cell subpopulation is primed and expanded with PRAME-derived peptides selected from Table 40 (SEQ ID NO: 389-398). In some embodiments, the donor cell source is HLA-DRB1*1501, and the PRAME targeted T-cell subpopulation is primed and expanded with PRAME-derived peptides comprising the peptides of Table 40 (SEQ ID NO: 389-398). In some embodiments, the donor cell source is HLA-DRB1*1501, and the PRAME targeted T-cell subpopulation is primed and expanded with PRAME-derived peptides comprising the peptides of Table 40 (SEQ ID NO: 389-398) and at least one additional set of peptides based on the donor cell source HLA-DR profile, wherein the at least one additional set of peptides are selected from the peptides of Tables 35-39. In some embodiments, the PRAME-derived peptides also include one or more sets of HLA-A and HLA-B restricted peptides selected from Tables 21-34 (SEQ ID NO: 200-338).

TABLE 40
PRAME HLA-DRB1*1501 (DR2b) Epitope Peptides

| SEQ ID NO: | Sequence |
|---|---|
| 389 | HLHLETFKAVLDGLD |
| 390 | PVPLESYEDIHGTLH |
| 391 | YISMSVWTSPRRLVE |
| 392 | PLFMAAFDGRHSQTL |
| 393 | LPTLAKFSPYLGQMI |
| 394 | EQYIAQFTSQFLSLQ |
| 395 | LTTLSFYGNSISISA |
| 396 | LAKFSPYLGQMINLR |
| 397 | MERRRLWGSIQSRYI |
| 398 | GSIQSRYISMSVWTS |

Survivin Antigenic Peptides

In some embodiments, the MUSTANG composition includes survivin specific T-cells. survivin specific T-cells can be generated as described below using one or more antigenic peptides to Survivin. In some embodiments, the Survivin specific T-cells are generated using one or more antigenic peptides to Survivin, or a modified or heteroclitic peptide derived from a survivin peptide. In some embodiments, survivin specific T-cells are generated using a survivin antigen library comprising a pool of peptides (for example 15 mers) containing amino acid overlap (for example 11 amino acids of overlap) between each sequence formed by scanning the protein amino acid sequence SEQ ID NO: 399 (UniProt KB-015392) for human baculoviral inhibitor of apoptosis repeat-containing 5 (Survivin):

MGAPTLPPAWQPFLKDHRISTFKNWPFLEGCACTPERMAEAGFIHCPTEN
EPDLQCFFCFKELEGWEPDDDPIEEHKKHSSGCAFLSVKKQFEELTLGEF
LKLDRERAKNKIAKETNNKKKEFEETAKKVRRAIEQLAAMD

Overlapping antigenic libraries are commercially available, for example, from JPT, for example, from JPT (Product Code: PM-Survivin (Pep Mix™ Human (Survivin)). In some embodiments, the survivin specific T-cells are generated using a commercially available overlapping antigenic library made up of survivin peptides.

In some embodiments, the survivin specific T-cells are generated using one or more antigenic peptides to survivin, or a modified or heteroclitic peptide derived from a Survivin peptide,

In some embodiments, the survivin specific T-cells are generated with peptides that recognize class I MHC molecules. In some embodiments, the survivin specific T-cells are generated with peptides that recognize class II MHC molecules. In some embodiments, the Survivin specific T-cells are generated with peptides that recognize both class I and class II MHC molecules.

In some embodiments, the survivin peptides used to prime and expand a T-cell subpopulation includes specifically selected HLA-restricted peptides generated by determining the HLA profile of the donor source, and including peptides derived from survivin that best match the donor's HLA. In some embodiments, the survivin peptides used to prime and expand a T-cell subpopulation are derived from HLA-restricted peptides selected from at least one or more of an

HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting survivin derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 41-47 , the HLA-B peptides are selected from the peptides of Tables 48-54, and the HLA-DR peptides are selected from the peptides of Tables 55-60. For example, if the donor cell source has an HLA profile that is HLA-A*01/*02:01; HLA-B*15:01/*18; and HLA-DRB1*0101/*0301, then the survivin peptides used to prime and expand the survivin specific T-cell subpopulation are restricted to the specific HLA profile, and may include the peptides identified in Table 41 (SEQ ID NO: 400-409) for HLA-A*01; Table 42 (SEQ ID NO: 410-419) for HLA-A*02:01; Table 50 (SEQ ID NO: 490-500) for HLA-B*15:01; Table 51 (SEQ ID NO: 501-510) for HLA-B*18; Table 55 (SEQ ID NO: 541-550) for HLA-DRB1*0101; and Table 56 (SEQ ID NO: 551-560) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the survivin targeted T-cell subpopulation is primed and expanded with one or more survivin-derived peptides selected from Table 41 (SEQ ID NO: 400-409). In some embodiments, the donor cell source is HLA-A*01, and the survivin targeted T-cell subpopulation is primed and expanded with survivin-derived peptides selected from Table 41 (SEQ ID NO: 400-409). In some embodiments, the donor cell source is HLA-A*01, and the survivin targeted T-cell subpopulation is primed and expanded with survivin-derived peptides comprising the peptides of Table 41 (SEQ ID NO: 400-409). In some embodiments, the donor cell source is HLA-A*01, and the survivin targeted T-cell subpopulation is primed and expanded with survivin-derived peptides comprising the peptides of Table 41 (SEQ ID NO: 400-409) and at least one additional set of peptides based on the donor cell source HLA-A profile, wherein the at least one additional set of peptides are selected from the peptides of Tables 42-47. In some embodiments, the survivin-derived peptides also include one or more sets of HLA-B and HLA-DR restricted peptides selected from Tables 48-60 (SEQ ID NO: 470-600).

TABLE 41
Survivin HLA-A*01 Epitope Peptides

| SEQ ID NO: | Sequence |
|---|---|
| 400 | PTENEPDLAQC |
| 401 | KLDRERAKNKI |
| 402 | LKDHRISTFKN |
| 403 | STFKNWPFLEG |
| 404 | DDDPIEEHKKH |
| 405 | PTENEPDLAQ |
| 406 | PTENEPDLA |
| 407 | LTLGEFLKL |
| 408 | LGEFLKLDR |
| 409 | KLDRERAKN |

In some embodiments, the donor cell source is HLA-A*02:01, and the survivin targeted T-cell subpopulation is primed and expanded with one or more survivin-derived peptides selected from Table 42 (SEQ ID NO: 410-419). In some embodiments, the donor cell source is HLA-A*02:01, and the survivin targeted T-cell subpopulation is primed and expanded with survivin-derived peptides selected from Table 42 (SEQ ID NO: 410-419). In some embodiments, the donor cell source is HLA-A*02:01, and the survivin targeted T-cell subpopulation is primed and expanded with survivin-derived peptides comprising the peptides of Table 42 (SEQ ID NO: 410-419). In some embodiments, the donor cell source is HLA-A*02:01, and the survivin targeted T-cell subpopulation is primed and expanded with survivin-derived peptides comprising the peptides of Table 42 (SEQ ID NO: 410-419) and at least one additional set of peptides based on the donor cell source HLA-A profile, wherein the at least one additional set of peptides are selected from the peptides of Tables 41, and 43-47. In some embodiments, the survivin-derived peptides also include one or more sets of HLA-B and HLA-DR restricted peptides selected from Tables 48-60 (SEQ ID NO: 470-600).

TABLE 42
Survivin HLA-A*02:01 Epitope Peptides

| SEQ ID NO: | Sequence |
|---|---|
| 410 | TLPPAWQPFL |
| 411 | ELTLGEFLKL |
| 412 | FLKDHRISTF |
| 413 | LTLGEFLKL |
| 414 | KVRRAIEQL |
| 415 | RAIEQLAAM |
| 416 | STFKNWPFL |
| 417 | FLKDHRIST |
| 418 | SVKKQFEEL |
| 419 | TLGEFLKLD |

In some embodiments, the donor cell source is HLA-A*03, and the survivin targeted T-cell subpopulation is primed and expanded with one or more survivin-derived peptides selected from Table 43 (SEQ ID NO: 420-429). In some embodiments, the donor cell source is HLA-A*03, and the survivin targeted T-cell subpopulation is primed and expanded with survivin-derived peptides selected from Table 43 (SEQ ID NO: 420-429). In some embodiments, the donor cell source is HLA-A*03, and the survivin targeted T-cell subpopulation is primed and expanded with survivin-derived peptides comprising the peptides of Table 43 (SEQ ID NO: 420-429). In some embodiments, the donor cell source is HLA-A*03, and the survivin targeted T-cell subpopulation is primed and expanded with survivin-derived peptides comprising the peptides of Table 43 (SEQ ID NO: 420-429) and at least one additional set of peptides based on the donor cell source HLA-A profile, wherein the at least one additional set of peptides are selected from the peptides of Tables 41-42 and 44-47. In some embodiments, the survivin-derived peptides also include one or more sets of HLA-B and HLA-DR restricted peptides selected from Tables 48-60 (SEQ ID NO: 470-600).

TABLE 43
Survivin HLA-A*03 Epitope Peptides

| SEQ ID NO: | Sequence |
|---|---|
| 420 | KLDRERAKNK |
| 421 | FLKDHRISTF |
| 422 | FLKLDRERAK |
| 423 | KIAKETNNKK |
| 424 | DLAQCFFCFK |
| 425 | ELTLGEFLK |
| 426 | KIAKETNNK |
| 427 | KVRRAIEQL |
| 428 | SGCAFLSVK |
| 429 | KLDRERAKN |

In some embodiments, the donor cell source is HLA-A*11:01, and the survivin targeted T-cell subpopulation is primed and expanded with one or more survivin-derived peptides selected from Table 44 (SEQ ID NO: 430-439). In some embodiments, the donor cell source is HLA-A*11:01, and the survivin targeted T-cell subpopulation is primed and expanded with survivin-derived peptides selected from Table 44 (SEQ ID NO: 430-439). In some embodiments, the donor cell source is HLA-A*11:01, and the survivin targeted T-cell subpopulation is primed and expanded with survivin-derived peptides comprising the peptides of Table 44 (SEQ ID NO: 430-439). In some embodiments, the donor cell source is HLA-A*11:01, and the survivin targeted T-cell subpopulation is primed and expanded with survivin-derived peptides comprising the peptides of Table 44 (SEQ ID NO: 430-439), and at least one additional set of peptides based on the donor cell source HLA-A profile, wherein the at least one additional set of peptides are selected from the peptides of Tables 41-43 and 45-47. In some embodiments, the survivin-derived peptides also include one or more sets of HLA-B and HLA-DR restricted peptides selected from Tables 48-60 (SEQ ID NO: 470-600).

TABLE 44
Survivin HLA-A*11:01 Epitope Peptides

| SEQ ID NO: | Sequence |
|---|---|
| 430 | SSGCAFLSVK |
| 431 | DLAQCFFCFK |
| 432 | SGCAFLSVKK |
| 433 | TLGEFLKLDR |
| 434 | STFKNWPFLE |
| 435 | KLDRERAKNK |
| 436 | KIAKETNNKK |
| 437 | SSGCAFLSV |
| 438 | GCAFLSVKK |
| 439 | ELTLGEFLK |

In some embodiments, the donor cell source is HLA-A*24:02, and the survivin targeted T-cell subpopulation is primed and expanded with one or more survivin-derived peptides selected from Table 45 (SEQ ID NO: 440-449). In some embodiments, the donor cell source is HLA-A*24:02, and the survivin targeted T-cell subpopulation is primed and expanded with survivin-derived peptides selected from Table 45 (SEQ ID NO: 440-449). In some embodiments, the donor cell source is HLA-A*24:02, and the survivin targeted T-cell subpopulation is primed and expanded with survivin-derived peptides comprising the peptides of Table 45 (SEQ ID NO: 440-449). In some embodiments, the donor cell source is HLA-A*24:02, and the survivin targeted T-cell subpopulation is primed and expanded with survivin-derived peptides comprising the peptides of Table 45 (SEQ ID NO: 440-449), and at least one additional set of peptides based on the donor cell source HLA-A profile, wherein the at least one additional set of peptides are selected from the peptides of Tables 41-44 and 46-47. In some embodiments, the survivin-derived peptides also include one or more sets of HLA-B and HLA-DR restricted peptides selected from Tables 48-60 (SEQ ID NO: 470-600).

TABLE 45
Survivin HLA-A24:02 Epitope Peptides

| SEQ ID NO: | Sequence |
|---|---|
| 440 | QFEELTLGEF |
| 441 | TLPPAWQPFL |
| 442 | PDLAQCFFCF |
| 443 | PTLPPAWQPF |
| 444 | NEPDLAQCFF |
| 445 | LSVKKQFEEL |
| 446 | ELTLGEFLKL |
| 447 | AFLSVKKQF |
| 448 | LTLGEFLKL |
| 449 | TLPPAWQPF |

In some embodiments, the donor cell source is HLA-A*26, and the survivin targeted T-cell subpopulation is primed and expanded with one or more survivin-derived peptides selected from Table 46 (SEQ ID NO: 450-459). In some embodiments, the donor cell source is HLA-A*26, and the survivin targeted T-cell subpopulation is primed and expanded with survivin-derived peptides selected from Table 46 (SEQ ID NO: 450-459). In some embodiments, the donor cell source is HLA-A*26, and the survivin targeted T-cell subpopulation is primed and expanded with survivin-derived peptides comprising the peptides of Table 46 (SEQ ID NO: 450-459). In some embodiments, the donor cell source is HLA-A*26, and the survivin targeted T-cell subpopulation is primed and expanded with survivin-derived peptides comprising the peptides of Table 46 (SEQ ID NO: 450-459) and at least one additional set of peptides based on the donor cell source HLA-A profile, wherein the at least one additional set of peptides are selected from the peptides of Tables 41-45 and 47. In some embodiments, the survivin-derived peptides also include one or more sets of HLA-B and HLA-DR restricted peptides selected from Tables 48-60 (SEQ ID NO: 470-600).

TABLE 46
Survivin HLA-A*26 Epitope Peptides

| SEQ ID NO: | Sequence |
|---|---|
| 450 | ELTLGEFLKL |
| 451 | ENEPDLAQCF |
| 452 | ETAKKVRRAI |
| 453 | ETNNKKKEFE |
| 454 | ETNNKKKEF |
| 455 | ETAKKVRRA |
| 456 | KVRRAIEQL |
| 457 | STFKNWPFL |
| 458 | EELTLGEFL |
| 459 | SVKKQFEEL |

In some embodiments, the donor cell source is HLA-A*68:01, and the survivin targeted T-cell subpopulation is primed and expanded with one or more survivin-derived peptides selected from Table 47 (SEQ ID NO: 460-469). In some embodiments, the donor cell source is HLA-A*68:01, and the survivin targeted T-cell subpopulation is primed and expanded with survivin-derived peptides selected from Table 47 (SEQ ID NO: 460-469). In some embodiments, the donor cell source is HLA-A*68:01, and the survivin targeted T-cell subpopulation is primed and expanded with survivin-derived peptides comprising the peptides of Table 47 (SEQ ID NO: 460-469). In some embodiments, the donor cell source is HLA-A*68:01, and the survivin targeted T-cell subpopulation is primed and expanded with survivin-derived peptides comprising the peptides of Table 47 (SEQ ID NO: 460-469), and at least one additional set of peptides based on the donor cell source HLA-A profile, wherein the at least one additional set of peptides are selected from the peptides of Tables 41-46. In some embodiments, the survivin-derived peptides also include one or more sets of HLA-B and HLA-DR restricted peptides selected from Tables 48-60 (SEQ ID NO: 470-600).

TABLE 47
Survivin HLA-A*68:01 Epitope Peptides

| SEQ ID NO: | Sequence |
|---|---|
| 460 | LTLGEFLKLDR |
| 461 | PAWQPFLKDHR |
| 462 | SSGCAFLSVKK |
| 463 | EFEETAKKVRR |
| 464 | ETAKKVRRAIE |
| 465 | DLAQCFFCFK |
| 466 | EETAKKVRR |
| 467 | ERAKNKIAK |
| 468 | ETAKKVRRA |
| 469 | ELTLGEFLK |

In some embodiments, the donor cell source is HLA-B*07:02, and the survivin targeted T-cell subpopulation is primed and expanded with one or more survivin-derived peptides selected from Table 48 (SEQ ID NO: 470-479). In some embodiments, the donor cell source is HLA-B*07:02, and the survivin targeted T-cell subpopulation is primed and expanded with survivin-derived peptides selected from Table 48 (SEQ ID NO: 470-479). In some embodiments, the donor cell source is HLA-B*07:02, and the survivin targeted T-cell subpopulation is primed and expanded with survivin-derived peptides comprising the peptides of Table 48 (SEQ ID NO: 470-479). In some embodiments, the donor cell source is HLA-B*07:02, and the survivin targeted T-cell subpopulation is primed and expanded with survivin-derived peptides comprising the peptides of Table 48 (SEQ ID NO: 470-479), and at least one additional set of peptides based on the donor cell source HLA-B profile, wherein the at least one additional set of peptides are selected from the peptides of Tables 49-54. In some embodiments, the survivin-derived peptides also include one or more sets of HLA-A and HLA-DR restricted peptides selected from Tables 41-47 and 55-60 (SEQ ID NO: 400-469 and 541-600).

TABLE 48
Survivin HLA-B*07:02 Epitope Peptides

| SEQ ID NO: | Sequence |
|---|---|
| 470 | LPPAWQPFL |
| 471 | CPTENEPDL |
| 472 | EPDLAQCFF |
| 473 | APTLPPAWQ |
| 474 | QPFLKDHRI |
| 475 | KHSSGCAFL |
| 476 | LTLGEFLKL |
| 477 | WPFLEGCACT |
| 478 | TPERMAEAGF |
| 479 | CPTENEPDLA |

In some embodiments, the donor cell source is HLA-B*08, and the survivin targeted T-cell subpopulation is primed and expanded with one or more survivin-derived peptides selected from Table 49 (SEQ ID NO: 480-489). In some embodiments, the donor cell source is HLA-B*08, and the survivin targeted T-cell subpopulation is primed and expanded with survivin-derived peptides selected from Table 49 (SEQ ID NO: 480-489). In some embodiments, the donor cell source is HLA-B*08, and the survivin targeted T-cell subpopulation is primed and expanded with survivin-derived peptides comprising the peptides of Table 49 (SEQ ID NO: 480-489). In some embodiments, the donor cell source is HLA-B*08, and the survivin targeted T-cell subpopulation is primed and expanded with survivin-derived peptides comprising the peptides of Table 49 (SEQ ID NO: 480-489) and at least one additional set of peptides based on the donor cell source HLA-B profile, wherein the at least one additional set of peptides are selected from the peptides of Tables 48 and 50-54. In some embodiments, the survivin-derived peptides also include one or more sets of HLA-A and HLA-DR restricted peptides selected from Tables 41-47 and 55-60 (SEQ ID NO: 400-469 and 541-600).

TABLE 49
Survivin HLA-B*08 Epitope Peptides

| SEQ ID NO: | Sequence |
|---|---|
| 480 | RAKNKIAKE |
| 481 | QPFLKDHRI |
| 482 | SVKKQFEEL |
| 483 | NNKKKEFEE |
| 484 | TAKKVRRAI |
| 485 | AKKVRRAI |
| 486 | FLSVKKQF |
| 487 | RAKNKIAK |
| 488 | RERAKNKI |
| 489 | VKKQFEEL |

In some embodiments, the donor cell source is HLA-B*15:01, and the survivin targeted T-cell subpopulation is primed and expanded with one or more survivin-derived peptides selected from Table 50 (SEQ ID NO: 490-500). In some embodiments, the donor cell source is HLA-B*15:01, and the survivin targeted T-cell subpopulation is primed and expanded with survivin-derived peptides selected from Table 50 (SEQ ID NO: 490-500). In some embodiments, the donor cell source is HLA-B*15:01, and the survivin targeted T-cell subpopulation is primed and expanded with survivin-derived peptides comprising the peptides of Table 50 (SEQ ID NO: 490-500). In some embodiments, the donor cell source is HLA-B*15:01, and the survivin targeted T-cell subpopulation is primed and expanded with survivin-derived peptides comprising the peptides of Table 50 (SEQ ID NO: 490-500) and at least one additional set of peptides based on the donor cell source HLA-B profile, wherein the at least one additional set of peptides are selected from the peptides of Tables 48-49 and 51-54. In some embodiments, the survivin-derived peptides also include one or more sets of HLA-A and HLA-DR restricted peptides selected from Tables 41-47 and 55-60 (SEQ ID NO: 400-469 and 541-600).

TABLE 50
Survivin HLA-B*15:01 (B62) Epitope Peptides

| SEQ ID NO: | Sequence |
|---|---|
| 490 | FLKDHRISTF |
| 491 | KQFEELTLGE |
| 492 | TLPPAWQPFL |
| 493 | ELEGWEPDDD |
| 495 | TLGEFLKLDR |
| 496 | TLPPAWQPF |
| 497 | DLAQCFFCF |
| 498 | KQFEELTLG |
| 499 | FLKDHRIST |
| 500 | KVRRAIEQL |

In some embodiments, the donor cell source is HLA-B*18, and the survivin targeted T-cell subpopulation is primed and expanded with one or more survivin-derived peptides selected from Table 51 (SEQ ID NO: 501-510). In some embodiments, the donor cell source is HLA-B*18, and the survivin targeted T-cell subpopulation is primed and expanded with survivin-derived peptides selected from Table 51 (SEQ ID NO: 501-510). In some embodiments, the donor cell source is HLA-B*18, and the survivin targeted T-cell subpopulation is primed and expanded with survivin-derived peptides comprising the peptides of Table 51 (SEQ ID NO: 501-510). In some embodiments, the donor cell source is HLA-B*18, and the survivin targeted T-cell subpopulation is primed and expanded with survivin-derived peptides comprising the peptides of Table 51 (SEQ ID NO: 501-510) and at least one additional set of peptides based on the donor cell source HLA-B profile, wherein the at least one additional set of peptides are selected from the peptides of Tables 48-50 and 52-54. In some embodiments, the survivin-derived peptides also include one or more sets of HLA-A and HLA-DR restricted peptides selected from Tables 41-47 and 55-60 (SEQ ID NO: 400-469 and 541-600).

TABLE 51
Survivin HLA-B*18 Epitope Peptides

| SEQ ID NO: | Sequence |
|---|---|
| 501 | EELTLGEFL |
| 502 | FEELTLGEF |
| 503 | NEPDLAQCF |
| 504 | PERMAEAGF |
| 505 | DLAQCFFCF |
| 506 | KELEGWEPD |
| 507 | EELTLGEF |
| 508 | EEHKKHSS |
| 509 | KELEGWEP |
| 510 | KQFEELTL |

In some embodiments, the donor cell source is HLA-B*27:05, and the survivin targeted T-cell subpopulation is primed and expanded with one or more survivin-derived peptides selected from Table 52 (SEQ ID NO: 511-520). In some embodiments, the donor cell source is HLA-B*27:05, and the survivin targeted T-cell subpopulation is primed and expanded with survivin-derived peptides selected from Table 52 (SEQ ID NO: 511-520). In some embodiments, the donor cell source is HLA-B*27:05, and the survivin targeted T-cell subpopulation is primed and expanded with survivin-derived peptides comprising the peptides of Table 52 (SEQ ID NO: 511-520). In some embodiments, the donor cell source is HLA-B*27:05, and the survivin targeted T-cell subpopulation is primed and expanded with survivin-derived peptides comprising the peptides of Table 52 (SEQ ID NO: 511-520) and at least one additional set of peptides based on the donor cell source HLA-B profile, wherein the at least one additional set of peptides are selected from the peptides of Tables 48-51 and 53-54. In some embodiments, the survivin-derived peptides also include one or more sets of HLA-A and HLA-DR restricted peptides selected from Tables 41-47 and 55-60 (SEQ ID NO: 400-469 and 541-600).

TABLE 52
Survivin HLA-B*27:05 Epitope Peptides

| SEQ ID NO: | Sequence |
|---|---|
| 511 | RRAIEQLAAM |
| 512 | GEFLKLDRER |
| 513 | ERMAEAGFIH |
| 514 | ERAKNKIAKE |
| 515 | KIAKETNNKK |
| 516 | ERAKNKIAK |
| 517 | DRERAKNKI |
| 518 | KEFEETAKK |
| 519 | ERMAEAGFI |
| 520 | GCAFLSVKK |

In some embodiments, the donor cell source is HLA-B*35:01, and the survivin targeted T-cell subpopulation is primed and expanded with one or more survivin-derived peptides selected from Table 53 (SEQ ID NO: 521-530). In some embodiments, the donor cell source is HLA-B*35:01, and the survivin targeted T-cell subpopulation is primed and expanded with survivin-derived peptides selected from Table 53 (SEQ ID NO: 521-530). In some embodiments, the donor cell source is HLA-B*35:01, and the survivin targeted T-cell subpopulation is primed and expanded with survivin-derived peptides comprising the peptides of Table 53 (SEQ ID NO: 521-530). In some embodiments, the donor cell source is HLA-B*35:01, and the survivin targeted T-cell subpopulation is primed and expanded with survivin-derived peptides comprising the peptides of Table 53 (SEQ ID NO: 521-530) and at least one additional set of peptides based on the donor cell source HLA-B profile, wherein the at least one additional set of peptides are selected from the peptides of Tables 48-52 and 54. In some embodiments, the survivin-derived peptides also include one or more sets of HLA-A and HLA-DR restricted peptides selected from Tables 41-47 and 55-60 (SEQ ID NO: 400-469 and 541-600).

TABLE 53
Survivin HLA-B*35:01 Epitope Peptides

| SEQ ID NO: | Sequence |
|---|---|
| 521 | TPERMAEAGF |
| 522 | LPPAWQPFLK |
| 523 | EPDDDPIEEH |
| 524 | LSVKKQFEEL |
| 525 | LPPAWQPFL |
| 526 | CPTENEPDL |
| 527 | EPDLAQCFF |
| 528 | QPFLKDHRI |
| 529 | TPERMAEAG |
| 530 | EPDDDPIEE |

In some embodiments, the donor cell source is HLA-B*58:02, and the survivin targeted T-cell subpopulation is primed and expanded with one or more survivin-derived peptides selected from Table 54 (SEQ ID NO: 531-540). In some embodiments, the donor cell source is HLA-B*58:02, and the survivin targeted T-cell subpopulation is primed and expanded with survivin-derived peptides selected from Table 54 (SEQ ID NO: 531-540). In some embodiments, the donor cell source is HLA-B*58:02, and the survivin targeted T-cell subpopulation is primed and expanded with survivin-derived peptides comprising the peptides of Table 54 (SEQ ID NO: 531-540). In some embodiments, the donor cell source is HLA-B*58:02, and the survivin targeted T-cell subpopulation is primed and expanded with survivin-derived peptides comprising the peptides of Table 54 (SEQ ID NO: 531-540) and at least one additional set of peptides based on the donor cell source HLA-B profile, wherein the at least one additional set of peptides are selected from the peptides of Tables 48-53. In some embodiments, the survivin-derived peptides also include one or more sets of HLA-A and HLA-DR restricted peptides selected from Tables 41-47 and 55-60 (SEQ ID NO: 400-469 and 541-600).

TABLE 54
Survivin HLA-B*58:02 Epitope Peptides

| SEQ ID NO: | Sequence |
|---|---|
| 531 | ETAKKVRRAI |
| 532 | PTLPPAWQPF |
| 533 | ISTFKNWPFL |
| 534 | LSVKKQFEEL |
| 535 | TAKKVRRAI |
| 536 | RAIEQLAAM |
| 537 | KVRRAIEQL |
| 538 | ISTFKNWPF |
| 539 | LTLGEFLKL |
| 540 | GAPTLPPAW |

In some embodiments, the donor cell source is HLA-DRB1*0101, and the survivin targeted T-cell subpopulation is primed and expanded with one or more survivin-derived peptides selected from Table 55 (SEQ ID NO: 541-550). In some embodiments, the donor cell source is HLA-DRB 1 *0101, and the survivin targeted T-cell subpopulation is primed and expanded with survivin-derived peptides selected from Table 55 (SEQ ID NO: 541-550). In some embodiments, the donor cell source is HLA-DRB1*0101, and the survivin targeted T-cell subpopulation is primed and expanded with survivin-derived peptides comprising the peptides of Table 55 (SEQ ID NO: 541-550). In some embodiments, the donor cell source is HLA-DRB1*0101, and the survivin targeted T-cell subpopulation is primed and expanded with survivin-derived peptides comprising the peptides of Table 55 (SEQ ID NO: 541-550) and at least one additional set of peptides based on the donor cell source HLA-DR profile, wherein the at least one additional set of peptides are selected from the peptides of Tables 56-60. In some embodiments, the survivin-derived peptides also include one or more sets of HLA-A and HLA-B restricted peptides selected from Tables 41-54 (SEQ ID NO: 400-540).

TABLE 55
Survivin HLA-DRB1*0101 Epitope Peptides

| SEQ ID NO: | Sequence |
|---|---|
| 541 | FFCFKELEGWEPDDD |
| 542 | FKNWPFLEGCACTPE |
| 543 | LGEFLKLDRERAKNK |
| 544 | NWPFLEGCACTPERM |
| 545 | KKQFEELTLGEFLKL |
| 546 | CTPERMAEAGFIHCP |
| 547 | FEELTLGEFLKLDRE |
| 548 | MGAPTLPPAWQPFLK |
| 549 | KKKEFEETAKKVRRA |
| 550 | AKKVRRAIEQLAAMD |

In some embodiments, the donor cell source is HLA-DRB1*0301, and the survivin targeted T-cell subpopulation is primed and expanded with one or more survivin-derived peptides selected from Table 56 (SEQ ID NO: 551-560). In some embodiments, the donor cell source is HLA-DRB1*0301, and the survivin targeted T-cell subpopulation is primed and expanded with survivin-derived peptides selected from Table 56 (SEQ ID NO: 551-560). In some embodiments, the donor cell source is HLA-DRB1*0301, and the survivin targeted T-cell subpopulation is primed and expanded with survivin-derived peptides comprising the peptides of Table 56 (SEQ ID NO: 551-560). In some embodiments, the donor cell source is HLA-DRB1*0301, and the survivin targeted T-cell subpopulation is primed and expanded with survivin-derived peptides comprising the peptides of Table 56 (SEQ ID NO: 551-560) and at least one additional set of peptides based on the donor cell source HLA-DR profile, wherein the at least one additional set of peptides are selected from the peptides of Tables 55 and 57-60. In some embodiments, the survivin-derived peptides also include one or more sets of HLA-A and HLA-B restricted peptides selected from Tables 41-54 (SEQ ID NO: 400-540).

TABLE 56
Survivin HLA-DRB1*0301 (DR17) Epitope Peptides

| SEQ ID NO: | Sequence |
|---|---|
| 551 | GEFLKLDRERAKNKI |
| 552 | WQPFLKDHRISTFKN |
| 553 | APTLPPAWQPFLKDH |
| 554 | DHRISTFKNWPFLEG |
| 555 | FEELTLGEFLKLDRE |
| 556 | PIENEPDLAQCFFCF |
| 557 | QPFLKDHRISTFKNW |
| 558 | GCAFLSVKKQFEELT |
| 559 | ELTLGEFLKLDRERA |
| 560 | AKKVRRAIEQLAAMD |

In some embodiments, the donor cell source is HLA-DRB1*0401, and the survivin targeted T-cell subpopulation is primed and expanded with one or more survivin-derived peptides selected from Table 57 (SEQ ID NO: 561-570). In some embodiments, the donor cell source is HLA-DRB 1 *0401, and the survivin targeted T-cell subpopulation is primed and expanded with survivin-derived peptides selected from Table 57 (SEQ ID NO: 561-570). In some embodiments, the donor cell source is HLA-DRB1*0401, and the survivin targeted T-cell subpopulation is primed and expanded with survivin-derived peptides comprising the peptides of Table 57 (SEQ ID NO: 561-570). In some embodiments, the donor cell source is HLA-DRB1*0401, and the survivin targeted T-cell subpopulation is primed and expanded with survivin-derived peptides comprising the peptides of Table 57 (SEQ ID NO: 561-570) and at least one additional set of peptides based on the donor cell source HLA-DR profile, wherein the at least one additional set of peptides are selected from the peptides of Tables 55-56 and 58-60. In some embodiments, the survivin-derived peptides also include one or more sets of HLA-A and HLA-B restricted peptides selected from Tables 41-54 (SEQ ID NO: 400-540).

TABLE 57
Survivin HLA-DRB1*0401 (DR4Dw4) Epitope Peptides

| SEQ ID NO: | Sequence |
|---|---|
| 561 | WQPFLKDHRISTFKN |
| 562 | LGEFLKLDRERAKNK |
| 563 | APTLPPAWQPFLKDH |
| 564 | KNKIAKETNNKKKEF |
| 565 | DHRISTFKNWPFLEG |
| 566 | GEFLKLDRERAKNKI |
| 567 | FLKLDRERAKNKIAK |
| 568 | AKKVRRAIEQLAAMD |
| 569 | FLKDHRISTFKNWPF |
| 570 | RMAEAGFIHCPTENE |

In some embodiments, the donor cell source is HLA-DRB1*0701, and the survivin targeted T-cell subpopulation is primed and expanded with one or more survivin-derived peptides selected from Table 58 (SEQ ID NO: 571-580). In some embodiments, the donor cell source is HLA-DRB 1 *0701, and the survivin targeted T-cell subpopulation is primed and expanded with survivin-derived peptides selected from Table 58 (SEQ ID NO: 571-580). In some embodiments, the donor cell source is HLA-DRB1*0701, and the survivin targeted T-cell subpopulation is primed and expanded with survivin-derived peptides comprising the peptides of Table 58 (SEQ ID NO: 571-580). In some embodiments, the donor cell source is HLA-DRB1*0701, and the survivin targeted T-cell subpopulation is primed and expanded with survivin-derived peptides comprising the peptides of Table 58 (SEQ ID NO: 571-580) and at least one additional set of peptides based on the donor cell source HLA-DR profile, wherein the at least one additional set of peptides are selected from the peptides of Tables 55-57 and 59-60. In some embodiments, the survivin-derived peptides also include one or more sets of HLA-A and HLA-B restricted peptides selected from Tables 41-54 (SEQ ID NO: 400-540).

TABLE 58
Survivin HLA-DRB1*0701 Epitope Peptides

| SEQ ID NO: | Sequence |
|---|---|
| 571 | AKKVRRAIEQLAAMD |
| 572 | APTLPPAWQPFLKDH |
| 573 | DHRISTFKNWPFLEG |
| 574 | LEGCACTPERMAEAG |
| 575 | EAGFIHCPTENEPDL |
| 576 | KKEFEETAKKVRRAI |
| 577 | AQCFFCFKELEGWEP |
| 578 | QCFFCFKELEGWEPD |
| 579 | LEGWEPDDDPIEEHK |
| 580 | KKQFEELTLGEFLKL |

In some embodiments, the donor cell source is HLA-DRB1*1101, and the survivin targeted T-cell subpopulation is primed and expanded with one or more survivin-derived peptides selected from Table 59 (SEQ ID NO: 581-590). In some embodiments, the donor cell source is HLA-DRB1*1101, and the survivin targeted T-cell subpopulation is primed and expanded with survivin-derived peptides selected from Table 59 (SEQ ID NO: 581-590). In some embodiments, the donor cell source is HLA-DRB1*1101, and the survivin targeted T-cell subpopulation is primed and expanded with survivin-derived peptides comprising the peptides of Table 59 (SEQ ID NO: 581-590). In some embodiments, the donor cell source is HLA-DRB1*1101, and the survivin targeted T-cell subpopulation is primed and expanded with survivin-derived peptides comprising the peptides of Table 59 (SEQ ID NO: 581-590) and at least one additional set of peptides based on the donor cell source HLA-DR profile, wherein the at least one additional set of peptides are selected from the peptides of Tables 55-58 and 60. In some embodiments, the survivin-derived peptides also include one or more sets of HLA-A and HLA-B restricted peptides selected from Tables 41-54 (SEQ ID NO: 400-540).

TABLE 59
Survivin HLA-DRB1*1101 Epitope Peptides

| SEQ ID NO: | Sequence |
|---|---|
| 581 | LGEFLKLDRERAKNK |
| 582 | GCAFLSVKKQFEELT |
| 583 | FFCFKELEGWEPDDD |
| 584 | DDPIEEHKKHSSGCA |
| 585 | KKEFEETAKKVRRAI |
| 586 | PPAWQPFLKDHRIST |
| 587 | WQPFLKDHRISTFKN |
| 588 | AWQPFLKDHRISTFK |
| 589 | AQCFFCFKELEGWEP |
| 590 | ISTFKNWPFLEGCAC |

In some embodiments, the donor cell source is HLA-DRB1*1501, and the survivin targeted T-cell subpopulation is primed and expanded with one or more survivin-derived peptides selected from Table 60 (SEQ ID NO: 591-600). In some embodiments, the donor cell source is HLA-DRB1*1501, and the survivin targeted T-cell subpopulation is primed and expanded with survivin-derived peptides selected from Table 60 (SEQ ID NO: 591-600). In some embodiments, the donor cell source is HLA-DRB1*1501, and the survivin targeted T-cell subpopulation is primed and expanded with survivin-derived peptides comprising the peptides of Table 60 (SEQ ID NO: 591-600). In some embodiments, the donor cell source is HLA-DRB1*1501, and the survivin targeted T-cell subpopulation is primed and expanded with survivin-derived peptides comprising the peptides of Table 60 (SEQ ID NO: 591-600) and at least one additional set of peptides based on the donor cell source HLA-DR profile, wherein the at least one additional set of peptides are selected from the peptides of Tables 55-59. In some embodiments, the survivin-derived peptides also include one or more sets of HLA-A and HLA-B restricted peptides selected from Tables 41-54 (SEQ ID NO: 400-540).

TABLE 60
Survivin HLA-DRB1*1501 (DR2b) Epitope Peptides

| SEQ ID NO: | Sequence |
|---|---|
| 591 | LGEFLKLDRERAKNK |
| 592 | GCAFLSVKKQFEELT |
| 593 | FFCFKELEGWEPDDD |
| 594 | DDPIEEHKKHSSGCA |
| 595 | KKEFEETAKKVRRAI |
| 596 | PPAWQPFLKDHRIST |
| 597 | WQPFLKDHRISTFKN |
| 598 | AWQPFLKDHRISTFK |
| 599 | AQCFFCFKELEGWEP |
| 600 | ISTFKNWPFLEGCAC |

NY-ESO-1 Antigenic Peptides

In some embodiments, the MUSTANG composition includes NY-ESO-1 (cancer/testis antigen 1) specific T-cells. NY-ESO-1 specific T-cells can be generated as described below using one or more antigenic peptides to NY-ESO-1. In some embodiments, the NY-ESO-1 specific T-cells are generated using one or more antigenic peptides to NY-ESO-1, or a modified or heteroclitic peptide derived from a NY-ESO-1 peptide. In some embodiments, NY-ESO-1 specific T-cells are generated using a NY-ESO-1 antigen library comprising a pool of peptides (for example 15 mers) containing amino acid overlap (for example 11 amino acids of overlap) between each sequence formed by scanning the protein amino acid sequence SEQ ID NO: 601 (UniProt KB-P78358) for

NY-ESO-1:

MQAEGRGTGGSTGDADGPGGPGIPDGPGGNAGGPGEAGATGGRGPRGAGA
ARASGPGGGAPRGPHGGAASGLNGCCRCGARGPESRLLEFYLAMPFATPM
EAELARRSLAQDAPPLPVPGVLLKEFTVSGNILTIRLTAADHRQLQLSIS
SCLQQLSLLMWITQCFLPVFLAQPPSGQRR.

Overlapping antigenic libraries are commercially available, for example, from JPT, for example, from JPT (Product Code: PM-NYE (Pep Mix™ Human (NY-ESO-1)). In some embodiments, the NY-ESO-1 specific T-cells are generated using a commercially available overlapping antigenic library made up of NY-ESO-1 peptides.

In some embodiments, the NY-ESO-1 specific T-cells are generated using one or more antigenic peptides to NY-ESO-1, or a modified or heteroclitic peptide derived from a NY-ESO-1 peptide. In some embodiments, the NY-ESO-1 specific T-cells are generated with peptides that recognize class I MHC molecules. In some embodiments, the NY-ESO-1 specific T-cells are generated with peptides that recognize class II MHC molecules. In some embodiments, the NY-ESO-1 specific T-cells are generated with peptides that recognize both class I and class II MEW molecules.

In some embodiments, the NY-ESO-1 peptides used to prime and expand a T-cell subpopulation includes specifically selected HLA-restricted peptides generated by determining the HLA profile of the donor source, and including peptides derived from NY-ESO-1 that best match the donor's HLA. In some embodiments, the NY-ESO-1 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting NY-ESO-1 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 61-67 , the HLA-B peptides are selected from the peptides of Tables 68-74, and the HLA-DR peptides are selected from the peptides of Tables 75-80. For example, if the donor cell source has an HLA profile that is HLA-A*01/*02:01; HLA-B*15:01/*18; and HLA-DRB1*0101/*0301, then the NY-ESO-1 peptides used to prime and expand the NY-ESO-1 specific T-cell subpopulation are restricted to the specific HLA profile, and may include the peptides identified in Table 61 (SEQ ID NO: 602-611) for HLA-A*01; Table 62 (SEQ ID NO: 612-621) for HLA-A*02:01; Table 70 (SEQ ID NO: 692-701) for HLA-B*15:01; Table 71 (SEQ ID NO: 702-711) for HLA-B*18; Table 75 (SEQ ID NO: 742-751) for HLA-DRB1*0101; and Table 76 (SEQ ID NO: 752-761) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the NY-ESO-1 targeted T-cell subpopulation is primed and expanded with one or more NY-ESO-1-derived peptides selected from Table 61 (SEQ ID NO: 602-611). In some embodiments, the donor cell source is HLA-A*01, and the NY-ESO-1 targeted T-cell subpopulation is primed and expanded with NY-ESO-1-derived peptides selected from Table 61 (SEQ ID NO: 602-611). In some embodiments, the donor cell source is HLA-A*01, and the NY-ESO-1 targeted T-cell subpopulation is primed and expanded with NY-ESO-1-derived peptides comprising the peptides of Table 61 (SEQ ID NO: 602-611). In some embodiments, the donor cell source is HLA-A*01, and the NY-ESO-1 targeted T-cell subpopulation is primed and expanded with NY-ESO-1-derived peptides comprising the peptides of Table 61 (SEQ ID NO: 602-611) and at least one additional set of peptides based on the donor cell source HLA-A profile, wherein the at least one additional set of peptides are selected from the peptides of Tables 62-67. In some embodiments, the NY-ESO-1-derived peptides also include one or more sets of HLA-B and HLA-DR restricted peptides selected from Tables 68-80 (SEQ ID NO: 672-801).

TABLE 61
NYESO1 HLA-A*01 Epitope Peptides

| SEQ ID NO: | Sequence |
|---|---|
| 602 | RGPESRLLEFY |
| 603 | AADHRQLQLSI |
| 604 | EAELARRSLAQ |
| 605 | GPESRLLEFY |
| 606 | AQDAPPLPVP |
| 607 | AADHRQLQLS |
| 608 | EAELARRSLA |
| 609 | PESRLLEFY |
| 610 | AQDAPPLPV |
| 611 | AADHRQLQL |

In some embodiments, the donor cell source is HLA-A*02:01, and the NY-ESO-1 targeted T-cell subpopulation is primed and expanded with one or more NY-ESO-1-derived peptides selected from Table 62 (SEQ ID NO: 612-621). In some embodiments, the donor cell source is HLA-A*02:01, and the NY-ESO-1 targeted T-cell subpopulation is primed and expanded with NY-ESO-1-derived peptides selected from Table 62 (SEQ ID NO: 612-621). In some embodiments, the donor cell source is HLA-A*02:01, and the NY-ESO-1 targeted T-cell subpopulation is primed and expanded with NY-ESO-1-derived peptides comprising the peptides of Table 62 (SEQ ID NO: 612-621). In some embodiments, the donor cell source is HLA-A*02:01, and the NY-ESO-1 targeted T-cell subpopulation is primed and expanded with NY-ESO-1-derived peptides comprising the peptides of Table 62 (SEQ ID NO: 612-621) and at least one additional set of peptides based on the donor cell source HLA-A profile, wherein the at least one additional set of peptides are selected from the peptides of Tables 61, and 63-67. In some embodiments, the NY-ESO-1-derived peptides also include one or more sets of HLA-B and HLA-DR restricted peptides selected from Tables 68-80 (SEQ ID NO: 672-801).

TABLE 62
NYESO1 HLA-A*02:01 Epitope Peptides

| SEQ ID NO: | Sequence |
|---|---|
| 612 | LLMWITQCFL |
| 613 | DAPPLPVPGV |
| 614 | RLLEFYLAMP |
| 615 | FTVSGNILTI |
| 616 | QLQLSISSCL |
| 617 | SLAQDAPPL |
| 618 | SISSCLQQL |
| 619 | RLLEFYLAM |
| 620 | TVSGNILTI |
| 621 | LMWITQCFL |

In some embodiments, the donor cell source is HLA-A*03, and the NY-ESO-1 targeted T-cell subpopulation is primed and expanded with one or more NY-ESO-1-derived peptides selected from Table 63 (SEQ ID NO: 622-631). In some embodiments, the donor cell source is HLA-A*03, and the NY-ESO-1 targeted T-cell subpopulation is primed and expanded with NY-ESO-1-derived peptides selected from Table 63 (SEQ ID NO: 622-631). In some embodiments, the donor cell source is HLA-A*03, and the NY-ESO-1 targeted T-cell subpopulation is primed and expanded with NY-ESO-1-derived peptides comprising the peptides of Table 63 (SEQ ID NO: 622-631). In some embodiments, the donor cell source is HLA-A*03, and the NY-ESO-1 targeted T-cell subpopulation is primed and expanded with NY-ESO-1-derived peptides comprising the peptides of Table 63 (SEQ ID NO: 622-631) and at least one additional set of peptides based on the donor cell source HLA-A profile, wherein the at least one additional set of peptides are selected from the peptides of Tables 61-62 and 64-67. In some embodiments, the NY-ESO-1-derived peptides also include one or more sets of HLA-B and HLA-DR restricted peptides selected from

Tables 68-80 (SEQ ID NO: 672-801).

TABLE 63
NYESO1 HLA-A*03 Epitope Peptides

| SEQ ID NO: | Sequence |
|---|---|
| 622 | PLPVPGVLLK |
| 623 | RLLEFYLAMP |
| 624 | ELARRSLAQD |
| 625 | TIRLTAADHR |
| 626 | RLTAADHRQL |
| 627 | QLSISSCLQQ |
| 628 | FLAQPPSGQR |
| 629 | TIRLTAADH |
| 630 | RLLEFYLAM |
| 631 | ELARRSLAQ |

In some embodiments, the donor cell source is HLA-A*11:01, and the NY-ESO-1 targeted T-cell subpopulation is primed and expanded with one or more NY-ESO-1-derived peptides selected from Table 64 (SEQ ID NO: 632-641). In some embodiments, the donor cell source is HLA-A*11:01, and the NY-ESO-1 targeted T-cell subpopulation is primed and expanded with NY-ESO-1-derived peptides selected from Table 64 (SEQ ID NO: 632-641). In some embodiments, the donor cell source is HLA-A*11:01, and the NY-ESO-1 targeted T-cell subpopulation is primed and expanded with NY-ESO-1-derived peptides comprising the peptides of Table 64 (SEQ ID NO: 632-641). In some embodiments, the donor cell source is HLA-A*11:01, and the NY-ESO-1 targeted T-cell subpopulation is primed and expanded with NY-ESO-1-derived peptides comprising the peptides of Table 64 (SEQ ID NO: 632-641), and at least one additional set of peptides based on the donor cell source HLA-A profile, wherein the at least one additional set of peptides are selected from the peptides of Tables 61-63 and 65-67. In some embodiments, the NY-ESO-1-derived peptides also include one or more sets of HLA-B and HLA-DR restricted peptides selected from Tables 68-80 (SEQ ID NO: 672-801).

TABLE 64
NYESO1 HLA-A*11:01 Epitope Peptides

| SEQ ID NO: | Sequence |
|---|---|
| 632 | ATPMEAELAR |
| 633 | PLPVPGVLLK |
| 634 | ASGPGGGAPR |
| 635 | TVSGNILTIR |
| 636 | GVLLKEFTVS |
| 637 | ASGLNGCCR |
| 638 | LPVPGVLLK |
| 639 | VSGNILTIR |
| 640 | FTVSGNILT |
| 641 | SSCLQQLSL |

In some embodiments, the donor cell source is HLA-A*24:02, and the NY-ESO-1 targeted T-cell subpopulation is primed and expanded with one or more NY-ESO-1-derived peptides selected from Table 65 (SEQ ID NO: 642-651). In some embodiments, the donor cell source is HLA-A*24:02, and the NY-ESO-1 targeted T-cell subpopulation is primed and expanded with NY-ESO-1-derived peptides selected from Table 65 (SEQ ID NO: 642-651). In some embodiments, the donor cell source is HLA-A*24:02, and the NY-ESO-1 targeted T-cell subpopulation is primed and expanded with NY-ESO-1-derived peptides comprising the peptides of Table 65 (SEQ ID NO: 642-651). In some embodiments, the donor cell source is HLA-A*24:02, and the NY-ESO-1 targeted T-cell subpopulation is primed and expanded with NY-ESO-1-derived peptides comprising the peptides of Table 65 (SEQ ID NO: 642-651), and at least one additional set of peptides based on the donor cell source HLA-A profile, wherein the at least one additional set of peptides are selected from the peptides of Tables 61-64 and 66-67. In some embodiments, the NY-ESO-1-derived peptides also include one or more sets of HLA-B and HLA-DR restricted peptides selected from Tables 68-80 (SEQ ID NO: 672-801).

TABLE 65
NYESO1 HLA-A*24:02 Epitope Peptides

| SEQ ID NO: | Sequence |
|---|---|
| 642 | PFATPMEAEL |
| 643 | PPLPVPGVLL |
| 644 | RGPESRLLEF |
| 645 | FYLAMPFATP |
| 646 | APPLPVPGVL |
| 647 | EFTVSGNIL |
| 648 | PPLPVPGVL |
| 649 | FYLAMPFAT |
| 650 | PLPVPGVLL |
| 651 | SCLQQLSLL |

In some embodiments, the donor cell source is HLA-A*26, and the NY-ESO-1 targeted T-cell subpopulation is primed and expanded with one or more NY-ESO-1-derived peptides selected from Table 66 (SEQ ID NO: 652-661). In some embodiments, the donor cell source is HLA-A*26, and the NY-ESO-1 targeted T-cell subpopulation is primed and expanded with NY-ESO-1-derived peptides selected from Table 66 (SEQ ID NO: 652-661). In some embodiments, the donor cell source is HLA-A*26, and the NY-ESO-1 targeted T-cell subpopulation is primed and expanded with NY-ESO-1-derived peptides comprising the peptides of Table 66 (SEQ ID NO: 652-661). In some embodiments, the donor cell source is HLA-A*26, and the NY-ESO-1 targeted T-cell subpopulation is primed and expanded with NY-ESO-1-derived peptides comprising the peptides of Table 66 (SEQ ID NO: 652-661) and at least one additional set of peptides based on the donor cell source HLA-A profile, wherein the at least one additional set of peptides are selected from the peptides of Tables 61-65 and 67. In some embodiments, the NY-ESO-1-derived peptides also include one or more sets of HLA-B and HLA-DR restricted peptides selected from Tables 68-80 (SEQ ID NO: 672-801).

TABLE 66
NYESO1 HLA-A*26 Epitope Peptides

| SEQ ID NO: | Sequence |
|---|---|
| 652 | PVPGVLLKEF |
| 653 | FTVSGNILTI |
| 654 | LSISSCLQQL |
| 655 | WITQCFLPVF |
| 656 | EFTVSGNIL |
| 657 | ITQCFLPVF |
| 658 | ESRLLEFYL |
| 659 | EAELARRSL |
| 660 | SISSCLQQL |
| 661 | TVSGNILTI |

In some embodiments, the donor cell source is HLA-A*68:01, and the NY-ESO-1 targeted T-cell subpopulation is primed and expanded with one or more NY-ESO-1-derived peptides selected from Table 67 (SEQ ID NO: 662-671). In some embodiments, the donor cell source is HLA-A*68:01, and the NY-ESO-1 targeted T-cell subpopulation is primed and expanded with NY-ESO-1-derived peptides selected from Table 67 (SEQ ID NO: 662-671). In some embodiments, the donor cell source is HLA-A*68:01, and the NY-ESO-1 targeted T-cell subpopulation is primed and expanded with NY-ESO-1-derived peptides comprising the peptides of Table 67 (SEQ ID NO: 662-671). In some embodiments, the donor cell source is HLA-A*68:01, and the NY-ESO-1 targeted T-cell subpopulation is primed and expanded with NY-ESO-1-derived peptides comprising the peptides of Table 67 (SEQ ID NO: 662-671), and at least one additional set of peptides based on the donor cell source HLA-A profile, wherein the at least one additional set of peptides are selected from the peptides of Tables 61-66. In some embodiments, the NY-ESO-1-derived peptides also include one or more sets of HLA-B and HLA-DR restricted peptides selected from Tables 68-80 (SEQ ID NO: 672-801).

TABLE 67
NYESO1 HLA-A*68:01 Epitope Peptides

| SEQ ID NO: | Sequence |
|---|---|
| 662 | ATPMEAELARR |
| 663 | FTVSGNILTIR |
| 664 | EAGATGGRGPR |
| 665 | LTIRLTAADHR |
| 666 | RASGPGGGAPR |
| 667 | TVSGNILTIR |
| 668 | ASGPGGGAPR |
| 669 | ATPMEAELAR |
| 670 | VSGNILTIR |
| 671 | PMEAELARR |

In some embodiments, the donor cell source is HLA-B*07:02, and the NY-ESO-1 targeted T-cell subpopulation is primed and expanded with one or more NY-ESO-1-derived peptides selected from Table 68 (SEQ ID NO: 672-681). In some embodiments, the donor cell source is HLA-B*07:02, and the NY-ESO-1 targeted T-cell subpopulation is primed and expanded with NY-ESO-1-derived peptides selected from Table 68 (SEQ ID NO: 672-681). In some embodiments, the donor cell source is HLA-B*07:02, and the NY-ESO-1 targeted T-cell subpopulation is primed and expanded with NY-ESO-1-derived peptides comprising the peptides of Table 68 (SEQ ID NO: 672-681). In some embodiments, the donor cell source is HLA-B*07:02, and the NY-ESO-1 targeted T-cell subpopulation is primed and expanded with NY-ESO-1-derived peptides comprising the peptides of Table 68 (SEQ ID NO: 672-681), and at least one additional set of peptides based on the donor cell source HLA-B profile, wherein the at least one additional set of peptides are selected from the peptides of Tables 69-74. In some embodiments, the NY-ESO-1-derived peptides also include one or more sets of HLA-A and HLA-DR restricted peptides selected from Tables 61-67 and 75-80 (SEQ ID NO: 602-671 and 742-801).

TABLE 68
NYESO1 HLA-B*07:02 Epitope Peptides

| SEQ ID NO: | Sequence |
|---|---|
| 672 | APRGPHGGAA |
| 673 | APPLPVPGVL |
| 674 | PPLPVPGVLL |
| 675 | GPHGGAASGL |
| 676 | GPRGAGAARA |
| 677 | APRGPHGGA |
| 678 | IPDGPGGNA |
| 679 | APPLPVPGV |
| 680 | PPLPVPGVL |
| 681 | GPGGPGIPD |

In some embodiments, the donor cell source is HLA-B*08, and the NY-ESO-1 targeted T-cell subpopulation is primed and expanded with one or more NY-ESO-1-derived peptides selected from Table 69 (SEQ ID NO: 682-691). In some embodiments, the donor cell source is HLA-B*08, and the NY-ESO-1 targeted T-cell subpopulation is primed and expanded with NY-ESO-1-derived peptides selected from Table 69 (SEQ ID NO: 682-691). In some embodiments, the donor cell source is HLA-B*08, and the NY-ESO-1 targeted T-cell subpopulation is primed and expanded with NY-ESO-1-derived peptides comprising the peptides of Table 69 (SEQ ID NO: 682-691). In some embodiments, the donor cell source is HLA-B*08, and the NY-ESO-1 targeted T-cell subpopulation is primed and expanded with NY-ESO-1-derived peptides comprising the peptides of Table 69 (SEQ ID NO: 682-691) and at least one additional set of peptides based on the donor cell source HLA-B profile, wherein the at least one additional set of peptides are selected from the peptides of Tables 68 and 70-74. In some embodiments, the NY-ESO-1-derived peptides also include one or more sets of HLA-A and HLA-DR restricted peptides selected from Tables 61-67 and 75-80 (SEQ ID NO: 602-671 and 742-801).

TABLE 69
NYESO1 HLA-B*08 Epitope Peptides

| SEQ ID NO: | Sequence |
|---|---|
| 682 | GPESRLLEF |
| 683 | AADHRQLQL |
| 684 | GARGPESRL |
| 685 | ESRLLEFYL |
| 686 | LLKEFTVSG |
| 687 | SLAQDAPPL |
| 688 | PLPVPGVLL |
| 689 | AELARRSL |
| 690 | LLKEFTVS |
| 691 | PLPVPGVL |

In some embodiments, the donor cell source is HLA-B*15:01, and the NY-ESO-1 targeted T-cell subpopulation is primed and expanded with one or more NY-ESO-1-derived peptides selected from Table 70 (SEQ ID NO: 692-701). In some embodiments, the donor cell source is HLA-B*15:01, and the NY-ESO-1 targeted T-cell subpopulation is primed and expanded with NY-ESO-1-derived peptides selected from Table 70 (SEQ ID NO: 692-701). In some embodiments, the donor cell source is HLA-B*15:01, and the NY-ESO-1 targeted T-cell subpopulation is primed and expanded with NY-ESO-1-derived peptides comprising the peptides of Table 70 (SEQ ID NO: 692-701). In some embodiments, the donor cell source is HLA-B*15:01, and the NY-ESO-1 targeted T-cell subpopulation is primed and expanded with NY-ESO-1-derived peptides comprising the peptides of Table 70 (SEQ ID NO: 692-701) and at least one additional set of peptides based on the donor cell source HLA-B profile, wherein the at least one additional set of peptides are selected from the peptides of Tables 68-69 and 71-74. In some embodiments, the NY-ESO-1-derived peptides also include one or more sets of HLA-A and HLA-DR restricted peptides selected from Tables 61-67 and 75-80 (SEQ ID NO: 602-671 and 742-801).

TABLE 70
NYESO1 HLA-B*15:01 (B62) Epitope Peptides

| SEQ ID NO: | Sequence |
|---|---|
| 692 | SLLMWITQCF |
| 693 | PVPGVLLKEF |
| 694 | LLEFYLAMPF |
| 695 | RLLEFYLAMP |
| 696 | VLLKEFTVSG |
| 697 | MQAEGRGTGG |
| 698 | ILTIRLTAAD |
| 699 | RQLQLSISSC |
| 700 | LLMWITQCF |
| 701 | LLKEFTVSG |

In some embodiments, the donor cell source is HLA-B*18, and the NY-ESO-1 targeted T-cell subpopulation is primed and expanded with one or more NY-ESO-1-derived peptides selected from Table 71 (SEQ ID NO: 702-711). In some embodiments, the donor cell source is HLA-B*18, and the NY-ESO-1 targeted T-cell subpopulation is primed and expanded with NY-ESO-1-derived peptides selected from Table 71 (SEQ ID NO: 702-711). In some embodiments, the donor cell source is HLA-B*18, and the NY-ESO-1 targeted T-cell subpopulation is primed and expanded with NY-ESO-1-derived peptides comprising the peptides of Table 71 (SEQ ID NO: 702-711). In some embodiments, the donor cell source is HLA-B*18, and the NY-ESO-1 targeted T-cell subpopulation is primed and expanded with NY-ESO-1-derived peptides comprising the peptides of Table 71 (SEQ ID NO: 702-711) and at least one additional set of peptides based on the donor cell source HLA-B profile, wherein the at least one additional set of peptides are selected from the peptides of Tables 68-70 and 72-74. In some embodiments, the NY-ESO-1-derived peptides also include one or more sets of HLA-A and HLA-DR restricted peptides selected from Tables 61-67 and 75-80 (SEQ ID NO: 602-671 and 742-801).

TABLE 71
NYESO1 HLA-B*18 Epitope Peptides

| SEQ ID NO: | Sequence |
|---|---|
| 702 | PESRLLEFY |
| 703 | LEFYLAMPF |
| 704 | MEAELARRS |
| 705 | ESRLLEFYL |
| 706 | VPGVLLKEF |
| 707 | ITQCFLPVF |
| 708 | PESRLLEF |
| 709 | AELARRSL |
| 710 | PGVLLKEF |
| 711 | MEAELARR |

In some embodiments, the donor cell source is HLA-B*27:05, and the NY-ESO-1 targeted T-cell subpopulation is primed and expanded with one or more NY-ESO-1-derived peptides selected from Table 72 (SEQ ID NO: 712-721). In some embodiments, the donor cell source is HLA-B*27:05, and the NY-ESO-1 targeted T-cell subpopulation is primed and expanded with NY-ESO-1-derived peptides selected from Table 72 (SEQ ID NO: 712-721). In some embodiments, the donor cell source is HLA-B*27:05, and the NY-ESO-1 targeted T-cell subpopulation is primed and expanded with NY-ESO-1-derived peptides comprising the peptides of Table 72 (SEQ ID NO: 712-721). In some embodiments, the donor cell source is HLA-B*27:05, and the NY-ESO-1 targeted T-cell subpopulation is primed and expanded with NY-ESO-1-derived peptides comprising the peptides of Table 72 (SEQ ID NO: 712-721) and at least one additional set of peptides based on the donor cell source HLA-B profile, wherein the at least one additional set of peptides are selected from the peptides of Tables 68-71 and 73-74. In some embodiments, the NY-ESO-1-derived peptides also include one or more sets of HLA-A and HLA-DR restricted peptides selected from Tables 61-67 and 75-80 (SEQ ID NO: 602-671 and 742-801).

TABLE 72
NYESO1 HLA-B*27:05 Epitope Peptides

| SEQ ID NO: | Sequence |
|---|---|
| 712 | SRLLEFYLAM |
| 713 | RGPESRLLEF |
| 714 | RSLAQDAPPL |
| 715 | GPHGGAASGL |
| 716 | RRSLAQDAPP |
| 717 | ARGPESRLL |
| 718 | IRLTAADHR |
| 719 | GARGPESRL |
| 720 | GRGTGGSTG |
| 721 | GATGGRGPR |

In some embodiments, the donor cell source is HLA-B*35:01, and the NY-ESO-1 targeted T-cell subpopulation is primed and expanded with one or more NY-ESO-1-derived peptides selected from Table 73 (SEQ ID NO: 722-731). In some embodiments, the donor cell source is HLA-B*35:01, and the NY-ESO-1 targeted T-cell subpopulation is primed and expanded with NY-ESO-1-derived peptides selected from Table 73 (SEQ ID NO: 722-731). In some embodiments, the donor cell source is HLA-B*35:01, and the NY-ESO-1 targeted T-cell subpopulation is primed and expanded with NY-ESO-1-derived peptides comprising the peptides of Table 73 (SEQ ID NO: 722-731). In some embodiments, the donor cell source is HLA-B*35:01, and the NY-ESO-1 targeted T-cell subpopulation is primed and expanded with NY-ESO-1-derived peptides comprising the peptides of Table 73 (SEQ ID NO: 722-731) and at least one additional set of peptides based on the donor cell source HLA-B profile, wherein the at least one additional set of peptides are selected from the peptides of Tables 68-72 and 74. In some embodiments, the NY-ESO-1-derived peptides also include one or more sets of HLA-A and HLA-DR restricted peptides selected from Tables 61-67 and 75-80 (SEQ ID NO: 602-671 and 742-801).

TABLE 73
NYESO1 HLA-B*35:01 Epitope Peptides

| SEQ ID NO: | Sequence |
|---|---|
| 722 | PPLPVPGVLL |
| 723 | GPESRLLEFY |
| 724 | GPHGGAASGL |
| 725 | APPLPVPGVL |
| 726 | MPFATPMEAE |
| 727 | PPLPVPGVL |
| 728 | GPESRLLEF |
| 729 | VPGVLLKEF |
| 730 | LQLSISSCL |
| 731 | LPVFLAQPP |

In some embodiments, the donor cell source is HLA-B*58:02, and the NY-ESO-1 targeted T-cell subpopulation is primed and expanded with one or more NY-ESO-1-derived peptides selected from Table 74 (SEQ ID NO: 732-741). In some embodiments, the donor cell source is HLA-B*58:02, and the NY-ESO-1 targeted T-cell subpopulation is primed and expanded with NY-ESO-1-derived peptides selected from Table 74 (SEQ ID NO: 732-741). In some embodiments, the donor cell source is HLA-B*58:02, and the NY-ESO-1 targeted T-cell subpopulation is primed and expanded with NY-ESO-1-derived peptides comprising the peptides of Table 74 (SEQ ID NO: 732-741). In some embodiments, the donor cell source is HLA-B*58:02, and the NY-ESO-1 targeted T-cell subpopulation is primed and expanded with NY-ESO-1-derived peptides comprising the peptides of Table 74 (SEQ ID NO: 732-741) and at least one additional set of peptides based on the donor cell source HLA-B profile, wherein the at least one additional set of peptides are selected from the peptides of Tables 68-73. In some embodiments, the NY-ESO-1-derived peptides also include one or more sets of HLA-A and HLA-DR restricted peptides selected from Tables 61-67 and 75-80 (SEQ ID NO: 602-671 and 742-801).

TABLE 74
NYESO1 HLA-B*58:02 Epitope Peptides

| SEQ ID NO: | Sequence |
|---|---|
| 732 | RSLAQDAPPL |
| 733 | GARGPESRLL |
| 734 | FTVSGNILTI |
| 735 | LSISSCLQQL |
| 736 | SSCLQQLSLL |
| 737 | VSGNILTIRL |
| 738 | ISSCLQQLSL |
| 739 | EAELARRSL |
| 740 | LTAADHRQL |
| 741 | ESRLLEFYL |

In some embodiments, the donor cell source is HLA-DRB1*0101, and the NY-ESO-1 targeted T-cell subpopulation is primed and expanded with one or more NY-ESO-1-derived peptides selected from Table 75 (SEQ ID NO: 742-751). In some embodiments, the donor cell source is HLA-DRB1*0101, and the NY-ESO-1 targeted T-cell subpopulation is primed and expanded with NY-ESO-1-derived peptides selected from Table 75 (SEQ ID NO: 742-751). In some embodiments, the donor cell source is HLA-DRB1*0101, and the NY-ESO-1 targeted T-cell subpopulation is primed and expanded with NY-ESO-1-derived peptides comprising the peptides of Table 75 (SEQ ID NO: 742-751). In some embodiments, the donor cell source is HLA-DRB1*0101, and the NY-ESO-1 targeted T-cell subpopulation is primed and expanded with NY-ESO-1-derived peptides comprising the peptides of Table 75 (SEQ ID NO: 742-751) and at least one additional set of peptides based on the donor cell source HLA-DR profile, wherein the at least one additional set of peptides are selected from the peptides of Tables 76-80. In some embodiments, the NY-ESO-1-derived peptides also include one or more sets of HLA-A and HLA-B restricted peptides selected from Tables 61-74 (SEQ ID NO: 602-741).

TABLE 75
NYESO1 HLA-DRB1*0101 Epitope Peptides

| SEQ ID NO: | Sequence |
|---|---|
| 742 | EFYLAMPFATPMEAE |
| 743 | SRLLEFYLAMPFATP |
| 744 | ATPMEAELARRSLAQ |
| 745 | GPGIPDGPGGNAGGP |
| 746 | LEFYLAMPFATPMEA |
| 747 | MPFATPMEAELARRS |
| 748 | LLMWITQCFLPVFLA |
| 749 | TQCFLPVFLAQPPSG |
| 750 | QCFLPVFLAQPPSGQ |
| 751 | YLAMPFATPMEAELA |

In some embodiments, the donor cell source is HLA-DRB1*0301, and the NY-ESO-1 targeted T-cell subpopulation is primed and expanded with one or more NY-ESO-1-derived peptides selected from Table 76 (SEQ ID NO: 752-761). In some embodiments, the donor cell source is HLA-DRB1*0301, and the NY-ESO-1 targeted T-cell subpopulation is primed and expanded with NY-ESO-1-derived peptides selected from Table 76 (SEQ ID NO: 752-761). In some embodiments, the donor cell source is HLA-DRB1*0301, and the NY-ESO-1 targeted T-cell subpopulation is primed and expanded with NY-ESO-1-derived peptides comprising the peptides of Table 76 (SEQ ID NO: 752-761). In some embodiments, the donor cell source is HLA-DRB1*0301, and the NY-ESO-1 targeted T-cell subpopulation is primed and expanded with NY-ESO-1-derived peptides comprising the peptides of Table 76 (SEQ ID NO: 752-761) and at least one additional set of peptides based on the donor cell source HLA-DR profile, wherein the at least one additional set of peptides are selected from the peptides of Tables 75 and 77-80. In some embodiments, the NY-ESO-1-derived peptides also include one or more sets of HLA-A and HLA-B restricted peptides selected from Tables 61-74 (SEQ ID NO: 602-741).

TABLE 76
NYESO1 HLA-DRB1*0301 (DR17) Epitope Peptides

| SEQ ID NO: | Sequence |
|---|---|
| 752 | LSLLMWITQCFLPVF |
| 753 | AMPFATPMEAELARR |
| 754 | QLSLLMWITQCFLPV |
| 755 | RRSLAQDAPPLPVPG |
| 756 | QLSISSCLQQLSLLM |
| 757 | SRLLEFYLAMPFATP |
| 758 | PLPVPGVLLKEFTVS |
| 759 | TIRLTAADHRQLQLS |
| 760 | HRQLQLSISSCLQQL |
| 761 | LMWITQCFLPVFLAQ |

In some embodiments, the donor cell source is HLA-DRB1*0401, and the NY-ESO-1 targeted T-cell subpopulation is primed and expanded with one or more NY-ESO-1-derived peptides selected from Table 77 (SEQ ID NO: 762-771). In some embodiments, the donor cell source is HLA-DRB1*0401, and the NY-ESO-1 targeted T-cell subpopulation is primed and expanded with NY-ESO-1-derived peptides selected from Table 77 (SEQ ID NO: 762-771). In some embodiments, the donor cell source is HLA-DRB1*0401, and the NY-ESO-1 targeted T-cell subpopulation is primed and expanded with NY-ESO-1-derived peptides comprising the peptides of Table 77 (SEQ ID NO: 762-771). In some embodiments, the donor cell source is HLA-DRB1*0401, and the NY-ESO-1 targeted T-cell subpopulation is primed and expanded with NY-ESO-1-derived peptides comprising the peptides of Table 77 (SEQ ID NO: 762-771) and at least one additional set of peptides based on the donor cell source HLA-DR profile, wherein the at least one additional set of peptides are selected from the peptides of Tables 75-76 and 78-80. In some embodiments, the NY-ESO-1-derived peptides also include one or more sets of HLA-A and HLA-B restricted peptides selected from Tables 61-74 (SEQ ID NO: 602-741).

TABLE 77
NYESO1 HLA-DRB1*0401 (DR4Dw4) Epitope Peptides

| SEQ ID NO: | Sequence |
|---|---|
| 762 | TIRLTAADHRQLQLS |
| 763 | LSLLMWITQCFLPVF |
| 764 | LLEFYLAMPFATPME |
| 765 | LKEFTVSGNILTIRL |
| 766 | ASGLNGCCRCGARGP |
| 767 | YLAMPFATPMEAELA |
| 768 | ATPMEAELARRSLAQ |
| 769 | PGVLLKEFTVSGNIL |
| 770 | GVLLKEFTVSGNILT |
| 771 | SGNILTIRLTAADHR |

In some embodiments, the donor cell source is HLA-DRB1*0701, and the NY-ESO-1 targeted T-cell subpopulation is primed and expanded with one or more NY-ESO-1-derived peptides selected from Table 78 (SEQ ID NO: 772-781). In some embodiments, the donor cell source is HLA-DRB1*0701, and the NY-ESO-1 targeted T-cell subpopulation is primed and expanded with NY-ESO-1-derived peptides selected from Table 78 (SEQ ID NO: 772-781). In some embodiments, the donor cell source is HLA-DRB1*0701, and the NY-ESO-1 targeted T-cell subpopulation is primed and expanded with NY-ESO-1-derived peptides comprising the peptides of Table 78 (SEQ ID NO: 772-781). In some embodiments, the donor cell source is HLA-DRB1*0701, and the NY-ESO-1 targeted T-cell subpopulation is primed and expanded with NY-ESO-1-derived peptides comprising the peptides of Table 78 (SEQ ID NO: 772-781) and at least one additional set of peptides based on the donor cell source HLA-DR profile, wherein the at least one additional set of peptides are selected from the peptides of Tables 75-77 and 79-80. In some embodiments, the NY-ESO-1-derived peptides also include one or more sets of HLA-A and HLA-B restricted peptides selected from Tables 61-74 (SEQ ID NO: 602-741).

TABLE 78
NYESO1 HLA-DRB1*0701 Epitope Peptides

| SEQ ID NO: | Sequence |
|---|---|
| 772 | HRQLQLSISSCLQQL |
| 773 | AMPFATPMEAELARR |
| 774 | VLLKEFTVSGNILTI |
| 775 | LKEFTVSGNILTIRL |
| 776 | FTVSGNILTIRLTAA |
| 777 | TIRLTAADHRQLQLS |
| 778 | QLSLLMWITQCFLPV |
| 779 | LSLLMWITQCFLPVF |
| 780 | YLAMPFATPMEAELA |
| 781 | SGNILTIRLTAADHR |

In some embodiments, the donor cell source is HLA-DRB1*1101, and the NY-ESO-1 targeted T-cell subpopulation is primed and expanded with one or more NY-ESO-1-derived peptides selected from Table 79 (SEQ ID NO: 782-791). In some embodiments, the donor cell source is HLA-DRB1*1101, and the NY-ESO-1 targeted T-cell subpopulation is primed and expanded with NY-ESO-1-derived peptides selected from Table 79 (SEQ ID NO: 782-791). In some embodiments, the donor cell source is HLA-DRB1*1101, and the NY-ESO-1 targeted T-cell subpopulation is primed and expanded with NY-ESO-1-derived peptides comprising the peptides of Table 79 (SEQ ID NO: 782-791). In some embodiments, the donor cell source is HLA-DRB1*1101, and the NY-ESO-1 targeted T-cell subpopulation is primed and expanded with NY-ESO-1-derived peptides comprising the peptides of Table 79 (SEQ ID NO: 782-791) and at least one additional set of peptides based on the donor cell source HLA-DR profile, wherein the at least one additional set of peptides are selected from the peptides of Tables 75-78 and 80. In some embodiments, the NY-ESO-1-derived peptides also include one or more sets of HLA-A and HLA-B restricted peptides selected from Tables 61-74 (SEQ ID NO: 602-741).

TABLE 79
NYESO1 HLA-DRB1*1101 Epitope Peptides

| SEQ ID NO: | Sequence |
|---|---|
| 782 | LEFYLAMPFATPMEA |
| 783 | TQCFLPVFLAQPPSG |
| 784 | ASGLNGCCRCGARGP |
| 785 | SGNILTIRLTAADHR |
| 786 | TIRLTAADHRQLQLS |
| 787 | MPFATPMEAELARRS |
| 788 | ATPMEAELARRSLAQ |
| 789 | TPMEAELARRSLAQD |
| 790 | PMEAELARRSLAQDA |
| 791 | LPVPGVLLKEFTVSG |

In some embodiments, the donor cell source is HLA-DRB1*1501, and the NY-ESO-1 targeted T-cell subpopulation is primed and expanded with one or more NY-ESO-1-derived peptides selected from Table 80 (SEQ ID NO: 792-801). In some embodiments, the donor cell source is HLA-DRB1*1501, and the NY-ESO-1 targeted T-cell subpopulation is primed and expanded with NY-ESO-1-derived peptides selected from T Table 80 (SEQ ID NO: 792-801). In some embodiments, the donor cell source is HLA-DRB1*1501, and the NY-ESO-1 targeted T-cell subpopulation is primed and expanded with NY-ESO-1-derived peptides comprising the peptides of Table 80 (SEQ ID NO: 792-801). In some embodiments, the donor cell source is HLA-DRB1*1501, and the NY-ESO-1 targeted T-cell subpopulation is primed and expanded with NY-ESO-1-derived peptides comprising the peptides of Table 80 (SEQ ID NO: 792-801) and at least one additional set of peptides based on the donor cell source HLA-DR profile, wherein the at least one additional set of peptides are selected from the peptides of Tables 75-79. In some embodiments, the NY-ESO-1-derived peptides also include one or more sets of HLA-A and HLA-B restricted peptides selected from Tables 61-74 (SEQ ID NO: 602-741).

TABLE 80
NYESO1 HLA-DRB1*1501 (DR2b) Epitope Peptides

| SEQ ID NO: | Sequence |
|---|---|
| 792 | SRLLEFYLAMPFATP |
| 793 | QCFLPVFLAQPPSGQ |
| 794 | ESRLLEFYLAMPFAT |
| 795 | YLAMPFATPMEAELA |
| 796 | PGVLLKEFTVSGNIL |
| 797 | GVLLKEFTVSGNILT |
| 798 | QLSLLMWITQCFLPV |
| 799 | MWITQCFLPVFLAQP |
| 800 | LLEFYLAMPFATPME |
| 801 | LKEFTVSGNILTIRL |

MAGE-A3 Antigenic Peptides

In some embodiments, the MUSTANG composition includes MAGE-A3 (Melanoma-associated antigen 3) specific T-cells. MAGE-A3 specific T-cells can be generated as described below using one or more antigenic peptides to MAGE-A3. In some embodiments, the MAGE-A3 specific T-cells are generated using one or more antigenic peptides to MAGE-A3, or a modified or heteroclitic peptide derived from a MAGE-A3 peptide. In some embodiments, MAGE-A3 specific T-cells are generated using a MAGE-A3 antigen library comprising a pool of peptides (for example 15 mers) containing amino acid overlap (for example 11 amino acids of overlap) between each sequence formed by scanning the protein amino acid sequence SEQ ID NO: 802 (UniProt KB-P43357) for MAGE-A3:

MPLEQRSQHCKPEEGLEARGEALGLVGAQAPATEEQEAASSSSTLVEVTL
GEVPAAESPDPPQSPQGASSLPTTMNYPLWSQSYEDSSNQEEEGPSTFPD
LESEFQAALSRKVAELVHFLLLKYRAREPVTKAEMLGSVVGNWQYFFPVI
LLIIVLATIAREGDCAPEEKIWEELSVLEVFEGREDSILGDPKKLLTQHF
VQENYLEYRQVPGSDPACYEFLWGPRALVETSYVKVLHHMVKISGGPHIS
YPPLHEWVLREGEE.

Overlapping antigenic libraries are commercially available, for example, from JPT, for example, from JPT (Product Code: PM-MAGEA3 (Pep Mix Human (MAGE-A3)). In some embodiments, the MAGE-A3 specific T-cells are generated using a commercially available overlapping antigenic library made up of MAGE-A3 peptides.

In some embodiments, the MAGE-A3 specific T-cells are generated using one or more antigenic peptides to MAGE-A3, or a modified or heteroclitic peptide derived from a MAGE-A3 peptide. In some embodiments, the MAGE-A3 specific T-cells are generated with peptides that recognize class I MHC molecules. In some embodiments, the MAGE-A3 specific T-cells are generated with peptides that recognize class II MHC molecules. In some embodiments, the MAGE-A3 specific T-cells are generated with peptides that recognize both class I and class II MHC molecules.

In some embodiments, the MAGE-A3 peptides used to prime and expand a T-cell subpopulation includes specifically selected HLA-restricted peptides generated by determining the HLA profile of the donor source, and including peptides derived from MAGE-A3 that best match the donor's HLA. In some embodiments, the MAGE-A3 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting MAGE-A3 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 81-87 , the HLA-B peptides are selected from the peptides of Tables 88-94, and the HLA-DR peptides are selected from the peptides of Tables 95-100. For example, if the donor cell source has an HLA profile that is HLA-A*01/*02:01; HLA-B*15:01/*18; and HLA-DRB1*0101/*0301, then the MAGE-A3 peptides used to prime and expand the MAGE-A3 specific T-cell subpopulation are restricted to the specific HLA profile, and may include the peptides identified in Table 81 (SEQ ID NO: 803-812) for HLA-A*01; Table 82 (SEQ ID NO: 813-822) for HLA-A*02:01; Table 90 (SEQ ID NO: 893-902) for HLA-B*15:01; Table 91 (SEQ ID NO: 903-912) for HLA-B*18; Table 95 (SEQ ID NO: 943-952) for HLA-DRB1*0101; and Table 96 (SEQ ID NO: 953-962) for HLA-DRB1*0301. In some embodiments, the mastermix of peptides includes both an overlapping peptide library and specifically selected HLA-restricted peptides generated by determining the HLA profile of the donor source.

In some embodiments, the donor cell source is HLA-A*01, and the MAGE-A3 targeted T-cell subpopulation is primed and expanded with one or more MAGE-A3-derived peptides selected from Table 81 (SEQ ID NO: 803-812). In some embodiments, the donor cell source is HLA-A*01, and the MAGE-A3 targeted T-cell subpopulation is primed and expanded with MAGE-A3-derived peptides selected from Table 81 (SEQ ID NO: 803-812). In some embodiments, the donor cell source is HLA-A*01, and the MAGE-A3 targeted T-cell subpopulation is primed and expanded with MAGE-A3-derived peptides comprising the peptides of Table 81 (SEQ ID NO: 803-812). In some embodiments, the donor cell source is HLA-A*01, and the MAGE-A3 targeted T-cell subpopulation is primed and expanded with MAGE-A3-derived peptides comprising the peptides of Table 81 (SEQ ID NO: 803-812) and at least one additional set of peptides based on the donor cell source HLA-A profile, wherein the at least one additional set of peptides are selected from the peptides of Tables 82-87. In some embodiments, the MAGE-A3-derived peptides also include one or more sets of HLA-B and HLA-DR restricted peptides selected from Tables 88-100

(SEQ ID NO: 873-1002).

TABLE 81
MAGEA3 HLA-A*01 Epitope Peptides

| SEQ ID NO: | Sequence |
|---|---|
| 803 | LMEVDPIGHLY |
| 804 | AELVHFLLLKY |
| 805 | QHFVQENYLEY |
| 806 | ASSLPTTMNY |
| 807 | ELVHFLLLKY |
| 808 | LTQHFVQENY |
| 809 | EVDPIGHLY |
| 810 | SSLPTTMNY |
| 811 | LVHFLLLKY |
| 812 | GSVVGNWQY |

In some embodiments, the donor cell source is HLA-A*02:01, and the MAGE-A3 targeted T-cell subpopulation is primed and expanded with one or more MAGE-A3-derived peptides selected from Table 82 (SEQ ID NO: 813-822). In some embodiments, the donor cell source is HLA-A*02:01, and the MAGE-A3 targeted T-cell subpopulation is primed and expanded with MAGE-A3-derived peptides selected from Table 82 (SEQ ID NO: 813-822). In some embodiments, the donor cell source is HLA-A*02:01, and the MAGE-A3 targeted T-cell subpopulation is primed and expanded with MAGE-A3-derived peptides comprising the peptides of Table 82 (SEQ ID NO: 813-822). In some embodiments, the donor cell source is HLA-A*02:01, and the MAGE-A3 targeted T-cell subpopulation is primed and expanded with MAGE-A3-derived peptides comprising the peptides of Table 82 (SEQ ID NO: 813-822) and at least one additional set of peptides based on the donor cell source HLA-A profile, wherein the at least one additional set of peptides are selected from the peptides of Tables 81, and 83-87. In some embodiments, the MAGE-A3-derived peptides also include one or more sets of HLA-B and HLA-DR restricted peptides selected from Tables 88-100 (SEQ ID NO: 873-1002).

TABLE 82
MAGEA3 HLA-A*02:01 Epitope Peptides

| SEQ ID NO: | Sequence |
|---|---|
| 813 | TLVEVTLGEV |
| 814 | ALVETSYVKV |
| 815 | GLLIIVLAII |
| 816 | AALSRKVAEL |
| 817 | LVFGIELMEV |
| 818 | ALSRKVAEL |
| 819 | LLIIVLAII |
| 820 | GLLIIVLAI |
| 821 | FLWGPRALV |
| 822 | KIWEELSVL |

In some embodiments, the donor cell source is HLA-A*03, and the MAGE-A3 targeted T-cell subpopulation is primed and expanded with one or more MAGE-A3-derived peptides selected from Table 83 (SEQ ID NO: 823-832). In some embodiments, the donor cell source is HLA-A*03, and the MAGE-A3 targeted T-cell subpopulation is primed and expanded with MAGE-A3-derived peptides selected from Table 83 (SEQ ID NO: 823-832). In some embodiments, the donor cell source is HLA-A*03, and the MAGE-A3 targeted T-cell subpopulation is primed and expanded with MAGE-A3-derived peptides comprising the peptides of Table 83 (SEQ ID NO: 823-832). In some embodiments, the donor cell source is HLA-A*03, and the MAGE-A3 targeted T-cell subpopulation is primed and expanded with MAGE-A3-derived peptides comprising the peptides of Table 83 (SEQ ID NO: 823-832) and at least one additional set of peptides based on the donor cell source HLA-A profile, wherein the at least one additional set of peptides are selected from the peptides of Tables 81-82 and 84-87. In some embodiments, the MAGE-A3-derived peptides also include one or more sets of HLA-B and HLA-DR restricted peptides selected from Tables 88-100 (SEQ ID NO: 873-1002).

TABLE 83
MAGEA3 HLA-A*03 Epitope Peptides

| SEQ ID NO: | Sequence |
|---|---|
| 823 | KYRAREPVTK |
| 824 | YVKVLHHMVK |
| 825 | QVPGSDPACY |
| 826 | LLGDNQIMPK |
| 827 | KLLTQHFVQE |
| 828 | FLWGPRALVE |
| 829 | ALVETSYVK |
| 830 | ALGLVGAQA |
| 831 | ELVHFLLLK |
| 832 | YRAREPVTK |

In some embodiments, the donor cell source is HLA-A*11:01, and the MAGE-A3 targeted T-cell subpopulation is primed and expanded with one or more MAGE-A3-derived peptides selected from Table 84 (SEQ ID NO: 833-842). In some embodiments, the donor cell source is HLA-A*11:01, and the MAGE-A3 targeted T-cell subpopulation is primed and expanded with MAGE-A3-derived peptides selected from Table 84 (SEQ ID NO: 833-842). In some embodiments, the donor cell source is HLA-A*11:01, and the MAGE-A3 targeted T-cell subpopulation is primed and expanded with MAGE-A3-derived peptides comprising the peptides of Table 84 (SEQ ID NO: 833-842). In some embodiments, the donor cell source is HLA-A*11:01, and the MAGE-A3 targeted T-cell subpopulation is primed and expanded with MAGE-A3-derived peptides comprising the peptides of Table 84 (SEQ ID NO: 833-842), and at least one additional set of peptides based on the donor cell source HLA-A profile, wherein the at least one additional set of peptides are selected from the peptides of Tables 81-83 and 85-87. In some embodiments, the MAGE-A3-derived peptides also include one or more sets of HLA-B and HLA-DR restricted peptides selected from Tables 88-100 (SEQ ID NO: 873-1002).

TABLE 84
MAGEA3 HLA-A*11:01 Epitope Peptides

| SEQ ID NO: | Sequence |
|---|---|
| 833 | ESEFQAALSR |
| 834 | YVKVLHHMVK |
| 835 | AELVHFLLLK |
| 836 | LIIVLAIIAR |
| 837 | ASSSSTLVEV |
| 838 | STLVEVTLGE |
| 839 | ELVHFLLLK |
| 840 | SVLEVFEGR |
| 841 | DSILGDPKK |
| 842 | ALVETSYVK |

In some embodiments, the donor cell source is HLA-A*24:02, and the MAGE-A3 targeted T-cell subpopulation is primed and expanded with one or more MAGE-A3-derived peptides selected from Table 85 (SEQ ID NO: 843-852). In some embodiments, the donor cell source is HLA-A*24:02, and the MAGE-A3 targeted T-cell subpopulation is primed and expanded with MAGE-A3-derived peptides selected from Table 85 (SEQ ID NO: 843-852). In some embodiments, the donor cell source is HLA-A*24:02, and the MAGE-A3 targeted T-cell subpopulation is primed and expanded with MAGE-A3-derived peptides comprising the peptides of Table 85 (SEQ ID NO: 843-852). In some embodiments, the donor cell source is HLA-A*24:02, and the MAGE-A3 targeted T-cell subpopulation is primed and expanded with MAGE-A3-derived peptides comprising the peptides of Table 85 (SEQ ID NO: 843-852), and at least one additional set of peptides based on the donor cell source HLA-A profile, wherein the at least one additional set of peptides are selected from the peptides of Tables 81-84 and 86-87. In some embodiments, the MAGE-A3-derived peptides also include one or more sets of HLA-B and HLA-DR restricted peptides selected from Tables 88-100 (SEQ ID NO: 873-1002).

TABLE 85
MAGEA3 HLA-A*24:02 Epitope Peptides

| SEQ ID NO: | Sequence |
|---|---|
| 843 | SYPPLHEWVL |
| 844 | LYIFATCLGL |
| 845 | VFEGREDSIL |
| 846 | KVAELVHFLL |
| 847 | TFPDLESEF |
| 848 | VFEGREDSI |
| 849 | EFLWGPRAL |
| 850 | VAELVHFLL |
| 851 | IFSKASSSL |
| 852 | AELVHFLLL |

In some embodiments, the donor cell source is HLA-A*26, and the MAGE-A3 targeted T-cell subpopulation is primed and expanded with one or more MAGE-A3-derived peptides selected from Table 86 (SEQ ID NO: 853-862). In some embodiments, the donor cell source is HLA-A*26, and the MAGE-A3 targeted T-cell subpopulation is primed and expanded with MAGE-A3-derived peptides selected from Table 86 (SEQ ID NO: 853-862). In some embodiments, the donor cell source is HLA-A*26, and the MAGE-A3 targeted T-cell subpopulation is primed and expanded with MAGE-A3-derived peptides comprising the peptides of Table 86 (SEQ ID NO: 853-862). In some embodiments, the donor cell source is HLA-A*26, and the MAGE-A3 targeted T-cell subpopulation is primed and expanded with MAGE-A3-derived peptides comprising the peptides of Table 86 (SEQ ID NO: 853-862) and at least one additional set of peptides based on the donor cell source HLA-A profile, wherein the at least one additional set of peptides are selected from the peptides of Tables 81-85 and 87. In some embodiments, the MAGE-A3-derived peptides also include one or more sets of HLA-B and HLA-DR restricted peptides selected from Tables 88-100 (SEQ ID NO: 873-1002).

TABLE 86
MAGEA3 HLA-A*26 Epitope Peptides

| SEQ ID NO: | Sequence |
|---|---|
| 853 | ELVHFLLLKY |
| 854 | EKIWEELSVL |
| 855 | EVFEGREDSI |
| 856 | EVTLGEVPAA |
| 857 | EVDPIGHLY |
| 858 | LVHFLLLKY |
| 859 | EVFEGREDS |
| 860 | KVAELVHFL |
| 861 | EPVTKAEML |
| 862 | SVVGNWQYF |

In some embodiments, the donor cell source is HLA-A*68:01, and the MAGE-A3 targeted T-cell subpopulation is primed and expanded with one or more MAGE-A3-derived peptides selected from Table 87 (SEQ ID NO: 863-872). In some embodiments, the donor cell source is HLA-A*68:01, and the MAGE-A3 targeted T-cell subpopulation is primed and expanded with MAGE-A3-derived peptides selected from Table 87 (SEQ ID NO: 863-872). In some embodiments, the donor cell source is HLA-A*68:01, and the MAGE-A3 targeted T-cell subpopulation is primed and expanded with MAGE-A3-derived peptides comprising the peptides of Table 87 (SEQ ID NO: 863-872). In some embodiments, the donor cell source is HLA-A*68:01, and the MAGE-A3 targeted T-cell subpopulation is primed and expanded with MAGE-A3-derived peptides comprising the peptides of Table 87 (SEQ ID NO: 863-872), and at least one additional set of peptides based on the donor cell source HLA-A profile, wherein the at least one additional set of peptides are selected from the peptides of Tables 81-86. In some embodiments, the MAGE-A3-derived peptides also include one or more sets of HLA-B and HLA-DR restricted peptides selected from Tables 88-100 (SEQ ID NO: 873-1002).

TABLE 87
MAGEA3 HLA-A*68:01 Epitope Peptides

| SEQ ID NO: | Sequence |
|---|---|
| 863 | LLIIVLAIIAR |
| 864 | ELVHFLLLKYR |
| 865 | ELSVLEVFEGR |
| 866 | LIIVLAIIAR |
| 867 | ESEFQAALSR |
| 868 | IIVLAIIAR |
| 869 | ELVHFLLLK |
| 870 | IVLAIIARE |
| 871 | SVLEVFEGR |
| 872 | DSILGDPKK |

In some embodiments, the donor cell source is HLA-B*07:02, and the MAGE-A3 targeted T-cell subpopulation is primed and expanded with one or more MAGE-A3-derived peptides selected from Table 88 (SEQ ID NO: 873-882). In some embodiments, the donor cell source is HLA-B*07:02, and the MAGE-A3 targeted T-cell subpopulation is primed and expanded with MAGE-A3-derived peptides selected from Table 88 (SEQ ID NO: 873-882). In some embodiments, the donor cell source is HLA-B*07:02, and the MAGE-A3 targeted T-cell subpopulation is primed and expanded with MAGE-A3-derived peptides comprising the peptides of Table 88 (SEQ ID NO: 873-882). In some embodiments, the donor cell source is HLA-B*07:02, and the MAGE-A3 targeted T-cell subpopulation is primed and expanded with MAGE-A3-derived peptides comprising the peptides of Table 88 (SEQ ID NO: 873-882), and at least one additional set of peptides based on the donor cell source HLA-B profile, wherein the at least one additional set of peptides are selected from the peptides of Tables 89-94. In some embodiments, the MAGE-A3-derived peptides also include one or more sets of HLA-A and HLA-DR restricted peptides selected from Tables 81-87 and 95-100 (SEQ ID NO: 803-872 and 943-1002).

TABLE 88
MAGEA3 HLA-B*07:02 Epitope Peptides

| SEQ ID NO: | Sequence |
|---|---|
| 873 | APEEKIWEEL |
| 874 | SPQGASSLPT |
| 875 | APATEEQEAA |
| 876 | DPIGHLYIFA |
| 877 | GPHISYPPL |
| 878 | LPTTMNYPL |
| 879 | EPVTKAEML |
| 880 | YPPLHEWVL |
| 881 | APATEEQEA |
| 882 | MPKAGLLII |

In some embodiments, the donor cell source is HLA-B*08, and the MAGE-A3 targeted T-cell subpopulation is primed and expanded with one or more MAGE-A3-derived peptides selected from Table 89 (SEQ ID NO: 883-892). In some embodiments, the donor cell source is HLA-B*08, and the MAGE-A3 targeted T-cell subpopulation is primed and expanded with MAGE-A3-derived peptides selected from Table 89 (SEQ ID NO: 883-892). In some embodiments, the donor cell source is HLA-B*08, and the MAGE-A3 targeted T-cell subpopulation is primed and expanded with MAGE-A3-derived peptides comprising the peptides of Table 89 (SEQ ID NO: 883-892). In some embodiments, the donor cell source is HLA-B*08, and the MAGE-A3 targeted T-cell subpopulation is primed and expanded with MAGE-A3-derived peptides comprising the peptides of Table 89 (SEQ ID NO: 883-892) and at least one additional set of peptides based on the donor cell source HLA-B profile, wherein the at least one additional set of peptides are selected from the peptides of Tables 88 and 90-94. In some embodiments, the MAGE-A3-derived peptides also include one or more sets of HLA-A and HLA-DR restricted peptides selected from Tables 81-87 and 95-100 (SEQ ID NO: 803-872 and 943-1002).

TABLE 89
MAGEA3 HLA-B*08 Epitope Peptides

| SEQ ID NO: | Sequence |
|---|---|
| 883 | ALSRKVAEL |
| 884 | EPVTKAEML |
| 885 | GLEARGEAL |
| 886 | LLKYRAREP |
| 887 | QIMPKAGLL |
| 888 | EARGEALGL |
| 889 | MPKAGLLII |
| 890 | LLKYRARE |
| 891 | QIMPKAGL |
| 892 | EEKIWEEL |

In some embodiments, the donor cell source is HLA-B*15:01, and the MAGE-A3 targeted T-cell subpopulation is primed and expanded with one or more MAGE-A3-derived peptides selected from Table 90 (SEQ ID NO: 893-902). In some embodiments, the donor cell source is HLA-B*15:01, and the MAGE-A3 targeted T-cell subpopulation is primed and expanded with MAGE-A3-derived peptides selected from Table 90 (SEQ ID NO: 893-902). In some embodiments, the donor cell source is HLA-B*15:01, and the MAGE-A3 targeted T-cell subpopulation is primed and expanded with MAGE-A3-derived peptides comprising the peptides of Table 90 (SEQ ID NO: 893-902). In some embodiments, the donor cell source is HLA-B*15:01, and the MAGE-A3 targeted T-cell subpopulation is primed and expanded with MAGE-A3-derived peptides comprising the peptides of Table 90 (SEQ ID NO: 893-902) and at least one additional set of peptides based on the donor cell source HLA-B profile, wherein the at least one additional set of peptides are selected from the peptides of Tables 88-89 and 91-94. In some embodiments, the MAGE-A3-derived peptides also include one or more sets of HLA-A and HLA-DR restricted peptides selected from Tables 81-87 and 95-100 (SEQ ID NO: 803-872 and 943-1002).

TABLE 90
MAGEA3 HLA-B*15:01 (B62) Epitope Peptides

| SEQ ID NO: | Sequence |
|---|---|
| 893 | NQEEEGPSTF |
| 894 | ELVHFLLLKY |
| 895 | QVPGSDPACY |
| 896 | SVVGNWQYFF |
| 897 | TQHFVQENY |
| 898 | LVHFLLLKY |
| 899 | FVQENYLEY |
| 900 | WQYFFPVIF |
| 901 | EVDPIGHLY |
| 902 | VVGNWQYFF |

In some embodiments, the donor cell source is HLA-B*18, and the MAGE-A3 targeted T-cell subpopulation is primed and expanded with one or more MAGE-A3-derived peptides selected from Table 91 (SEQ ID NO: 903-912). In some embodiments, the donor cell source is HLA-B*18, and the MAGE-A3 targeted T-cell subpopulation is primed and expanded with MAGE-A3-derived peptides selected from Table 91 (SEQ ID NO: 903-912). In some embodiments, the donor cell source is HLA-B*18, and the MAGE-A3 targeted T-cell subpopulation is primed and expanded with MAGE-A3-derived peptides comprising the peptides of Table 91 (SEQ ID NO: 903-912). In some embodiments, the donor cell source is HLA-B*18, and the MAGE-A3 targeted T-cell subpopulation is primed and expanded with MAGE-A3-derived peptides comprising the peptides of Table 91 (SEQ ID NO: 903-912) and at least one additional set of peptides based on the donor cell source HLA-B profile, wherein the at least one additional set of peptides are selected from the peptides of Tables 88-90 and 92-94. In some embodiments, the MAGE-A3-derived peptides also include one or more sets of HLA-A and HLA-DR restricted peptides selected from Tables 81-87 and 95-100 (SEQ ID NO: 803-872 and 943-1002).

TABLE 91
MAGEA3 HLA-B*18 Epitope Peptides

| SEQ ID NO: | Sequence |
|---|---|
| 903 | EELSVLEVF |
| 904 | QEEEGPSTF |
| 905 | LESEFQAAL |
| 906 | PEEKIWEEL |
| 907 | AELVHFLLL |
| 908 | VETSYVKVL |
| 909 | EEEGPSTF |
| 910 | EEKIWEEL |
| 911 | AELVHFLL |
| 912 | LEARGEAL |

In some embodiments, the donor cell source is HLA-B*27:05, and the MAGE-A3 targeted T-cell subpopulation is primed and expanded with one or more MAGE-A3-derived peptides selected from Table 92 (SEQ ID NO: 913-922). In some embodiments, the donor cell source is HLA-B*27:05, and the MAGE-A3 targeted T-cell subpopulation is primed and expanded with MAGE-A3-derived peptides selected from Table 92 (SEQ ID NO: 913-922). In some embodiments, the donor cell source is HLA-B*27:05, and the MAGE-A3 targeted T-cell subpopulation is primed and expanded with MAGE-A3-derived peptides comprising the peptides of Table 92 (SEQ ID NO: 913-922). In some embodiments, the donor cell source is HLA-B*27:05, and the MAGE-A3 targeted T-cell subpopulation is primed and expanded with MAGE-A3-derived peptides comprising the peptides of Table 92 (SEQ ID NO: 913-922) and at least one additional set of peptides based on the donor cell source HLA-B profile, wherein the at least one additional set of peptides are selected from the peptides of Tables 88-91 and 93-94. In some embodiments, the MAGE-A3-derived peptides also include one or more sets of HLA-A and HLA-DR restricted peptides selected from Tables 81-87 and 95-100 (SEQ ID NO: 803-872 and 943-1002).

TABLE 92
MAGEA3 HLA-B*27:05 Epitope Peptides

| SEQ ID NO: | Sequence |
|---|---|
| 913 | AREPVTKAEM |
| 914 | SRKVAELVHF |
| 915 | SEFQAALSRK |
| 916 | RALVETSYVK |
| 917 | YRAREPVTK |
| 918 | PRALVETSY |
| 919 | SRKVAELVH |
| 920 | YFFPVIFSK |
| 921 | KAGLLIIVL |
| 922 | DSILGDPKK |

In some embodiments, the donor cell source is HLA-B*35:01, and the MAGE-A3 targeted T-cell subpopulation is primed and expanded with one or more MAGE-A3-derived peptides selected from Table 93 (SEQ ID NO: 923-932). In some embodiments, the donor cell source is HLA-B*35:01, and the MAGE-A3 targeted T-cell subpopulation is primed and expanded with MAGE-A3-derived peptides selected from Table 93 (SEQ ID NO: 923-932). In some embodiments, the donor cell source is HLA-B*35:01, and the MAGE-A3 targeted T-cell subpopulation is primed and expanded with MAGE-A3-derived peptides comprising the peptides of Table 93 (SEQ ID NO: 923-932). In some embodiments, the donor cell source is HLA-B*35:01, and the MAGE-A3 targeted T-cell subpopulation is primed and expanded with MAGE-A3-derived peptides comprising the peptides of Table 93 (SEQ ID NO: 923-932) and at least one additional set of peptides based on the donor cell source HLA-B profile, wherein the at least one additional set of peptides are selected from the peptides of Tables 88-92 and 94. In some embodiments, the MAGE-A3-derived peptides also include one or more sets of HLA-A and HLA-DR restricted peptides selected from Tables 81-87 and 95-100 (SEQ ID NO: 803-872 and 943-1002).

TABLE 93
MAGEA3 HLA-B*35:01 Epitope Peptides

| SEQ ID NO: | Sequence |
|---|---|
| 923 | APEEKIWEEL |
| 924 | GPRALVETSY |
| 925 | DPKKLLTQHF |
| 926 | EPVTKAEML |
| 927 | LPTTMNYPL |
| 928 | VPGSDPACY |
| 929 | YPPLHEWVL |
| 930 | GPHISYPPL |
| 931 | DPIGHLYIF |
| 932 | MPKAGLLII |

In some embodiments, the donor cell source is HLA-B*58:02, and the MAGE-A3 targeted T-cell subpopulation is primed and expanded with one or more MAGE-A3-derived peptides selected from Table 94 (SEQ ID NO: 933-942). In some embodiments, the donor cell source is HLA-B*58:02, and the MAGE-A3 targeted T-cell subpopulation is primed and expanded with MAGE-A3-derived peptides selected from Table 94 (SEQ ID NO: 933-942). In some embodiments, the donor cell source is HLA-B*58:02, and the MAGE-A3 targeted T-cell subpopulation is primed and expanded with MAGE-A3-derived peptides comprising the peptides of Table 94 (SEQ ID NO: 933-942). In some embodiments, the donor cell source is HLA-B*58:02, and the MAGE-A3 targeted T-cell subpopulation is primed and expanded with MAGE-A3-derived peptides comprising the peptides of Table 94 (SEQ ID NO: 933-942) and at least one additional set of peptides based on the donor cell source HLA-B profile, wherein the at least one additional set of peptides are selected from the peptides of Tables 88-93. In some embodiments, the MAGE-A3-derived peptides also include one or more sets of HLA-A and HLA-DR restricted peptides selected from Tables 81-87 and 95-100 (SEQ ID NO: 803-872 and 943-1002).

TABLE 94
MAGEA3 HLA-B*58:02 Epitope Peptides

| SEQ ID NO: | Sequence |
|---|---|
| 933 | KVAELVHFLL |
| 934 | KASSSLQLVF |
| 935 | SSSTLVEVTL |
| 936 | FSKASSSLQL |
| 937 | KAGLLIIVL |
| 938 | KVAELVHFL |
| 939 | SSTLVEVTL |
| 940 | SSLQLVFGI |
| 941 | KVLHHMVKI |
| 942 | SSLPTTMNY |

In some embodiments, the donor cell source is HLA-DRB1*0101, and the MAGE-A3 targeted T-cell subpopulation is primed and expanded with one or more MAGE-A3-derived peptides selected from Table 95 (SEQ ID NO: 943-952). In some embodiments, the donor cell source is HLA-DRB1*0101, and the MAGE-A3 targeted T-cell subpopulation is primed and expanded with MAGE-A3-derived peptides selected from Table 95 (SEQ ID NO: 943-952). In some embodiments, the donor cell source is HLA-DRB1*0101, and the MAGE-A3 targeted T-cell subpopulation is primed and expanded with MAGE-A3-derived peptides comprising the peptides of Table 95 (SEQ ID NO: 943-952). In some embodiments, the donor cell source is HLA-DRB1*0101, and the MAGE-A3 targeted T-cell subpopulation is primed and expanded with MAGE-A3-derived peptides comprising the peptides of Table 95 (SEQ ID NO: 943-952) and at least one additional set of peptides based on the donor cell source HLA-DR profile, wherein the at least one additional set of peptides are selected from the peptides of Tables 96-100. In some embodiments, the MAGE-A3-derived peptides also include one or more sets of HLA-A and HLA-B restricted peptides selected from Tables 81-94 (SEQ ID NO: 803-942).

TABLE 95
MAGEA3 HLA-DRB1*0101 Epitope Peptides

| SEQ ID NO: | Sequence |
|---|---|
| 943 | PACYEFLWGPRALVE |
| 944 | YLEYRQVPGSDPACY |
| 945 | AGLLIIVLAIIAREG |
| 946 | GEALGLVGAQAPATE |
| 947 | QYFFPVIFSKASSSL |
| 948 | SSSLQLVFGIELMEV |
| 949 | EVTLGEVPAAESPDP |
| 950 | HHMVKISGGPHISYP |
| 951 | HFLLLKYRAREPVTK |
| 952 | ETSYVKVLHHMVKIS |

In some embodiments, the donor cell source is HLA-DRB1*0301, and the MAGE-A3 targeted T-cell subpopulation is primed and expanded with one or more MAGE-A3-derived peptides selected from Table 96 (SEQ ID NO: 953-962). In some embodiments, the donor cell source is HLA-DRB1*0301, and the MAGE-A3 targeted T-cell subpopulation is primed and expanded with MAGE-A3-derived peptides selected from Table 96 (SEQ ID NO: 953-962). In some embodiments, the donor cell source is HLA-DRB1*0301, and the MAGE-A3 targeted T-cell subpopulation is primed and expanded with MAGE-A3-derived peptides comprising the peptides of Table 96 (SEQ ID NO: 953-962). In some embodiments, the donor cell source is HLA-DRB1*0301, and the MAGE-A3 targeted T-cell subpopulation is primed and expanded with MAGE-A3-derived peptides comprising the peptides of Table 96 (SEQ ID NO: 953-962) and at least one additional set of peptides based on the donor cell source HLA-DR profile, wherein the at least one additional set of peptides are selected from the peptides of Tables 95 and 97-100. In some embodiments, the MAGE-A3-derived peptides also include one or more sets of HLA-A and HLA-B restricted peptides selected from Tables 81-94 (SEQ ID NO: 803-942).

TABLE 96
MAGEA3 HLA-DRB1*0301 (DR17) Epitope Peptides

| SEQ ID NO: | Sequence |
|---|---|
| 953 | EDSILGDPKKLLTQH |
| 954 | IELMEVDPIGHLYIF |
| 955 | YDGLLGDNQIMPKAG |
| 956 | FPDLESEFQAALSRK |
| 957 | GPSTFPDLESEFQAA |
| 958 | LGSVVGNWQYFFPVI |
| 959 | ASSLPTTMNYPLWSQ |
| 960 | VAELVHFLLLKYRAR |
| 961 | CLGLSYDGLLGDNQI |
| 962 | SRKVAELVHFLLLKY |

In some embodiments, the donor cell source is HLA-DRB1*0401, and the MAGE-A3 targeted T-cell subpopulation is primed and expanded with one or more MAGE-A3-derived peptides selected from Table 97 (SEQ ID NO: 963-972). In some embodiments, the donor cell source is HLA-DRB1*0401, and the MAGE-A3 targeted T-cell subpopulation is primed and expanded with MAGE-A3-derived peptides selected from Table 97 (SEQ ID NO: 963-972). In some embodiments, the donor cell source is HLA-DRB1*0401, and the MAGE-A3 targeted T-cell subpopulation is primed and expanded with MAGE-A3-derived peptides comprising the peptides of Table 97 (SEQ ID NO: 963-972). In some embodiments, the donor cell source is HLA-DRB1*0401, and the MAGE-A3 targeted T-cell subpopulation is primed and expanded with MAGE-A3-derived peptides comprising the peptides of Table 97 (SEQ ID NO: 963-972) and at least one additional set of peptides based on the donor cell source HLA-DR profile, wherein the at least one additional set of peptides are selected from the peptides of Tables 95-96 and 98-100. In some embodiments, the MAGE-A3-derived peptides also include one or more sets of HLA-A and HLA-B restricted peptides selected from Tables 81-94 (SEQ ID NO: 803-942).

TABLE 97
MAGEA3 HLA-DRB1*0401 (DR4Dw4) Epitope Peptides

| SEQ ID NO: | Sequence |
|---|---|
| 963 | PSTFPDLESEFQAAL |
| 964 | ESEFQAALSRKVAEL |
| 965 | QYFFPVIFSKASSSL |
| 966 | PVIFSKASSSLQLVF |
| 967 | ETSYVKVLHHMVKIS |
| 968 | FPDLESEFQAALSRK |
| 969 | SRKVAELVHFLLLKY |
| 970 | LMEVDPIGHLYIFAT |
| 971 | TSYVKVLHHMVKISG |
| 972 | WQYFFPVIFSKASSS |

In some embodiments, the donor cell source is HLA-DRB1*0701, and the MAGE-A3 targeted T-cell subpopulation is primed and expanded with one or more MAGE-A3-derived peptides selected from Table 98 (SEQ ID NO: 973-982). In some embodiments, the donor cell source is HLA-DRB1*0701, and the MAGE-A3 targeted T-cell subpopulation is primed and expanded with MAGE-A3-derived peptides selected from Table 98 (SEQ ID NO: 973-982). In some embodiments, the donor cell source is HLA-DRB1*0701, and the MAGE-A3 targeted T-cell subpopulation is primed and expanded with MAGE-A3-derived peptides comprising the peptides of Table 98 (SEQ ID NO: 973-982). In some embodiments, the donor cell source is HLA-DRB1*0701, and the MAGE-A3 targeted T-cell subpopulation is primed and expanded with MAGE-A3-derived peptides comprising the peptides of Table 98 (SEQ ID NO: 973-982) and at least one additional set of peptides based on the donor cell source HLA-DR profile, wherein the at least one additional set of peptides are selected from the peptides of Tables 95-97 and 99-100. In some embodiments, the MAGE-A3-derived peptides also include one or more sets of HLA-A and HLA-B restricted peptides selected from Tables 81-94 (SEQ ID NO: 803-942).

TABLE 98
MAGEA3 HLA-DRB1*0701 Epitope Peptides

| SEQ ID NO: | Sequence |
|---|---|
| 973 | ESEFQAALSRKVAEL |
| 974 | ASSLPTTMNYPLWSQ |
| 975 | ATCLGLSYDGLLGDN |
| 976 | QYFFPVIFSKASSSL |
| 977 | FPVIFSKASSSLQLV |
| 978 | PVIFSKASSSLQLVF |
| 979 | GHLYIFATCLGLSYD |
| 980 | LEVFEGREDSILGDP |
| 981 | PRALVETSYVKVLHH |
| 982 | HISYPPLHEWVLREG |

In some embodiments, the donor cell source is HLA-DRB1*1101, and the MAGE-A3 targeted T-cell subpopulation is primed and expanded with one or more MAGE-A3-derived peptides selected from Table 99 (SEQ ID NO: 983-992). In some embodiments, the donor cell source is HLA-DRB1*1101, and the MAGE-A3 targeted T-cell subpopulation is primed and expanded with MAGE-A3-derived peptides selected from Table 99 (SEQ ID NO: 983-992). In some embodiments, the donor cell source is HLA-DRB1*1101, and the MAGE-A3 targeted T-cell subpopulation is primed and expanded with MAGE-A3-derived peptides comprising the peptides of Table 99 (SEQ ID NO: 983-992). In some embodiments, the donor cell source is HLA-DRB1*1101, and the MAGE-A3 targeted T-cell subpopulation is primed and expanded with MAGE-A3-derived peptides comprising the peptides of Table 99 (SEQ ID NO: 983-992) and at least one additional set of peptides based on the donor cell source HLA-DR profile, wherein the at least one additional set of peptides are selected from the peptides of Tables 95-98 and 100. In some embodiments, the MAGE-A3-derived peptides also include one or more sets of HLA-A and HLA-B restricted peptides selected from Tables 81-94 (SEQ ID NO: 803-942).

TABLE 99
MAGEA3 HLA-DRB1*1101 Epitope Peptides

| SEQ ID NO: | Sequence |
|---|---|
| 983 | VKVLHHMVKISGGPH |
| 984 | WQYFFPVIFSKASSS |
| 985 | PACYEFLWGPRALVE |
| 986 | ETSYVKVLHHMVKIS |
| 987 | SRKVAELVHFLLLKY |
| 988 | ELVHFLLLKYRAREP |
| 989 | QYFFPVIFSKASSSL |
| 990 | YLEYRQVPGSDPACY |
| 991 | TSYVKVLHHMVKISG |
| 992 | SEFQAALSRKVAELV |

In some embodiments, the donor cell source is HLA-DRB1*1501, and the MAGE-A3 targeted T-cell subpopulation is primed and expanded with one or more MAGE-A3-derived peptides selected from Table 100 (SEQ ID NO: 993-1002). In some embodiments, the donor cell source is HLA-DRB1*1501, and the MAGE-A3 targeted T-cell subpopulation is primed and expanded with MAGE-A3-derived peptides selected from Table 100 (SEQ ID NO: 993-1002). In some embodiments, the donor cell source is HLA-DRB1*1501, and the MAGE-A3 targeted T-cell subpopulation is primed and expanded with MAGE-A3-derived peptides comprising the peptides of Table 100 (SEQ ID NO: 993-1002). In some embodiments, the donor cell source is HLA-DRB1*1501, and the MAGE-A3 targeted T-cell subpopulation is primed and expanded with MAGE-A3-derived peptides comprising the peptides of Table 100 (SEQ ID NO: 993-1002) and at least one additional set of peptides based on the donor cell source HLA-DR profile, wherein the at least one additional set of peptides are selected from the peptides of Tables 95-99. In some embodiments, the MAGE-A3-derived peptides also include one or more sets of HLA-A and HLA-B restricted peptides selected from Tables 81-94 (SEQ ID NO: 803-942).

TABLE 100
MAGEA3 HLA-DRB1*1501 (DR2b) Epitope Peptides

| SEQ ID NO: | Sequence |
|---|---|
| 993 | GSVVGNWQYFFPVIF |
| 994 | HFLLLKYRAREPVTK |
| 995 | IGHLYIFATCLGLSY |
| 996 | VAELVHFLLLKYRAR |
| 997 | SSSLQLVFGIELMEV |
| 998 | GIELMEVDPIGHLYI |
| 999 | TCLGLSYDGLLGDNQ |
| 1000 | DNQIMPKAGLLIIVL |
| 1001 | AGLLIIVLAIIAREG |
| 1002 | LSVLEVFEGREDSIL |

Epstein-Barr Virus (EBV) Strain B95-8 LMP1 Antigenic Peptides

In some embodiments, the MUSTANG composition includes Epstein-Barr Virus (EBV) Strain B95-8 LMP1 specific T-cells. LMP1 specific T-cells can be generated as described below using one or more antigenic peptides to LMP1. In some embodiments, the LMP1 specific T-cells are generated using one or more antigenic peptides to LMP1, or a modified or heteroclitic peptide derived from a LMP1 peptide. In some embodiments, LMP1 specific T-cells are generated using a LMP1 antigen library comprising a pool of peptides (for example 15 mers) containing amino acid overlap (for example 11 amino acids of overlap) between each sequence formed by scanning the protein amino acid sequence SEQ ID NO: 1003 (UniProt KB-P03230) for EBV Strain B95-8 LMP 1:

MEHDLERGPPGPRRPPRGPPLSSSLGLALLLLLLALLFWLYIVMSDWTGG
ALLVLYSFALMLIIIILIIFIFRRDLLCPLGALCILLLMITLLLIALWNL
HGQALFLGIVLFIFGCLLVLGIWIYLLEMLWRLGATIWQLLAFFLAFFLD
LILLIIALYLQQNWWTLLVDLLWLLLFLAILIWMYYHGQRHSDEHHHDDS
LPHPQQATDDSGHESDSNSNEGRHHLLVSGAGDGPPLCSQNLGAPGGGPD
NGPQDPDNTDDNGPQDPDNTDDNGPHDPLPQDPDNTDDNGPQDPDNTDDN
GPHDPLPHSPSDSAGNDGGPPQLTEEVENKGGDQGPPLMTDGGGGHSHDS
GHGGGDPHLPTLLLGSSGSGGDDDDPHGPVQLSYYD.

In some embodiments, the LMP1 specific T-cells are generated using one or more antigenic peptides to LMP1, or a modified or heteroclitic peptide derived from a LMP1 peptide. In some embodiments, the LMP1 specific T-cells are generated with peptides that recognize class I MHC molecules. In some embodiments, the LMP1 specific T-cells are generated with peptides that recognize class II MHC molecules. In some embodiments, the LMP1 specific T-cells are generated with peptides that recognize both class I and class II MHC molecules.

In some embodiments, the LMP1 peptides used to prime and expand a T-cell subpopulation includes specifically selected HLA-restricted peptides generated by determining the HLA profile of the donor source, and including peptides derived from LMP1 that best match the donor's HLA. In some embodiments, the LMP1 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting LMP1 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 101-106. In some embodiments, the mastermix of peptides includes both an overlapping peptide library and specifically selected HLA-restricted peptides generated by determining the HLA profile of the donor source.

In some embodiments, the donor cell source is HLA-A*01, and the LMP1 targeted T-cell subpopulation is primed and expanded with one or more LMP1-derived peptides selected from Table 101 (SEQ ID NO: 1004-1008). In some embodiments, the donor cell source is HLA-A*01, and the LMP1 targeted T-cell subpopulation is primed and expanded with LMP1-derived peptides selected from from Table 101 (SEQ ID NO: 1004-1008). In some embodiments, the donor cell source is HLA-A*01, and the LMP1 targeted T-cell subpopulation is primed and expanded with LMP 1-derived peptides comprising the peptides of from Table 101 (SEQ ID NO: 1004-1008). In some embodiments, the donor cell source is HLA-A*01, and the LMP1 targeted T-cell subpopulation is primed and expanded with LMP1-derived peptides comprising the peptides of from Table 101 (SEQ ID NO: 1004-1008) and at least one additional set of peptides based on the donor cell source HLA-A profile, wherein the at least one additional set of peptides are selected from the peptides of Tables 102-106. In some embodiments, the LMP1-derived peptides also include one or more sets of HLA-B and HLA-DR restricted peptides.

TABLE 101
EBV Strain B95-8 LMP1 HLA-A*01 Epitope Peptides

| SEQ ID NO: | Sequence |
|---|---|
| 1004 | LLALLFWLY |
| 1005 | WTGGALLVLY |
| 1006 | LLLLALLFWLY |
| 1007 | MSDWTGGALLV |
| 1008 | DWTGGALLVLY |

In some embodiments, the donor cell source is HLA-A*02:01, and the LMP1 targeted T-cell subpopulation is primed and expanded with one or more LMP1 -derived peptides selected from Table 102 (SEQ ID NO: 1009-1013). In some embodiments, the donor cell source is HLA-A*02:01, and the LMP1 targeted T-cell subpopulation is primed and expanded with LMP 1-derived peptides selected from Table 102 (SEQ ID NO: 1009-1013). In some embodiments, the donor cell source is HLA-A*02:01, and the LMP1 targeted T-cell subpopulation is primed and expanded with LMP1-derived peptides comprising the peptides of Table 102 (SEQ ID NO: 1009-1013). In some embodiments, the donor cell source is HLA-A*02:01, and the LMP1 targeted T-cell subpopulation is primed and expanded with LMP1-derived peptides comprising the peptides of Table 102 (SEQ ID NO: 1009-1013) and at least one additional set of peptides based on the donor cell source HLA-A profile, wherein the at least one additional set of peptides are selected from the peptides of Tables 101, and 103-106. In some embodiments, the LMP1-derived peptides also include one or more sets of HLA-B and HLA-DR restricted peptides.

TABLE 102
EBV Strain B95-8 LMP1 HLA-A*02:01 Epitope Peptides

| SEQ ID NO: | Sequence |
|---|---|
| 1009 | ALLLLLLAL |
| 1010 | LLLLLLALL |
| 1011 | YLLEMLWRL |
| 1012 | GLALLLLLL |
| 1013 | LLLALLFWL |

In some embodiments, the donor cell source is HLA-A*03, and the LMP1 targeted T-cell subpopulation is primed and expanded with one or more LMP1-derived peptides selected from Table 103 (SEQ ID NO: 1014-1018). In some embodiments, the donor cell source is HLA-A*03, and the LMP1 targeted T-cell subpopulation is primed and expanded with LMP1-derived peptides selected from Table 103 (SEQ ID NO: 1014-1018). In some embodiments, the donor cell source is HLA-A*03, and the LMP1 targeted T-cell subpopulation is primed and expanded with LMP1-derived peptides comprising the peptides of Table 103 (SEQ ID NO: 1014-1018). In some embodiments, the donor cell source is HLA-A*03, and the LMP1 targeted T-cell subpopulation is primed and expanded with LMP1-derived peptides comprising the peptides of Table 103 (SEQ ID NO: 1014-1018) and at least one additional set of peptides based on the donor cell source HLA-A profile, wherein the at least one additional set of peptides are selected from the peptides of Tables 101-102 and 104-106. In some embodiments, the LMP1-derived peptides also include one or more sets of HLA-B and HLA-DR restricted peptides.

TABLE 103
EBV Strain B95-8 LMP1 HLA-A*03 Epitope Peptides

| SEQ ID NO: | Sequence |
|---|---|
| 1014 | ALFLGIVLF |
| 1015 | QLLAFFLAF |
| 1016 | LLLLLALLF |
| 1017 | MLWRLGATI |
| 1018 | QLTEEVENK |

In some embodiments, the donor cell source is HLA-A*11:01, and the LMP1 targeted T-cell subpopulation is primed and expanded with one or more LMP 1-derived peptides selected from Table 104 (SEQ ID NO: 1019-1023). In some embodiments, the donor cell source is HLA-A*11:01, and the LMP1 targeted T-cell subpopulation is primed and expanded with LMP 1-derived peptides selected from Table 104 (SEQ ID NO: 1019-1023). In some embodiments, the donor cell source is HLA-A*11:01, and the LMP1 targeted T-cell subpopulation is primed and expanded with LMP1-derived peptides comprising the peptides of Table 104 (SEQ ID NO: 1019-1023). In some embodiments, the donor cell source is HLA-A*11:01, and the LMP1 targeted T-cell subpopulation is primed and expanded with LMP1-derived peptides comprising the peptides of Table 104 (SEQ ID NO: 1019-1023), and at least one additional set of peptides based on the donor cell source HLA-A profile, wherein the at least one additional set of peptides are selected from the peptides of Tables 101-103 and 105-106. In some embodiments, the LMP1-derived peptides also include one or more sets of HLA-B and HLA-DR restricted peptides.

TABLE 104
EBV Strain B95-8 LMP1 HLA-A*11:01 Epitope Peptides

| SEQ ID NO: | Sequence |
|---|---|
| 1019 | SSLGLALLL |
| 1020 | IILIIFIFR |
| 1021 | SSSLGLALLL |
| 1022 | IIILIIFIFR |
| 1023 | ESDSNSNEGR |

In some embodiments, the donor cell source is HLA-A*24:02, and the LMP1 targeted T-cell subpopulation is primed and expanded with one or more LMP 1-derived peptides selected from Table 105 (SEQ ID NO: 1024-1028). In some embodiments, the donor cell source is HLA-A*24:02, and the LMP1 targeted T-cell subpopulation is primed and expanded with LMP 1-derived peptides selected from Table 105 (SEQ ID NO: 1024-1028). In some embodiments, the donor cell source is HLA-A*24:02, and the LMP1 targeted T-cell subpopulation is primed and expanded with LMP1-derived peptides comprising the peptides of Table 105 (SEQ ID NO: 1024-1028). In some embodiments, the donor cell source is HLA-A*24:02, and the LMP1 targeted T-cell subpopulation is primed and expanded with LMP1-derived peptides comprising the peptides of Table 105 (SEQ ID NO: 1024-1028), and at least one additional set of peptides based on the donor cell source HLA-A profile, wherein the at least one additional set of peptides are selected from the peptides of Tables 101-104 and 106. In some embodiments, the LMP1-derived peptides also include one or more sets of HLA-B and HLA-DR restricted peptides.

TABLE 105
EBV Strain B95-8 LMP1 HLA-A*24:02 Epitope Peptides

| SEQ ID NO: | Sequence |
|---|---|
| 1024 | LYSFALMLI |
| 1025 | FFLDLILLI |
| 1026 | IFIFRRDLL |
| 1027 | IYLLEMLWRL |
| 1028 | LYLQQNWWTL |

In some embodiments, the donor cell source is HLA-A*26, and the LMP1 targeted T-cell subpopulation is primed and expanded with one or more LMP1-derived peptides selected from Table 106 (SEQ ID NO: 1029-1033). In some embodiments, the donor cell source is HLA-A*26, and the LMP1 targeted T-cell subpopulation is primed and expanded with LMP1-derived peptides selected from Table 106 (SEQ ID NO: 1029-1033). In some embodiments, the donor cell source is HLA-A*26, and the LMP1 targeted T-cell subpopulation is primed and expanded with LMP1-derived peptides comprising the peptides of Table 106 (SEQ ID NO: 1029-1033). In some embodiments, the donor cell source is HLA-A*26, and the LMP1 targeted T-cell subpopulation is primed and expanded with LMP1-derived peptides comprising the peptides of Table 106 (SEQ ID NO: 1029-1033) and at least one additional set of peptides based on the donor cell source HLA-A profile, wherein the at least one additional set of peptides are selected from the peptides of Tables 101-105. In some embodiments, the LMP1-derived peptides also include one or more sets of HLA-B and HLA-DR restricted peptides.

TABLE 106
EBV Strain B95-8 LMP1 HLA-A*26 Epitope Peptides

| SEQ ID NO: | Sequence |
|---|---|
| 1029 | DLILLIIAL |
| 1030 | ATIWQLLAF |
| 1031 | LIIIILIIF |
| 1032 | EVENKGGDQ |
| 1033 | LVDLLWLLLF |

Human Papillomavirus (HPV) Strain 16 E6 Antigenic Peptides

In some embodiments, the MUSTANG composition includes Human Papillomavirus (HPV) Strain 16 E6 specific T-cells. E6 specific T-cells can be generated as described below using one or more antigenic peptides to E6. In some embodiments, the E6 specific T-cells are generated using one or more antigenic peptides to E6, or a modified or heteroclitic peptide derived from a E6 peptide. In some embodiments, E6 specific T-cells are generated using a E6 antigen library comprising a pool of peptides (for example 15 mers) containing amino acid overlap (for example 11 amino acids of overlap) between each sequence formed by scanning the protein amino acid sequence SEQ ID NO: 1034 (UniProt KB-P03126) for HPV Strain 16-8 E6:

MHQKRTAMFQDPQERPRKLPQLCTELQTTIHDIILECVYCKQQLLRREVY
DFAFRDLCIVYRDGNPYAVCDKCLKFYSKISEYRHYCYSLYGTTLEQQYN
KPLCDLLIRCINCQKPLCPEEKQRHLDKKQRFHNIRGRWTGRCMSCCRSS
RTRRETQL.

In some embodiments, the E6 specific T-cells are generated using one or more antigenic peptides to E6, or a modified or heteroclitic peptide derived from a E6 peptide. In some embodiments, the E6 specific T-cells are generated with peptides that recognize class I MHC molecules. In some embodiments, the E6 specific T-cells are generated with peptides that recognize class II MEW molecules. In some embodiments, the E6 specific T-cells are generated with peptides that recognize both class I and class II MHC molecules.

In some embodiments, the E6 peptides used to prime and expand a T-cell subpopulation includes specifically selected HLA-restricted peptides generated by determining the HLA profile of the donor source, and including peptides derived from E6 that best match the donor's HLA. In some embodiments, the E6 peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213. https://doi.org/10.1007/s002510050595.

As provided herein, the HLA profile of a donor cell source can be determined, and T-cell subpopulations targeting E6 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides, wherein the HLA-A matched peptides are selected from the peptides of Tables 281-287 , the HLA-B peptides are selected from the peptides of Tables 288-294, and the HLA-DR peptides are selected from the peptides of Tables 295-280. For example, if the donor cell source has an HLA profile that is HLA-A*01/*02:01; HLA-B*15:01/*18; and HLA-DRB1*0101/*0301, then the E6 peptides used to prime and expand the E6 specific T-cell subpopulation are restricted to the specific HLA profile, and may include the peptides identified in Tables 107-111. In some embodiments, the mastermix of peptides includes both an overlapping peptide library and specifically selected HLA-restricted peptides generated by determining the HLA profile of the donor source.

In some embodiments, the donor cell source is HLA-A*01, and the E6 targeted T-cell subpopulation is primed and expanded with one or more E6-derived peptides selected from Table 107 (SEQ ID NO: 1035-1039). In some embodiments, the donor cell source is HLA-A*01, and the E6 targeted T-cell subpopulation is primed and expanded with E6-derived peptides selected from from Table 107 (SEQ ID NO: 1035-1039). In some embodiments, the donor cell source is HLA-A*01, and the E6 targeted T-cell subpopulation is primed and expanded with E6-derived peptides comprising the peptides of from Table 107 (SEQ ID NO: 1035-1039). In some embodiments, the donor cell source is HLA-A*01, and the E6 targeted T-cell subpopulation is primed and expanded with E6-derived peptides comprising the peptides of from Table 107 (SEQ ID NO: 1035-1039) and at least one additional set of peptides based on the donor cell source HLA-A profile, wherein the at least one additional set of peptides are selected from the peptides of Tables 108-111. In some embodiments, the E6-derived peptides also include one or more sets of HLA-B and HLA-DR restricted peptides.

TABLE 107
HPV Strain 16 E6 HLA-A*01 Epitope Peptides

| SEQ ID NO: | Sequence |
|---|---|
| 1035 | YAVCDKCLKFY |
| 1036 | SEYRHYCYSLY |
| 1037 | CKQQLLRREVY |
| 1038 | IHDIILECVY |
| 1039 | YSKISEYRHY |

In some embodiments, the donor cell source is HLA-A*02:01, and the E6 targeted T-cell subpopulation is primed and expanded with one or more E6 -derived peptides selected from Table 108 (SEQ ID NO: 1040-1044). In some embodiments, the donor cell source is HLA-A*02:01, and the E6 targeted T-cell subpopulation is primed and expanded with MAGE-A3-derived peptides selected from Table 108 (SEQ ID NO: 1040-1044). In some embodiments, the donor cell source is HLA-A*02:01, and the E6 targeted T-cell subpopulation is primed and expanded with E6 -derived peptides comprising the peptides of Table 108 (SEQ ID NO: 1040-1044). In some embodiments, the donor cell source is HLA-A*02:01, and the E6 targeted T-cell subpopulation is primed and expanded with E6-derived peptides comprising the peptides of Table 108 (SEQ ID NO: 1040-1044) and at least one additional set of peptides based on the donor cell source HLA-A profile, wherein the at least one additional set of peptides are selected from the peptides of Tables 106 and 108-111. In some embodiments, the E6-derived peptides also include one or more sets of HLA-B and HLA-DR restricted peptides.

TABLE 108
HPV Strain 16 E6 HLA-A*02:01 Epitope Peptides

| SEQ ID NO: | Sequence |
|---|---|
| 1040 | TIHDIILECV |
| 1041 | QLCTELQTTI |
| 1042 | PLCDLLIRCI |
| 1043 | KLPQLCTEL |
| 1044 | QLCTELQTT |

In some embodiments, the donor cell source is HLA-A*03, and the E6 targeted T-cell subpopulation is primed and expanded with one or more E6-derived peptides selected from Table 109 (SEQ ID NO: 1045-1049). In some embodiments, the donor cell source is HLA-A*03, and the E6 targeted T-cell subpopulation is primed and expanded with E6-derived peptides selected from Table 109 (SEQ ID NO: 1045-1049). In some embodiments, the donor cell source is HLA-A*03, and the E6 targeted T-cell subpopulation is primed and expanded with E6-derived peptides comprising the peptides of Table 109 (SEQ ID NO: 1045-1049). In some embodiments, the donor cell source is HLA-A*03, and the E6 targeted T-cell subpopulation is primed and expanded with E6-derived peptides comprising the peptides of Table 109 (SEQ ID NO: 1045-1049) and at least one additional set of peptides based on the donor cell source HLA-A profile, wherein the at least one additional set of peptides are selected from the peptides of Tables 106-108 and 110-111. In some embodiments, the E6-derived peptides also include one or more sets of HLA-B and HLA-DR restricted peptides.

TABLE 109
HPV Strain 16 E6 HLA-A*03 Epitope Peptides

| SEQ ID NO: | Sequence |
|---|---|
| 1045 | LLIRCINCQK |
| 1046 | DIILECVYCK |
| 1047 | CVYCKQQLLR |
| 1048 | SLYGTTLEQQ |
| 1049 | IVYRDGNPY |

In some embodiments, the donor cell source is HLA-A*11:01, and the E6 targeted T-cell subpopulation is primed and expanded with one or more E6-derived peptides selected from Table 110 (SEQ ID NO: 1050-1054). In some embodiments, the donor cell source is HLA-A*11:01, and the E6 targeted T-cell subpopulation is primed and expanded with E6-derived peptides selected from Table 110 (SEQ ID NO: 1050-1054). In some embodiments, the donor cell source is HLA-A*11:01, and the E6 targeted T-cell subpopulation is primed and expanded with E6-derived peptides comprising the peptides of Table 110 (SEQ ID NO: 1050-1054). In some embodiments, the donor cell source is HLA-A*11:01, and the E6 targeted T-cell subpopulation is primed and expanded with E6-derived peptides comprising the peptides of Table 110 (SEQ ID NO: 1050-1054), and at least one additional set of peptides based on the donor cell source HLA-A profile, wherein the at least one additional set of peptides are selected from the peptides of Tables 106-109 and 111. In some embodiments, the E6-derived peptides also include one or more sets of HLA-B and HLA-DR restricted peptides.

TABLE 110
HPV Strain 16 E6 HLA-A*11:01 Epitope Peptides

| SEQ ID NO: | Sequence |
|---|---|
| 1050 | CVYCKQQLLR |
| 1051 | GTTLEQQYNK |
| 1052 | DIILECVYCK |
| 1053 | AFRDLCIVYR |
| 1054 | WTGRCMSCCR |

In some embodiments, the donor cell source is HLA-A*24:02, and the E6 targeted T-cell subpopulation is primed and expanded with one or more E6-derived peptides selected from Table 111 (SEQ ID NO: 1055-1059). In some embodiments, the donor cell source is HLA-A*24:02, and the E6 targeted T-cell subpopulation is primed and expanded with E6-derived peptides selected from Table 111 (SEQ ID NO: 1055-1059). In some embodiments, the donor cell source is HLA-A*24:02, and the E6 targeted T-cell subpopulation is primed and expanded with E6-derived peptides comprising the peptides of Table 111 (SEQ ID NO: 1055-1059). In some embodiments, the donor cell source is HLA-A*24:02, and the E6 targeted T-cell subpopulation is primed and expanded with E6-derived peptides comprising the peptides of Table 111 (SEQ ID NO: 1055-1059), and at least one additional set of peptides based on the donor cell source HLA-A profile, wherein the at least one additional set of peptides are selected from the peptides of Tables 106-110. In some embodiments, the E6-derived peptides also include one or more sets of HLA-B and HLA-DR restricted peptides.

TABLE 111
HPV Strain 16 E6 HLA-A*24:02 Epitope Peptides

| SEQ ID NO: | Sequence |
|---|---|
| 1055 | QYNKPLCDLL |
| 1056 | QDPQERPRKL |
| 1057 | LCPEEKQRHL |
| 1058 | VYDFAFRDL |
| 1059 | PYAVCDKCL |

### Ratio of T-Cell Subpopulations in Lymphocytic Cell Compositions

The lymphocytic cell composition of the present disclosure is comprised of one or more (or three or more, or four or more, or five or more) T-cell components comprising two or more (or three or more, or four or more, or five or more) T-cell subpopulations each targeting a single TAA. The T-cell subpopulations used to create the lymphocytic cell composition can be combined in a single dosage form for administration, or each administered separately, wherein the separate T-cell subpopulations collectively comprise the lymphocytic cell composition. In one embodiment, the lymphocytic cell composition comprises one or more T-cell components comprising T-cell subpopulations in a ratio or percentage reflective or correlative of the relative identified TAA expression profile of the patient. In one embodiment, the T-cell subpopulations of each T-cell component used to create the lymphocytic cell composition are in about an equal ratio. In one embodiment, the lymphocytic cell composition comprises one or more T-cell components that comprise two or more T-cell subpopulations, wherein each T-cell subpopulation is specific for a different TAA.

The ratios of the T-cell subpopulations for each T-cell component in the lymphocytic cell composition may be selected based on the knowledge of the patient's tumor characteristics or the healthcare provider's best judgement. In one embodiment, the lymphocytic cell composition comprises one or more T-cell components that comprise two or more T-cell subpopulations (i) at least about 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, or 85% of a first T-cell subpopulation and (ii) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, or 55% of a second T-cell subpopulation, wherein the percentage adds to 100% by weight. In one embodiment, the percentage of the first and second T-cell subpopulations is based on the TAA expression profile of a malignancy or tumor such that the percentage of the first and second T-cell subpopulations correlates with the TAA expression profile of the tumor.

Each T-cell component of the lymphocytic cell composition can include two, three, four, five, or more T-cell subpopulations. The T-cell subpopulations for each T-cell component can be included in the lymphocytic cell composition in about an equal ratio, or in a ratio that reflects the individual TAA expression as determined by the patient's TAA expression profile, or in an alternative ratio. In an alternative embodiment, the T-cell subpopulations can be included in a ratio that reflects a greater percentage of T-cell subpopulations directed to known TAAs which show high immunogenicity.

In a particular embodiment, the lymphocytic cell composition comprises one or more T-cell components that comprise two or more T-cell subpopulations, wherein the first T-cell subpopulation is specific to PRAMS and the second T-cell subpopulation is selected from the group consisting of WT1, survivin, NY-ESO-1 and MAGE-A3.

In a particular embodiment, the lymphocytic cell composition comprises one or more T-cell components that comprise two or more T-cell subpopulations, wherein the first T-cell subpopulation to survivin and the second T-cell subpopulation is selected from the group consisting of WT1, NY-ESO-1 and MAGE-A3.

In a particular embodiment, the lymphocytic cell composition comprises one or more T-cell components that comprise two or more T-cell subpopulations, wherein the first T-cell subpopulation is specific to WT1 and the second T-cell subpopulation is selected from the group consisting of NY-ESO-1 and MAGE-A3.

In a particular embodiment, the lymphocytic cell composition comprises one or more T-cell components that comprise two or more T-cell subpopulations, wherein the first T-cell subpopulation is specific to NY-ESO-1 and the second T-cell subpopulation is specific to MAGE-A3.

In one embodiment, the lymphocytic cell composition comprises one or more T-cell components that comprise a first T-cell subpopulation, a second T-cell subpopulation, and a third T-cell subpopulation, wherein each T-cell subpopulation is specific for a different TAA. In one embodiment, the T-cell subpopulations used to create the MUSTANG are in about an equal ratio.

The ratios of the T-cell subpopulations in the lymphocytic cell composition for each T-cell component may be selected based on the knowledge of the patient's tumor characteristics or the healthcare provider's best judgement. In one embodiment, the lymphocytic cell composition comprises one or more T-cell components that comprise three T-cell subpopulations, wherein the each T-cell component that comprises three T-cell subpopulations comprises (i) at least about 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, or 85% of the first T-cell subpopulation, (ii) at least about 5%, 6%, 7%, 8%, 9%, 10%, 11%, 12%, 13%, 14%, 15%, 16%, 17%, 18%, 19%, 20%, 21%, 22%, 23%, 24%, or 25% of the second T-cell subpopulation and (iii) at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35% of the third T-cell subpopulation, wherein the percentage adds to 100% by weight. In one embodiment, the percentage of the T-cell subpopulations is based on the TAA expression profile of a malignancy or tumor such that the percentage of the first, second, and third T-cell subpopulations for each T-cell component of the lymphocytic cell composition cocorrelates with the TAA expression profile of the tumor.

In one embodiment, the TAA is selected from survivin, MAGE-A3, NY-ESO-1, PRAME, and WT1.

In a particular embodiment, the lymphocytic cell composition comprises one or more T-cell components that comprise three or more T-cell subpopulations, wherein the first T-cell subpopulation is specific to PRAME, the second T-cell subpopulation is specific to WT1, and the third T-cell subpopulation is selected from the group consisting of survivin, NY-ESO-1 and MAGE-A3.

In another particular embodiment, the lymphocytic cell composition comprises one or more T-cell components that comprise three or more T-cell subpopulations, wherein the first T-cell subpopulation is specific to PRAME, the second T-cell subpopulation is specific to NY-ESO-1, and the third T-cell subpopulation is specific to MAGE-A3.

In another particular embodiment, the lymphocytic cell composition comprises one or more T-cell components that comprise three or more T-cell subpopulations, wherein the first T-cell subpopulation composition is specific to WT1, the second T-cell subpopulation is specific to NY-ESO-1, and the third T-cell subpopulation is specific to MAGE-A3.

In one embodiment, the lymphocytic cell composition comprises one or more T-cell components that comprise a first T-cell subpopulation, a second T-cell subpopulation, a third T-cell subpopulation, and a fourth T-cell subpopulation, wherein each T-cell subpopulation is specific for a different TAA. In one embodiment, the T-cell subpopulations used to create the MUSTANG are in about an equal ratio.

The ratios of the T-cell subpopulations in the lymphocytic cell composition for each T-cell component may be selected based on the knowledge of the patient's tumor characteristics or the healthcare provider's best judgement. In one embodiment, the lymphocytic cell composition comprises one or more T-cell components that comprise three T-cell subpopulations, wherein the each T-cell component that comprises three T-cell subpopulations comprises (i) at least about 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, or 85% of the first T-cell subpopulation, (ii) at least about 5%, 6%, 7%, 8%, 9%, 10%, 11%, 12%, 13%, 14%, 15%, 16%, 17%, 18%, 19%, 20%, 21%, 22%, 23%, 24%, or 25%, 26%, 27%, 28%, 29%, 30%, 31%, 32%, 33%, 34%, 35%, 36%, 37%, 38%, 39%, 40%, 41%, 42%, 43%, 44%, or 45% of the second T-cell subpopulation, (iii) at least about 5%, 6%, 7%, 8%, 9%, 10%, 11%, 12%, 13%, 14%, 15%, 16%, 17%, 18%, 19%, 20%, 21%, 22%, 23%, 24%, or 25%, 26%, 27%, 28%, 29%, 30%, 31%, 32%, 33%, 34%, 35%, 36%, 37%, 38%, 39%, 40%, 41%, 42%, 43%, 44%, or 45% of the third T-cell subpopulation, and (iv) at least about 5%, 6%, 7%, 8%, 9%, 10%, 11%, 12%, 13%, 14%, 15%, 16%, 17%, 18%, 19%, 20%, 21%, 22%, 23%, 24%, or 25%, 26%, 27%, 28%, 29%, 30%, 31%, 32%, 33%, 34%, 35%, 36%, 37%, 38%, 39%, 40%, 41%, 42%, 43%, 44%, or 45% of the fourth T-cell subpopulation, wherein the percentage adds to 100% by weight. In one embodiment, the percentage of the T-cell subpopulations is based on the TAA expression profile of a malignancy or tumor such that the percentage of the first, second, third and fourth T-cell subpopulations for each T-cell component of the lymphocytic cell composition correlates with the TAA expression profile of the tumor. In one embodiment, the T-cell subpopulations are specific to a TAA selected from survivin, MAGE-A3, NY-ESO-1, PRAME, and WT1.

In a particular embodiment, the lymphocytic cell composition comprises one or more T-cell components that comprise four or more T-cell subpopulations, wherein the first T-cell subpopulation is specific to PRAME, the second T-cell subpopulation is specific to WT1, the third T-cell subpopulation is survivin and the fourth T-cell subpopulation is selected from the group consisting of MAGE-A3 and NY-ESO-1.

In a further embodiment, the lymphocytic cell composition comprises one or more T-cell components that comprise four or more T-cell subpopulations, wherein the first T-cell subpopulation is specific to PRAME, the second T-cell subpopulation is specific to WT1, the third T-cell subpopulation is specific to NY-ESO-1 and the fourth T-cell subpopulation is specific to MAGE-A3.

In a still further embodiment, the lymphocytic cell composition comprisses one or more T-cell components that comprise four or more T-cell subpopulations, wherein the first T-cell subpopulation is specific to PRAME, the second T-cell subpopulation is specific to survivin, the third T-cell subpopulation is specific to NY-ESO-1, and the fourth T-cell subpopulation is specific to MAGE-A3.

In one embodiment, the lymphocytic cell composition comprises one or more T-cell components that comprise a first T-cell subpopulation, a second T-cell subpopulation, a third T-cell subpopulation, a fourth T-cell subpopulation, and a fifth T-cell subpopulation, wherein each T-cell subpopulation is specific for a different tumor-associated antigen. In one embodiment, the T-cell subpopulations used to create the MUSTANG are in about an equal ratio.

The ratios of the T-cell subpopulations in the lymphocytic cell composition for each T-cell component may be selected based on the knowledge of the patient's tumor characteristics or the healthcare provider's best judgement. In one embodiment, the lymphocytic cell composition comprises one or more T-cell components that comprise three T-cell subpopulations, wherein the each T-cell component that comprises three T-cell subpopulations comprises (i) at least about 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% of the first T-cell subpopulation, (ii) at least about 5%, 6%, 7%, 8%, 9%, 10%, 11%, 12%, 13%, 14%, 15%, 16%, 17%, 18%, 19%, 20%, 21%, 22%, 23%, 24%, or 25%, 26%, 27%, 28%, 29%, 30%, 31%, 32%, 33%, 34%, 35%, 36%, 37%, 38%, 39%, or 40% of the second T-cell subpopulation, (iii) at least about 5%, 6%, 7%, 8%, 9%, 10%, 11%, 12%, 13%, 14%, 15%, 16%, 17%, 18%, 19%, 20%, 21%, 22%, 23%, 24%, or 25%, 26%, 27%, 28%, 29%, 30%, 31%, 32%, 33%, 34%, 35%, 36%, 37%, 38%, 39%, or 40% of the third T-cell subpopulation, (iv) at least about 5%, 6%, 7%, 8%, 9%, 10%, 11%, 12%, 13%, 14%, 15%, 16%, 17%, 18%, 19%, 20%, 21%, 22%, 23%, 24%, or 25%, 26%, 27%, 28%, 29%, 30%, 31%, 32%, 33%, 34%, 35%, 36%, 37%, 38%, 39%, or 40% of the fourth T-cell subpopulation and (v) at least about 5%, 6%, 7%, 8%, 9%, 10%, 11%, 12%, 13%, 14%, 15%, 16%, 17%, 18%, 19%, 20%, 21%, 22%, 23%, 24%, or 25%, 26%, 27%, 28%, 29%, 30%, 31%, 32%, 33%, 34%, 35%, 36%, 37%, 38%, 39%, or 40% of the fifth T-cell subpopulation, wherein the percentage adds to 100% by weight. In one embodiment, the percentage of the T-cell subpopulations is based on the TAA expression profile of a malignancy or tumor such that the percentage of the first, second, third, fourth and fifth T-cell subpopulations for each T-cell component of the lymphocytic cell composition correlates with the TAA expression profile of the tumor. In one embodiment, each of the five T-cell subpopulations are specific to survivin, MAGE-A3, NY-ESO-1, PRAME, and WT1, respectively.

In one embodiment, the lymphocytic cell composition comprises one or more T-cell components that comprise five or more T-cell subpopulations, wherein the first T-cell subpopulation is specific to PRAME, the second T-cell subpopulation is specific to WT1, the third T-cell subpopulation is specific to survivin, the fourth T-cell subpopulation is specific to MAGE-A3 and the fifth T-cell subpopulation is specific to NY-ESO-1.

In one embodiment, the mononuclear cell sample from which the T-cell subpopulations are isolated is derived from the human to which the composition is also administered (autologous).

In one embodiment, the mononuclear cell sample from which the T-cell subpopulations are isolated is derived from a cell donor (allogeneic). In certain embodiments, the allogeneic T-cell subpopulation composition has at least one HLA allele or HLA allele combination in common with the patient. In certain embodiments, the allogeneic T-cell subpopulation composition has more than one HLA allele or HLA allele combination in common with the patient. In certain embodiments, the tumor-associated antigen activity of the lymphocytic cell composition is through at least one HLA allele or HLA allele combination in common with the patient. In certain embodiments, the allogeneic T-cell subpopulations comprising the lymphocytic cell composition are recognized through the same shared HLA restriction. In certain embodiments, the allogeneic T-cell subpopulations comprising the lymphocytic cell composition are recognized through different shared HLA restrictions.

In another aspect, the present disclosure provides a method of treating a disease or disorder comprising administering an effective amount of the lymphocytic cell composition disclosed herein to a patient, typically a human in need thereof.

In one embodiment, the method further comprises isolating a mononuclear cell sample from the patient, typically a human to which the lymphocytic cell composition is administered (autologous), wherein the lymphocytic cell composition is made from the mononuclear cell sample.

In one embodiment, the method further comprises isolating a mononuclear cell sample from a cell donor (allogeneic), wherein the lymphocytic cell composition is made from the mononuclear cell sample. In certain embodiments, the allogeneic lymphocytic cell composition has at least one HLA allele or HLA allele combination in common with the patient. In certain embodiments, the allogeneic lymphocytic cell composition has more than one HLA allele or HLA allele combination in common with the patient. In certain embodiments, the TAA activity of the lymphocytic cell composition is through at least one HLA allele or HLA allele combination in common with the patient. In certain embodiments, the TAA activity of the lymphocytic cell composition is through more than one HLA allele or HLA allele combination in common with the patient. In certain embodiments, the allogeneic T-cell subpopulations comprising the lymphocytic cell composition are recognized through the same shared HLA restriction. In certain embodiments, the allogeneic T-cell subpopulations comprising the lymphocytic cell composition are recognized through different shared HLA restrictions. In certain embodiments the lymphocytic cell composition selected has the most shared HLA alleles or allele combinations and the highest TAA specificity.

In certain embodiments, the method further comprises selecting the lymphocytic cell composition based on the TAA expression profile of the malignancy or tumor of the patient. In certain embodiments, the method further comprises selecting the lymphocytic cell composition based on the levels of circulating TAA-specific T-cells present in the patient after administration of a lymphocytic cell composition. Methods of measuring the levels of circulating TAA-specific T-cells present in the patient are known in the art and non-limiting exemplary methods include Elispot assay, TCR sequencing, intracellular cytokine staining, and through the uses of MHC-peptide multimers.

Method of Treating a Patient with a Tumor by Administering a Lymphocytic Cell Composition

The present disclosure includes a method to treat a patient with a tumor, typically a human, by administering an effective amount of a lymphocytic cell composition described herein.

The dose administered may vary. In some embodiments, the lymphocytic cell composition is administered to a patient, such as a human in a dose ranging from 1×10⁶ cells/m² to 1×10⁸ cells/m². The dose can be a single dose, for example, comprising the combination of all of the T-cell subpopulations of each T-cell component of a lymphocytic cell composition, or multiple separate doses, wherein each dose comprises a T-cell component, with each dose, in some embodiments, comprising a separate T-cell subpopulation for each T-cell component and the collective separate doses of T-cell compnents (and in some embodiments each T-cell subpopulation) comprise the total lymphocytic cell composition. In some embodiments, the lymphocytic cell composition dosage is 1×10⁶ cells/m², 2×10⁶ cells/m², 3×10⁶ cells/m², 4×10⁶ cells/m², 5×10⁶ cells/m², 6×10⁶ cells/m², 7×10⁶ cells/m², 8×10⁶ cells/m², 9×10⁶ cells/m², 1×10⁷ cells/m², 2×10⁷ cells/m², 3×10⁷ cells/m², 4×10⁷ cells/m², 5×10⁷ cells/m², 6×10⁷ cells/m², 7×10⁷ cells/m², 8×10⁷ cells/m², 9×10⁷ cells/m², or 1×10⁸ cells/m².

The lymphocytic cell composition may be administered by any suitable method. In some embodiments, the lymphocytic cell composition is administered to a patient, such as a human as an infusion and in a particular embodiment, an infusion with a total volume of 1 to 10 cc. In some embodiments, the lymphocytic cell composition is administered to a patient as a 1, 2, 3, 4, 5, 6, 7, 8, 9, or 10 cc infusion. In some embodiments, the lymphocytic cell composition when present as an infusion is administered to a patient over 10, 20, 30, 40, 50, 60 or more minutes to the patient in need thereof.

In one embodiment, a patient receiving an infusion has vital signs monitored before, during, and 1-hour post infusion of the lymphocytic cell composition. In certain embodiments, patients with stable disease (SD), partial response (PR), or complete response (CR) up to 6 weeks after initial infusion may be eligible to receive additional infusions, for example, 1, 2, 3, 4, 5, 6, 7, 8, 9 or 10 additional infusions several weeks apart, for example, up to about 2, 3, 4, 5, 6, 7, 8, 9 or 10 weeks apart.

### Determining the Tumor-Associated Antigen Expression Profile

Determining a TAA expression profile can be performed by any method known in the art.

Non-limiting exemplary methods for determining a tumor-associated antigen expression profile can be found in Ding et al., Cancer Bio. Med. 9:73-76 (2012); Qin et al. 2009, supra; and Weber et al. 2009, supra. In one embodiment, TAA expression profiles are generated from a sample collected from a patient with a malignancy or tumor. In one embodiment, the sample is selected from a group consisting of blood, bone marrow, and tumor biopsy.

In one embodiment, the TAA expression profile is determined from a blood sample of a patient with a malignancy or tumor. In one embodiment, the TAA expression profile is determined from a bone marrow sample of a patient with a malignancy or tumor. In one embodiment, the TAA expression profile is determined from a tumor biopsy sample of a patient with a malignancy or tumor.

In one embodiment, genetic material is extracted from the sample collected from a patient with a malignancy or tumor. In one embodiment, the genetic material is selected from a group consisting of total RNA, messenger RNA and genomic DNA.

After extraction of genetic material, quantitative reverse transcriptase polymerase chain reaction (qPCR) is performed on the genetic material utilizing primers developed from TAAs of interest.

The patient's tumor cells can be checked for reactivity against activated T-cell subpopulations and/or the lymphocytic cell composition of the present disclosure using any known methods, including cytotoxicity assays described herein.

### Determining the Levels of Circulating TAA-Specific T-Cells

Determining the levels of circulating TAA-specific T-cells after infusion of the lymphocytic cell composition can be performed by any method known in the art. Non-limiting exemplary methods for determining levels of circulating TAA-specific T-cells include Elispot assay, intracellular cytokine staining, multimer analysis, and TCR sequencing and can be found in Chapuis et al., Sci. Transl. Med. 5(174):174ra27 (2013); and Hanley et al., Sci. Transl. Med. 7(285):285ra63 (2015), which are incorporated herein by reference. In one embodiment, levels of circulating TAA-specific T-cells is determined from a sample collected from a patient with a malignancy or tumor treated with a lymphocytic cell composition. In one embodiment, the sample is selected from a group consisting of blood, peripheral blood mononuclear cells, and bone marrow.

In one embodiment, the levels of circulating TAA-specific T-cells is determined from a blood sample of a patient with a malignancy or tumor treated with a lymphocytic cell composition. In one embodiment, the levels of circulating TAA-specific T-cells is determined from a peripheral blood mononuclear cell sample of a patient with a malignancy or tumor treated with a lymphocytic cell composition. In one embodiment, the levels of circulating TAA-specific T-cells is determined from a bone marrow sample of a patient with a malignancy or tumor treated with a lymphocytic cell composition.

In one embodiment, the levels of circulating TAA-specific T-cells is determined using an Elispot assay. In one embodiment, the levels of circulating TAA-specific T-cells is determined using an intracellular cytokine staining assay. In one embodiment, the levels of circulating TAA-specific T-cells is determined using multimer analysis. In one embodiment, the levels of circulating TAA-specific T-cells is determined by TCR sequencing.

### Hematological and Solid Tumors Targeted for Treatment

The lymphocytic cell composition described herein can be used to treat a patient with a solid or hematological tumor.

Lymphoid neoplasms are broadly categorized into precursor lymphoid neoplasms and mature T-cell, B-cell or natural killer cell (NK) neoplasms. Chronic leukemias are those likely to exhibit primary manifestations in blood and bone marrow, whereas lymphomas are typically found in extramedullary sites, with secondary events in the blood or bone. Over 79,000 new cases of lymphoma were estimated in 2013. Lymphoma is a cancer of lymphocytes, which are a type of white blood cell. Lymphomas are categorized as Hodgkin's or non-Hodgkin's. Over 48,000 new cases of leukemias were expected in 2013.

In one embodiment, the disease or disorder is a hematological malignancy selected from a group consisting of leukemia, lymphoma and multiple myeloma.

In one embodiment, the methods described herein can be used to treat a leukemia. For example, the patient such as a human may be suffering from an acute or chronic leukemia of a lymphocytic or myelogenous origin, such as, but not limited to: Acute lymphoblastic leukemia (ALL); Acute myelogenous leukemia (AML); Chronic lymphocytic leukemia (CLL); Chronic myelogenous leukemia (CML); juvenile myelomonocytic leukemia (JMML); hairy cell leukemia (HCL); acute promyelocytic leukemia (a subtype of AML); large granular lymphocytic leukemia; or Adult T-cell chronic leukemia. In one embodiment, the patient suffers from an acute myelogenous leukemia, for example an undifferentiated AML (MO); myeloblastic leukemia (M1); with/without minimal cell maturation); myeloblastic leukemia (M2; with cell maturation); promyelocytic leukemia (M3 or M3 variant [M3V]); myelomonocytic leukemia (M4 or M4 variant with eosinophilia [M4E]); monocytic leukemia (M5); erythroleukemia (M6); or megakaryoblastic leukemia (M7).

In a particular embodiment, the hematological malignancy is a lymphoma or lymphocytic or myelocytic proliferation disorder or abnormality. In one embodiment, the lymphoma is a non-Hodgkin's lymphoma. In one embodiment, the lymphoma is a Hodgkin's lymphoma.

In some aspects, the methods described herein can be used to treat a patient such as a human, with a Non-Hodgkin's Lymphoma such as, but not limited to: an AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; or Waldenstrom's Macroglobulinemia.

Alternatively, the methods described herein can be used to treat a patient, such as a human, with a Hodgkin's Lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin Lymphoma; or Nodular Lymphocyte Predominant HL.

Alternatively, the methods described herein can be used to treat a patient, for example a human, with specific B-cell lymphoma or proliferative disorder such as, but not limited to: multiple myeloma; Diffuse large B cell lymphoma; Follicular lymphoma; Mucosa-Associated Lymphatic Tissue lymphoma (MALT); Small cell lymphocytic lymphoma; Mediastinal large B cell lymphoma; Nodal marginal zone B cell lymphoma (NMZL); Splenic marginal zone lymphoma (SMZL); Intravascular large B-cell lymphoma; Primary effusion lymphoma; or Lymphomatoid granulomatosis; B-cell prolymphocytic leukemia; Hairy cell leukemia; Splenic lymphoma/leukemia, unclassifiable; Splenic diffuse red pulp small B-cell lymphoma; Hairy cell leukemia-variant; Lymphoplasmacytic lymphoma; Heavy chain diseases, for example, Alpha heavy chain disease, Gamma heavy chain disease, Mu heavy chain disease; Plasma cell myeloma; Solitary plasmacytoma of bone; Extraosseous plasmacytoma; Primary cutaneous follicle center lymphoma; T cell/histiocyte rich large B-cell lymphoma; DLBCL associated with chronic inflammation; Epstein-Barr virus (EBV)+ DLBCL of the elderly; Primary mediastinal (thymic) large B-cell lymphoma; Primary cutaneous DLBCL, leg type; ALK+ large B-cell lymphoma; Plasmablastic lymphoma; Large B-cell lymphoma arising in HHV8-associated multicentric; Castleman disease; B-cell lymphoma, unclassifiable, with features intermediate between diffuse large B-cell lymphoma; or B-cell lymphoma, unclassifiable, with features intermediate between diffuse large B-cell lymphoma and classical Hodgkin lymphoma.

Abnormal proliferation of T-cells, B-cells, and/or NK-cells can result in a wide range of cancers. A host, for example a human, afflicted with any of these disorders can be treated with an effective amount of the TAA-L composition as described herein to achieve a decrease in symptoms (a palliative agent) or a decrease in the underlying disease (a disease modifying agent).

Alternatively, the methods described herein can be used to treat a patient, such as a human, with a hematological malignancy, for example but not limited to T-cell or NK-cell lymphoma, for example, but not limited to: peripheral T-cell lymphoma; anaplastic large cell lymphoma, for example anaplastic lymphoma kinase (ALK) positive, ALK negative anaplastic large cell lymphoma, or primary cutaneous anaplastic large cell lymphoma; angioimmunoblastic lymphoma; cutaneous T-cell lymphoma, for example mycosis fungoides, Sézary syndrome, primary cutaneous anaplastic large cell lymphoma, primary cutaneous CD30⁺ T-cell lymphoproliferative disorder; primary cutaneous aggressive epidermotropic CD8⁺ cytotoxic T-cell lymphoma; primary cutaneous gamma-delta T-cell lymphoma; primary cutaneous small/medium CD4⁺ T-cell lymphoma, and lymphomatoid papulosis; Adult T-cell Leukemia/Lymphoma (ATLL); Blastic NK-cell Lymphoma; Enteropathy-type T-cell lymphoma; Hematosplenic gamma-delta T-cell Lymphoma; Lymphoblastic Lymphoma; Nasal NK/T-cell Lymphomas; Treatment-related T-cell lymphomas; for example lymphomas that appear after solid organ or bone marrow transplantation; T-cell prolymphocytic leukemia; T-cell large granular lymphocytic leukemia; Chronic lymphoproliferative disorder of NK-cells; Aggressive NK cell leukemia; Systemic EBV+ T-cell lymphoproliferative disease of childhood (associated with chronic active EBV infection); Hydroa vacciniforme-like lymphoma; Adult T-cell leukemia/lymphoma; Enteropathy-associated T-cell lymphoma; Hepatosplenic T-cell lymphoma; or Subcutaneous panniculitis-like T-cell lymphoma.

In one embodiment, the lymphocytic cell composition disclosed herein is used to treat a patient with a selected hematopoietic malignancy either before or after hematopoietic stem cell transplantation (HSCT). In some embodiments, the lymphocytic cell composition is used to treat a patient with a selected hematopoietic malignancy after HSCT. In one embodiment, the lymphocytic cell composition is used to treat a patient with a selected hematopoietic malignancy up to about 30, 35, 40, 45, or 50 days after HSCT. In one embodiment, the lymphocytic cell composition is used to treat a patient with a selected hematopoietic malignancy after neutrophil engraftment during the period following HSCT. In some embodiments, the lymphocytic cell composition is used to treat a patient with a selected hematopoietic malignancy before HSCT, such as one week, two weeks, three weeks or more before HSCT.

In some aspects, the tumor is a solid tumor. In one embodiment, the solid tumor is Wilms Tumor. In one embodiment, the solid tumor is osteosarcoma. In one embodiment, the solid tumor is Ewing sarcoma. In one embodiment, the solid tumor is neuroblastoma. In one embodiment, the solid tumor is soft tissue sarcoma. In one embodiment, the solid tumor is rhabdomyosarcoma.

Non-limiting examples of tumors that can be treated according to the present disclosure include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast, triple negative breast cancer, HER2-negative breast cancer, HER2-positive breast cancer, male breast cancer, late-line metastatic breast cancer, progesterone receptor-negative breast cancer, progesterone receptor-positive breast cancer, recurrent breast cancer), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), glioblastoma multiforme, head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

### Administration of Lymphocytic Cell Compositions

Methods for administration of cells for adoptive cell therapy are known and may be used in connection with the provided methods and lymphocytic cell compositions. For example, adoptive T-cell therapy methods are described, e.g., in US Patent Application Publication No. 2003/0170238; U.S. Pat. No. 4,690,915; Rosenberg, Nat. Rev. Clin. Oncol. 8(10):577-85 (2011); Themeli et al., Nat. Biotechnol. 31(10):928-33 (2013); Tsukahara et al., Biochem. Biophys. Res. Commun. 438(1):84-89 (2013); Davila etal., PLoS ONE 8(4):e61338 (2013).

The administration of the lymphocytic cell composition may vary. In one aspect, the lymphocytic cell composition may be administered to a subject such as a human at an interval selected from once every 1 week, 2 weeks, 3 weeks, 4 weeks, 5 weeks, 6 weeks, 7 weeks, 8 weeks, or more after the initial administration of the lymphocytic cell composition. In a typical embodiment, the lymphocytic cell composition is administered in an initial dose then at every 4 weeks thereafter. In one embodiment, the lymphocytic cell composition may be administered repetitively to 1, 2, 3, 4, 5, 6, or more times after the initial administration of the composition. In a typical embodiment, the lymphocytic cell composition is administered repetitively up to 10 more times after the initial administration of the lymphocytic cell composition. In an alternative embodiment, the lymphocytic cell composition is administered more than 10 times after the initial administration of the lymphocytic cell composition.

In one embodiment, a TAA expression profile of the malignancy or tumor of the subject, for example, a human is performed prior to the initial administration of the lymphocytic cell composition. In one embodiment, a TAA expression profile of the malignancy or tumor of the patient is performed prior to each subsequent administration of the lymphocytic cell composition, allowing for the option to adjust the lymphocytic cell composition. In one embodiment, the lymphocytic cell composition of subsequent administrations remains the same as the initial administration. In one embodiment, the lymphocytic cell composition of subsequent administrations is changed based on the change in the TAA expression profile of the malignancy or tumor of the patient.

In some embodiments, the lymphocytic cell composition is administered to a subject in the form of a pharmaceutical composition, such as a composition comprising the cells or cell populations and a pharmaceutically acceptable carrier or excipient. The pharmaceutical compositions in some embodiments additionally comprise other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the agents are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The choice of carrier in the pharmaceutical composition may be determined in part by the by the particular method used to administer the cell composition. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition.

In addition, buffering agents in some aspects are included in the composition. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins 21st ed. (May 1, 2005).

In some embodiments, the pharmaceutical composition comprises the lymphocytic cell composition in an amount that is effective to treat or prevent the disease or condition, such as a therapeutically effective or prophylactically effective amount. Thus, in some embodiments, the methods of administration include administration of the lymphocytic cell composition at effective amounts. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In some embodiments, the lymphocytic cell composition is administered at a desired dosage, which in some aspects includes a desired dose or number of cells and/or a desired ratio of T-cell subpopulations. Thus, the dosage of cells in some embodiments is based on a total number of cells (or number per m² or per kg body weight) and a desired ratio of the individual populations or sub-types. In some embodiments, the dosage of cells is based on a desired total number (or number per m² or per kg of body weight) of cells in the individual populations or of individual cell types. In some embodiments, the dosage is based on a combination of such features, such as a desired number of total cells, desired ratio, and desired total number of cells in the individual populations.

In some embodiments, the lymphocytic cell composition is administered at or within a tolerated difference of a desired dose of total cells, such as a desired dose of T-cells. In some aspects, the desired dose is a desired number of cells, a desired number of cells per unit of body surface area or a desired number of cells per unit of body weight of the subject to whom the cells are administered, e.g., cells/m² or cells/kg. In some aspects, the desired dose is at or above a minimum number of cells or minimum number of cells per unit of body surface area or body weight. In some aspects, among the total cells, administered at the desired dose, the individual populations or sub-types are present at or near a desired output ratio as described herein, e.g., within a certain tolerated difference or error of such a ratio.

In some embodiments, the cells are administered at or within a tolerated difference of a desired dose. In some aspects, the desired dose is a desired number of cells, or a desired number of such cells per unit of body surface area or body weight of the subject to whom the cells are administered, e.g., cells/m² or cells/kg. In some aspects, the desired dose is at or above a minimum number of cells of the population, or minimum number of cells of the population per unit of body surface area or body weight.

Thus, in some embodiments, the dosage is based on a desired fixed dose of total cells and a desired ratio, and/or based on a desired fixed dose of two or more, e.g., each, of the individual T-cell subpopulations. Thus, in some embodiments, the dosage is based on a desired fixed or minimum dose of T-cell subpopulations and a desired ratio thereof.

In certain embodiments, lymphocytic cell composition is administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual T-cell subpopulations of cells is within a range of between at or about 10⁴ and at or about 10⁹ cells/meter² (m²) body surface area, such as between 10⁵ and 10⁶ cells/m² body surface area, for example, at or about 1×10⁵ cells/m², 1.5×10⁵ cells/m², 2×10⁵ cells/m², or 1×10⁶ cells/m² body surface area. For example, in some embodiments, the cells are administered at, or within a certain range of error of, between at or about 10⁴ and at or about 10⁹ T cells/meter² (m²) body surface area, such as between 10⁵ and 10⁶ T cells/m² body surface area, for example, at or about 1×10⁵ T cells/m², 1.5×10⁵ T cells/m², 2×10⁵ T cells/m², or 1×10⁶ T cells/m² body surface area.

In some embodiments, the cells are administered at or within a certain range of error of between at or about 10⁴ and at or about 10⁹ cells/meter² (m²) body weight, such as between 10⁵ and 10⁶ cells/m² body weight, for example, at or about 1×10⁵ cells/m², 1.5×10⁵ cells/m², 2×10⁵ cells/kg, or 1×10⁶ cells/m² body surface area.

### Product Release Testing and Characterization of T-Cell Subpopulations

Prior to infusion, the lymphocytic cell composition may be characterized for safety and release testing. Product release testing, also known as lot or batch release testing, is an important step in the quality control process of drug substances and drug products. This testing verifies that a T-cell subpopulation and/or lymphocytic cell composition meets a pre-determined set of specifications. Pre-determined release specifications for T-cell subpopulations and lymphocytic cell composition include confirmation that the cell product is >70% viable, has <5.0 EU/ml of endotoxin, is negative for aerobic, anaerobic, fungal pathogens and mycoplasma, and lacks reactivity to allogeneic PHA blasts, for example, with less than 10% lysis to PHA blasts. The phenotype of the lymphocytic cell composition may be determined with requirements for clearance to contain, in one non-limiting embodiment, <2% dendritic cells and <2% B cells. The HLA identity between the lymphocytic cell composition and the donor is also confirmed.

Antigen specificity of the T-cell subpopulations can be tested via an Interferon-y Enzyme-Linked Immunospot (IFNγ ELISpot) assay. Other cytokines can also be utilized to measure antigen specificity including TNFa and IL-4. Pre-stimulated effector cells and target cells pulsed with the TAA of interest are incubated in a 96-well plate (pre-incubated with anti-INF-γ antibody) at an E/T ratio of 1:2. They are compared with no-TAA control, an irrelevant peptide not used for T-cell generation, and SEB as a positive control. After washing, the plates are incubated with a biotinylated anti-IFN-γ antibody. Spots are detected by incubating with streptavidin-coupled alkaline phosphastase and substrate. Spot forming cells (SFCs) are counted and evaluated using an automated plate reader.

The phenotype of the lymphocytic cell composition can be determined by extracellular antibody staining with anti-CD3, CD4, CD8, CD45, CD19, CD16, CD56, CD14, CD45, CD83, HLA-DR, TCRαγ, TCRγδ and analyzed on a flow cytometer. Annexin-V and PI antibodies can be used as viability controls, and data analyzed with FlowJo Flow Cytometry software (Treestar, Ashland, Oreg., USA).

The lytic capacity of T-cell subpopulations can be evaluated via ⁵¹Chromium (⁵¹Cr) and Europium (Eu)-release cytotoxicity assays to test recognition and lysis of target cells by the T-cell subpopulations and lymphocytic cell compositions.

Typically, activated primed T-cells (effector cells) can be tested against ⁵¹Cr-labeled target cells at effector-to-target ratios of, for example, 40:1, 20:1, 10:1, and 5:1. Cytolytic activity can be determined by measuring ⁵¹Cr release into the supernatant on a gamma-counter. Spontaneous release is assessed by incubating target cells alone, and maximum lysis by adding 1% Triton X-100. Specific lysis was calculated as: specific lysis (%)=(experimental release−spontaneous release)/(maximum release−spontaneous release)×100.

Europium-release assays can also be utilized to measure the lytic capacity of T-cell subpopulations and lymphocytic cell composition. This is a non-radioactive alternative to the conventional Chromium-51 (⁵¹Cr) release assay and works on the same principle as the radioactive assay. Target cells are first loaded with an acetoxymethyl ester of BATDA. The ligand penetrates the cell membrane quickly. Within the cell, the ester bonds are hydrolyzed to form a hydrophilic ligand (TDA), which no longer passes through the cell membrane. If cells are lysed by an effector cell, TDA is released outside the cell into the supernatant. Upon addition of Europium solution to the supernatant, Europium can form a highly fluorescent and stable chelate with the released TDA (EuTDA). The measured fluorescence signal correlates directly with the number of lysed cells in the cytotoxicity assay. Specific lysis was calculated as: specific lysis (%)=(experimental release−spontaneous release)/(maximum release−spontaneous release)×100.

### Monitoring

Following administration of the cells, the biological activity of the administered cell populations in some embodiments is measured, e.g., by any of a number of known methods. Parameters to assess include specific binding of a T-cell or other immune cell to antigen, in vivo, e.g., by imaging, or ex vivo, e.g., by ELISA or flow cytometry. In certain embodiments, the ability of the administered cells to destroy target cells can be measured using any suitable method known in the art, such as cytotoxicity assays described in, for example, Kochenderfer et al., J. Immunother. 32(7):689-702 (2009); and Herman etal., J. Immunol. Methods 285(1):25-40 (2004), all incorporated herein by reference. In certain embodiments, the biological activity of the cells is measured by assaying expression and/or secretion of one or more cytokines, such as IFNγ, IL-2, and TNF. In some aspects the biological activity is measured by assessing clinical outcome, such as reduction in tumor burden or load.

### Combination Therapies

In one aspect, lymphocytic cell compositions disclosed herein can be beneficially administered in combination with another therapeutic regimen for beneficial, additive, or synergistic effects.

In one embodiment, the lymphocytic cell composition is administered in combination with another therapy to treat a hematological malignancy. In one embodiment, the lymphocytic cell composition is administered in combination with another therapy to treat a solid tumor. The second therapy can be a pharmaceutical or a biologic agent (for example an antibody) to increase the efficacy of treatment with a combined or synergistic approach.

In one embodiment, the additional therapy is a monoclonal antibody (MAb). Some MAbs stimulate an immune response that destroys tumor cells. Similar to the antibodies produced naturally by B cells, these MAbs “coat” the tumor cell surface, triggering its destruction by the immune system. FDA-approved MAbs of this type include rituximab, which targets the CD20 antigen found on non-Hodgkin lymphoma cells, and alemtuzumab, which targets the CD52 antigen found on B-cell chronic lymphocyticleukemia (CLL) cells. Rituximab may also trigger cell death (apoptosis) directly. Another group of MAbs stimulates an antitumor immune response by binding to receptors on the surface of immune cells and inhibiting signals that prevent immune cells from attacking the body's own tissues, including tumor cells. Other MAbs interfere with the action of proteins that are necessary for tumor growth. For example, bevacizumab targets vascular endothelial growth factor (VEGF), a protein secreted by tumor cells and other cells in the tumor's microenvironment that promotes the development of tumor blood vessels. When bound to bevacizumab, VEGF cannot interact with its cellular receptor, preventing the signaling that leads to the growth of new blood vessels. Similarly, cetuximab and panitumumab target the epidermal growth factor receptor (EGFR). MAbs that bind to cell surface growth factor receptors prevent the targeted receptors from sending their normal growth-promoting signals. They may also trigger apoptosis and activate the immune system to destroy tumor cells. Another group of tumor therapeutic MAbs are the immunoconjugates. These MAbs, which are sometimes called immunotoxins or antibody-drug conjugates, consist of an antibody attached to a cell-killing substance, such as a plant or bacterial toxin, a chemotherapy drug, or a radioactive molecule. The antibody latches onto its specific antigen on the surface of a tumor cell, and the cell-killing substance is taken up by the cell. FDA-approved conjugated MAbs that work this way include 90Y-ibritumomab tiuxetan, which targets the CD20 antigen to deliver radioactive yttrium-90 to B-cell non-Hodgkin lymphoma cells; ¹³¹I-tositumomab, which targets the CD20 antigen to deliver radioactive ¹³¹I to non-Hodgkin lymphoma cells.

In one embodiment, the additional agent is an immune checkpoint inhibitor (ICI), for example, but not limited to PD-1 inhibitors, PD-L1 inhibitors, PD-L2 inhibitors, CTLA-4 inhibitors, LAG-3 inhibitors, TIM-3 inhibitors, and V-domain Ig suppressor of T-cell activation

(VISTA) inhibitors, or combinations thereof.

In one embodiment, the immune checkpoint inhibitor is a PD-1 inhibitor that blocks the interaction of PD-1 and PD-L1 by binding to the PD-1 receptor, and in turn inhibits immune suppression. In one embodiment, the immune checkpoint inhibitor is a PD-1 immune checkpoint inhibitor selected from nivolumab (Opdivo®), pembrolizumab (Keytruda®), pidilizumab, AMP-224 (AstraZeneca and MedImmune), PF-06801591 (Pfizer), MEDI0680 (AstraZeneca), PDR001 (Novartis), REGN2810 (Regeneron), MGA012 (MacroGenics), BGB-A3 17 (BeiGene) SHR-12-1 (Jiangsu Hengrui Medicine Company and Incyte Corporation), TSR-042 (Tesaro), and the PD-L1/VISTA inhibitor CA-170 (Curis Inc.).

In one embodiment, the immune checkpoint inhibitor is the PD-1 immune checkpoint inhibitor nivolumab (Opdivo®) administered in an effective amount for the treatment of Hodgkin's lymphoma. In another aspect of this embodiment, the immune checkpoint inhibitor is the PD-1 immune checkpoint inhibitor pembrolizumab (Keytruda®) administered in an effective amount. In an additional aspect of this embodiment, the immune checkpoint inhibitor is the PD-1 immune checkpoint inhibitor pidilizumab (Medivation) administered in an effective amount for refractory diffuse large B-cell lymphoma (DLBCL).

In one embodiment, the immune checkpoint inhibitor is a PD-L1 inhibitor that blocks the interaction of PD-1 and PD-L1 by binding to the PD-L1 receptor, and in turn inhibits immune suppression. PD-L1 inhibitors include, but are not limited to, atezolizumab, durvalumab, KNO35CA-170 (Curis Inc.), and LY3300054 (Eli Lilly).

In one embodiment, the immune checkpoint inhibitor is the PD-L1 immune checkpoint inhibitor atezolizumab (Tecentriq®) administered in an effective amount. In another aspect of this embodiment, the immune checkpoint inhibitor is durvalumab (AstraZeneca and Medlmmune) administered in an effective. In yet another aspect of the embodiment, the immune checkpoint inhibitor is KN035 (Alphamab). An additional example of a PD-L1 immune checkpoint inhibitor is BMS-936559 (Bristol-Myers Squibb), although clinical trials with this inhibitor have been suspended as of 2015.

In one aspect of this embodiment, the immune checkpoint inhibitor is a CTLA-4 immune checkpoint inhibitor that binds to CTLA-4 and inhibits immune suppression. CTLA-4 inhibitors include, but are not limited to, ipilimumab, tremelimumab (AstraZeneca and MedImmune), AGEN1884 and AGEN2041 (Agenus).

In one embodiment, the CTLA-4 immune checkpoint inhibitor is ipilimumab (Yervoy®) administered in an effective amount

In another embodiment, the immune checkpoint inhibitor is a LAG-3 immune checkpoint inhibitor. Examples of LAG-3 immune checkpoint inhibitors include, but are not limited to, BMS-986016 (Bristol-Myers Squibb), GSK2831781 (GlaxoSmithKline), IMP321 (Prima BioMed), LAG525 (Novartis), and the dual PD-1 and LAG-3 inhibitor MGD013 (MacroGenics). In yet another aspect of this embodiment, the immune checkpoint inhibitor is a TIM-3 immune checkpoint inhibitor. A specific TIM-3 inhibitor includes, but is not limited to, TSR-022 (Tesaro).

Other immune checkpoint inhibitors for use in combination with the compositions described herein include, but are not limited to, B7-H3/CD276 immune checkpoint inhibitors such as MGA217, indoleamine 2,3-dioxygenase (IDO) immune checkpoint inhibitors such as Indoximod and INCB024360, killer immunoglobulin-like receptors (KIRs) immune checkpoint inhibitors such as Lirilumab (BMS-986015), carcinoembryonic antigen cell adhesion molecule (CEACAM) inhibitors (e.g., CEACAM-1, -3 and/or -5). Exemplary anti-CEACAM-1 antibodies are described in WO 2010/125571, WO 2013/082366 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In other embodiments, the anti-CEACAM antibody binds to CEACAM-5 as described in, e.g., Zheng et al., PLoS One 5(9):e12529 (DOI:10: 1371/journal.pone.0021146) (2010), or cross-reacts with CEACAM-1 and CEACAM-5 as described in, e.g., WO 2013/054331 and US 2014/0271618. Still other checkpoint inhibitors can be molecules directed to B and T lymphocyte attenuator molecule (BTLA), for example as described in Zhang et al., Clin. Exp. Immunol. 2011 163(1):77-87 (2010).

Current chemotherapeutic drugs that may be used in combination with the lymphocytic cell composition described herein include those used to treat AML including cytarabine (cytosine arabinoside or ara-C) and the anthracycline drugs (such as daunorubicin/daunomycin, idarubicin, and mitoxantrone). Some of the other chemo drugs that may be used to treat AML include: Cladribine (Leustatin®, 2-CdA), Fludarabine (Fludara®), Topotecan, Etoposide (VP-16), 6-thioguanine (6-TG), Hydroxyurea (Hydrea®), Corticosteroid drugs, such as prednisone or dexamethasone (Decadron®), Methotrexate (MTX), 6-mercaptopurine (6-MP), Azacitidine (Vidaza®), Decitabine (Dacogen®). Additional drugs include dasatinib and checkpoint inhibitors such as novolumab, Pembrolizumab, and atezolizumab.

Current chemotherapeutic drugs that may be used in combination with the lymphocytic cell compositionn described herein include those used for CLL and other lymphomas including: purine analogs such as fludarabine (Fludara®), pentostatin (Nipent®), and cladribine (2-CdA, Leustatin®), and alkylating agents, which include chlorambucil (Leukeran®) and cyclophosphamide (Cytoxan®) and bendamustine (Treanda®). Other drugs sometimes used for CLL include doxorubicin (Adriamycin®), methotrexate, oxaliplatin, vincristine (Oncovin®), etoposide (VP-16), and cytarabine (ara-C). Other drugs include Rituximab (Rituxan), Obinutuzumab (Gazyva™), Ofatumumab (Arzerra®), Alemtuzumab (Campath®) and Ibrutinib (Imbruvica™)

Current chemotherapeutic drugs that may be used in combination with the lymphocytic cell composition described herein include those used for CML including: Interferon, imatinib (Gleevec), the chemo drug hydroxyurea (Hydrea®), cytarabine (Ara-C), busulfan, cyclophosphamide (Cytoxan®), and vincristine (Oncovin®). Omacetaxine (Synribo®) is a chemo drug that was approved to treat CML that is resistant to some of the TKIs now in use.

Current chemotherapeutic drugs that may be used in combination with the lymphocytic cell composition described herein include those used for CMML, for example, Deferasirox (Exjade®), cytarabine with idarubicin, cytarabine with topotecan, and cytarabine with fludarabine, Hydroxyurea (hydroxycarbamate, Hydrea®), azacytidine (Vidaza®) and decitabine (Dacogen®). Current chemotherapeutic drugs that may be used in combination with the lymphocytic cell composition described herein include those used for multiple myeloma include Pomalidomide (Pomalyst®), Carfilzomib (Kyprolis™), Everolimus (Afinitor®), dexamethasone (Decadron), prednisone and methylprednisolone (Solu-medrol®) and hydrocortisone.

Current chemotherapeutic drugs that may be used in combination with the lymphocytic cell composition described herein include those used for Hodgkin's disease include Brentuximab

WO 2020/146434 PCT/US2020/012639 vedotin (Adcetris™): anti-CD-30, Rituximab, Adriamycin® (doxorubicin), Bleomycin, Vinblastine, Dacarbazine (DTIC).

Current chemotherapeutic drugs that may be used in combination with the lymphocytic cell composition described herein include those used for Non-Hodgkin's disease include Rituximab (Rituxan®), Ibritumomab (Zevalin®), tositumomab (Bexxar®), Alemtuzumab (Campath®) (CD52 antigen), Ofatumumab (Arzerra®), Brentuximab vedotin (Adcetris®) and Lenalidomide (Revlimid®).

Current chemotherapeutic drugs that may be used in combination with the lymphocytic cell composition described herein include those used for:

B-cell Lymphoma, for example:

Diffuse large B-cell lymphoma: CHOP (cyclophosphamide, doxorubicin, vincristine, and prednisone), plus the monoclonal antibody rituximab (Rituxan). This regimen, known as R-CHOP, is usually given for about 6 months.

Primary mediastinal B-cell lymphoma: R-CHOP.

Follicular lymphoma: rituximab (Rituxan) combined with chemo, using either a single chemo drug (such as bendamustine or fludarabine) or a combination of drugs, such as the CHOP or CVP (cyclophosphamide, vincristine, prednisone regimens. The radioactive monoclonal antibodies, ibritumomab (Zevalin) and tositumomab (Bexxar) are also possible treatment options. For patients who may not be able to tolerate more intensive chemo regimens, rituximab alone, milder chemo drugs (such as chlorambucil or cyclophosphamide).

Chronic lymphocytic leukemia/small lymphocytic lymphoma: R-CHOP.

Mantle cell lymphoma: fludarabine, cladribine, or pentostatin; bortezomib (Velcade) and lenalidomide (Revlimid) and ibrutinib (Imbruvica).

Extranodal marginal zone B-cell lymphoma—mucosa-associated lymphoid tissue (MALT) lymphoma: rituximab; chlorambucil or fludarabine or combinations such as CVP, often along with rituximab.

Nodal marginal zone B-cell lymphoma: rituximab (Rituxan) combined with chemo, using either a single chemo drug (such as bendamustine or fludarabine) or a combination of drugs, such as the CHOP or CVP (cyclophosphamide, vincristine, prednisone regimens. The radioactive monoclonal antibodies, ibritumomab (Zevalin) and tositumomab (Bexxar) are also possible treatment options. For patients who may not be able to tolerate more intensive chemo regimens, rituximab alone, milder chemo drugs (such as chlorambucil or cyclophosphamide).

Splenic marginal zone B-cell lymphoma: rituximab; patients with Hep C-anti-virals.

Burkitt lymphoma: methotrexate; hyper-CVAD—cyclophosphamide, vincristine, doxorubicin (also known as Adriamycin), and dexamethasone. Course B consists of methotrexate and cytarabine; CODOX-M—cyclophosphamide, doxorubicin, high-dose methotrexate/ifosfamide, etoposide, and high-dose cytarabine; etoposide, vincristine, doxorubicin, cyclophosphamide, and prednisone (EPOCH)

Lymphoplasmacytic lymphoma—rituximab.

Hairy cell leukemia—cladribine (2-CdA) or pentostatin; rituximab; interferon-alfa

T-cell lymphomas, for example:

Precursor T-lymphoblastic lymphoma/leukemia—cyclophosphamide, doxorubicin (Adriamycin), vincristine, L-asparaginase, methotrexate, prednisone, and, sometimes, cytarabine (ara-C). Because of the risk of spread to the brain and spinal cord, a chemo drug such as methotrexate is also given into the spinal fluid.

Skin lymphomas: Gemcitabine Liposomal doxorubicin (Doxil); Methotrexate; Chlorambucil; Cyclophosphamide; Pentostatin; Etoposide; Temozolomide; Pralatrexate; R-CHOP.

Angioimmunoblastic T-cell lymphoma: prednisone or dexamethasone.

Extranodal natural killer/T-cell lymphoma, nasal type: CHOP.

Anaplastic large cell lymphoma: CHOP; pralatrexate (Folotyn), targeted drugs such as bortezomib (Velcade) or romidepsin (Istodax), or immunotherapy drugs such as alemtuzumab (Campath) and denileukin diftitox (Ontak).

Primary central nervous system (CNS) lymphoma—methotrexate; rituximab.

A more general list of suitable chemotherapeutic agents includes, but are not limited to, radioactive molecules, toxins, also referred to as cytotoxins or cytotoxic agents, which includes any agent that is detrimental to the viability of cells, agents, and liposomes or other vesicles containing chemotherapeutic compounds. Examples of suitable chemotherapeutic agents include but are not limited to 1-dehydrotestosterone, 5-fluorouracil decarbazine, 6-mercaptopurine, 6-thioguanine, actinomycin D, adriamycin, aldesleukin, alkylating agents, allopurinol sodium, altretamine, amifostine, anastrozole, anthramycin (AMC)), anti-mitotic agents, cis-dichlorodiamine platinum (II) (DDP) cisplatin), diamino dichloro platinum, anthracyclines, antibiotics, antis, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), Chlorambucil, Cisplatin, Cladribine, Colchicin, conjugated estrogens, Cyclophosphamide, Cyclothosphamide, Cytarabine, Cytarabine, cytochalasin B, Cytoxan, Dacarbazine, Dactinomycin, dactinomycin (formerly actinomycin), daunorubicin HCl, daunorucbicin citrate, denileukin diftitox, Dexrazoxane, Dibromomannitol, dihydroxy anthracin dione, Docetaxel, dolasetron mesylate, doxorubicin HCl, dronabinol, E. coli L-asparaginase, emetine, epoetin-a, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCl, glucocorticoids, goserelin acetate, gramicidin D, granisetron HCl, hydroxyurea, idarubicin HCl, ifosfamide, interferon a-2b, irinotecan HCl, letrozole, leucovorin calcium, leuprolide acetate, levamisole HCl, lidocaine, lomustine, maytansinoid, mechlorethamine HCl, medroxyprogesterone acetate, megestrol acetate, melphalan HCl, mercaptipurine, mesna, methotrexate, methyltestosterone, mithramycin, mitomycin C, mitotane, mitoxantrone, nilutamide, octreotide acetate, ondansetron HCl, paclitaxel, pamidronate disodium, pentostatin, pilocarpine HCl, plimycin, polifeprosan 20 with carmustine implant, porfimer sodium, procaine, procarbazine HCl, propranolol, rituximab, sargramostim, streptozotocin, tamoxifen, taxol, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

Additional therapeutic agents that can be administered in combination with the lymphocytic cell compositions disclosed herein can include bevacizumab, sutinib, sorafenib, 2-methoxyestradiol, finasunate, vatalanib, vandetanib, aflibercept, volociximab, etaracizumab, cilengitide, erlotinib, cetuximab, panitumumab, gefitinib, trastuzumab, atacicept, rituximab, alemtuzumab, aldesleukine, atlizumab, tocilizumab, temsirolimus, everolimus, lucatumumab, dacetuzumab, atiprimod, natalizumab, bortezomib, carfilzomib, marizomib, tanespimycin, saquinavir mesylate, ritonavir, nelfinavir mesylate, indinavir sulfate, belinostat, panobinostat, mapatumumab, lexatumumab, oblimersen, plitidepsin, talmapimod, enzastaurin, tipifarnib, perifosine, imatinib, dasatinib, lenalidomide, thalidomide, simvastatin, and celecoxib.

In one aspect, the lymphocytic cell compositions disclosed herein are administered in combination with at least one immunosuppressive agent. The immunosuppressive agent may be selected from the group consisting of a calcineurin inhibitor, e.g. a cyclosporin or an ascomycin, e.g. Cyclosporin A (NEORAL®), tacrolimus, a mTOR inhibitor, e.g. rapamycin or a derivative thereof, e.g. Sirolimus (RAPAMUNE®), Everolimus (Certican®), temsirolimus, biolimus-7, biolimus-9, a rapalog, e.g. azathioprine, campath 1H, a S1P receptor modulator, e.g. fingolimod or an analogue thereof, an anti-IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3®), Prednisone, ATGAM®, THYMOGLOBULIN®, Brequinar Sodium, 15-deoxyspergualin, tresperimus, Leflunomide ARAVA®, anti-CD25, anti-IL2R, Basiliximab (SIMULECT®), Daclizumab (ZENAPAX®), mizorbine, methotrexate, dexamethasone, pimecrolimus (Elidel®), abatacept, belatacept, etanercept (Enbrel®), adalimumab (Humira®), infliximab (Remicade®), an anti-LFA-1 antibody, natalizumab (Antegren®), Enlimomab, ABX-CBL, antithymocyte immunoglobulin, siplizumab, and efalizumab.

In one aspect, the lymphocytic cell composition described herein can be administered in combination with at least one anti-inflammatory agent. The anti-inflammatory agent can be a steroidal anti-inflammatory agent, a nonsteroidal anti-inflammatory agent, or a combination thereof. In some embodiments, anti-inflammatory drugs include, but are not limited to, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof.

In one aspect, the lymphocytic cell composition described herein can be administered in combination with at least one immunomodulatory agent.

### Methods of Manufacturing Lymphocytic Cell Compositions

T-cell subpopulations specific for a single TAA to be combined into the lymphocytic cell compositions for therapeutic administration described herein can be generated using any known method in the art or as described herein. Activated T-cell subpopulations that recognize at least one epitope of an antigen of a tumor can be generated by any method known in the art or as described herein. Non-limiting exemplary methods of generating activated T-cell subpopulations that recognize at least one epitope of an antigen of a tumor can be found in, for example Shafer et al., Leuk Lymphoma 51(5):870-80 (2010); Cruz et al., Clin. Cancer Res. 17(22):7058-66 (2011); Quintarelli et al., Blood 117(12):3353-62 (2011); and Chapuis et al. 2013, supra, all incorporated herein by reference.

Generally, generating the T-cell subpopulations of the lymphocytic cell compositions of the present disclosure may involve (i) collecting a peripheral blood mononuclear cell product from a donor; (ii) determining the HLA subtype of the mononuclear cell product; (iii) separating the monocytes and the lymphocytes of the mononuclear cell product; (iv) generating and maturing dendritic cells (DCs) from the monocytes; (v) pulsing the DCs with a TAA; (vi) optionally carrying out a CD45RA+ selection to isolate naïve lymphocytes; (vii) stimulating the naïve lymphocytes with the peptide-pulsed DCs in the presence of a cytokine cocktail; (viii) repeating the T cell stimulation with fresh peptide-pulsed DCs or other peptide-pulsed antigen presenting cells in the presence of a cytokinor (ix) subjecting the cells to a selection protocol which isolates the desired specific lymphocytic cell subsets into discrete populations; (x) optionally further expanding one or more of the discrete lymphocytic cell subset populations to derive sufficient numbers to arrive at a fixed ratio described herein suitable for administration at a total cell population described herein; (xi) recombining the discrete cell populations to provide a cell composition at the fixed ratios described herein, or in an alternative embodiment, optionally keeping the discrete lymphocytic cell subsets separate wherein the population is suitable for inclusion in a kit suitable for administration to a patient, wherein each discrete lymphocytic cell subset is at a cell population corresponding to a cell composition fixed ratio described herein collectively; and optionally (xii) cryopreserving for future use.

Collecting a Peripheral Blood Mononuclear Cell Product from a Donor

The generation of T-cell subpopulations to be specific to a single TAA generally requires a peripheral blood mononuclear cell (PBMC) product from a donor, either an allogeneic or autologous donor, as a starting material. Isolation of PBMCs is well known in the art. Non-limiting exemplary methods of isolating PBMCs are provided in Grievink et al., Biopreserv. Biobank. 14(5):410-15 (2016), which is incorporated herein by reference. The PBMC product can be isolated from whole blood, an apheresis sample, a leukapheresis sample, or a bone marrow sample provided by a donor. In one embodiment, the starting material is an apheresis sample, which provides a large number of initially starting mononuclear cells, potentially allowing a large number of different T-cell subpopulations to be generated. In one embodiment, the PBMC product is isolated from a sample containing peripheral blood mononuclear cells (PBMCs) provided by a donor. In one embodiment, the donor is a healthy donor. In one embodiment, the PBMC product is derived from cord blood. In one embodiment, the donor is the same donor providing stem cells for a hematopoietic stem cell transplant (HSCT).

### Determining HLA Subtype

When the T-cell subpopulations are generated from an allogeneic, healthy donor, the HLA subtype profile of the donor source is determined and characterized. Determining HLA subtype (i.e., typing the HLA loci) can be performed by any method known in the art. Non-limiting exemplary methods for determining HLA subtype can be found in Lange et al., BMC Genomics 15:63 (2014); Erlich, Tissue Antigens 80:1-11 (2012); Bontadini, Methods 56:471-76 (2012); Dunn, Int. J. Immunogenet. 38:463-73 (2011); and Hurley, C. K., “DNA-based typing of HLA for transplantation.” in Leffell, M. S., et al., eds., Handbook of Human Immunology, 1997. Boca Raton: CRC Press, each independently incorporated herein by reference. In some embodiments, the HLA-subtyping of each donor source is as complete as possible.

In one embodiment, the determined HLA subtypes include at least 4 HLA loci, preferably HLA-A, HLA-B, HLA-C, and HLA-DRB1. In one embodiment, the determined HLA subtypes include at least 6 HLA loci. In one embodiment, the determined HLA subtypes include at least 6 HLA loci. In one embodiment, the determined HLA subtypes include all of the known HLA loci. In general, typing more HLA loci is preferable, since the more HLA loci that are typed, the more likely the allogeneic T-cell subpopulations selected will have highest activity relative to other allogeneic T-cell subpopulations that have HLA alleles or HLA allele combinations in common with the patient or the diseased cells in the patient.

Separating the Monocytes and the Lymphocytes of the Peripheral Blood Mononuclear Cell Product

In general, the PBMC product may be separated into various cell-types, for example, into platelets, red blood cells, lymphocytes, and monocytes, and the lymphocytes and monocytes retained for initial generation of the T-cell subpopulations. The separation of PBMCs is known in the art. Non-limiting exemplary methods of separating monocytes and lymphocytes include Vissers et al., J. Immunol. Methods 110(2):203-07 (1988); and Wahl et al., Curr. Protoc. Immunol. 7.6A.1-7.6A.10 (2005), which are incorporated herein by reference. For example, the separation of the monocytes can occur by plate adherence, by CD14+ selection, or other known methods. The monocyte fraction is generally retained in order to generate dendritic cells used as an antigen presenting cell in the T-cell subpopulation manufacture. The lymphocyte fraction of the PBMC product can be cryopreserved until needed, for example, aliquots of the lymphocyte fraction (˜5×10⁷ cells) can be cryopreserved separately for both Phytohemagglutinin (PHA) Blast expansion and T-cell subpopulation generation.

### Generating Dendritic Cells

The generation of mature dendritic cells used for antigen presentation to prime T-cells is well known in the art. Non-limiting exemplary methods are included in Nair et al., Curr. Protoc. Immunol. 0 7: Unit7.32. doi:10.1002/0471142735.im0732s99 (2012); and Castiello et al., Cancer Immunol. Immunother. 60(4):457-66 (2011), which are incorporated herein by reference. For example, the monocyte fraction can be plated into a closed system bioreactor such as the Quantum Cell Expansion System, and the cells allowed to adhere for 2-4 hours at which point 1,000 U/mL of IL-4 and 800 U/mL GM-CSF can be added. The concentration of GM-CSF and IL-4 can be maintained. The dendritic cells can be matured using a cytokine cocktail. In one embodiment the cytokine cocktail consists of LPS (30 ng/mL), IL-4 (1,000 U/mL), GM-C SF (800 U/mL), TNF-α (10 ng/mL), IL-6 (100 ng/mL), and IL-1β (10 ng/mL). The dendritic cell maturation generally occurs in 2 to 5 days. In one embodiment, the adherent DCs are harvested and counted using a hemocytometer. In one embodiment, a portion of the DCs are cryopreserved for additional further stimulations.

### Pulsing the Dendritic Cells

The non-mature and mature dendritic cells are pulsed with one or more peptides, of a single TAA. For example, the dendritic cells can be pulsed using one or more peptides, for example specific epitopes and/or a pepmix. Methods of pulsing a dendritic cell with a TAA are known. For example, about 100 ng of one or more peptides of the TAA, for example a peptide library (PepMix), can be added per 10 million dendritic cells and incubated for about 30 to 120 minutes.

### Naïve T-Cell Selection of Lymphocytes

In order to increase the potential number of specific TAA activated T-cells and reduce T-cells that target other antigens, it is preferable to utilize naïve T-cells as a starting material. To isolate naïve T-cells, the lymphocytes can undergo a selection, for example CD45RA+ cells selection. CD45RA+ cell selection methods are generally known in the art. Non-limiting exemplary methods are found in Richards et al., Immunology 91(3):331-39 (1997); and McBreen et al., J. Virol. 75(9):4091-4102 (2001), which are incorporated herein by reference. For example, to select for CD45RA⁺ cells, the cells can be labeled using 1 vial of CD45RA microbeads from Miltenyi Biotec per 1×10¹¹ cells after 5-30 minutes of incubation with 100 mL of CliniMACS buffer and approximately 3 mL of 10% human IVIG, 10 μg/mL DNAase I, and 200 mg/mL of magnesium chloride. After 30 minutes, cells will be washed sufficiently and resuspended in 20 mL of CliniMACS buffer. The bag will then be set up on the CLINIMACS Plus device and the selection program can be run according to manufacturer's recommendations. After the program is completed, cells can be counted, washed and resuspended in “CTL Media” consisting of 44.5% EHAA Click's, 44.5% Advanced RPMI, 10% Human Serum, and 1% GlutaMAX.

Stimulating Naïve T cells with Peptide-Pulsed Dendritic Cells

Prior to stimulating naïve T-cells with the dendritic cells, in some embodiments, the DCs are irradiated, for example, at 25 Gy. The DCs and naïve T-cells are then co-cultured. The naïve T-cells can be co-cultured in a ratio range of DCs to T cells of about 1:5-1:50, for example, about 1:5; about 1:10, about 1:15, about 1:20, about 1:25, about 1:30, about 1:35, about 1:40, about 1:45, or about 1:50. The DCs and T-cells are generally co-cultured with cytokines. In one embodiment, the cytokines are selected from a group consisting of IL-6 (100 ng/mL), IL-7 (10 ng/mL), IL-15 (5 ng/mL), IL-12 (10 ng/mL), and IL-21 (10 ng/mL).

### Second T-Cell Stimulation

In some embodiments, the T-cell subpopulations are further stimulated with one or additional stimulation procedures. The additional stimulation can be performed with, for example, fresh DCs pulsed with the same peptides as used in the first stimulation, similarly to as described above. In one embodiment, the cytokines used during the second stimulation are selected from a group consisting of IL-7 (10 ng/mL) and IL-2 (100 U/mL).

Alternatively, peptide-pulsed PHA blasts can be used as the antigen presenting cell. The use of peptide-pulsed PHA blasts to stimulate and expand T-cells are well known in the art Non-limiting exemplary methods can be found in Weber et al. 2013, supra; and Ngo et al. 2014, supra, which are incorporated herein by reference. The peptide-pulsed PHA blasts can be used to expand the T-cell subpopulation in a ratio range of PHA blasts to expanded T cells of about 10:1-1:10. For example, the ratio of PHA blasts to T cells can be about 10:1, between 10:1 and 9:1, between 9:1 and 8:1, between 8:1 and 7:1, between 7:1 and 6:1, between 6:1 and 5:1, between 5:1 and 4:1, between 4:1 and 3:1, between 3:1 and 2:1, between 2:1 and 1:1, between 1:1 and 1:2, between 1:2 and 1:3, between 1:3 and 1:4, between 1:4 and 1:5, between 1:5 and 1:6, between 1:6 and 1:7, between 1:7 and 1:8, between 1:8 and 1:9, between 1:9 and 1:10. In general, cytokines are included in the co-culture, and are selected from the group consisting of IL-7 (10 ng/mL) and IL-2 (100 U/mL).

### Additional T-Cell Expansion and T-Cell Subpopulation Harvest

Additional T cell stimulations may be necessary to generate the necessary number of T-cell subpopulations for use in the lymphocytic cell composition. Following any stimulation and expansion, the T-cell subpopulations are harvested, washed, and concentrated. In one embodiment, a solution containing a final concentration of 10% dimethyl sulfoxide (DMSO), 50% human serum albumin (HSA), and 40% Hank's Balanced Salt Solution (HBSS) will then be added to the cryopreservation bag. In one embodiment, the T-cell subpopulation will be cryopreserved in liquid nitrogen.

Further Characterization of the T-cell Subpopulation

The T-cell subpopulations for use in the lymphocytic cell composition of the present disclosure are HLA-typed and can be further characterized prior to use or inclusion in the lymphocytic cell composition. For example, each of the T-cell subpopulations may be further characterized by, for example, one or more of i) determining the TAA specificity of the T-cell subpopulation; ii) identifying the tumor associated antigen epitope(s) the T-cell subpopulation is specific to; iii) determining whether the T-cell subpopulation includes MHC Class I or Class II restricted subsets or a combination of both; iv) correlating antigenic activity through the T-cell's corresponding HLA-allele; and v) characterizing the T-cell subpopulation's immune effector subtype concentration, for example, the population of effector memory cells, central memory cells, γδ T-cells, CD8+, CD4+, NKT-cell.

Methods for separating mixed cell populations into discrete cell subtypes are well known in the art. For example, affinity column chromatography can be utilized to positively select desired cells by their interaction with the column media. For examples of prior positive selections by column chromatography see: Godfrey, H. P., & Gell, P. G. (1976). Separation by column chromatography of cells active in delayed-onset hypersensitivities. Immunology, 30(5), 695-703 and Xiao F. et al. (2005) Cell column chromatography: a new research tool to quantify cerebral cell volume changes following chemically-induced anoxia/re-oxygenation; in Intracranial Pressure and Brain Monitoring XII. Acta Neurochirurgica Supplementum, vol 95. Springer, Vienna; all incorporated herein by reference.

Enzymatic techniques for isolating cell populations are also useful and widely known. For examples of prior enzymatic positive selections see: Sugita, N. et. al., (2016). Optimization of human mesenchymal stem cell isolation from synovial membrane: Implications for subsequent tissue engineering effectiveness. Regenerative Therapy, 5, 79-85; incorporated herein by reference.

The cell population may also be separated by cell sorting. For a review of cell sorting and various other techniques see Syverud BC, Lee JD, VanDusen KW, et al. (2014) Isolation and purification of satellite cells for skeletal muscle tissue engineering. J Regen Med. 3(2), incorporated herein by reference. In one embodiment the cells are sorted by flow cytometry. Non-limiting examples of instruments to achieve flow cytometry include fluorescence activated cell sorters and automacs seperators with or without detection techniques associate with their use. Determining the Tumor Associated Antigen Specificity of the T-Cell Subpopulation

The T-cell subpopulations of the lymphocytic cell composition can be further characterized by determining each T-cell subpopulation's specificity for its targeted tumor antigen. Specificity can be determined using any known procedure, for example, an ELISA based immunospot assay (ELISpot). In one embodiment, tumor-associated antigen specificity of the T-cell subpopulation is determined by ELISpot assay. ELISpot assays are widely used to monitor adaptive immune responses in both humans and animals. The method was originally developed from the standard ELISA assay to measure antibody secretion from B cells (Czerkinsky et al., J. Immunol. Methods 65:109-21 (1983)), which is incorporated herein by reference. The assay has since been adapted to detect secreted cytokines from T cells, for example IFN-γ, and is an essential tool for understanding the helper T cell response.

A T-cell ELISpot assay generally comprises the following steps:

i) a capture antibody specific for the chosen analyte, for example IFN-γ, is coated onto a PVDF plate;

ii) the plate is blocked, usually with a serum;

iii) the T-cell subpopulation is added along with the specific, targeted tumor associated antigen;

iv) plates are incubated and secreted cytokines, for example IFN-γ, are captured by the immobilized antibody on the PVDF surface;

v) after washing, a biotinylated detection antibody is added to allow detection of the captured cytokine; and

vi) the secreted cytokine is visualized using an avidin-HRP or avidin-ALP conjugate and a colored precipitating substrate.

Each colored spot represents a cytokine secreting cell. The spots can be counted by eye or by using an automated plate-reader. Many different cytokines can be detected using this method including IL-2, IL-4, IL-17, IFN γ, TNFα, and granzyme B. The size of the spot is an indication of the per cell productivity and the avidity of the binding. The higher the avidity of the T cell recognition the higher the productivity resulting in large, well-defined spots.

### Identifying the TAA Epitope(s) the T-Cell Subpopulation is Specific to

The T-cell subpopulations of the lymphocytic cell composition can be further characterized by identifying the specific TAA epitope or epitopes to which the T-cell subpopulation is specific to. This may be especially useful when more than one TAA peptide is used to prime the T-cell subpopulation. Determining TAA epitope specificity is generally known in the art. Non-limiting exemplary methods include Ohminami et al. 2000, supra; Oka et al. 2000, supra; and Bachinsky et al. 2005, supra, which are each incorporated herein by reference. For example, to identify the epitopes with TAA specific activity antigen peptide libraries can be grouped into pools in which each peptide is represented in two or more pools as a quick screening tool in an Elispot assay, and the pools showing activity determined. Common peptides represented in both pools can then be further screened to identify the specific peptide epitopes which show activity.

### Determining the T-cell Subpopulation's MHC-Class I or Class II Restricted Subsets

The T-cell subpopulations of the lymphocytic cell composition can be further characterized by determining the subpopulation's MHC Class I or Class II subset restriction response. This is done to determine whether epitope recognition is mediated by CD8+ (class I) or CD4+ (class II)

T-cells. General methods for determining the MHC Class I or Class II response are generally known in the art. A non-limiting exemplary method is found in Weber et al. 2013, supra, which is incorporated herein by reference. For example, to determine HLA restriction response, T-cells can be pre-incubated with class I or II blocking antibodies for 1 hour before the addition of antigen peptides in an ELISPOT assay using autologous peptide-pulsed PHA blasts as targets with unpulsed PHA blasts as a control. IFNT-secretion is measured in the presence of each blocking antibody. If, when pre-incubated with a class I blocking antibody, IFNT-secretion is reduced to background levels then this is indicative of a class I restriction and the epitope recognition is mediated by CD8⁺ T-cells. If, when pre-incubated with a class II blocking antibody, IFN_{γ}-secretion is reduced to background levels then this is indicative of a class II restriction and the epitope recognition is mediated by CD4⁺ T cells.

The direct detection of antigen-specific T-cells using tetramers of soluble peptide-major histocompatibility complex (pMHC) molecules is widely used in both basic and clinical immunology. Tetrameric complexes of HLA molecules can be used to stain antigen-specific T cells in FACS analysis. In vitro synthesized soluble HLA-peptide complexes are used as tetrameric complexes to stain antigen specific T cells in FACS analysis (Altman et al., Science 274:94-96 (1996)). T-cell subpopulations specific for TAAs are stained with CD8 fluorescein isothiocyanate (FITC) and with phycoerythrin (PE)-labeled MHC pentamers at various timepoints during in vitro stimulation. Antigen specificity is measured by flow cytometry.

Correlating Antigenic Activity through the T-Cell's Corresponding HLA-Allele

The T-cell subpopulation can be further characterized by correlating antigenic activity through the T-cell subpopulation's corresponding HLA-allele. Correlating antigenic activity through the corresponding HLA-allele can be done using any known method. For example, in one embodiment, a HLA restriction assay is used to determine antigen activity through a corresponding allele. Methods to determine T-cell restriction are known in the art and involve inhibition with locus specific antibodies, followed by antigen presentation assays (ELISPOT) with panels of cell lines matched or mismatched at the various loci of interest (see, e.g., Oseroff et al., J. Immunol. 185(2):943-55 (2010); Oseroff et al., J. Immunol. 189(2):679-88 (2012); Wang, Curr. Protoc. Immunol. Chap. 20, page 10 (2009); Wilson et al., J. Virol. 75(9):4195-4207 (2001)), each independently incorporated herein by reference. Because epitope binding to HLA class II molecules is absolutely necessary (but not sufficient) for T cell activation, data from in vitro HLA binding assays has also been useful to narrow down the possible restrictions (Arlehamn et al., J. Immunol. 188(10):5020-31 (2012)). This is usually accomplished by testing a given epitope for binding to the specific HLA molecules expressed in a specific donor and eliminating from further consideration HLA molecules to which the epitope does not bind. To determine the HLA restriction of the identified epitope, T cells can be plated in an IFN-γ ELISPOT assay with TAA peptide pulsed PHA blasts that match at a single allele, measuring the strongest antigen activity, and identifying the corresponding allele.

Characterizing the T-cell Subpopulation's Immune Effector Subtype Concentration The T-cell subpopulation is likely to be made up of different lymphocytic cell subsets, for example, a combination of CD4⁺ T-cells, CD8⁺ T-cells, CD3⁺/CD56⁺ Natural Killer T-cells (CD3⁺ NKT), and TCR γδ T-cells (γδ T-cells). In particular, the T-cell subpopulation likely include at least CD4⁺ T-cells and CD8⁺ T-cells that have been primed and are capable of targeting a single specific TAA for tumor killing and/or cross presentation. The T-cell subpopulation may further comprise activated γδ T-cells and/or activated CD3⁺/CD56⁺ NKT cells capable of mediating anti-tumor responses. Accordingly, the T-cell subpopulation may be further characterized by determining the population of various lymphocytic subtypes, and the further classification of such subtypes, for example, by determining the presence or absence of certain clusters of differentiation (CD) markers, or other cell surface markers, expressed by the cells and determinative of cell subtype.

In one embodiment, the T-cell subpopulation may be analyzed to determine CD8⁺ T-cell population, CD4⁺, T-cell population, γδ T-cell population, NKT-cell population, and other populations of lymphocytic subtypes. For example, the population of CD4⁺ T-cells within the T-cell subpopulation may be determined, and the CD4⁺ T-cell subtypes further determined. For example, the CD4⁺ T-cell population may be determined, and then further defined, for example, by identifying the population of T-helper 1 (Th1), T-helper 2 (Th2), T-helper 17 (Th17), regulatory T cell (Treg), follicular helper T-cell (Tfh), and T-helper 9 (Th9). Likewise, the other lymphocytic subtypes comprising the T-cell subpopulation can be determined and further characterized.

In addition, the T-cell subpopulation can be further characterized, for example, for the presence, or lack thereof, of one or more markers associated with, for example, maturation or exhaustion. T cell exhaustion (Tex) is a state of dysfunction that results from persistent antigen and inflammation, both of which commonly occur in tumor tissue. The reversal or prevention of exhaustion is a major area of research for tumor immunotherapy. Tex cell populations can be analyzed using multiple phenotypic parameters, either alone or in combination. Hallmarks commonly used to monitor T cell exhaustion are known in the art and include, but are not limited to, programmed cell death-1 (PD-1), CTLA-4/CD152 (Cytotoxic T-Lymphocyte Antigen 4), LAG-3 (Lymphocyte activation gene-3; CD223), TIM-3 (T cell immunoglobulin and mucin domain-3), 2B4/CD244/SLAMF4, CD160, and TIGIT (T cell Immunoreceptor with Ig and ITIM domains).

The T-cell subpopulations of the described compositions described herein can be subjected to further selection, if desired. For example, a particular T-cell subpopulation for inclusion in a lymphocytic cell composition described herein can undergo further selection through depletion or enriching for a sub-population. For example, following priming, expansion, and selection, the cells can be further selected for other cluster of differentiation (CD) markers, either positively or negatively. For example, following selection of for example CD4⁺ T-cells, the CD4⁺ T-cells can be further subjected to selection for, for example, a central memory T-cells (Tcm). For example, the enrichment for CD4⁺ Tcm cells comprises negative selection for cells expression a surface marker present on naïve T-cells, such as CD45RA, or positive selection for cells expressing a surface marker present on Tcm cells and not present on naïve T-cells, for example CD45RO, CD62L, CCR7, CD27, CD127, and/or CD44. In addition, the T-cell subpopulations described herein can be further selected to eliminate cells expressing certain exhaustion markers, for example, programmed cell death-1 (PD-1), CTLA-4/CD152 (Cytotoxic T-Lymphocyte Antigen 4), LAG-3 (Lymphocyte activation gene-3; CD223), TIM-3 (T-cell immunoglobulin and mucin domain-3), 2B4/CD244/SLAMF4, CD160, and TIGIT (T-cell Immunoreceptor with Ig and ITIM domains)

Methods for characterizing lymphocytic cell subtypes are well known in the art, for example flow cytometry, which is described in Pockley et al., Curr. Protoc. Toxicol. 66:18.8.1-34 (2015), which is incorporated herein by reference.

Identifying the Lymphocytic Cell composition Most Suitable for Administration

Characterization of each T-cell subpopulation composition allows for the selection of the most appropriate T-cell subpopulations for inclusion in the lymphocytic cell composition for any given subject. The goal is to match the product with the subject that has the both the highest HLA match and greatest TAA activity through the greatest number of shared alleles. In one embodiment, the T-cell subpopulation has at least one shared allele or allele combination with TAA activity through that allele or allele combination. In one embodiment, the T-cell subpopulation has greater than one shared allele or allele combination with TAA activity through that allele or allele combination. In one embodiment, the T-cell subpopulation with the most shared alleles or allele combinations and highest specificity through those shared alleles and allele combinations is provided to a subject in need thereof. For example, if T-cell subpopulation 1 is a 5/8 HLA match with the patient with TAA activity through 3 shared alleles or allele combinations while T-cell subpopulation 2 is a 6/8 HLA match with the subject with TAA activity through 1 shared allele the skilled practitioner would select T-cell subpopulation 1 as it has TAA activity through a greater number of shared alleles.

### Testing T-cell Subpopulations or Lymphocytic Cell Composition Reactivity Against Subject's Tumor

The cytolytic activity of an activated T-cell subpopulation or the lymphocytic cell composition against a subject's tumor can be evaluated. A method of testing reactivity of T-cell subpopulations against tumor cells are well known. Non-limiting exemplary methods include Jedema et al., Blood 103:2677-82 (2004); Noto et al., J. Vis. Exp. (82):51105 (2013); and Baumgaertner etal., Bio-protocol “Chromium-51 (51Cr) Release Assay to Assess Human T Cells for Functional Avidity and Tumor Cell Recognition” 6(16):e1906 (2016). For example, the T-cell subpopulation can be incubated with the patient's tumor and the percent lysis of the tumor cells determined. For example, a biopsy or blood sample will be collected from the patient. Target cells from the patient are fluorescence labeled with carboxyfluorescein succinimidyl ester (CFSE, Invitrogen), peptide-pulsed and incubated with activated T-cell subpopulations or lymphocytic cell composition at a 40:1 effector-to-target ratios for 6-8 hrs. Ethidium homodimer (Invitrogen) is added after incubation to stain dead cells. Samples are acquired on a BD Fortessa Flow Cytometer. The number of live target cells is determined by gating on carboxyfluorescein succinimidyl ester-positive, ethidium homodimer-negative cells, and used to calculate cytolytic activity as follows: Lysis (%)=100−((live target cells/sample/live target cells control)×100).

T-cell subpopulations or lymphocytic cell compositions with the highest levels of reactivity against a patient's tumor can be selected for administration to the subject, providing a higher likelihood of successful therapeutic efficacy.

### Banked T-Cell Subpopulations Directed to Single Tumor Associated Antigens

The establishment of a T-cell subpopulation bank comprising discrete, characterized T-cell subpopulations for selection and inclusion in a lymphocytic cell composition bypasses the need for an immediately available donor and eliminates the wait required for autologous T-cell production. Preparing T-cell subpopulations directed to specific, known tumor antigens by using donors, for example healthy volunteers or cord blood, allows the production and banking of T-cell subpopulations readily available for administration. Because the T-cell subpopulations are characterized, the selection of suitable T-cell subpopulations can be quickly determined based on minimal information from the patient, for example HLA-subtype and, optionally TAA expression profile.

From a single donor a T-cell composition can be generated for use in multiple patients who share HLA alleles that have activity towards a specific TAA. The T-cell subpopulation bank of the present disclosure includes a population of T-cell subpopulations which have been characterized as described herein. For example, the T-cell subpopulations of the bank are characterized as to HLA-subtype and one or more of i) TAA specificity of the T-cell subpopulation; ii) TAA epitope(s) the T-cell subpopulation is specific to; iii) T-cell subpopulation MHC Class I and Class II restricted subsets; iv) antigenic activity through the T-cell's corresponding HLA-allele; and v) immune effector subtype concentration, for example, the population of effector memory cells, central memory cells, γδ T-cells, CD8⁺, CD4⁺, NKT-cell.

In one embodiment, the present disclosure is a method of generating a T-cell subpopulation bank comprising: (i) obtaining eligible donor samples; (ii) generating two or more T-cell subpopulations specific to a single TAA; (iii) characterizing the T-cell subpopulation; (iv) cryopreserving the T-cell subpopulation; and (v) generating a database of T-cell subpopulation composition characterization data. In one embodiment, the T-cell subpopulations are stored according to their donor source. In one embodiment, the T-cell subpopulations are stored by TAA specificity. In one embodiment, the T-cell subpopulations are stored by human leukocyte antigen (HLA) subtype and restrictions.

The banked T-cell subpopulations described herein are used to comprise a lymphocytic cell composition for administration to a tumor patient following the determination of the patient's HLA subtype and, optionally, TAA expression profile of the patient's tumor.

### EXAMPLES

### Example 1

1.1. Cell Source. We will use healthy donors selected for HLA compatibility with the AML patient. If available, peripheral blood mononuclear cells from these donors will be screened to assess for their response to the tumor antigens WT1, PRAME, and survivin.
1.2. Generation of Antigen Presenting Cells. We will generate monocyte-derived dendritic cells by separating the monocyte population using CD14+ magnetic cell sorting. We will first count the PBMC, wash with MACS buffer and centrifuge. We will then add add CD14 microbeads and incubate at room temperature for 20 minutes, agitating the pellet every 5 minutes. We will wash the pellet in MACS buffer and centrifuge, and run in a prewet LS column. We will save the effluent containing the CD14 negative fraction and freeze; the CD14 positive fraction is plated in tissue culture plates, and grown in the presence of GMCSF and IL4. We will pulse the cells with WT1, PRAME, and survivin pepmix. After two days, cells will be matured with LPS, IFNg, IL1b, TNFa, and IL6. We will again pulse the cells with WT1, PRAMS, and survivin pepmix.
1.3. T Cell Expansion. One to to two days after DC maturation, DC will be harvested by gentle scraping of plates with transfer pipette. These cells will be plated with thawed CD14-negative cells at a ratio of 1 DC: 5 T cells. The T cells will be given a cytokine cocktail which may include IL6, IL7, IL12, IL18, IL15 and IL21. They will be fed with fresh media with cytokines if confluent. T cells will again be stimulated at least one additional time with the same monocyte-derived DCs (new collection), potentially supplemented with PHA blasts.
1.4. Testing Specificity. We will use multicolor ELISPOT (IFNg, TNFa, perforin, granzyme), luminex, and intracellular cytokine staining to determine cell responses against (i) antigens, (ii) autologous peptide pulsed PHA blasts, (iii) singly matched allogeneic peptide pulsed PHA blasts, (iv) tumor cells
1.5. Testing Phenotype. We will use flow cytometry to determine expression of activation markers, population markers, memory markers, and exhaustion markers.
1.6. Testing Migration. We will determine whether our cells actively migrate to the tumor site by testing their migration through transwell assays towards the relevant cell lines (eg THP1 for AML). We will also profile the chemokines secreted by the tumor and determine whether our manufactured products express the relevant chemokine receptors on the surface by flow cytometry.
1.7. Tumor Killing. We will determine whether our cells lyse tumor by subjecting them to chromium release assays and to co culture assays.

### Example 2. Alternative Generation of MUSTANG/Fixed Ratio Compositions

TAA-specific T-cell lines can be generated from total human blood peripheral mononuclear cells (Step 1) separated into multiple donor pools. Matured dendritic cells (DCs) are harvested from each pool and used as antigen presenting cells (APCs). Each pool of APCs is peptide-pulsed with a different TAA peptide libraries. One pool is pulsed with a WT1 peptide library, one pool is pulsed with a Survivin peptide library, and one pool is pulsed with a PRAMS peptide library (Step 2). T-cells in each pool are initially stimulated using a cytokine mix containing IL-7, IL-12, IL-15, IL-6, and IL-27 (Step 3). Subsequent stimulations (Steps 4 and 5) are performed using irradiated DCs or irradiated phytohemagglutinin (PHA) blasts. The resultant single-TAA T-cell subpopulations will be tested for antigen specificity using the process outlined in Example 2. The in vitro anti-tumor activity of the MUSTANG composition can be determined using the process described in Example 3. Additional characterization of the TAA CTLs include identification of epitopes with TAA activity, determining the HLA restriction response, and performing a HLA restriction assay to determine antigen activity through a corresponding allele. To determine the composition, a blood or biopsy sample of the patient is provided which is used to determine the HLA subtype and antigen expression profile of a subject with a hematological malignancy or tumor. The MUSTANG composition is selected from the available single-TAA CTLs based on the highest antigen specificity through shared alleles. If desired each single-TAA CTL can be separated by iterative cytometry (Step 6). First the activated CD3⁺ NKT-cells can be separated from the single-TAA CTL by using a label targeting CD56. This positive fraction of CD3⁺ NKT-cells can be further purified by iteratively targeting CD3. The CD4⁺ T-cells can then be purified from the negative fraction by targeting CD4. Similarly, the CD8⁺ T-cells and TCRγδ⁺ gamma-delta T-cells can be purified from the negative fraction by targeting CD8 and TCRγδ respectively. In some embodiments, the antibodies with different labels are used to create more than two fractions per cytometry step and thus decrease the number of steps necessary. In some embodiments, instead of cytometry the purifications are conducted by chromatography or another technique known in the art. In some embodiments, the cytometer is programed to produce fractions with the desired ratio of cells. In some embodiments, specific ratios of antigen specific cell types are combined in a specific ratio of both antigen and T-cell type. Experimental procedures for each of these steps are provided below.

### Step 1. Isolation of Mononuclear Cells

Heparinized peripheral blood or apheresis product will be collected either from the HSCT donor or a healthy donor and separated into multiple pools depending how many antigens are used. The heparinized peripheral blood or apheresis product from each pool will be diluted in an equal volume of warm RPMI 1641 (Invitrogen) or PBS. In a 50 mL centrifuge tube, 10-15 mL of Lymphoprep (Axis-Shield) will be overlayed with 20-30 mL of diluted blood from each pool. The mixtures will be centrifuged at 800×g for 20 minutes or 400×g for 40 minutes at ambient temperature, ensuring that acceleration and deceleration are set to “1” to prevent disrupting the interface. 1 mL of plasma aliquots are saved and stored at -80° C. for each pool. The peripheral blood mononuclear cell (PBMC) interface is harvested into an equal volume of RPMI 1640, centrifuged at 450×g for 10 minutes at ambient temperature, and the supernatant is aspirated. The pellets are loosened and the cells are resuspended in a volume of RPMI 1640 or PBS that yields an estimated 10×10⁶ cells/mL from each pool. An aliquot of cells is removed for counting using 50% red cell lysis buffer or Trypan blue and using a hemocytometer. The PBMCs from each pool are saved for DC generation using adherence (Step 2 below) and non-adherent cells are cryopreserved for use at initiation.

### Step 2. Dendritic Cell (DC) Generation

PBMCs from each pool are centrifuged at 400×g for 5 minutes at ambient temperature, and the supernatant is aspirated. The cells are resuspended at approximately 5×10⁶ cells/mL in CellGenix DC medium containing 2 mM of Glutamax (Invitrogen), and the cells from each pool are plated in a separate 6-well plate (2mL/well). The PBMC non-adherent fraction is removed after 1-2 hours, and the wells are rinsed with 2-5 mL of CellGenix DC medium or PBS and added to the harvested medium/non-adherent fraction. The non-adherent fraction from each pool is saved for later cryopreservation. 2 mL of DC medium containing 1,000 U/mL of IL-4 (R&D Systems) and 800 U/mL GM-CSF (CNMC Pharmacy) is added back to each pool of adherent cells. All surrounding wells are filled with approximately 2 mL of sterile water or PBS to maintain the humidity within each plate, and the plate(s) are placed in the incubator at 37° C. and 5% CO₂. On day 3 to 4, the cells from each pool are fed with 1,000 U/mL IL-4 and 800 U/mL GM-CSF. On day 5 to 6, the DCs from each pool are matured in 2mL/well of DC medium containing lipopolysaccharide (LPS, Sigma) (30 ng/mL), IL-4 (1,000 U/mL), GM-CSF (800 U/mL), TNF-α (10 ng/mL, R&D Systems), IL-6 (100 ng/mL, CellGenix), and IL-1(3 (10 ng/mL, R&D Systems). The mature DCs from each pool are harvested on day 7 to 8 by gentle resuspension. The cells are counted using a hemocytometer. The DCs from each pool are transferred to separate 15 mL centrifuge tubes and centrifuged for 5 minutes at 400×g at ambient temperature. The supernatants are aspirated, and the pellets are resuspended by finger flicking. 100 μL of Pepmix Mastermix per 1-5×10⁶ cells is added to the DCs. A separate PepMix will be used for each pool. In one pool Survivin PepMix is used. In one pool WT1 PepMix is used. In one pool PRAME PepMix is used. The DCs and Pepmixes are mixed and transferred to the incubator. The mixtures are incubated for 60-90 minutes at 37° C. and 5% CO₂.

### Step 3. T-cell Population Initiation

After pulsing with Pepmix, each DC pool is irradiated at 25 Gy. The DCs are washed with

DC medium and centrifuged at 400×g for 5 minutes at ambient temperature. The supernatant is aspirated, and the wash step is repeated twice more. The cells are counted using a hemocytometer. The DCs are resuspended at 2-4×10⁵ cells/mL of CTL medium with 10% human serum (HS, Valley) for initiation. 1 mL of irradiated DCs/well are plated in a 24-well tissue culture treated plate. Repeat for each additional DC pool.

Previously-frozen PBMCs from Step 1 are thawed at 37° C. and diluted in 10 mL of warm medium/1 mL of frozen cells. The PBMCs from each pool are centrifuged at 400×g for 5 minutes at ambient temperature and resuspended in 5-10 mL of medium and a cell count is performed using a hemocytometer. The PBMCs from each pool are resuspended at 2×10⁶ cells/mL. DCs and PBMCs are recombined in the plate to stimulate CTL at a 1:10 to 1:5 ratio of DCs: CTL. Cytokines

IL-7, IL-15, IL-6, and IL-12 are added to achieve a final concentration of IL-7 (10 ng/mL, R&D Systems)), IL-15 (5 ng/mL, CellGenix), IL-6 (100 ng/mL, CellGenix), and IL-12 (10 ng/mL, R&D Systems). All surrounding wells are filled with approximately 2 mL of PBS to maintain humidity within the plate. The cells are cultured in the incubator at 37° C. and 5% CO₂ for 7 to 8 days. A one-half medium change is performed on day 4 to 5, with the wells being split 1:1 if nearly confluent.

### Step 4. Second T-Cell Stimulation in 24-Well Plate

The second stimulation of each pool of T-cells is performed using either PepMix-Pulsed Autologous DCs (Procedure A) or PepMix-Pulsed Autologous Phytohemagglutinin (PHA) Blasts (Procedure B) as antigen presenting cells.

### Procedure A: Stimulation Using PepMix-Pulsed Autologous DCs as Antigen Presenting Cells (APCs)

After each pool is pulsed with a different Pepmix (PRAME, WT1, and Survivin Pepmixes; JPT Peptide Technologies), DCs from each pool are irradiated at 25 Gy. The DCs from each pool are washed with DC medium and centrifuged at 400×g for 5 minutes at ambient temperature. The supernatants are aspirated and the wash step is repeated twice more. The cells are counted using a hemocytometer. The DCs from each pool are resuspended at 0.5-2×10⁵ cells/mL of CTL medium with 10% HS (Valley) for initiation. For each DC pool, plate 1 mL of irradiated DCs/well (0.5-2×10⁵ cells) in a 24-well tissue culture treated plate. T-cells are counted using a hemocytometer. The cells are resuspended at 1×10⁶ cells/mL of T-cells medium supplemented with IL-7 (10 ng/mL final concentration, R&D Systems)) and IL-2 (100 U/mL final concentration, Proleukin) and 1 mL is aliquoted per well of the 24-well plate. The cells are cultured in the incubator at 37° C. and 5% CO₂ for 3 to 4 days. The medium is changed with IL-2 (˜100 U/mL final concentration, Proleukin) and cultured for another 3 to 4 days. Cells can be frozen after the second stimulation.

### Procedure B: Stimulation Using PepMix-Pulsed Autologous Phytohemagglutinin (PHA) Blasts as APCs

Autologous PHA blasts from each pool are harvested on day 7 by gentle resuspension, and cells are counted using a hemocytometer. The PHA blasts from each pool are transferred to separate 15 mL centrifuge tubes and centrifuged for 5 minutes at 400×g at ambient temperature. The supernatants are aspirated and the pellets are resuspended by finger flicking. 100 μL of appropriate PepMix Mastermix (200 ng/peptide in 200 μL; PRAME, WT1, and Survivin Pepmixes; JPT Peptide Technologies) is added to PHA blasts per 1-10×10⁶ cells. One different PepMix is added to each PHA blast pool. The PHA blasts are incubated for 30-60 minutes. The PHA blasts from each pool are resuspended in 5-10 mL of medium and irradiated at 50 Gy (or 100 Gy if used in G-rex). The PHA blasts are washed with CTL medium and centrifuged at 400×g for 5 minutes at ambient temperature. The supernatants are aspirated and the washing steps are repeated twice more. A cell count is performed using a hemocytometer. The PHA blasts from each pool are resuspended at 0.5×10⁶ cells/mL of CTL medium to re-stimulate T-cells at an approximate ratio of 1:1 PHA blasts: T-cell. The T-cells from each pool are counted using a hemocytometer. The T-cells from each pool are resuspended at 0.5×10⁶ cells/mL of CTL medium supplemented with IL-7 (100 ng/mL final concentration; R&D Systems) and IL-2 (100 U/mL final concentration; Proleukin). One well of only PHA blasts is maintained as an irradiation control. The cells are cultured in the incubator at 37° C. and 5% CO₂ for 3 to 4 days. The medium is changed with IL-2 (100 U/mL final concentration; Proleukin) and the cells are cultured for another 3 to 4 days.

### Step 5. Third T-Cell Stimulation in G-Rex10 Using PHA Blasts as APCs

Autologous PHA blasts from each pool are harvested on day 7 by gentle resuspension, and cells are counted using a hemocytometer. The PHA blasts from each pool are transferred to separate 15 mL centrifuge tubes and centrifuged for 5 minutes at 400×g at ambient temperature. The supernatants are aspirated, and the pellets are resuspended by finger flicking. 100 μL of appropriate PepMix Mastermix (200 ng/peptide in 200 μL; PRAME, WT1, and Survivin Pepmixes; JPT Peptide Technologies) is added to PHA blasts per 1-10×10⁶ cells. One different PepMix is added to each PHA blast pool. The PHA blasts are incubated for 30-60 minutes. The PHA blasts from each pool are resuspended in 5-10 mL of medium and irradiated at 50 Gy (or 100 Gy if used in G-rex). The PHA blasts are washed with CTL medium and centrifuged at 400×g for 5 minutes at ambient temperature. The supernatants are aspirated and the washing steps are repeated twice more. A cell count is performed using a hemocytometer. The PHA blasts from each pool are resuspended at 0.5×10⁶ cells/mL of CTL medium to re-stimulate T-cells at an approximate ratio of 1:1 PHA blasts: T-cell. The T-cells from each pool are counted using a hemocytometer. The T-cells from each pool are resuspended at 0.5×10⁶ cells/mL of CTL medium supplemented with IL-7 (100 ng/mL final concentration; R&D Systems) and IL-2 (100 U/mL final concentration; Proleukin). One well of only PHA blasts is maintained as an irradiation control. The cells are cultured in the incubator at 37° C. and 5% CO₂ for 3 to 4 days. The medium is changed with IL-2 (100 U/mL final concentration; Proleukin) and the cells are cultured for another 3 to 4 days.

### Step 6. Specific Cell Separations

If desired each MUSTANG composition can be separated by iterative cytometry into pools of specific T-cell types. First the CD3⁺ NKT-cells can be separated from the T-cell subpopulation by using a label targeting CD56. This positive fraction of CD3⁺ NKT-cells can be further purified by iteratively targeting CD3. The CD4⁺ T-cells can then be purified from the negative fraction by targeting CD4. Similarly, the CD8⁺ T-cells and TCRγδ⁺ gamma-delta T-cells can be purified from the negative fraction by targeting CD8 and TCRγδ respectively. In some embodiments, the antibodies with different labels are used to create more than two fractions per cytometry step and thus decrease the number of steps necessary. In some embodiments, instead of cytometry the purifications are conducted by chromatography or another technique known in the art. In some embodiments, the cytometer is programed to produce fractions with the desired ratio of cells.

### Example 3. ELISPOT Plating and Development for Analysis of T-Cell Function

Peptide recognition for TAA subpopulations specific to survivin, PRAME, and/or WT1 can be tested in an IFN-γ-enzyme-linked immunospot (ELISpot) assay. Recognition of the single antigens is tested as compared with no-peptide media control (SEB 90%), CTL none, and actin. The 3-day procedure for performing the ELISpot assay is detailed below.

Day 1: ELISPOT Plate Preparation

ELISPOT coating buffer is prepared by dissolving 1.59 g Na₂CO₃ to one liter of sterile water followed by sterile filtration. INFγ-capture antibody (Ab) solution is prepared by added 100 μL IFN-γ mAB 1-D1K (MabTech) to every 10 mL ELISPOT coating buffer. 35 μL of 70% ethanol is added to each well of a 96-well filtration plate (Millipore) using a 200 μL multichannel pipette. The ethanol is dumped, and the plate is immediately washed two times with 150 μL PBS. The last PBS wash is dumped, and 100 μL of Ab solution is immediately added to each well. The plate edges are wrapped in parafilm to prevent evaporation, and the plates sit for a minimum of 6 hours at 4° C. These coated plates are stable at 4° C. for up to 4 weeks.

Day 2: ELISPOT Cell Plating

ELISPOT media is prepared by combining 250 mL RPMI, 12.5 mL human serum (HS), and 2.5 mL sterile-filtered GlutaMAX. The coating buffer in the 96-well plate is dumped, and the wells are washed two times with 150 μL PBS. 100 μL of ELISPOT media is added to each well, and the plate is placed in the incubator at 37° C. for a minimum of one hour.

While the plate is incubating, peptide pools are prepared in a 24-well plate. The following peptide pools are prepared using 250 μL of ELISPOT media and 2.5 μL peptide: PBMC; Actin; Staphylococcal enterotoxin B (SEB; dosed at 1.0 μL peptide); PRAME; Survivin; and WT1. The cells are harvested and counted using a hemocytometer. 4.0×10⁶ cells are aliquoted and centrifuged at 400×g for 5 minutes and supernatant removed. The cells are resuspended in ELISPOT media to ensure 2.5×10⁵ cell/100 μL media. The ELISPOT media is dumped from the plate after incubation, and 100 μL of cells are placed in the appropriate wells. 100 μL of peptide pool is mixed in the appropriate wells and incubated at 37° C. overnight.

Day 3: ELISPOT Plate Development

The cells are decanted from the plate, and the plate is washed six times with PBS/0.05% Tween 20 solution. Biotin buffer is prepared by adding 2.5 g bovine serum albumin (BSA) powder to 500 mL PBS followed by sterile filtering. The biotinylated antibody solution is prepared by adding 10 μL mAb 7-B6 (MabTech) to every 10 mL of Biotin buffer. The last plate wash is decanted and 100 μL of biotinylated antibody solution is added to each well. The plate is incubated at 37° C. for 1 to 2 hours. The biotinylated antibody solution is decanted, and the plate is washed six times with PBS/0.05% Tween 20 solution. 100 μL of Avidin-Peroxidase Complex (APC) solution is added to each well using a multichannel pipette. The plate is covered with foil and sat at room temperature for 1 to 2 hours. The 3-amino-9-ethylcarbazole (AEC) substrate solution is prepared while the plate is incubating by dissolving the AEC tablet in 2.5 mL of dimethylformamide in a 50 mL centrifuge tube, adding 47.5 mL acetate buffer (prepared by mixing 4.6 mL 0.1 N acetic acid, 11 mL 0.1 M sodium acetate, and 46.9 mL sterile water) and 25 μL hydrogen peroxide, and mixing by inverting. The APC solution is decanted, and the plate is washed three times with plain PBS solution. 100 μL of AEC substrate solution is then added to each well, the plate is covered in foil, and incubated for 4 minutes. The AEC solution is decanted, and plate development is halted by rinsing with vigorously running water. The plate backing is removed, the membranes are rinsed with water, and the plate is firmly tapped against a paper towel to remove any excess water. The plates are dried by placing them upside down with no lip on a hood grate. Upon drying, the plates are wrapped in paper towel and stored in a dark place to prevent bleaching of spots. Spot-forming cells (SFCs) are counted and evaluated using an automated plate reader system (Karl Zeiss).

### Example 4. Antileukemic Activity Against Partially HLA-Matched AML blasts

To evaluate the antileukemic activity of single-TAAmix-specific T-cells and MUSTANG compositions in vitro, T-cells can be cocultured with primary leukemia blast samples matched in at least one HLA-antigen (range 1-3), including pairs, which are matched solely at HLA class II alleles. Where available, AML blast samples are evaluated for expression of MAGE-A3 and PRAME. As control for nonspecific lysis or allogeneic reactivity, cytotoxic T-cell lines with irrelevant specificity (viral antigens) generated from the same donor can be used in all experiments.

### Example 5. Isolation of Naïve T Cells

In some embodiments, the isolation methods described above can be used to isolate naïve T cells from a healthy subject or donor.

In some embodiments, a selection step can be performed. The selection step can involve the following method: PBMCs were isolated using Ficoll as described above. Adherent cells were used for DC generation, and T-cell-containing nonadherent cells were frozen for use on day 7. After thawing, nonadherent cells were labeled for immunomagnetic selection of CD45RA+ cells, washed, and then selected by MACS®. The CD45RA+ cells were then resuspended in 45% RPMI (Hyclone) and 45% CLICKS (Irvine Scientific) with 10% Human Serum plus GlutaMAX™ (T-cell medium). Cells were resuspended at 2x106 /ml and cocultured with autologous, Pepmix-pulsed DCs at a ratio of 20 PBMCs to 1 DC in the presence of the cytokines 10 ng/ml IL-7 and IL-12, (R&D Systems) and 5 ng/mL IL-15 (CellGenix). Cultures were restimulated on days 10 and 17 with irradiated (40 Gy), pp65 Pepmix-pulsed autologous LCLs at a responder-to-stimulator ratio of 4:1 plus IL-15 (5 ng/ml) on day 10 and 50 U/mL IL-2 (Proleukin) on days 17 and day 20. To confirm the origin of the pp65-specific T-cell populations CD45RA/CCR7 double positive and double negative T-cell populations were sorted by flow cytometry and were stimulated with pp65-Pepmix-pulsed DCs followed by pp65-pepmix-pulsed LCLs.

In some embodiments, the method of isolating naïve T cells does not comprise the depletion steps described above. Thus, in some embodiments T cells can be selected by using the CD45RA+ marker without depleting memory, or CD45RO+ cells.

In some embodiments, both selecting for CD45RA+ cells and depleting CD45RO+ cells can be performed.

This specification has been described with reference to embodiments of the present disclosure. The present disclosure has been described with reference to assorted embodiments, which are illustrated by the accompanying Examples. The present compositions can, however, be embodied in different forms and should not be construed as limited to the embodiments set forth herein. Given the teaching herein, one of ordinary skill in the art will be able to modify the present disclosure for a desired purpose and such variations are considered within the scope of the present disclosure.

1. An isolated lymphocytic cell composition comprising about a fixed ratio of activated CD4⁺ T-cells, activated CD8⁺ T-cells, and activated CD3⁺ NKT-cells, wherein the CD4⁺ T-cells and CD8⁺ T-cells have been primed ex vivo against one or more tumor associated antigens (TAAs) or viral associated tumor antigens (VATAs), and wherein one or more of the activated CD4⁺ T-cells, activated CD8⁺ T-cells, and activated CD3⁺ NKT-cells comprise a fixed ratio of two or more separately primed and expanded cell subpopulations, each cell subpopulation having (i) specificity for a single tumor associated antigen and (ii) a different single tumor associated antigen specificity from all other cell subpopulations in the composition.

2. The isolated lymphocytic cell composition of claim 1, wherein the fixed ratio of activated CD4⁺ T-cells, activated CD8⁺ T-cells, and activated CD3⁺ NKT-cells comprising comprises:
(i) between about 15% and about 25% CD4⁺ T-cells;
(ii) between about 45% and about 55% CD8⁺ T-cells; and
(iii) between about 25% and about 35% CD3⁺ NKT-cells; and
wherein the CD4⁺ T-cells and CD8⁺ T-cells have been primed ex vivo against one or more tumor associated antigens (TAAs) or viral associated tumor antigens (VATAs); and
wherein one or more of the activated CD4⁺ T-cells, activated CD8⁺ T-cells, and activated CD3⁺ NKT-cells comprise a fixed ratio of two or more separately primed and expanded cell subpopulations, each cell subpopulation having (i) specificity for a single tumor associated antigen and (ii) a different single tumor associated antigen specificity from all other cell subpopulations in the composition.

3.-4. (canceled)

5. The isolated lymphocytic cell composition of claim 1, wherein the fixed ratio of activated CD4⁺ T-cells, activated CD8⁺ T-cells, and activated CD3⁺ NKT-cells comprising:
(i) between about 10% and about 20% CD4⁺ T-cells;
(ii) between about 25% and about 35% CD8⁺ T-cells; and
(iii) between about 10% and about 20% CD3⁺ NKT-cells; and
wherein the CD4⁺ T-cells and CD8⁺ T-cells have been primed ex vivo against one or more tumor associated antigens (TAAs) or viral associated tumor antigens (VATAs); and
wherein one or more of the activated CD4⁺ T-cells, activated CD8⁺ T-cells, and activated CD3⁺ NKT-cells comprise a fixed ratio of two or more separately primed and expanded cell subpopulations, each cell subpopulation having (i) specificity for a single tumor associated antigen and (ii) a different single tumor associated antigen specificity from all other cell subpopulations in the composition.

6.-10. (canceled)

11. The isolated lymphocytic cell composition of claim 1, wherein one or more of the single tumor associated antigens is chosen from one or a combination of: PRAME, survivin, WT1, NY-ESO-1, and MAGE-A3.

12.-17. (canceled)

18. The isolated lymphocytic cell composition of claim 1, wherein the tumor is a hematological malignancy or a solid tumor.

19.-28. (canceled)

29. The isolated lymphocytic cell composition of claim 1, wherein the cell subpopulations are derived from an allogeneic donor or cord blood.

30.-154. (canceled)

155. An isolated lymphocytic cell composition comprising a fixed of activated αβ T-cells, activated γδ T-cells, and activated CD3+ NKT-cells,
wherein the αβ T-cells have been primed ex vivo against two or more tumor associated antigens (TAAs) or viral associated tumor antigens (VATAs);
wherein the αβ T-cells are comprised of two or more subpopulations;
wherein each αβ T-cell subpopulation is specific for a single TAA or VATA;
wherein each αβ T-cell subpopulation is specific for a different TAA or VATA than any another αβ T-cells subpopulation in the composition; and,
wherein each of the αβ T-cell subpopulations are primed and expanded separately from each other.

156. The lymphocytic cell composition of claim 155, wherein the composition comprises a 1:1:1 ratio (+/−5%) of activated αβ T-cells, activated γδ T-cells, and activated CD3+ NKT-cells.

157. The lymphocytic cell composition of claim 155, wherein the composition comprises:
(i) between about 25% and about 35% αβ T-cells,
(ii) between about 25% and about 35% γδ T-cells, and
(iii) between about 35% and about 45% CD3+ NKT-cells.

158.-159. (canceled)

160. The lymphocytic cell composition of claim 155, wherein the composition comprises:
(i) between about 35% and about 45% αβ T-cells,
(ii) between about 30% and about 40% γδ T-cells, and
(iii) between about 10% and 20% CD3+ NKT-cells.

161.-163. (canceled)

164. The isolated lymphocytic cell composition of claim 155, wherein one or more of the single tumor associated antigens chosen from one or a combination of: is selected from the group consisting of PRAME, survivin, WT1, NY-ESO-1, and MAGE-A3.

165.-171. (canceled)

172. The isolated lymphocytic cell composition of claim 155, wherein the cell subpopulations are derived from an allogeneic donor or from cord blood.

173.-174. (canceled)

175. The isolated lymphocytic cell composition of claim 155, wherein the composition comprises at least about 60% CD4+ Th1-cells.

176. The isolated lymphocytic cell composition claim 155, wherein the composition comprises less than about 5% CD4+ Treg-cells.

177. The isolated lymphocytic cell composition claim 155, wherein the γδ T-cells are at least about 70% Vγ9Vδ2 T-cells.

178.-179. (canceled)

180. A method of treating a malignancy or tumor, comprising administering an effective amount of the isolated lymphocytic cell composition of claim 1 to a patient with a tumor.

181. The method of claim 180, wherein the tumor is a hematological malignancy.

182. The method of claim 181, wherein the hematological malignancy is selected from the group consisting of: leukemia, lymphoma, and multiple myeloma.

183. The method of claim 180, wherein the tumor is a solid tumor.

184. (canceled)

185. The method of claim 180, wherein the isolated lymphocytic cell composition has at least one HLA allele or HLA allele combination in common with the patient.

186.-189. (canceled)
